(19) United States
(12) Patent Application Publication
(10) Pub. No.: US 2021/0147507 A1
(43) Pub. Date: May 20, 2021

(54) METHODS TO AUGMENT OR ALTER SIGNAL TRANSDUCTION

(71) Applicant: Bellicum Pharmaceuticals, Inc., Houston, TX (US)

(72) Inventors: Joseph Henri Bayle, Houston, TX (US); Wei-Chun Chang, Horsham, PA (US); Aaron Edward Foster, Houston, TX (US); David Michael Spencer, Frisco, TX (US)

(21) Appl. No.: 16/611,772

(22) PCT Filed: May 8, 2018
(86) PCT No.: PCT/US2018/031689

§ 371 Date: Nov. 7, 2019

Related U.S. Application Data
(60) Provisional application No. 62/503,565, filed on May 9, 2017.

(51) Int. Cl. C07K 14/705; C12N 5/0783; C12N 9/90; C07K 16/30

(52) U.S. Cl. CPC C07K 14/70578; C12N 5/0636; C12N 5/0646; C12N 9/90; C07K 2319/33; C07K 16/3069; C07K 2319/02; C07K 2317/622; C12Y 502/00

(57) ABSTRACT

The technology relates generally to the field of immunology and relates in part to compositions and methods for activating cells, including, for example T cells that express chimeric antigen receptors or recombinant TCRs, and reducing cytotoxicity, using chimeric polypeptides including MyD88 and signaling domains of receptor mediators of costimulation.

### RELATED APPLICATIONS

This application is a United States National Stage of International Patent Application No. PCT/US2018/031689, filed May 8, 2018, which claims the benefit of Priority is claimed to U.S. Provisional Patent Application Ser. No. 62/503,565, filed May 9, 2017, entitled “Methods to Augment or Alter Signal Transduction,” which is referred to and incorporated by reference thereof, in its entirety.

### SEQUENCE LISTING

This application incorporates by reference a Sequence Listing with this application as an ASCII text file entitiled “14562-061-999_SEQ_LISTING” created on Jul. 10, 2020, and having a size of 206,355 bytes.

### FIELD

The technology relates generally to the field of immunology and relates in part to compositions and methods for activating cells, including, for example T cells that express chimeric antigen receptors or recombinant TCRs, and reducing cytotoxicity, using chimeric polypeptides including MyD88 and signaling domains of receptor mediators of costimulation.

### BACKGROUND

Immune cell activation is an important step in the protective immunity against pathogenic microorganisms (e.g., viruses, bacteria, and parasites), foreign proteins, and harmful chemicals in the environment, and also as immunity against cancer and other hyperproliferative diseases. Immune cells, for example, T cells, express receptors on their surfaces, for example, T cell receptors, that recognize antigens presented on the surface of cells. Immune cells may also be modified to express chimeric antigen receptors, which are artificial receptors designed to convey antigen specificity to immune cells, or recombinant T cell receptors. During a normal immune response, binding of these antigens to the receptors initiates intracellular changes leading to T cell activation. The signal to initiate the intracellular changes is transmitted through the cell membrane through signal transduction. Augmenting or altering signal transduction may have an effect on T cell activation.

### SUMMARY

Immune cells may be modified by transfection or transduction of the cells so that they express chimeric antigen receptors (CARs) or recombinant TCRs (rTCRs) that recognize a target antigen. Immune cells include, for example, T cells, NK cells, NK-T cells, invariant NK-T cells, gamma delta (γδ) T cells, and tumor infiltrating lymphocytes. The anti-target and antitumor efficacy of these engineered immune cells is dependent on their survival and in vivo expansion following adoptive transfer. Although including costimulatory domains, such as CD28 and 4-1BB in chimeric antigen receptors has enhanced T cell expansion, the alteration of signal transduction by these costimulatory domains may lead to severe cytotoxicity.

Provided herein are compositions and methods to augment or alter signal transduction in immune cells. The compositions and methods provide the ability to activate and enhance the survival and expansion of the modified immune cells, while reducing certain cytotoxic effects.

Expression of recombinant vectors encoding gene fusions between a truncation allele of MyD88 (myeloid differentiation primary response 88) and the intracellular signaling domains of certain transmembrane receptors generates signaling nodes that enhance the signaling capacity of MyD88 itself. These chimeric signaling polypeptides may have constitutive activity, or may include multimeric ligand binding regions that, upon binding to a multimeric ligand induce multimerization and activation of the chimeric signaling polypeptide. Immune cells may express the chimeric signaling polypeptide as part of a chimeric antigen receptor polypeptide, or the chimeric signaling polypeptide may be expressed as a separate polypeptide from the antigen recognition polypeptide, for example, the CAR (chimeric antigen receptor) or rTCR (recombinant T cell receptor).

Provided in certain embodiments are nucleic acids comprising a promoter operably linked to a polynucleotide encoding an inducible chimeric signaling polypeptide, wherein the polypeptide comprises a multimeric ligand binding region that binds to a multimeric ligand; a MyD88 polypeptide or a truncated MyD88 polypeptide lacking a TIR domain; and a costimulatory polypeptide cytoplasmic signaling region with the proviso that the costimulatory polypeptide cytoplasmic signaling region is not CD40. In some embodiments, the costimulatory polypeptide cytoplasmic signaling region is selected from the group consisting of CD27, CD28, ICOS, 4-1BB, RANK/TRANCE-R, OX40, CD30, TweakR, TAC1, BCMA and HVEM cytoplasmic signaling regions and is not a CD40 polypeptide. In other embodiments, the costimulatory polypeptide cytoplasmic signaling region is selected from the group consisting of CD27, CD28, ICOS, 4-1BB, RANK/TRANCE-R, and OX40 cytoplasmic signaling regions, and is not a CD40 polypeptide. In other embodiments, the costimulatory polypeptide cytoplasmic signaling region is selected from the group consisting of CD27, CD28, ICOS, 4-1BB, RANT/TRANCE-R, and OX40, and the inducible chimeric signaling polypeptide further comprises a CD40 polypeptide lacking the extracellular region.

Also provided in certain embodiments are nucleic acids comprising a promoter operably linked to a polynucleotide encoding a chimeric signaling polypeptide, wherein the polypeptide comprises a MyD88 polypeptide or a truncated MyD88 polypeptide lacking a TIR domain; and a costimulatory polypeptide cytoplasmic signaling region with the proviso that the costimulatory polypeptide cytoplasmic signaling region is not CD40. In other embodiments, the costimulatory polypeptide cytoplasmic signaling region is selected from the group consisting of CD27, CD28, ICOS, 4-1BB, RANK/TRANCE-R, and OX40 cytoplasmic signaling regions, and is not a CD40 polypeptide. In other embodiments, the costimulatory polypeptide cytoplasmic signaling region is selected from the group consisting of CD27, CD28, ICOS, 4-1BB, RANT/TRANCE-R, and OX40, and the inducible chimeric signaling polypeptide further comprises a CD40 polypeptide lacking the extracellular region.

Costimulatory polypeptide cytoplasmic signaling regions of the inducible chimeric signaling polypeptides and chimeric signaling polypeptides herein may, for example, activate the NF-κB pathway, and are selected from non-CD40 NF-κB inducers such as, for example, CD28 or TNFR family members.

Also provided in some embodiments are nucleic acids comprising a polynucleotide encoding an inducible chimeric signaling polypeptide, wherein the chimeric signaling polypeptide comprises functional domains, or functional regions. Functional domains or functional regions may be selected from the group consisting of MyD88 polypeptides or truncated MyD88 polypeptides, costimulatory polypeptide cytoplasmic signaling regions, multimeric ligand binding regions, and membrane targeting regions. In some embodiments, the functional domains consist of a) one or more multimeric ligand binding regions that bind to a multimeric ligand; b) a MyD88 polypeptide or a truncated MyD88 polypeptide lacking the TIR domain; and c) a costimulatory polypeptide cytoplasmic signaling region. Thus, in some embodiments, the MyD88 polypeptide domain comprises a full length MyD88 polypeptide, in some embodiments, the MyD88 polypeptide domain comprises a truncated MyD88 polypeptide lacking the TIR domain, in some embodiments, the truncated MyD88 polypeptide comprises a polypeptide that comprises the amino acid sequence of SEQ ID NO: 2, in some embodiments, the truncated MyD88 polypeptide consists of the amino acid sequence of SEQ ID NO: 2. Also, in some embodiments, the MyD88 polypeptide domain consists of a full length MyD88 polypeptide, in some embodiments, the MyD88 polypeptide domain consists of a truncated MyD88 polypeptide lacking the TIR domain, in some embodiments, the truncated MyD88 polypeptide consists of a polypeptide that comprises the amino acid sequence of SEQ ID NO: 2. The chimeric signaling polypeptides may also comprise additional polypeptides, which may also be referred to as non-functional polypeptides, such as, for example, 2A polypeptides, marker polypeptides, and linker polypeptides. In some embodiments, the multimeric ligand binding regions comprise FKBP12 variant polypeptides of the present application, such as, for example, FKBP12 variant polypeptides having amino acid substitutions at position 36, and, for example, FKBP12v36. In some embodiments, functional domain (a) comprises two FKBP12 variant polypeptides, such as, for example, FKBP12 variant polypeptides having amino acid substitutions at position 36, and, for example, FKBP12v36. In some examples domain b) comprises a truncated MyD88 polypeptide lacking the TIR domain, such as, for example, the truncated MyD88 polypeptides of the present application. In some embodiments, the costimulatory polypeptide of domain (c) is selected from the group consisting of CD27, CD28, ICOS, 4-1BB, RANK/TRANCE-R, OX40, CD30, TweakR, TAC1, BCMA and HVEM cytoplasmic signaling regions, or a functional fragment thereof. In other embodiments, the costimulatory polypeptide cytoplasmic signaling region is selected from the group consisting of CD27, CD28, ICOS, 4-1BB, RANK/TRANCE-R, and OX40 cytoplasmic signaling regions, and is not a CD40 polypeptide. In other embodiments, the costimulatory polypeptide cytoplasmic signaling region is selected from the group consisting of CD27, CD28, ICOS, 4-1BB, RANT/TRANCE-R, and OX40, or a functional fragment thereof. In some embodiments, the costimulatory polypeptide cytoplasmic signaling region consists of a cytoplasmic signaling region of a costimulatory polypeptide selected from the group consisting of CD27, CD28, ICOS, 4-1BB, RANT/TRANCE-R and OX40, or a functional fragment thereof. In some embodiments, the costimulatory polypeptide cytoplasmic signaling region comprises a cytoplasmic signaling region of a costimulatory polypeptide selected from the group consisting of CD28, ICOS, 4-1BB, and OX40. In some embodiments, the costimulatory polypeptide cytoplasmic signaling region consists of a cytoplasmic signaling region of a costimulatory polypeptide selected from the group consisting of CD28, ICOS, 4-1BB, and OX40.

In some embodiments, a nucleic acid is provided, comprising a promoter operably linked to a polynucleotide encoding an inducible chimeric signaling polypeptide, wherein the polypeptide comprises a) one or more multimeric ligand binding regions that bind to a multimeric ligand; b) a MyD88 polypeptide or a truncated MyD88 polypeptide lacking the TIR domain; and c) a costimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions.

In some embodiments, the modified cells comprise a chimeric signaling polypeptide that does not comprise a membrane-targeting region. In some embodiments, the modified cells comprise a chimeric signaling polypeptide that comprises no membrane-targeting region. In some embodiments, the chimeric signaling polypeptide does not have a membrane-targeting region, for example, in some embodiments, the chimeric signaling polypeptide does not have, or does not comprise a myristoylation region, palmitoylation region, prenylation region, or transmembrane region. In some embodiments, the chimeric signaling polypeptide does not have a functional membrane-targeting region.

In some embodiments, the modified cells and nucleic acids comprise a polynucleotide that encodes a chimeric signaling polypeptide that does not comprise a membrane-targeting region. In some embodiments, the modified cells and nucleic acids comprise a polynucleotide that encodes a chimeric signaling polypeptide that comprises no membrane-targeting region. In some embodiments, the modified cells and nucleic acids comprise a polynucleotide that encodes a chimeric signaling polypeptide that does not have a membrane-targeting region, for example, in some embodiments, the chimeric signaling polypeptide does not have, or does not comprise a myristoylation region, palmitoylation region, prenylation region, or transmembrane region. In some embodiments, the modified cells and nucleic acids comprise a polynucleotide that encodes a chimeric signaling polypeptide that does not have a functional membrane-targeting region.

In some embodiments, the nucleic acid comprises a polynucleotide coding for a chimeric signaling polypeptide or an inducible chimeric signaling polypeptide that further comprises a membrane targeting region. In some embodiments, the membrane targeting region is selected from the group consisting of a myristoylation region, a palmitoylation region, a prenylation region, and transmembrane sequences of receptors. In some embodiments, the membrane-targeting region is a myristoylation region.

In some embodiments, the modified cells comprise an inducible chimeric signaling polypeptide that does not comprise a membrane-targeting region. In some embodiments, the modified cells comprise an inducible chimeric signaling polypeptide that comprises no membrane-targeting region. In some embodiments, the chimeric signaling polypeptide does not have a membrane-targeting region, for example, in some embodiments, the chimeric signaling polypeptide does not have, or does not comprise a myristoylation region, palmitoylation region, prenylation region, or transmembrane region. In some embodiments, the chimeric signaling polypeptide does not have a functional membrane-targeting region.

In some embodiments, the modified cells and nucleic acids comprise a polynucleotide that encodes an inducible chimeric signaling polypeptide that does not comprise a membrane-targeting region. In some embodiments, the modified cells and nucleic acids comprise a polynucleotide that encodes an inducible chimeric signaling polypeptide that comprises no membrane-targeting region. In some embodiments, the modified cells and nucleic acids comprise a polynucleotide that encodes an inducible chimeric signaling polypeptide that does not have a membrane-targeting region, for example, in some embodiments, the chimeric signaling polypeptide does not have, or does not comprise a myristoylation region, palmitoylation region, prenylation region, or transmembrane region. In some embodiments, the modified cells and nucleic acids comprise a polynucleotide that encodes an inducible chimeric signaling polypeptide that does not have a functional membrane-targeting region.

In some embodiments, the nucleic acid further comprises a polynucleotide encoding a chimeric Caspase-9 polypeptide comprising a multimeric ligand binding region and a Caspase-9 polypeptide.

Also provided in some embodiments are inducible chimeric signaling polypeptides or chimeric signaling polypeptides encoded by a nucleic acid of the present embodiments. Provided in some embodiments are modified cells, wherein the cell is transduced or transfected with a nucleic acid of any one of the present embodiments. In some embodiments, the cell is also transduced or transfected with a nucleic acid comprising a polynucleotide coding for a heterologous protein, a marker polypeptide, a chimeric antigen receptor, a recombinant T cell receptor. In some embodiments, the cell is selected from the group consisting of T cell, tumor infiltrating lymphocyte, NK-T cell, invariant NK-T cell, gamma delta T cell, and NK cell. In some embodiments, the cell is a T cell, in some embodiments, the cell is an invariant NK-T cell, in some embodiments, the cell is a gamma delta T cell, in some embodiments, the cell is a NK cell. Also provided in certain embodiments are methods for expressing an inducible chimeric signaling polypeptide, or a chimeric signaling polypeptide in a cell, comprising contacting a nucleic acid of any one of the present embodiments with a cell under conditions in which the nucleic acid is incorporated into the cell, whereby the cell expresses the inducible chimeric signaling polypeptide or the chimeric signaling polypeptide from the incorporated nucleic acid.

Provided in certain embodiments are methods for stimulating a cell-mediated immune response in a subject, comprising administering a modified cell transfected or transduced with a nucleic acid that expresses an inducible chimeric signaling polypeptide of the present embodiments to the subject; and administering an effective amount of a multimeric ligand that binds to the multimeric ligand binding region to stimulate a cell-mediated immune response in the subject. In some embodiments, the modified cell expresses a chimeric antigen receptor, an inducible chimeric antigen receptor polypeptide, or a recombinant T cell receptor, that binds to a target cell. In some embodiments, the target cell is a tumor cell. In some embodiments, the number or concentration of target cells in the subject is reduced following administration of the modified cell and the multimeric ligand. In some embodiments, the methods further comprise measuring the number or concentration of target cells in a first sample obtained from the subject before administering the modified cell or ligand, measuring the number or concentration of target cells in a second sample obtained from the subject after administration of the modified cell and ligand, and determining an increase or decrease of the number or concentration of target cells in the second sample compared to the number or concentration of target cells in the first sample. In some embodiments, an additional dose of ligand is administered to the subject. In some embodiments, an effective amount of multimeric ligand is an amount effective to reduce the number or concentration of target cells or to reduce the symptoms of cytotoxicity. In some embodiments, the cell-mediated response is a T cell-mediated response, a NK-cell mediated response, or a NK-T cell mediated response.

Also provided in some embodiments are methods for treating a subject having a disease or condition associated with expression of a target antigen, comprising administering a multimeric ligand that binds to a multimeric ligand binding region, wherein modified T cells circulating in the subject express (i) an inducible chimeric signaling polypeptide of the preset embodiments and chimeric antigen receptor that binds to the target antigen; or (ii) an inducible chimeric antigen receptor polypeptide of the present embodiments that binds to the target antigen, wherein the target antigen is present on target cells circulating in the subject; and wherein the number or concentration of target cells in the subject is reduced following administration of the multimeric ligand. In some embodiments, the target antigen is expressed by a tumor cell, and the chimeric antigen receptor or the inducible chimeric antigen receptor polypeptide binds to the tumor cell. In some embodiments, following administration of the multimeric ligand, the number or concentration of target cells in the subject is determined, and (i) the administration of the multimeric ligand is discontinued or (ii) an additional dose of multimeric ligand is administered that is lower than the previous dose of multimeric ligand administered. In some embodiments, following administration of the multimeric ligand, the number or concentration of target cells in the subject is determined, and an additional dose of multimeric ligand is administered that is higher than the previous dose of multimeric ligand administered. In some embodiments, the additional dose of multimeric ligand is greater than the previous dose, in some embodiments, the additional dose of multimeric ligand is 10%, 20%, 30%, 40%, 50%, 60%, 70%, 80%, 90%, 100%, 125%, 150%, 175%, 200%, 225%, 250%, 275%, 300%, 400%, 500%, 600%, 700%, 800%, or 1000% greater than the previous dose.

In some embodiments, the multimeric ligand that binds to the multimeric ligand binding region is rimiducid or AP21087

Also provided in some embodiments are methods for stimulating a cell-mediated immune response in a subject, comprising administering an effective amount of modified cells that have been transduced or transfected with a nucleic acid of the present embodiments that express a chimeric signaling polypeptide of the present embodiments to the subject. In some embodiments, the cell-mediated immune response is directed against a target cell. In some embodiments, the modified cell comprises a chimeric antigen receptor, a chimeric antigen receptor polypeptide of the present embodiments, or a recombinant T cell receptor, that binds to an antigen on a target cell. In some embodiments, the target cell is a tumor cell. In some embodiments, the number or concentration of target cells in the subject is reduced following administration of the modified cells. In some embodiments, an additional dose of modified cells is administered to the subject. In some embodiments, the -mediated response is a T cell-mediated response, a NK cell-mediated response, or a NK-T cell-mediated response. Also provided in certain embodiments are methods for providing anti-tumor immunity to a subject, comprising administering to the subject an effective amount of a modified cell that expresses a chimeric signaling polypeptide of any one of the present embodiments. Also provided in certain embodiments are methods for treating a subject having a disease or condition associated with an elevated expression of a target antigen, comprising administering to the subject an effective amount of a modified cell of any one of the present embodiments. In some embodiments, the target antigen is a tumor antigen. Also provided in certain embodiments are methods for reducing the size of a tumor in a subject, comprising administering a modified cell of any one of the present embodiments to the subject, wherein the modified cell comprises a chimeric antigen receptor, a chimeric antigen receptor polypeptide of the present embodiments, or a recombinant T cell receptor, comprising an antigen recognition moiety that binds to an antigen on the tumor.

In some embodiments, the modified cell comprises a chimeric Caspase-9 polypeptide comprising a multimeric ligand binding region and a Caspase-9 polypeptide. In some embodiments, the method further comprises administering a multimeric ligand that binds to the multimeric ligand binding region to the subject following administration of the modified cells to the subject. In some embodiments, after administration of the multimeric ligand, the number of modified cells comprising the chimeric Caspase-9 polypeptide is reduced.

In some embodiments of the present methods, the subject has been diagnosed as having a tumor. In some embodiments, the subject has cancer. In some embodiments, the subject has a solid tumor. In some embodiments, the cancer is present in the blood or bone marrow of the subject. In some embodiments, the subject has a blood or bone marrow disease. In some embodiments, the subject has been diagnosed with any condition or condition that can be alleviated by stem cell transplantation. In some embodiments, the subject has been diagnosed with sickle cell anemia or metachromatic leukodystrophy. In some embodiments, the subject has been diagnosed with a condition selected from the group consisting of a primary immune deficiency condition, hemophagocytosis lymphohistiocytosis (HAH) or other hemophagocytic condition, an inherited marrow failure condition, a hemoglobinopathy, a metabolic condition, and an osteoclast condition. In some embodiments, the subject has been diagnosed with a disease or condition selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/IL-10 Receptor Deficiency, GATA 2 deficiency, X-linked lymphoproliferative disease (XAP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis. In some embodiments, the subject has been diagnosed with leukemia. In some embodiments, the subject has been diagnosed with an infection of viral etiology selected from the group consisting HIV, influenza, Herpes, viral hepatitis, Epstein Bar, polio, viral encephalitis, measles, chicken pox, Cytomegalovirus (CMV), adenovirus (ADV), HHV-6 (human herpesvirus 6, I), and Papilloma virus, or has been diagnosed with an infection of bacterial etiology selected from the group consisting of pneumonia, tuberculosis, and syphilis, or has been diagnosed with an infection of parasitic etiology selected from the group consisting of malaria, trypanosomiasis, leishmaniasis, trichomoniasis, and amoebiasis.

The methods and compositions discussed herein refer, in some examples, to the expression of nucleic acids in cells, following transfection or transduction of the cells with the nucleic acids that encode the chimeric signaling polypeptides provided herein. The nucleic acids may be expressed, in some embodiments, in T cells, NK cells, and NK-T cells, and in some embodiments, in tumor infiltrating lymphocytes. By “tumor infiltrating lymphocytes” is meant to exclude antigen presenting cells, such as, for example, dendritic cells, B cells, and macrophages. In yet other embodiments, the nucleic acids may be expressed in antigen presenting cells, such as, for example, dendritic cells. In some embodiments, the nucleic acids may be expressed in non-lymphocytic hematopoietic cells, or non-hematopoietic cells, such as, for example, macrophages, melanoma cells, fibroblasts, and keratinocytes.

In some embodiments, the membrane targeting region is selected from the group consisting of a myristoylation region, palmitoylation region, prenylation region, and transmembrane sequences of receptors. In some embodiments, the membrane targeting region is a myristoylation region. In some embodiments, the multimeric ligand binding region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof. In some embodiments, the multimeric ligand binding region is an FKBP12 region. In some embodiments, the multimeric ligand is an FK506 dimer or a dimeric FK506 analog ligand. In some embodiments, the ligand is rimiducid (AP1903). In some embodiments, the cell is administered to the subject by intravenous, intradermal, subcutaneous, intratumor, intraprotatic, or intraperitoneal administration.

In some embodiments, the prostate cancer is selected from the group consisting of metastatic, metastatic castration resistant, metastatic castration sensitive, regionally advanced, and localized prostate cancer. In some embodiments, at least two doses of the cell and the ligand are administered to the subject. In some embodiments, the cell is a dendritic cell. In some embodiments, the methods further comprise administering a chemotherapeutic agent. In some embodiments, the composition, ligand, and the chemotherapeutic agent are administered in an amount effective to treat cancer, such as, for example, prostate cancer, in the subject. In some embodiments, the composition or the nucleotide sequences, the ligand, and the chemotherapeutic agent are administered in an amount effective to treat cancer in the subject. In some embodiments, the chemotherapeutic agent is selected from the group consisting of carboplatin, estramustine phosphate (Emcyt), and thalidomide. In some embodiments, the chemotherapeutic agent is a taxane. The taxane may be, for example, selected from the group consisting of docetaxel (Taxotere), paclitaxel, and cabazitaxel. In some embodiments, the taxane is docetaxel. In some embodiments, the chemotherapeutic agent is administered at the same time or within one week after the administration of the cell, nucleic acid or the ligand. In other embodiments, the chemotherapeutic agent is administered after the administration of the ligand. In other embodiments, the chemotherapeutic agent is administered from 1 to 4 weeks or from 1 week to 1 month, 1 week to 2 months, or 1 week to 3 months after the administration of the ligand. In other embodiments, the methods further comprise administering the chemotherapeutic agent from 1 to 4 weeks, or from 1 week to 1 month, 1 week to 2 months, or 1 week to 3 months before the administration of the cell or nucleic acid. In some embodiments, the chemotherapeutic agent is administered at least 2 weeks before administering the cell or nucleic acid. In some embodiments, the chemotherapeutic agent is administered at least 1 month before administering the cell or nucleic acid. In some embodiments, the chemotherapeutic agent is administered after administering the multimeric ligand. In some embodiments, the chemotherapeutic agent is administered at least 2 weeks after administering the multimeric ligand. In some embodiments, wherein the chemotherapeutic agent is administered at least 1 month after administering the multimeric ligand.

In some embodiments, the methods further comprise administering two or more chemotherapeutic agents. In some embodiments, the chemotherapeutic agents are selected from the group consisting of carboplatin, Estramustine phosphate, and thalidomide. In some embodiments, at least one chemotherapeutic agent is a taxane. The taxane may be, for example, selected from the group consisting of docetaxel, paclitaxel, and cabazitaxel. In some embodiments, the taxane is docetaxel. In some embodiments, the chemotherapeutic agents are administered at the same time or within one week after the administration of the cell, nucleic acid or the ligand. In other embodiments, the chemotherapeutic agents are administered after the administration of the ligand. In other embodiments, the chemotherapeutic agents are administered from 1 to 4 weeks or from 1 week to 1 month, 1 week to 2 months, or 1 week to 3 months after the administration of the ligand. In other embodiments, the methods further comprise administering the chemotherapeutic agents from 1 to 4 weeks or from 1 week to 1 month, 1 week to 2 months, or 1 week to 3 months before the administration of the cell or nucleic acid.

In some embodiments, the subject is a mammal. In some embodiments, the subject is a human.

Certain embodiments are described further in the following description, examples, claims and drawings.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIGS. 1A and 1B. FIG. 1A provides schematics of examples of inducible chimeric signaling polypeptide constructs co-expressed with chimeric antigen receptors. Signals from the TCRζ moiety of the chimeric antigen receptor activate the T cell and stimulate target cell killing. These signals synergize with costimulatory signals to generate a robust proliferative capacity and production of the T cell cytokine IL-2. Costimulatory signals inducibly generated by iMX where X a signaling domain derived from a costimulatory receptor augment cytokine production and T cell survival and persistence. Q represents the epitope from CD34 bound by Q-bend 10 monoclonal antibody used to mark transduced CAR-T cells, and 8stm represents the transmembrane domain linking the intracellular signaling domain with the extracellular scFv targeting domain. FIG. 1B provides a schematic representation of examples of retroviral inducible chimeric signaling polypeptide expression constructs. Cistrons for MyD88-X are expressed with a 2A cotranslational cleavage peptide 5′ to the CAR-encoding cistrons.

FIGS. 2A-2F. Induction of signaling activity by chemical induction of protein dimerization: synergism of MyD88 and CD40 signaling activity. HEK-293 cells were transfected with a reporter plasmid responsive to NF-κB activity and one of the indicated constructs of FIG. 2A. After 24 hours incubation cells were split to 96 well plates in the presence of increasing concentrations of the FKBP12 dimerizing ligand rimiducid. FIGS. 2B-2E provide graphs of the Secreted Alkaline Phosphatase (SeAP) reporter activity determined from cell supernatants after 24 hours. FIG. 2F provides a bar graph of the basal level of reporter activity without drug stimulation.

FIGS. 3A-3D. Fusion of alternate signaling domains with Inducible MyD88 changes the parameters of signaling activity with rimiducid addition. 293T cells were transfected with NF-κB SeAP reporter and inducible chimeric signaling polypeptide constructs provided in FIG. 1B. Signaling activity at a range of concentrations of the dimerizing ligand rimiducid were determined. FIG. 3A provides a graph of NF-κB SEAP activity of MyD88 fusions as labeled. FIG. 3B provides a graph of NF-κB SEAP activity of MyD88 fusions as labeled, FIG. 3C provides a bar graph of basal NF-κB SEAP activity. FIG. 3D provides a graph of NF-κB SEAP activity including the activity of the chimeric polypeptides, including the multimeric ligand binding region and the cytoplasmic signaling regions of various polypeptides, but without fusion to MyD88. No inducible signaling was observed using constructs that did not include MyD88.

FIGS. 4A and 4B. Coculture of T cells that express a CAR that recognizes PSCA and an inducible chimeric signaling polypeptide with HPAC pancreatic cancer cells. FIG. 4A provides a graph of inducible CAR-T killing in the absence of rimiducid. FIG. 4B provides a graph of inducible CAR-T killing in the presence of 1 nM rimiducid. HPAC cells express a fluorescent marker and individual cell numbers were visualized and quantitated with an Incucyte incubator/microscope/scanner (Essen Biosciences). Individual points are cell quantitations from eight fields taken every three hours for 7 days. Rimiducid did not affect the cell growth of the tumor or T cell in the Not Transduced (NT) population of primary T cells (FIG. 4A) but augmented T cell efficacy in T cells that expressed the inducible chimeric signaling polypeptide (FIG. 4B). Note the difference in scale between the two graphs.

FIGS. 5A-5D provide bar graphs showing cytokine production from cells that express inducible chimeric signaling polypeptides and a CAR that recognizes PSCA. T cells were incubated at an E:T ratio of 1:5 with HPAC tumor cells in the presence or absence of 1 nM rimiducid or in the absence of tumor target. FIG. 5A is a bar graph showing production of IL-2. FIG. 5B is a bar graph showing production of IP-10. FIG. 5C is a bar graph showing production of IL-5. FIG. 5D is a bar graph showing production of MCP-1. The bars are provided in each graph in the same order as in the corresponding legend.

FIGS. 6A-6C provide bar graphs showing cytokine production from cells that express inducible chimeric signaling polypeptides and a CAR that recognizes PSCA. T cells were incubated at an E:T ratio of 1:5 with HPAC tumor cells in the presence or absence of 1 nM rimiducid or in the absence of tumor target. FIG. 6A is a bar graph showing production of IFN-γ. FIG. 6B is a bar graph showing production of MIP-1a. FIG. 6C is a bar graph showing production of TNF-β. The bars are provided in each graph in the same order as in the corresponding legend.

FIGS. 7A-7B provide bar graphs showing that the production of inflammatory cytokines is markedly reduced by replacement of CD40 with alternative signaling domains. T cells that express inducible chimeric signaling polypeptides and a CAR that recognizes PSCA were incubated at an E:T ratio of 1:5 with HPAC tumor cells in the presence or absence of 1 nM rimiducid or in the absence of tumor target. FIG. 7A is a bar graph showing production of IL-6. FIG. 7B is a bar graph showing production of TNF-α. The bars are provided in each graph in the same order as in the corresponding legend.

FIGS. 8A-8C. FIG. 8A provides schematics of retroviral vectors that encode chimeric MyD88/CD40 (MC) polypeptides, or chimeric signaling polypeptides. Retroviral vector BP414 encodes a first generation CAR without MC. BP813 and 844 encode first generation CARs and chimeric MC polypeptides, where the MC polypeptides either include a myristoylation region (813) or do not include a myristoylation region (844), where an inducible Caspase-9 polypeptide is co-expressed. In these vectors, MC is encoded 3′ to the same first generation CAR separated by a P2A sequence. BP2103 and BP2104 replace CD40 with the 4-1BB signaling domain as a fusion with MyD88. BP2103 and BP2104 encode first generation CARs and chimeric MyD88/4-1BB (MyBB) polypeptides, where the MyBB polypeptides either include a myristoylation region (2103) or do not include a myristoylation region (2104). FIG. 8B provides a bar graph of control of GFP labeled Daudi tumor target cells. FIG. 8C provides a bar graph showing proliferation of RFP-labeled CAR-T cells. Daudi lymphoma cells express a fluorescent GFP marker and individual cell numbers were visualized and quantitated with an Incucyte incubator/microscope/scanner (Essen Biosciences). CAR-T cells were marked red with RFP alone (RFP) and as a cotransduction with CAR-T encoding viruses. Individual points were cell quantitations from eight fields taken every three hours for 7 days.

FIGS. 9A-9D provide bar graphs showing that the production of inflammatory cytokines is markedly reduced by replacement of CD40 with MyBB. The T cells expressed a CAR that recognized CD19, as well as an inducible Caspase-9 polypeptide, and the MC polypeptide or chimeric MC polypeptide as labeled. “d” as in “dMC” or “dMB,” for example, indicates that the polypeptide lacks a membrane targeting region. In some examples of the present application, “d” as in “dMC” or “dMC” is referred to as “Δ” as in “ΔMC” or “ΔMB.” The T cells were incubated at an E:T ratio of 1:5 or 1:25 with Daudi tumor cells and the level of the indicated cytokines released at 24 hours determined. MyD88-CD40 (MC) was the costimulatory polypeptide that promoted maximal IL-2 levels at 1:5 E:T while MyD88-4-1BB (MB) did not secrete appreciable levels of the inflammatory cytokine IL-6. FIG. 9A is a bar graph showing IL-2 production. FIG. 9B is a bar graph showing IFNγ production. FIG. 9C is a bar graph showing IL-6 production. FIG. 9D is a bar graph showing TNFα production. The bars are provided in each graph in the same order as in the corresponding legend.

FIG. 10 provides charts representing the relative expansion of CD4⁺ and CD8⁺ in cultured CAR-T cells carrying iMyD88 fusions. Cells were cultured with IL-2 for three weeks and a proportion was isolated and stained with antibodies to CD4, CD8 and CD3. The relative proportion of CD4 and CD8 positivity was quantitated on a Galleos flow cytometer with Kaluza software.

FIG. 11 provides charts representing the relative expansion and differentiation of CD4⁺ T cell subtypes in cultured chimeric antigen-expressing T cells carrying iMyD88 fusions. Cells were cultured with IL-2 for three weeks and a proportions was isolated and stained with labeled antibodies to CD4, CD8, CCR6, CXCR3 and CD3. The relative proportion of CCR6hi/CXCR3lo, CCR6lo/CXCR3lo and CCR6lo/CXCR3hi was quantitated on a Galleos flow cytometer with Kaluza software. CD8 cells were excluded from the analysis.

FIG. 12 provides charts representing the differentiation of CD8⁺ CAR-T cells to memory cell lineages in response to signaling from introduced iMyD88 fusions. Cells were cultured with IL-2 for three weeks and a proportions was isolated and stained with labeled antibodies to CD34 (transduced cells), CD4, CD8, CD45-RO (a splice form present in differentiated memory cells only), CCR7 and CD3. The relative proportion of CCR7hi/CD45ROhi, CCR7lo/CD45ROhi, CCR7hi/CD45ROlo and CCR7lo/CD45ROlo was quantitated on a Galleos flow cytometer with Kaluza software. CD4 cells were excluded from the analysis.

FIG. 13 provides charts representing the differentiation of CD4⁺ CAR-T cells to memory cell lineages in response to signaling from introduced iMyD88 fusions. Cells were cultured with IL-2 for three weeks and a proportions was isolated and stained with labeled antibodies to CD34 (transduced cells), CD4, CD8, CD45-RO (a splice form present in differentiated memory cells only), CCR7 and CD3. The relative proportion of CCR7hi/CD45ROhi, CCR7lo/CD45ROhi, CCR7hi/CD45ROlo and CCR7lo/CD45ROlo was quantitated on a Galleos flow cytometer with Kaluza software. CD8 cells were excluded from the analysis.

FIGS. 14A-14T provide plasmid maps for plasmids discussed in the Examples, including Plasmids A-T.

FIG. 15A and FIG. 15B are bar graphs of IL-2 and IL-6 secretion in modified cells.

FIG. 16A and FIG. 16B are bar graphs of IFN-γ secretion in modified cells.

FIGS. 17A-17D are graphs of costimulatory activity of modified cells in a tumor cell killing assay. FIG. 17A: right side of graph, in order, top to bottom: 1475+Rim; NT+Rim; NT; 1475, 2206+Rim and 2206. FIG. 17B: right side of graph, in order, top to bottom: NT; 2209 and 2209+Rim; 2205 and 2205+Rim. FIG. 17C: right side of graph, in order, top line: NT; bottom line: 1849, 1849+Rim, 1850, and 1850+Rim. FIG. 17D: right side of graph, in order, top line: NT; bottom line: 2202, 2202+Rim, 2203, 2203+Rim.

FIG. 18A and FIG. 18B are bar graphs of the activation state of modified cells.

FIG. 19A and FIG. 19B are bar graphs of IL-2 and IL-6 secretion in modified cells.

FIG. 20A and FIG. 20B are graphs of signal enhanced tumor killing by modified cells.

FIG. 21A and FIG. 21B are schematics of examples of costimulatory polypeptide RANK signaling regions.

FIG. 22 is a line graph of control of HPAC tumor outgrowth with myristoylated inducible 4-1BB.

### DETAILED DESCRIPTION

MyD88 (encoded by myeloid differentiation primary response gene 88) is a crucial mediator of signals downstream of several receptors, notably the Toll-like Receptors (TLRs) that direct a part of innate immune responses. Fusions of a truncated MyD88 polypeptide, lacking the TIR domain with the intracellular domain of CD40 (“MC”) to produce a chimeric polypeptide amplifies certain signals directed by MyD88. When T cells are transfected or transduced with nucleic acids that encode MC, in combination with a Chimeric Antigen Receptor (CAR), MC delivers potent costimulatory signals that enhance T cell growth, persistence, and cytotoxic activity against cells specifically targeted by the CAR. (see, for example, U.S. patent application Ser. No. 14/842,710, titled Costimulation of Chimeric Antigen Receptors by MyD88 and CD40 polypeptide, by Spencer, D., et al, filed Sep. 1, 2015, published as US-2016-0058857A1 on Mar. 3, 2016; and International Patent Application PCT/US2015/047957, filed Dec. 14, 2015, published as WO/2016/036746 on Mar. 10, 2016, all incorporated herein by reference in their entireties).

Provided herein are chimeric signaling polypeptides where the truncated MyD88 polypeptide has also been fused with signaling domains of receptor mediators of costimulation, such as, for example, CD28, 4-1BB, OX40, or ICOS. The chimeric signaling polypeptides may be expressed as part of an inducible chimeric signaling polypeptide, or as a chimeric signaling polypeptide having constitutive activity.

In some embodiments, inducible chimeric signaling polypeptides may comprise a truncated MyD88 polypeptide lacking the TIR domain, a cytoplasmic signaling domain selected from the group consisting of CD28, 4-1BB, OX-40, and ICOS, and a multimeric ligand binding region such as an FKBP12 multimeric ligand binding region, for example, a wild type FKBP12 multimeric ligand binding region (Fwt) or a FKBP12 variant polypeptide that is inducible with the dimerizing small molecule AP1903 (rimiducid) or AP20187, such as, for example, a FKBP12 variant polypeptide that has an amino acid substitution at amino acid 36, substituting a different amino acid for the phenylalanine residue at position 36, for example, valine (FKBP12v36, Fv). The inducible forms of these fusions generate differential activity to transduce activating signals to the NF-κB family of transcription factors when activated with rimiducid. NF-κB is a key mediator of costimulation, cell survival and cytokine production.

The capacity of these MyD88 fusions to support CAR-T cell-mediate attack of tumor cells was compared. Because some cytokines secreted by CAR-T cells produce toxic effects, or cytotoxicity, in cancer patients, the cytokines secreted by the CAR-T cells that expressed the MyD88 fusions were also assessed.

Inducible Caspase 9 (iC9). This proapoptotic switch includes a fusion of caspase-9 with FKBP12 or derivatives. It is latent in the absence of ligand but drives dimerization of the Initiator caspase, caspase-9, from the intrinsic pathway for cell apoptosis. Dimerization leads to caspase-9 activation, cleavage and activation of the effector caspase, caspase-3, and rapid cell death by apoptosis. Inducible Caspase-9 has utility as a safety switch in cell therapies to block toxic responses.

Rapamycin and rapalog sensitive switches—Rapamycin is a macrolide that binds with subnanomolar affinity with FKBP12 and simultaneously with the target of rapamycin mTOR. An 89-amino acid domain derived from mTOR, FRB, is sufficient to dimerize with an FKBP12-rapamycin complex. Fusion of FKBP in tandem with FRB together with a signaling domain facilitates homodimerization and activates signaling in the presence of the heterodimerizer rapamycin or analogs of rapamycin, generically termed rapalogs. Rapamycin and certain rapalogs are cell-permeable, stable in vivo and bind their targets with high affinity and specificity.

Rapamycin/rapalog-sensitive switches iRC9 and iRMC—Fusion of FKBP12-FRB to the amino terminus of Caspase-9 generates a rapamycin-sensitive safety switch that operates with high efficiency and dose sensitivity. Fusion of FKBP12-FRB with MyD88/CD40 generates a rapamycin or rapalog sensitive costimulatory switch. The FKBP and FRB components can be put in tandem in either a FRB-FKBP or FKBP-FRB orientation and can be fused with the MC signaling components at the amino or carboxy terminus.

FKBP12-allele specific binding by rimiducid. Rimiducid binds with high affinity (˜0.1 nM) to the valine 36 allele of FKBP12 but with low affinity (˜500 nM) to the wild-type phenylalanine 36 FKBP12 allele. Rapamycin and rapalogs can bind to either FKBP allele.

Non-immunosuppressive C7-rapamycin analogs. The natural target of rapamycin, mTOR is essential for cell growth and rapamycin is immunosuppressive at low dose (˜1 nM). Rapalogs replacing the methoxy group at C7 with groups that have more bulk, typified by BPC015, bind with mTOR with low affinity. Mutation of the FRB in iRC9 or iRMC (or similar) to substitute threonine 2098 with leucine accommodates the derivatized rapalog and permits high affinity dimerization and signaling.

Orthogonal Use of Rimiducid and Rapamycin Sensitive Switches to Generate Dual Switch CAR-T Cells.

iRC9 contains the rimiducid-insensitive F36 allele of FKBP12 and can be coexpressed with iMC in T cells. Doses of rimiducid capable of activating iMC and driving costimulation are incapable of activating the proapoptotic iRC9 switch. Rapamycin or rapalogs can activate the safety switch. Similarly, coexpression of iRMC with iC9 can generate dual switch CAR-T cells with the opposite specificity. Rapalogs are obligate heterodimerizers and can bind to but not activate the iC9 switch containing only FKBP12. Rimiducid can dimerize and activate the safety switch.

Provided herein are inducible and constitutive chimeric truncated MyD88 polypeptides that, when expressed in, for example, CAR-T cells, produce significantly fewer of certain toxic inflammatory cytokines than CAR-T cells that express an inducible or constitutive MyD88-CD40 chimeric polypeptide, while retaining potent or even enhanced tumor cell killing. Also provided are modified chimeric antigen receptors, where the chimeric antigen receptor polypeptide comprises inducible or constitutive chimeric truncated MyD88 polypeptides.

The specificity of downstream signaling from different MyD88 fusions to cellular (mainly transcriptional) targets may thereby permit a clinician to select a profile of cytokine release tailored to the tumor type or its location. Different MyD88 fusions affected the relative proliferation and survival of helper and cytotoxic CAR-T cells and the differentiation of each class into memory T cells over time. This capacity may be important for maintaining surveillance for relapse of tumors initially destroyed by T cell immunotherapy.

The inducible and constitutive chimeric signaling polypeptides provided herein may be used, for example, to induce or increase an immune response alone, or in combination with chimeric antigen receptors (CARs), which allows the immune response to be specifically directed against particular tumor cells. The controlled T cell activation methods avoid many of the toxic side effects of earlier CAR-based treatments.

Also provided herein are immune cells, such as, for example, activated T cells that express an inducible or constitutive chimeric signaling polypeptide. The activated cells may be used to increase the immune response against a disease, or to treat cancer by, for example, reducing the size of a tumor. Therapeutic courses of treatment using the activated T cells and activated CAR T cells may be monitored by determining the size and vascularity of tumors by various imaging modalities (e.g. CT, bonescan, MRI, PET scans, Trofex scans), by various standard blood biomarkers (e.g. PSA, Circulating Tumor Cells), or by serum levels of various inflammatory, hypoxic cytokines, or other factors in the treated patient.

The inducible chimeric signaling polypeptides discussed herein allow for a sustained, modulated control of a chimeric antigen receptor (CAR) that is co-expressed in the cell. The full activation of the antigen-specific T cell, designed to target a cellular antigen implicated in a disease or condition, using an inducible chimeric signaling polypeptide is dependent on the administration of a ligand inducer. The ligand inducer activates the CAR-expressing cell by multimerizing the inducible chimeric signaling polypeptides, which, in turn, activates NF-κB signaling and other intracellular signaling, pathways, which activates the cell, for example, a T cell, a tumor-infiltrating lymphocyte, a natural killer cell, or a natural killer T cell. In the absence of the ligand inducer, the T cell is quiescent, or has a basal level of activity. Dosing of the ligand determines the rate and magnitude of the CAR-expressing T cell proliferation and activation. Selection of the appropriate inducible chimeric signaling polypeptide may include consideration of the level of basal activity that may be produced in the cell.

Full activation and tumor cell killing remains dependent on antigen recognition and additional activation of NFAT via CD3 zeta signaling. Once a complete response (CR) is achieved, the dosing of the ligand may be ceased. If the disease or condition reoccurs, the ligand dosing may be reinitiated, leading to re-expansion and reactivation of quiescent, tumor-target, T cells. Where it is appropriate to maintain CAR-T cells that express the inducible or constitutive chimeric signaling polypeptides, a clinician may select the appropriate chimeric signaling polypeptide while considering both the level of activation upon induction, and any basal level of cytokine expression and possible cytotoxicity.

Costimulation in T-cells that express chimeric antigen receptors by MyD88 and CD40 polypeptides is discussed in U.S. patent application Ser. No. 14/842,710, filed Sep. 1, 2015, published as US2016-0058857-A1 on Mar. 3, 2016, entitled “Costimulation of Chimeric Antigen Receptors by MyD88 and CD40 Polypeptides.” The entire content of the foregoing application is incorporated herein by reference in its entirety, including all text, tables and drawings, for all purposes.

Non-limiting examples of chimeric polypeptides useful for inducing cell activation, and related methods for inducing CAR-T cell activation including, for example, expression constructs, methods for constructing vectors, and assays for activity or function, may also be found in the following patents and patent applications, each of which is incorporated by reference herein in its entirety for all purposes. U.S. patent application Ser. No. 14/210,034, filed Mar. 13, 2014, entitled METHODS FOR CONTROLLING T CELL PROLIFERATION, published Sep. 25, 2014 as US2014-0286987-A1; International Patent Application No. PCT/US2014/026734, filed Mar. 13, 2014, published Sep. 25, 2014 as WO2014/151960, by Spencer et al.; U.S. patent application Ser. No. 14/622,018, filed Feb. 13, 2014, entitled METHODS FOR ACTIVATING T CELLS USING AN INDUCIBLE CHIMERIC POLYPEPTIDE, published Feb. 18, 2016 as US2016-0046700-A1; International Patent Application No. PCT/US2015/015829, filed Feb. 13, 2015, published Aug. 20, 2015 as WO2015/123527; U.S. patent application Ser. No. 10/781,384, filed Feb. 18, 2004, entitled INDUCED ACTIVATION OF DENDRITIC CELLS, published Oct. 21, 2004 as US2004-0209836-A1, issued Jun. 29, 2008 as U.S. Pat. No. 7,404,950, by Spencer et al.; International Patent Application No. PCT/US2004/004757, filed Feb. 18, 2004, published Mar. 24, 2005 as WO2004/073641A3; U.S. patent application Ser. No. 12/445,939, filed Oct. 26, 2010, entitled METHODS AND COMPOSITIONS FOR GENERATING AN IMMUNE RESPONSE BY INDUCING CD40 AND PATTERN RECOGNITION RECEPTORS AND ADAPTORS THEREOF, published Feb. 10, 2011 as US2011-0033388-A1, issued Apr. 8, 2014 as U.S. Pat. No. 8,691,210, by Spencer et al.; International Patent Application No. PCT/US2007/081963, filed Oct. 19, 2007, published Apr. 24, 2008 as WO2008/049113; U.S. patent application Ser. No. 13/763,591, filed Feb. 8, 2013, entitled METHODS AND COMPOSITIONS FOR GENERATING AN IMMUNE RESPONSE BY INDUCING CD40 AND PATTERN RECOGNITION RECEPTOR ADAPTERS, published Mar. 27, 2014 as US2014-0087468-A1, issued Apr. 19, 2016 as U.S. Pat. No. 9,315,559, by Spencer et al.; International Patent Application No. PCT/US2009/057738, filed Sep. 21, 2009, published Mar. 25, 2010 as WO201033949; U.S. patent application Ser. No. 13/087,329, filed Apr. 14, 2011, entitled METHODS FOR TREATING SOLID TUMORS, published Nov. 24, 2011 as US2011-0287038-A1, by Slawin et al.; International Patent Application No. PCT/US2011/032572, filed Apr. 14, 2011, published Oct. 20, 2011 as WO2011/130566, by Slawin et al; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1., by Bayle et al.; International Patent Application No. PCT/US2016/066371, filed Dec. 13, 2016, published Jun. 22, 2017 as WO2017/106185, by Bayle et al.; and U.S. Provisional Patent Application No. 62/503,565, filed May 9, 2017, entitled METHODS TO AUGMENT OR ALTER SIGNAL TRANSDUCTION, by Bayle et al., each of which is incorporated by reference herein in its entirety for all purposes.

Non-limiting examples of chimeric polypeptides useful for inducing cell death or apoptosis, and related methods for inducing cell death or apoptosis, including expression constructs, methods for constructing vectors, assays for activity or function, and multimerization of the chimeric polypeptides by contacting cells that express inducible chimeric polypeptides with a multimeric compound, or a pharmaceutically acceptable salt thereof, that binds to the multimerizing region of the chimeric polypeptides both ex vivo and in vivo, administration of expression vectors, cells, or multimeric compounds described herein, or pharmaceutically acceptable salts thereof, to subjects, and administration of multimeric compounds described herein, or pharmaceutically acceptable salts thereof, to subjects who have been administered cells that express the inducible chimeric polypeptides, may also be found in the following patents and patent applications, each of which is incorporated by reference herein in its entirety for all purposes. U.S. patent application Ser. No. 13/112,739, filed May 20, 2011, entitled METHODS FOR INDUCING SELECTIVE APOPTOSIS, published Nov. 24, 2011, as US2011-0286980-A1, issued Jul. 28, 2015 as U.S. Pat. No. 9,089,520; U.S. patent application Ser. No. 13/792,135, filed Mar. 10, 2013, entitled MODIFIED CASPASE POLYPEPTIDES AND USES THEREOF, published Sep. 11, 2014 as US2014-0255360-A1, issued Sep. 6, 2016 as U.S. Pat. No. 9,434,935, by Spencer et al.; International Patent Application No. PCT/US2014/022004, filed Mar. 7, 2014, published Oct. 9, 2014 as WO2014/16438; U.S. patent application Ser. No. 14/296,404, filed Jun. 4, 2014, entitled METHODS FOR INDUCING PARTIAL APOPTOSIS USING CASPASE POLYPEPTIDES, published Jun. 2, 2016 as US2016-0151465-A1, by Slawin et al; International Application No. PCT/US2014/040964 filed Jun. 4, 2014, published as WO2014/197638 on Feb. 5, 2015, by Slawin et al.; U.S. patent application Ser. No. 14/640,553, filed Mar. 6, 2015, entitled CASPASE POLYPEPTIDES HAVING MODIFIED ACTIVITY AND USES THEREOF, published Nov. 19, 2015 as US2015-0328292-A1; International Patent Application No. PCT/US2015/019186, filed Mar. 6, 2015, published Sep. 11, 2015 as WO2015/134877, by Spencer et al.; U.S. patent application Ser. No. 14/968,737, filed Dec. 14, 2015, entitled METHODS FOR CONTROLLED ELIMINATION OF THERAPEUTIC CELLS, published Jun. 16, 2016 as US2016-0166613-A1, by Spencer et al.; International Patent Application No. PCT/US2015/065629 filed Dec. 14, 2015, published Jun. 23, 2016 as WO2016/100236, by Spencer et al.; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1., by Bayle et al.; and International Patent Application No. PCT/US2016/066371, filed Dec. 13, 2016, published Jun. 22, 2017 as WO2017/106185, by Bayle et al., each of which is incorporated by reference herein in its entirety for all purposes. Multimeric compounds described herein, or pharmaceutically acceptable salts thereof, may be used essentially as discussed in examples provided in these publications, and other examples provided herein.

Chimeric Antigen Receptors and Cell Therapy

Chimeric antigen receptors (CARs) are artificial receptors designed to convey antigen specificity to T cells. They generally include an antigen-specific component, a transmembrane component, and an intracellular component selected to activate the T cell and provide specific immunity. Chimeric antigen receptor-expressing T cells may be used in various therapies, including cancer therapies. While effective against tumors, in some cases these therapies have led to side effects due, in part to non-specific attacks on healthy tissue. A method for controllable T cell therapy is needed that provides a strong immunotherapeutic response and avoids toxic side effects.

The antitumor efficacy from immunotherapy with T cells engineered to express chimeric antigen receptors (CARs) has steadily improved as CAR molecules have incorporated additional signaling domains to increase their potency. T cells transduced with first generation CARs, containing only the CD3ζ intracellular signaling molecule, have demonstrated poor persistence and expansion in vivo following adoptive transfer (Till B G, Jensen M C, Wang J, et al: CD20-specific adoptive immunotherapy for lymphoma using a chimeric antigen receptor with both CD28 and 4-1BB domains: pilot clinical trial results. Blood 119:3940-50, 2012; Pule M A, Savoldo B, Myers G D, et al: Virus-specific T cells engineered to coexpress tumor-specific receptors: persistence and antitumor activity in individuals with neuroblastoma. Nat Med 14:1264-70, 2008; Kershaw M H, Westwood J A, Parker L L, et al: A phase I study on adoptive immunotherapy using gene-modified T cells for ovarian cancer. Clin Cancer Res 12:6106-15, 2006), as tumor cells often lack the requisite costimulating molecules necessary for complete T cell activation. Second generation CAR T cells were designed to improve proliferation and survival of the cells. Second generation CAR T cells that incorporate the intracellular costimulating domains from either CD28 or 4-1BB (Carpenito C, Milone M C, Hassan R, et al: Control of large, established tumor xenografts with genetically retargeted human T cells containing CD28 and CD137 domains. Proc Natl Acad Sci USA 106:3360-5, 2009; Song D G, Ye Q, Poussin M, et al: CD27 costimulation augments the survival and antitumor activity of redirected human T cells in vivo. Blood 119:696-706, 2012), show improved survival and in vivo expansion following adoptive transfer, and more recent clinical trials using anti-CD19 CAR-modified T cells containing these costimulating molecules have shown remarkable efficacy for the treatment of CD19+ leukemia. (Kalos M, Levine B L, Porter D L, et al: T cells with chimeric antigen receptors have potent antitumor effects and can establish memory in patients with advanced leukemia. Sci Trans Med 3:95ra73, 2011; Porter D L, Levine B L, Kalos M, et al: Chimeric antigen receptor-modified T cells in chronic lymphoid leukemia. N Engl J Med 365:725-33, 2011; Brentjens R J, Davila M L, Riviere I, et al: CD19-targeted T cells rapidly induce molecular remissions in adults with chemotherapy-refractory acute lymphoblastic leukemia. Sci Transl Med 5:177ra38, 2013).

In one example of cell therapy, T cells transduced with a nucleic acid encoding a chimeric antigen receptor have been administered to patients to treat cancer (Zhong, X.-S., (2010) Molecular Therapy 18:413-420). For example, T cells expressing a chimeric antigen receptor based on the humanized monoclonal antibody Trastuzumab (Herceptin) has been used to treat cancer patients. Adverse events are possible, however, and in at least one reported case, the therapy had fatal consequences to the patient (Morgan, R. A., et al., (2010) Molecular Therapy 18:843-851). Transducing the cells with a controllable inducible safety switch, as presented herein, would provide a safety switch that could stop the adverse event from progressing, by stopping the administration of the ligand inducer. Although a low level basal activity might remain, removing the presence of the inducer should drastically reduce, if not cease, the symptoms of the adverse event.

CAR-T Cells and Cytotoxicity

T cells expressing chimeric antigen receptors (CARs) have shown long-term efficacy for the treatment of some types of cancer, however, toxicity associated with excessive T cell activation, such as cytokine release syndrome (CRS) remain a concern. Steroids or incorporation of a suicide gene (e.g., inducible caspase-9, HSV-TK, CD20, truncated EGFR) within the vector can be used to improve the safety profile, but these current approaches may reduce the level of or terminate the therapy and hence may impair efficacy. More recently, an IL-6 receptor blockade has been used to manage CRS; however, this strategy may be less effective when direct T cell cytotoxicity is responsible for tissue damage. Additionally, CAR-T cell efficacy has been more limited in solid tumors due to poor CAR-T cell survival, activation and proliferation, presumably due to the more profound inhibitory effects of the tumor microenvironment. Thus, strategies that allow controlled expansion and survival of tumor-targeted T cells would maximize therapeutic potency while minimizing toxicities.

T cells bearing first generation CARs, including a tumor antigen-specific, single-chain variable fragment (scFv) domain and the T cell receptor (TCR)-associated CD3ζ intracellular signaling molecule, fail to persist or expand in vivo, as tumor cells often lack the requisite costimulatory molecules necessary for complete T cell activation. Second generation CAR-T cells that incorporate potent intracellular costimulatory domains, like CD28 or 4-1BB, show improved survival and in vivo expansion following adoptive transfer. Several studies have engineered CAR-T cells with healthy tissue-activated inhibitory domains or have employed a tumor-sensing approach by separating costimulatory domains and CD3ζ on CARs with different antigen targets to limit “on-target, off-tumor” toxicities. While these approaches may improve tumor specificity, they rely on often unpredictable cell autonomous factors. In contrast, physician-enabled approaches to control T cell amplification and elimination in vivo would facilitate patient-tailored therapy coordinated with clinical course, potentially avoiding acute or long-term therapy-associated toxicities.

In general, T cell therapy has involved the difficulty of poor in vivo expansion of the infused cells. One way this issue has been addressed is by administering high doses of IL-2 to the patient. This therapy helps T cell growth and anti-tumor function, but is also very toxic to the patient. This has generally been used in melanoma as high dose IL-2 is considered a standard-of-care therapy for that disease. Most other T cell therapy applications have not used IL-2 with T cell therapy due to toxic effects. Another issue arising in T cell therapy is the poor engraftment and persistence of infused T cells (also a function of in vivo proliferation), which has been addressed by lymphodepleting conditioning prior to T cell infusion. Investigators generally use chemotherapy (cyclophosphamide in particular) to achieve this, although some use antibodies including Campath. Conditioning appears to greatly facilitate T cell therapy through creating lymphoid “space” and depleting regulatory immune cells that compete for growth and survival factors. However, it is very toxic to the patient, completely ablates normal immune cells (e.g. pathogen-specific) and cannot be readily used for some types of cancer or older patients. In addition, use of a lymphodepleting regimen might push a T cell therapy toward a “procedure” rather than a standalone therapeutic.

T cell therapy has largely been considered a boutique therapy since each patient needs to have a unique cell product manufactured for them. Conventional T cell therapies (generated by repetitive antigen stimulation or isolation of tumor infiltrating lymphocytes (TILs) are not reproducible in their specificity or function and lead to extremely variable results, and in some cases the inability to produce a product for treatment. Gene transfer of natural or chimeric T cell receptors has started to solve this problem (where highly tumor specific T cells can be generated in less than 2 weeks), but it is apparent that gene-modified T cells can function differently than naturally occurring T cells. In addition, highly specific CAR T cells or T cells expressing optimized TCR alpha and beta chains can cause off-target toxicity, necessitating the inclusion of a suicide gene.

The most basic components of a chimeric antigen receptor (CAR) include the following components. The variable heavy (V_{H}) and light (V_{L}) chains for a tumor-specific monoclonal antibody are fused in-frame with the CD3 zeta chain (ζ) from the T cell receptor complex. The V_{H} and V_{L} are generally connected together using a flexible glycine-serine linker, and then attached to the transmembrane domain by a spacer (CH₂CH₃) to extend the scFv away from the cell surface so that it can interact with tumor antigens.

Following transduction, T cells now express the CAR on their surface, and upon contact and ligation with a tumor antigen, signal through the CD3 zeta chain inducing cytotoxicity and cellular activation.

Investigators have noted that activation of T cells through CD3 zeta is sufficient to induce a tumor-specific killing, but is insufficient to induce T cell proliferation and survival. Early clinical trials using T cells modified with CARs expressing only the zeta chain showed that gene-modified T cells exhibited poor survival and proliferation in vivo. These constructs are termed 1st generation CARs.

As costimulation through the B7 axis is necessary for complete T cell activation, investigators added the costimulatory polypeptide CD28 signaling domain to the CAR construct. This region generally contains the transmembrane region (in place of the CD3 zeta version) and the YMNM motif for binding PI3K and Lck. In vivo comparisons between T cells expressing CARs with only zeta or CARs with both zeta and CD28 demonstrated that CD28 enhanced expansion in vivo, in part due to increased IL-2 production following activation. The inclusion of CD28 is called a 2nd generation CAR.

The use of costimulatory polypeptides 4-1BB or OX40 in CAR design has further improved T cell survival and efficacy. 4-1BB in particular appears to greatly enhance T cell proliferation and survival. This 3rd generation design (with 3 signaling domains) has been used in PSMA CARs (Zhong X S, et al., Mol Ther. 2010 February; 18(2):413-20), and in CD19 CARs, most notably for the treatment of CLL (Milone, M. C., et al., (2009) Mol. Ther. 17:1453-1464; Kalos, M., et al., Sci. Transl. Med. (2011) 3:95ra73; Porter, D., et al., (2011) N. Engl. J. Med. 365: 725-533). These cells showed impressive function in 3 patients, expanding more than a 1000-fold in vivo, and resulted in sustained remission in all three patients.

While others have explored additional signaling molecules from tumor necrosis factor (TNF)-family proteins, such as OX40 and 4-1BB, called “third generation” CART cells, (Finney H M, Akbar A N, Lawson A D: Activation of resting human primary T cells with chimeric receptors: costimulation from CD28, inducible costimulator, CD134, and CD137 in series with signals from the TCR zeta chain. J Immunol 172:104-13, 2004; Guedan S, Chen X, Madar A, et al: ICOS-based chimeric antigen receptors program bipolar TH17/TH1 cells. Blood, 2014), other molecules which induce T cell signaling distinct from the CD3ζ nuclear factor of activated T cells (NFAT) pathway may provide necessary costimulation for T cell survival and proliferation, and possibly endow CAR T cells with additional, valuable functions, not supplied by more conventional costimulating molecules. Some second and third-generation CAR T cells have been implicated in patient deaths, due to cytokine storm and tumor lysis syndrome caused by highly activated T cells.

However, as CARs have improved in their anti-tumor effects, they have also become more dangerous. There have been two high-profile deaths using 2nd and 3rd generation CARs, which is high considering only a handful of patients have been treated. These deaths resulted from sepsis due to cytokine storm and tumor lysis syndrome caused by highly activated T cells (Morgan, R. A., et al. (2010) Mol. Ther. 14:843-851).

T cell receptor signaling can be induced using a chemical inducer of dimerization (CID) in combination with a chimeric receptor that includes a multimerization region or multimeric ligand binding region that binds to the CID, T cells were engineered to express the CD3 zeta chain, which was linked with 1, 2, or 3 FKBP fragments. The cells expressed the chimeric receptor, and demonstrated CID-dependent T cell activation (Spencer, D. M., et al., Science, 1993. 262: p. 1019-1024). Inducible chimeric stimulating molecules that comprise a CD40 polypeptide lacking the extracellular domain may be used to stimulate the activity of first generation CAR-T cells.

Dendritic cells (DCs) may be activated by chemical induction of dimerization (CID) using a small molecule (i.e., rimiducid/AP1903)-response chimeric signaling molecule, comprising the “universal” Toll-like receptor (TLR) adapter, MyD88, and the TNF family member, CD40²⁵.

Chemical Induction of Dimerization—Dual Switch

Chemical Induction of Dimerization (CID) with small molecules is an effective technology used to generate switches of protein function to alter cell physiology. A high specificity, efficient dimerizer is rimiducid (AP1903), which has two identical, protein-binding surfaces arranged tail-to-tail, each with high affinity and specificity for a mutant of FKBP12: FKBP12(F36V) (FKBP12v36, Fᵥ₃₆ or Fᵥ), Attachment of one or more Fᵥ domains onto one or more cell signaling molecules that normally rely on homodimerization can convert that protein to rimiducid control. Homodimerization with rimiducid is used in the context of an inducible caspase safety switch, and an inducible activation switch for cellular therapy, where MyD88 and a costimulatory polypeptide cytoplasmic region are used to stimulate immune activity.

Another CID that may be used to activate the inducible chimeric signaling polypeptides is based on a heterodimerizer, such as Rapamycin, or a rapamycin analog (“rapalog”). In these embodiments, the multimeric ligand binding region provided in the inducible chimeric signaling polypeptides, or the multimeric ligand binding region provided in the inducible chimeric caspase polypeptides binds to Rapamycin or a rapalog and does not bind to rimiducid. Rapamycin binds to FKBP12, and its variants, and can induce heterodimerization of signaling domains that are fused to FKBP12 by binding to both FKBP12 and to polypeptides that contain the FKBP-rapamycin-binding (FRB) domain of mTOR.

In some embodiments, a dual switch is provided where the nucleic acid that encodes the inducible chimeric signaling polypeptide also encodes an inducible chimeric caspase polypeptide, for example, an inducible chimeric caspase 9 polypeptide. In some embodiments, modified cells are provided that express an inducible chimeric signaling polypeptide and an inducible chimeric caspase polypeptide, for example, an inducible chimeric caspase 9 polypeptide. The multimeric ligand binding regions provided in these two distinct polypeptides are different. In one example, the inducible chimeric signaling polypeptide comprises an FRB multimeric ligand binding domain, and the inducible chimeric caspase 9 polypeptide comprises an FKBP12 variant that binds to rimiducid. In this example, a dual control system is provided. Contacting the cells with rapamycin or a rapalog induces the immune cell activity by multimerizing the inducible chimeric signaling polypeptide. Contacting the cells with rimiducid induces apoptosis by multimerizing the inducible chimeric caspase polypeptide. In other embodiments, a rapamycin or rapalog-inducible pro-apoptotic polypeptide, such as, for example, Caspase-9 or a rapamycin or rapalog-inducible chimeric signaling polypeptide, such as, for example, MyD88/4-1BB, OX40, ICOS, or CD28, (iM-X) is used in combination with a rimiducid-inducible pro-apoptotic polypeptide, such as, for example, Caspase-9, or a rimiducid-inducible iM-X, to produce dual switches. These dual switches can be used to control both cell proliferation and activity, and apoptosis selectively by administration of either of two distinct ligand inducers.

The multimerizing regions, such as FKBP12/FRB, FRB/FKBP12, and FKBP12v36, may be located amino terminal to the pro-apoptotic polypeptide or signaling polypeptide, or, in other examples, may be located carboxyl terminal to the pro-apoptotic polypeptide or signaling polypeptide. Additional polypeptides, such as, for example, linker polypeptides, stem polypeptides, spacer polypeptides, or in some examples, marker polypeptides, may be located between the multimerizing region and the pro-apoptotic polypeptide or costimulatory polypeptide, in the chimeric polypeptides.

As used here, the term “rapalog” is meant as an analog of the natural antibiotic rapamycin. Certain rapalogs in the present embodiments have properties such as stability in serum, a poor affinity to wildtype FRB (and hence the parent protein, mTOR, leading to reduction or elimination of immunosuppressive properties), and a relatively high affinity to a mutant FRB domain. For commercial purposes, in certain embodiments, the rapalogs have useful scaling and production properties. Examples of rapalogs include, but are not limited to, S-o,p-dimethoxyphenyl (DMOP)-rapamycin: EC₅₀ (wt FRB (K2095 T2098 W2101)˜1000 nM), EC₅₀ (FRB-KLW˜5 nM) Luengo J I (95) Chem & Biol 2:471-81; Luengo J I (94) J. Org Chem 59:6512-6513; U.S. Pat. No. 6,187,757; R-Isopropoxyrapamycin: EC₅₀ (wt FRB (K2095 T2098 W2101)˜300 nM), EC50 (FRB-PLF˜8.5 nM); Liberles S (97) PNAS 94: 7825-30; and S-Butanesulfonamidorap (AP23050): EC₅₀ (wt FRB (K2095 T2098 W2101)˜2.7 nM), EC₅₀ (FRB-KTF˜>200 nM) Bayle (06) Chem & Bio. 13: 99-107; C7-Isobutyloxyrapamycin; 40-(S)-Fluoro-Rapamycin; 40-(S)-Chloro-Rapamycin; 40-(S)-Bromo-Rapamycin; 40-(S)-lodo-Rapamycin; 40-(S)-Amino-Rapamycin; 40-(S)-Fluoro-7-(S)-DMOP-Rapamycin; 40-(S)-Chloro-7-(S)-DMOP-Rapamycin; 40-(S)-lodo-7-(S)-DMOP-Rapamycin; 40-(S)-Azide-7-(S)-DMOP-Rapamycin; 40-(R)-p-Bromomethylbenzoyl-Rapamycin; 40-(R)-p-Chloromethylbenzoyl-Rapamycin; 40-(R)-((4-methylpiperazin-1-yl)p-methylbenzoyl)-Rapamycin; di-p-Bromomethylbenzoyl-Rapamycin; and 40-(S)—N-(3-(4-methylpiperazin-1-yl)propyl)-Rapamycinamine (see, e.g., U.S. Patent Application Publication No. US2017/0166877, which is incorporated by reference herein), R and S C7-ethyloxyrapamycin, R and S C7-isopropyloxyrapamycin, R and S C7-isobutylrapamycin, R and S ethylcarbamaterapamycin, R and S C7-phenylcarbamaterapamycin, R and S C7-(3-methyl)indole rapamycin, temsirolimus, everolimus, zotarolimus, and R and S C7-(7-methyl)indole rapamycin.

The term “FRB” refers to the FKBP12-Rapamycin-Binding (FRB) domain (residues 2015-2114 encoded within mTOR), and analogs thereof. In certain embodiments, FRB variants are provided. The properties of an FRB variant are stability (some variants are more labile than others) and ability to bind to various rapalogs. Based on the crystal structure conjugated to rapamycin, there are 3 key rapamycin-interacting residues that have been most analyzed, K2095, T2098, and W2101. Mutation of all three leads to an unstable protein that can be stabilized in the presence of rapamycin or some rapalogs. This feature can be used to further increase the signal: noise ratio in some applications. Examples of mutants are discussed in Bayle et al (06) Chem & Bio 13: 99-107; Stankunas et al (07) Chembiochem 8:1162-1169; and Liberles S (97) PNAS 94:7825-30). Examples of FRB regions of the present embodiments include, but are not limited to, KLW (with L2098); KTF (with F2101); and KLF (L2098, F2101). Heterodimerization is discussed in, for example, Belshaw, P., et al., PNAS 93:4604-4607 (1996). Additional compositions and methods are discussed, for example, in U.S. patent application Ser. No. 14/968,737, titled Methods for Controlled Elimination of Therapeutic Cells by Spencer, D., et al., filed Dec. 14, 2015, published as US-2016-0166613A1 on Jun. 16, 2016; International Patent Application PCT/US2015/065629, published as WO2016/100236 on Jun. 23, 2016; U.S. patent application Ser. No. 14/968,853 titled Methods for Controlled Activation or Elimination of Therapeutic Cells, by Spencer, D., et al., filed Dec. 14, 2015, published as US-2016-0175359A1 on Jun. 23, 2016; International Patent Application PCT/US2015/065646 filed Dec. 14, 2015, published as WO2016/100241 on Sep. 15, 2016; International Patent Application PCT/US2015/065629, filed Dec. 14, 2015, published as WO2016/100236 on Jun. 23, 2016; International Patent Application PCT/US2016/066371, filed Dec. 13, 2016, titled Dual Controls for Therapeutic Cell Activation or Elimination, by Bayle, J. H., et al.; and U.S. patent application Ser. No. 15/377,776, filed Dec. 13, 2016, titled Dual Controls for Therapeutic Cell Activation or Elimination, by Bayle, J. H., et al., each of which is hereby incorporated by reference herein in its entirety.

The ligands used are capable of binding to two or more of the ligand binding domains. The chimeric proteins may be able to bind to more than one ligand when they contain more than one ligand binding domain. The ligand is typically a non-protein or a chemical. Exemplary ligands include, but are not limited to FK506 (e.g., FK1012).

Other ligand binding regions may be, for example, dimeric regions, or modified ligand binding regions with a wobble substitution, such as, for example, FKBP12(V36): The human 12 kDa FK506-binding protein with an F36 to V substitution, the complete mature coding sequence (amino acids 1-107), provides a binding site for synthetic dimerizer drug rimiducid (Jemal, A. et al., CA Cancer J. Clinic. 58, 71-96 (2008); Scher, H. I. and Kelly, W. K., Journal of Clinical Oncology 11, 1566-72 (1993)). Two tandem copies of the protein may also be used in the construct so that higher-order oligomers are induced upon cross-linking by rimiducid.

FKBP12 variants may also be used in the FKBP12/FRB multimerizing regions. Variants used in these fusions, in some embodiments, will bind to rapamycin, or rapalogs, but will bind to less affinity to rimiducid than, for example, FKBP12v36. Examples of FKBP12 variants include those from many species, including, for example, yeast. In one embodiment, the FKBP12 variant is FKBP12.6 (calstablin).

Other heterodimers are contemplated in the present application. In one embodiment, a calcineurin-A polypeptide, or region may be used in place of the FRB multimerizing region. In some embodiments, the first unit of the first multimerizing region is a calcineurin-A polypeptide. In some embodiments, the first unit of the first multimerizing region is a calcineurin-A polypeptide region and the second unit of the first multimerizing region is a FKBP12 or FKBP12 variant multimerizing region. In some embodiments, the first unit of the first multimerizing region is a FKBP12 or FKBP12 variant multimerizing region and the second unit of the first multimerizing region is a calcineurin-A polypeptide region. In these embodiments, the first ligand comprises, for example, cyclosporine.

Immune Cell Therapy and Inducible Chimeric Signaling Polypeptides

In some embodiments, T cells are modified to express a chimeric antigen receptor that comprises a single chain antibody variable fragment (scFv) fused with a transmembrane domain containing linker region and an intracellular domain derived from the CD3 zeta component. In natural T cells signals from CD3zeta drive the initial activation of the T cell through signaling to the NF-ATc transcription factor. These signals are necessary to drive target cell killing in cytotoxic T lymphocytes and synergize with costimulatory signaling pathways to drive the robust cell proliferation of T cell immune response. The T cells may be modified by transduction or transfection with a nucleic acid that expresses the CAR in the absence of any coding region for a chimeric signaling polypeptide. Or, in other examples, the polynucleotide that encodes the CAR may be provided as part of a nucleic acid that also comprises a polynucleotide that encodes a chimeric signaling polypeptide.

For inducible costimulation, the CAR-T cells are also modified, for example, by transfection or transduction of the cells with a nucleic acid that expresses an inducible chimeric signaling polypeptide. For example, in some embodiments, the polypeptide is inducible based on a drug inducible mediator of costimulatory signaling in which FKBP12 in two copies is fused with MyD88. FKBP12 is a small (107 amino acid) prolylyl isomerase that is also the ligand for the natural antibiotic and immunosuppressant macrolides rapamycin, FK-506 and ascomycin. In certain embodiments, a single mutant of FKBP12 substituting valine at amino acid 36 for phenylalanine (Fv) confers inducibility to Fv fusions with the synthetic ligand rimiducid (AP1903) by homodimerization of FKBP12 moieties. MyD88 is critical mediator of signals in the innate immune response downstream of Toll-Like Receptors (TLR) typically in myeloid cells but also in lymphocytes. These signals activate transcription regulators including the family of NF-κB factors.

Rimiducid is a tail-to-tail linkage of a high affinity synthetic ligand specific for Fv and not wild-type FKBP12. It is a dimerizing ligand because it can simultaneously bind with two Fv moieties. The drug-directed dimerizing event thereby juxtaposes the fused MyD88 moieties which initiates robust signal transduction. This is demonstrated in retroviral construct 1810 and is denoted iM. The Fv-MyD88 fusion is linked with a second costimulatory signaling domain derived from the intracellular domain of CD40 to generate iMC. iMC has been utilized to activate proliferation and cytokine production in myeloid cells and in CAR-T cells and retroviral vector BP2212 is an example of an iMC-CAR expression construct.

Alternative signaling domains, when fused with iMyD88, generate distinct signaling outcomes. Construct BP1798 expresses an inducible fusion of MyD88 with the intracellular domain of CD28, the canonical costimulatory receptor for the CD80/CD86 ligands of antigen presenting cells. Construct BP1799 fuses iMyD88 with the intracellular signaling domain of 4-1BB (also called CD137, a costimulatory receptor present in activated T cells). Construct BP1801 expresses iMyD88 fused with the signaling domain of OX40. OX40 is a member of the Tumor Necrosis Factor Receptor (TNFR) superfamily that signal to NF-κB through TRAF proteins. Construct BP1802 expresses iMyD88 fused with the signaling domain of ICOS (Inducible COSstimulator, also called CD278) a member of the CD28 family which signals to NF-κB through a mechanism distinct from TNF-β family members. Construct BP1800 expresses iMyD88 fused with the signaling domains of OX40 and CD28.

Immune Cell Therapy and Constitutive Chimeric Signaling Polypeptides

Immune cell therapies may also be designed to provide constitutively active therapy, such as constitutively active CAR-T cells, but provide an inducible safety switch, to stop, or reduce the level of, the therapy when needed. In some embodiments, immune cells, such as CAR-T cells, express a chimeric antigen receptor, and a chimeric signaling polypeptide comprising a truncated MyD88 polypeptide and a stimulating polypeptide. In this format, the multimeric ligand binding region is not fused with the truncated MyD88 polypeptide. High level costimulation is provided constitutively through an alternate mechanism in which a leaky 2A cotranslational sequence, for example one derived from porcine teschovirus-1 (P2A), is used to separate the CAR from the chimeric MyD88 polypeptide. When the chimeric MyD88 polypeptide is a MyD88-CD40 polypeptide, most MC remains cytosolic but the leakiness in the P2A sequence retains a portion (estimated to be about 10%) of MC fused with the CAR and thereby expressed at the plasma membrane. This membrane proximal expression produces a high level of signaling activity.

In some embodiments, the modified cells comprise a non-inducible chimeric polypeptide that is not induced by contact with a ligand inducer, or dimerizer, or CID, such as, for example, rimiducid AP20187, or AP1510. In some embodiments, the modified cells comprise a chimeric polypeptide that does not bind rapamycin, a rapalog, rimiducid, AP20187, or AP1510. In some embodiments, the modified cells comprise a chimeric polypeptide that does not comprise a multimeric ligand binding region, and does not comprise, for example, an FKBP12 polypeptide region or an FRB region, or variants thereof. In some embodiments, the chimeric polypeptide does not have a multimeric ligand binding region, and does not have an FKBP12 polypeptide region, or FRB region, or variants thereof. In some embodiments, the chimeric polypeptide does not have a functional multimeric ligand binding region.

The inducible component in these modified cells is an inducible Caspase-9 polypeptide, for example, an Fv fusion with caspase 9 (iC9) that rapidly induces apoptosis, or programmed cell death, in a rimiducid dependent fashion. This iC9 safety switch can thereby be deployed to block adverse events that may result from CAR-T therapy such as graft versus host disease or cytokine release syndrome. In animal studies inducible caspase-9 polypeptide-expressing T cells containing MyD88-CD40 (MC) produce robust anti-tumor effects that may have toxic effects on the animals that necessitate rimiducid treatment to remove the most active CAR-T cells. The toxic effects are consistent with a cytokine release syndrome that is likely due to excessive production of inflammatory cytokines such as TNF-α and IL-6.

Also provided herein are chimeric signaling polypeptides that do not include a multimeric ligand binding region. These polypeptides provide constitutive T cell activation activity; the polypeptides may be provided in immune cells, such as T cells, in which an inducible apoptotic polypeptide, such as Caspase 9 may be expressed.

Thus, in some embodiments, provided are modified cells comprising chimeric signaling polypeptides, and nucleic acids comprising polynucleotides that encode chimeric signaling polypeptides. The modified cells may further comprise a chimeric antigen receptor or a recombinant T cell receptor. In some embodiments, the nucleic acid that comprises a polynucleotide that encodes a chimeric signaling polypeptide comprises a polynucleotide that encodes a chimeric antigen receptor or a recombinant T cell receptor.

The modified cells may be, for example, selected from the group consisting of T cells, NK cells, invariant NK-T cells, and gamma delta T cells.

Chimeric signaling polypeptides provided in the present embodiments include, for example, inducible chimeric signaling polypeptides comprising regions provided in Table 1. Costimulatory polypeptide cytoplasmic signaling regions include polypeptides and sequences provided herein, for purposes of the present application. In the following table, a MyD88 polypeptide region that is not truncated, such as, for example, a MyD88 polypeptide that comprises the TIR domain may be used in place of a truncated MyD88 polypeptide region lacking the TIR domain. The chimeric signling polypeptides of Table 1 may lack a membrane-targeting region, such as, for example, a myristoylation region. The order of the regions provided in the following table may vary; that is, the multimerizing region may be provided either at the amino terminal or carboxyl terminal portion of the polypeptide relative to the signaling region. The first and second ligand binding regions may further be provided in either order, and the truncated MyD88 polypeptide and costimulatory polypeptide cytoplasmic signaling regions may be provided in either order relative to the multimerizing region. In some embodiments, the FKBP12v36 polypeptide of Table 1 may be substituted with any FKBP12 variant polypeptide having an amino acid substitution at position 36 other than phenylalanine to valine, that binds to AP1903, AP20187, rapamycin, or a rapalog. In some embodiments, the FRB variant polypeptide region may be FRB_{L}, as shown in Table 1. In some embodiments, the FRB variant polypeptide is selected from, but is not limited to, the group consisting of KLW (with L2098); KTF (with F2101); and KLF (L2098, F2101).

In some embodiments, the costimulatory polypeptide cytoplasmic signaling region, or co-activation polypeptide cytoplasmic signaling region is selected from the group consisting of CD28, 4-1BB, OX40, ICOS, BCMA, CD27, CD30, CD122, GITR S180A, GITR EEE191RVV, HVEM, TWEAKR, RANK, RANK TRAF6, RANK TRAF 2/5, RANK HCR-TRAF2/5, CD40-HCR-CD40, SOD1, SOD2, TAC1, SOD3, RANK88, IL15Ra, BTN3A1 (B30.2 domain), CD86, CD74Ra, Dectin, ITA4, ITGA5, TCL1A, CTLA4, TIM1, TREMBL2, CD40-HCR-CD40, and DPP4 cytoplasmic signaling regions. In some embodiments, the costimulatory polypeptide cytoplasmic signaling region, or co-activation polypeptide cytoplasmic signaling region is selected from the group consisting of BCMA, CD27, CD30, CD122, GITR S180A, GITR EEE191RVV, HVEM, TWEAKR, RANK, RANK TRAF6, RANK TRAF 2/5, RANK HCR-TRAF2/5, SOD1, SOD2, TAC1, SOD3, RANK88, IL15Ra, BTN3A1 (B30.2 domain), CD86, CD74Ra, Dectin, ITA4, ITGA5, TCL1A, CTLA4, TIM1, TREMBL2, and DPP4 cytoplasmic signaling regions. In some embodiments, the costimulatory polypeptide cytoplasmic signaling region, or co-activation polypeptide cytoplasmic signaling region is selected from the group consisting of CD28, 4-1BB, OX40, ICOS, BCMA, CD27, CD30, CD122, GITR S180A, GITR EEE191RVV, HVEM, TWEAKR, RANK, RANK TRAF6, RANK TRAF 2/5, RANK HCR-TRAF2/5, CD40-HCR-CD40, SOD1, SOD2, TAC1, SOD3, RANK88, IL15Ra, BTN3A1 (B30.2 domain), CD86, CD74Ra, Dectin, ITA4, ITGA5, TCL1A, CTLA4, TIM1, TREMBL2, and DPP4 cytoplasmic signaling regions. In some embodiments, the costimulatory polypeptide cytoplasmic signaling region, or co-activation polypeptide cytoplasmic signaling region is selected from the group consisting of RANK, RANK TRAF6, RANK TRAF 2/5, RANK HCR-TRAF2/5, and RANK88 cytoplasmic signaling regions. In some embodiments, the costimulatory polypeptide cytoplasmic signaling region, or co-activation polypeptide cytoplasmic signaling region is selected from the group consisting of RANK, RANK TRAF6, RANK TRAF 2/5, RANK HCR-TRAF2/5, CD40-HCR-CD40, and RANK88 cytoplasmic signaling regions. In some embodiments, the costimulatory polypeptide cytoplasmic signaling region is a 4-1BB cytoplasmic polypeptide signaling region.

TABLE 1

| | | Signaling region | |
|---|---|---|---|
| | | Truncated | |
| | | MyD88 | |
| | | polypeptide | Costimulatory |
| Multimerizing Region | | region lacking | polypeptide |
| First Ligand | Second Ligand | the TIR domain | cytoplasmic |
| Binding Region | Binding Region | (tMyD88) | signaling region |
| FKBP12v36 | FKBP12v36 | tMyD88 | CD28 |
| FKBP12v36 | FKBP12v36 | tMyD88 | 4-1BB |
| FKBP12v36 | FKBP12v36 | tMyD88 | OX40 |
| FKBP12v36 | FKBP12v36 | tMyD88 | ICOS |
| FKBP12v36 | FKBP12v36 | tMyD88 | BCMA |
| FKBP12v36 | FKBP12v36 | tMyD88 | CD27 |
| FKBP12v36 | FKBP12v36 | tMyD88 | CD30 |
| FKBP12v36 | FKBP12v36 | tMyD88 | CD122 |
| FKBP12v36 | FKBP12v36 | tMyD88 | GITR S180A |
| FKBP12v36 | FKBP12v36 | tMyD88 | GITR |
| | | | EEE191RVV |
| FKBP12v36 | FKBP12v36 | tMyD88 | HVEM |
| FKBP12v36 | FKBP12v36 | tMyD88 | TWEAKR |
| FKBP12v36 | FKBP12v36 | tMyD88 | RANK |
| FKBP12v36 | FKBP12v36 | tMyD88 | RANK TRAF6 |
| FKBP12v36 | FKBP12v36 | tMyD88 | RANK TRAF 2/5 |
| FKBP12v36 | FKBP12v36 | tMyD88 | RANK HCR- |
| | | | TRAF2/5 |
| FKBP12v36 | FKBP12v36 | tMyD88 | CD40-HCR-CD40 |
| FKBP12v36 | FKBP12v36 | tMyD88 | SOD1 |
| FKBP12v36 | FKBP12v36 | tMyD88 | SOD2 |
| FKBP12v36 | FKBP12v36 | tMyD88 | Costimulatory |
| | | | polypeptide |
| | | | cytoplasmic |
| | | | signaling region |
| | | | selected from the |
| | | | group consisting |
| | | | of TAC1, SOD3, |
| | | | RANK88, IL15Ra, |
| | | | BTN3A1 (B30.2 |
| | | | domain), CD86, |
| | | | CD74Ra, Dectin, |
| | | | ITA4, ITGA5, |
| | | | TCL1A, CTLA4, |
| | | | TIM1, TREMBL2, |
| | | | CD40-HCR-CD40, |
| | | | and DPP4 |
| FKBP12wt | FKBP12v36 | tMyD88 | CD28 |
| FKBP12wt | FKBP12v36 | tMyD88 | 4-1BB |
| FKBP12wt | FKBP12v36 | tMyD88 | OX40 |
| FKBP12wt | FKBP12v36 | tMyD88 | ICOS |
| FKBP12wt | FKBP12v36 | tMyD88 | BCMA |
| FKBP12wt | FKBP12v36 | tMyD88 | CD27 |
| FKBP12wt | FKBP12v36 | tMyD88 | CD30 |
| FKBP12wt | FKBP12v36 | tMyD88 | CD122 |
| FKBP12wt | FKBP12v36 | tMyD88 | GITR S180A |
| FKBP12wt | FKBP12v36 | tMyD88 | GITR |
| | | | EEE191RVV |
| FKBP12wt | FKBP12v36 | tMyD88 | HVEM |
| FKBP12wt | FKBP12v36 | tMyD88 | TWEAKR |
| FKBP12wt | FKBP12v36 | tMyD88 | RANK |
| FKBP12wt | FKBP12v36 | tMyD88 | RANK TRAF6 |
| FKBP12wt | FKBP12v36 | tMyD88 | RANK TRAF 2/5 |
| FKBP12wt | FKBP12v36 | tMyD88 | RANK HCR- |
| | | | TRAF2/5 |
| FKBP12wt | FKBP12v36 | tMyD88 | CD40-HCR-CD40 |
| FKBP12wt | FKBP12v36 | tMyD88 | SOD1 |
| FKBP12wt | FKBP12v36 | tMyD88 | SOD2 |
| FKBP12wt | FKBP12v36 | tMyD88 | Costimulatory |
| | | | polypeptide |
| | | | cytoplasmic |
| | | | signaling region |
| | | | selected from the |
| | | | group consisting |
| | | | of TAC1, SOD3, |
| | | | RANK88, IL15Ra, |
| | | | BTN3A1 (B30.2 |
| | | | domain), CD86, |
| | | | CD74Ra, Dectin, |
| | | | ITA4, ITGA5, |
| | | | TCL1A, CTLA4, |
| | | | TIM1, TREMBL2, |
| | | | CD40-HCR-, |
| | | | CD40, and DPP4 |
| FKBP12wt | FKBP12wt | tMyD88 | CD28 |
| FKBP12wt | FKBP12wt | tMyD88 | 4-1BB |
| FKBP12wt | FKBP12wt | tMyD88 | OX40 |
| FKBP12wt | FKBP12wt | tMyD88 | ICOS |
| FKBP12wt | FKBP12wt | tMyD88 | BCMA |
| FKBP12wt | FKBP12wt | tMyD88 | CD27 |
| FKBP12wt | FKBP12wt | tMyD88 | CD30 |
| FKBP12wt | FKBP12wt | tMyD88 | CD122 |
| FKBP12wt | FKBP12wt | tMyD88 | GITR S180A |
| FKBP12wt | FKBP12wt | tMyD88 | GITR |
| | | | EEE191RVV |
| FKBP12wt | FKBP12wt | tMyD88 | HVEM |
| FKBP12wt | FKBP12wt | tMyD88 | TWEAKR |
| FKBP12wt | FKBP12wt | tMyD88 | RANK |
| FKBP12wt | FKBP12wt | tMyD88 | RANK TRAF6 |
| FKBP12wt | FKBP12wt | tMyD88 | RANK TRAF 2/5 |
| FKBP12wt | FKBP12wt | tMyD88 | RANK HCR- |
| | | | TRAF2/5 |
| FKBP12wt | FKBP12wt | tMyD88 | CD40-HCR-CD40 |
| FKBP12wt | FKBP12wt | tMyD88 | SOD1 |
| FKBP12wt | FKBP12wt | tMyD88 | SOD2 |
| FKBP12wt | FKBP12wt | tMyD88 | Costimulatory |
| | | | polypeptide |
| | | | cytoplasmic |
| | | | signaling region |
| | | | selected from the |
| | | | group consisting |
| | | | of TAC1, SOD3, |
| | | | RANK88, IL15Ra, |
| | | | BTN3A1 (B30.2 |
| | | | domain), CD86, |
| | | | CD74Ra, Dectin, |
| | | | ITA4, ITGA5, |
| | | | TCL1A, CTLA4, |
| | | | TIM1, TREMBL2, |
| | | | CD40-HCR- |
| | | | CD40, and DPP4 |
| FRB | FKBP12v36 | tMyD88 | CD28 |
| FRB | FKBP12v36 | tMyD88 | 4-1BB |
| FRB | FKBP12v36 | tMyD88 | OX40 |
| FRB | FKBP12v36 | tMyD88 | ICOS |
| FRB | FKBP12v36 | tMyD88 | BCMA |
| FRB | FKBP12v36 | tMyD88 | CD27 |
| FRB | FKBP12v36 | tMyD88 | CD30 |
| FRB | FKBP12v36 | tMyD88 | CD122 |
| FRB | FKBP12v36 | tMyD88 | GITR S180A |
| FRB | FKBP12v36 | tMyD88 | GITR |
| | | | EEE191RVV |
| FRB | FKBP12v36 | tMyD88 | HVEM |
| FRB | FKBP12v36 | tMyD88 | TWEAKR |
| FRB | FKBP12v36 | tMyD88 | RANK |
| FRB | FKBP12v36 | tMyD88 | RANK TRAF6 |
| FRB | FKBP12v36 | tMyD88 | RANK TRAF 2/5 |
| FRB | FKBP12v36 | tMyD88 | RANK HCR- |
| | | | TRAF2/5 |
| FRB | FKBP12v36 | tMyD88 | CD40-HCR-CD40 |
| FRB | FKBP12v36 | tMyD88 | SOD1 |
| FRB | FKBP12v36 | tMyD88 | SOD2 |
| FRB | FKBP12v36 | tMyD88 | Costimulatory |
| | | | polypeptide |
| | | | cytoplasmic |
| | | | signaling region |
| | | | selected from the |
| | | | group consisting |
| | | | of TAC1, SOD3, |
| | | | RANK88, IL15Ra, |
| | | | BTN3A1 (B30.2 |
| | | | domain), CD86, |
| | | | CD74Ra, Dectin, |
| | | | ITA4, ITGA5, |
| | | | TCL1A, CTLA4, |
| | | | TIM1, TREMBL2, |
| | | | CD40-HCR, |
| | | | CD40DPP4 |
| FRB | FKBP12wt | tMyD88 | CD28 |
| FRB | FKBP12wt | tMyD88 | 4-1BB |
| FRB | FKBP12wt | tMyD88 | OX40 |
| FRB | FKBP12wt | tMyD88 | ICOS |
| FRB | FKBP12wt | tMyD88 | BCMA |
| FRB | FKBP12wt | tMyD88 | CD27 |
| FRB | FKBP12wt | tMyD88 | CD30 |
| FRB | FKBP12wt | tMyD88 | CD122 |
| FRB | FKBP12wt | tMyD88 | GITR S180A |
| FRB | FKBP12wt | tMyD88 | GITR |
| | | | EEE191RVV |
| FRB | FKBP12wt | tMyD88 | HVEM |
| FRB | FKBP12wt | tMyD88 | TWEAKR |
| FRB | FKBP12wt | tMyD88 | RANK |
| FRB | FKBP12wt | tMyD88 | RANK TRAF6 |
| FRB | FKBP12wt | tMyD88 | RANK TRAF 2/5 |
| FRB | FKBP12wt | tMyD88 | RANK HCR- |
| | | | TRAF2/5 |
| FRB | FKBP12wt | tMyD88 | CD40-HCR-CD40 |
| FRB | FKBP12wt | tMyD88 | SOD1 |
| FRB | FKBP12wt | tMyD88 | SOD2 |
| FRB | FKBP12wt | tMyD88 | Costimulatory |
| | | | polypeptide |
| | | | cytoplasmic |
| | | | signaling region |
| | | | selected from the |
| | | | group consisting |
| | | | of TAC1, SOD3, |
| | | | RANK88, IL15Ra, |
| | | | BTN3A1 (B30.2 |
| | | | domain), CD86, |
| | | | CD74Ra, Dectin, |
| | | | ITA4, ITGA5, |
| | | | TCL1A, CTLA4, |
| | | | TIM1, TREMBL2, |
| | | | CD40-HCR- |
| | | | CD40, and DPP4 |
| FRB_{L} | FKBP12v36 | tMyD88 | CD28 |
| FRB_{L} | FKBP12v36 | tMyD88 | 4-1BB |
| FRB_{L} | FKBP12v36 | tMyD88 | OX40 |
| FRB_{L} | FKBP12v36 | tMyD88 | ICOS |
| FRB_{L} | FKBP12v36 | tMyD88 | BCMA |
| FRB_{L} | FKBP12v36 | tMyD88 | CD27 |
| FRB_{L} | FKBP12v36 | tMyD88 | CD30 |
| FRB_{L} | FKBP12v36 | tMyD88 | CD122 |
| FRB_{L} | FKBP12v36 | tMyD88 | GITR S180A |
| FRB_{L} | FKBP12v36 | tMyD88 | GITR |
| | | | EEE191RVV |
| FRB_{L} | FKBP12v36 | tMyD88 | HVEM |
| FRB_{L} | FKBP12v36 | tMyD88 | TWEAKR |
| FRB_{L} | FKBP12v36 | tMyD88 | RANK |
| FRB_{L} | FKBP12v36 | tMyD88 | RANK TRAF6 |
| FRB_{L} | FKBP12v36 | tMyD88 | RANK TRAF 2/5 |
| FRB_{L} | FKBP12v36 | tMyD88 | RANK HCR- |
| | | | TRAF2/5 |
| FRB_{L} | FKBP12v36 | tMyD88 | CD40-HCR-CD40 |
| FRB_{L} | FKBP12v36 | tMyD88 | SOD1 |
| FRB_{L} | FKBP12v36 | tMyD88 | SOD2 |
| FRB_{L} | FKBP12v36 | tMyD88 | Costimulatory |
| | | | polypeptide |
| | | | cytoplasmic |
| | | | signaling region |
| | | | selected from the |
| | | | group consisting |
| | | | of TAC1, SOD3, |
| | | | RANK88, IL15Ra, |
| | | | BTN3A1 (B30.2 |
| | | | domain), CD86, |
| | | | CD74Ra, Dectin, |
| | | | ITA4, ITGA5, |
| | | | TCL1A, CTLA4, |
| | | | TIM1, TREMBL2, |
| | | | CD40-HCR- |
| | | | CD40, and DPP4 |
| FRB_{L} | FKBP12wt | tMyD88 | CD28 |
| FRB_{L} | FKBP12wt | tMyD88 | 4-1BB |
| FRB_{L} | FKBP12wt | tMyD88 | OX40 |
| FRB_{L} | FKBP12wt | tMyD88 | ICOS |
| FRB_{L} | FKBP12wt | tMyD88 | BCMA |
| FRB_{L} | FKBP12wt | tMyD88 | CD27 |
| FRB_{L} | FKBP12wt | tMyD88 | CD30 |
| FRB_{L} | FKBP12wt | tMyD88 | CD122 |
| FRB_{L} | FKBP12wt | tMyD88 | GITR S180A |
| FRB_{L} | FKBP12wt | tMyD88 | GITR |
| | | | EEE191RVV |
| FRB_{L} | FKBP12wt | tMyD88 | HVEM |
| FRB_{L} | FKBP12wt | tMyD88 | TWEAKR |
| FRB_{L} | FKBP12wt | tMyD88 | RANK |
| FRB_{L} | FKBP12wt | tMyD88 | RANK TRAF6 |
| FRB_{L} | FKBP12wt | tMyD88 | RANK TRAF 2/5 |
| FRB_{L} | FKBP12wt | tMyD88 | RANK HCR- |
| | | | TRAF2/5 |
| FRB_{L} | FKBP12wt | tMyD88 | CD40-HCR-CD40 |
| FRB_{L} | FKBP12wt | tMyD88 | SOD1 |
| FRB_{L} | FKBP12wt | tMyD88 | SOD2 |
| FRB_{L} | FKBP12wt | tMyD88 | Costimulatory |
| | | | polypeptide |
| | | | cytoplasmic |
| | | | signaling region |
| | | | selected from the |
| | | | group consisting |
| | | | of TAC1, SOD3, |
| | | | RANK88, IL15Ra, |
| | | | BTN3A1 (B30.2 |
| | | | domain), CD86, |
| | | | CD74Ra, Dectin, |
| | | | ITA4, ITGA5, |
| | | | TCL1A, CTLA4, |
| | | | TIM1, TREMBL2, |
| | | | CD40-HCR- |
| | | | CD40, and DPP4 |

Chimeric signaling polypeptides provided in the present embodiments include, for example, inducible chimeric signaling polypeptides comprising regions provided in Table 2. Costimulatory polypeptide cytoplasmic signaling regions include polypeptides and sequences provided herein, for purposes of the present application. In the following table, a MyD88 polypeptide region that is not truncated, such as, for example, a MyD88 polypeptide that comprises the TIR domain may be used in place of a truncated MyD88 polypeptide region lacking the TIR domain. The chimeric signling polypeptides of Table 2 may comprise a membrane-targeting region, such as, for example, a myristoylation region. A myristoylation region is indicated as “myr” in Table 2, however, it is understood that the chimeric signaling polypeptides may comprise any appropriate membrane targeting region, such as a membrane-targeting region provided herein. The order of the regions provided in the following table may vary; that is, the multimerizing region may be provided either at the amino terminal or carboxyl terminal portion of the polypeptide relative to the signaling region. The first and second ligand binding regions may further be provided in either order, and the truncated MyD88 polypeptide and costimulatory polypeptide cytoplasmic signaling regions may be provided in either order relative to the multimerizing region. In some embodiments, the FKBP12v36 polypeptide of Table 2 may be substituted with any FKBP12 variant polypeptide having an amino acid substitution at position 36 other than phenylalanine to valine, that binds to AP1903, AP20187, rapamycin, or a rapalog. In some embodiments, the FRB variant polypeptide region may be FRB_{L}, as shown in Table 2. In some embodiments, the FRB variant polypeptide is selected from, but is not limited to, the group consisting of KLW (with L2098); KTF (with F2101); and KLF (L2098, F2101).

TABLE 2

| | | Signaling region | | |
|---|---|---|---|---|
| | | Truncated | | |
| | | MyD88 | | |
| | | polypeptide | Costimulatory | Membrane-targeting |
| Multimerizing Region | | region lacking | polypeptide | Region |
| First Ligand | Second Ligand | the TIR domain | cytoplasmic | Membrane-targeting |
| Binding Region | Binding Region | (tMyD88) | signaling region | region |
| FKBP12v36 | FKBP12v36 | tMyD88 | CD28 | Myr |
| FKBP12v36 | FKBP12v36 | tMyD88 | 4-1BB | Myr |
| FKBP12v36 | FKBP12v36 | tMyD88 | OX40 | Myr |
| FKBP12v36 | FKBP12v36 | tMyD88 | ICOS | Myr |
| FKBP12v36 | FKBP12v36 | tMyD88 | BCMA | Myr |
| FKBP12v36 | FKBP12v36 | tMyD88 | CD27 | Myr |
| FKBP12v36 | FKBP12v36 | tMyD88 | CD30 | Myr |
| FKBP12v36 | FKBP12v36 | tMyD88 | CD122 | Myr |
| FKBP12v36 | FKBP12v36 | tMyD88 | GITR S180A | Myr |
| FKBP12v36 | FKBP12v36 | tMyD88 | GITR | Myr |
| | | | EEE191RVV | |
| FKBP12v36 | FKBP12v36 | tMyD88 | HVEM | Myr |
| FKBP12v36 | FKBP12v36 | tMyD88 | TWEAKR | Myr |
| FKBP12v36 | FKBP12v36 | tMyD88 | RANK | Myr |
| FKBP12v36 | FKBP12v36 | tMyD88 | RANK TRAF6 | Myr |
| FKBP12v36 | FKBP12v36 | tMyD88 | RANK TRAF | Myr |
| | | | 2/5 | |
| FKBP12v36 | FKBP12v36 | tMyD88 | RANK HCR- | Myr |
| | | | TRAF2/5 | |
| FKBP12v36 | FKBP12v36 | tMyD88 | CD40-HCR- | Myr |
| | | | CD40 | |
| FKBP12v36 | FKBP12v36 | tMyD88 | SOD1 | Myr |
| FKBP12v36 | FKBP12v36 | tMyD88 | SOD2 | Myr |
| FKBP12v36 | FKBP12v36 | tMyD88 | Costimulatory | Myr |
| | | | polypeptide | |
| | | | cytoplasmic | |
| | | | signaling region | |
| | | | selected from | |
| | | | the group | |
| | | | consisting of | |
| | | | TAC1, SOD3, | |
| | | | RANK88, | |
| | | | IL15Ra, | |
| | | | BTN3A1 (B30.2 | |
| | | | domain), CD86, | |
| | | | CD74Ra, | |
| | | | Dectin, ITA4, | |
| | | | ITGA5, TCL1A, | |
| | | | CTLA4, TIM1, | |
| | | | TREMBL2, | |
| | | | CD40-HCR, | |
| | | | CD40DPP4 | |
| FKBP12wt | FKBP12v36 | tMyD88 | CD28 | Myr |
| FKBP12wt | FKBP12v36 | tMyD88 | 4-1BB | Myr |
| FKBP12wt | FKBP12v36 | tMyD88 | OX40 | Myr |
| FKBP12wt | FKBP12v36 | tMyD88 | ICOS | Myr |
| FKBP12wt | FKBP12v36 | tMyD88 | BCMA | Myr |
| FKBP12wt | FKBP12v36 | tMyD88 | CD27 | Myr |
| FKBP12wt | FKBP12v36 | tMyD88 | CD30 | Myr |
| FKBP12wt | FKBP12v36 | tMyD88 | CD122 | Myr |
| FKBP12wt | FKBP12v36 | tMyD88 | GITR S180A | Myr |
| FKBP12wt | FKBP12v36 | tMyD88 | GITR | Myr |
| | | | EEE191RVV | |
| FKBP12wt | FKBP12v36 | tMyD88 | HVEM | Myr |
| FKBP12wt | FKBP12v36 | tMyD88 | TWEAKR | Myr |
| FKBP12wt | FKBP12v36 | tMyD88 | RANK | Myr |
| FKBP12wt | FKBP12v36 | tMyD88 | RANK TRAF6 | Myr |
| FKBP12wt | FKBP12v36 | tMyD88 | RANK TRAF | Myr |
| | | | 2/5 | |
| FKBP12wt | FKBP12v36 | tMyD88 | RANK HCR- | Myr |
| | | | TRAF2/5 | |
| FKBP12wt | FKBP12v36 | tMyD88 | CD40-HCR- | Myr |
| | | | CD40 | |
| FKBP12wt | FKBP12v36 | tMyD88 | SOD1 | Myr |
| FKBP12wt | FKBP12v36 | tMyD88 | SOD2 | Myr |
| FKBP12wt | FKBP12v36 | tMyD88 | Costimulatory | Myr |
| | | | polypeptide | |
| | | | cytoplasmic | |
| | | | signaling region | |
| | | | selected from | |
| | | | the group | |
| | | | consisting of | |
| | | | TAC1, SOD3, | |
| | | | RANK88, | |
| | | | IL15Ra, | |
| | | | BTN3A1 (B30.2 | |
| | | | domain), CD86, | |
| | | | CD74Ra, | |
| | | | Dectin, ITA4, | |
| | | | ITGA5, TCL1A, | |
| | | | CTLA4, TIM1, | |
| | | | TREMBL2, | |
| | | | CD40-HCR- | |
| | | | CD40, and | |
| | | | DPP4 | |
| FKBP12wt | FKBP12wt | tMyD88 | CD28 | Myr |
| FKBP12wt | FKBP12wt | tMyD88 | 4-1BB | Myr |
| FKBP12wt | FKBP12wt | tMyD88 | OX40 | Myr |
| FKBP12wt | FKBP12wt | tMyD88 | ICOS | Myr |
| FKBP12wt | FKBP12wt | tMyD88 | BCMA | Myr |
| FKBP12wt | FKBP12wt | tMyD88 | CD27 | Myr |
| FKBP12wt | FKBP12wt | tMyD88 | CD30 | Myr |
| FKBP12wt | FKBP12wt | tMyD88 | CD122 | Myr |
| FKBP12wt | FKBP12wt | tMyD88 | GITR S180A | Myr |
| FKBP12wt | FKBP12wt | tMyD88 | GITR | Myr |
| | | | EEE191RVV | |
| FKBP12wt | FKBP12wt | tMyD88 | HVEM | Myr |
| FKBP12wt | FKBP12wt | tMyD88 | TWEAKR | Myr |
| FKBP12wt | FKBP12wt | tMyD88 | RANK | Myr |
| FKBP12wt | FKBP12wt | tMyD88 | RANK TRAF6 | Myr |
| FKBP12wt | FKBP12wt | tMyD88 | RANK TRAF | Myr |
| | | | 2/5 | |
| FKBP12wt | FKBP12wt | tMyD88 | RANK HCR- | Myr |
| | | | TRAF2/5 | |
| FKBP12wt | FKBP12wt | tMyD88 | CD40-HCR- | Myr |
| | | | CD40 | |
| FKBP12wt | FKBP12wt | tMyD88 | SOD1 | Myr |
| FKBP12wt | FKBP12wt | tMyD88 | SOD2 | Myr |
| FKBP12wt | FKBP12wt | tMyD88 | Costimulatory | Myr |
| | | | polypeptide | |
| | | | cytoplasmic | |
| | | | signaling region | |
| | | | selected from | |
| | | | the group | |
| | | | consisting of | |
| | | | TAC1, SOD3, | |
| | | | RANK88, | |
| | | | IL15Ra, | |
| | | | BTN3A1 (B30.2 | |
| | | | domain), CD86, | |
| | | | CD74Ra, | |
| | | | Dectin, ITA4, | |
| | | | ITGA5, TCL1A, | |
| | | | CTLA4, TIM1, | |
| | | | TREMBL2, | |
| | | | CD40-HCR- | |
| | | | CD40, and | |
| | | | DPP4 | |
| FRB | FKBP12v36 | tMyD88 | CD28 | Myr |
| FRB | FKBP12v36 | tMyD88 | 4-1BB | Myr |
| FRB | FKBP12v36 | tMyD88 | OX40 | Myr |
| FRB | FKBP12v36 | tMyD88 | ICOS | Myr |
| FRB | FKBP12v36 | tMyD88 | BCMA | Myr |
| FRB | FKBP12v36 | tMyD88 | CD27 | Myr |
| FRB | FKBP12v36 | tMyD88 | CD30 | Myr |
| FRB | FKBP12v36 | tMyD88 | CD122 | Myr |
| FRB | FKBP12v36 | tMyD88 | GITR S180A | Myr |
| FRB | FKBP12v36 | tMyD88 | GITR | Myr |
| | | | EEE191RVV | |
| FRB | FKBP12v36 | tMyD88 | HVEM | Myr |
| FRB | FKBP12v36 | tMyD88 | TWEAKR | Myr |
| FRB | FKBP12v36 | tMyD88 | RANK | Myr |
| FRB | FKBP12v36 | tMyD88 | RANK TRAF6 | Myr |
| FRB | FKBP12v36 | tMyD88 | RANK TRAF | Myr |
| | | | 2/5 | |
| FRB | FKBP12v36 | tMyD88 | RANK HCR- | Myr |
| | | | TRAF2/5 | |
| FRB | FKBP12v36 | tMyD88 | CD40-HCR- | Myr |
| | | | CD40 | |
| FRB | FKBP12v36 | tMyD88 | SOD1 | Myr |
| FRB | FKBP12v36 | tMyD88 | SOD2 | Myr |
| FRB | FKBP12v36 | tMyD88 | Costimulatory | Myr |
| | | | polypeptide | |
| | | | cytoplasmic | |
| | | | signaling region | |
| | | | selected from | |
| | | | the group | |
| | | | consisting of | |
| | | | TAC1, SOD3, | |
| | | | RANK88, | |
| | | | IL15Ra, | |
| | | | BTN3A1 (B30.2 | |
| | | | domain), CD86, | |
| | | | CD74Ra, | |
| | | | Dectin, ITA4, | |
| | | | ITGA5, TCL1A, | |
| | | | CTLA4, TIM1, | |
| | | | TREMBL2, | |
| | | | CD40-HCR- | |
| | | | CD40, and | |
| | | | DPP4 | |
| FRB | FKBP12wt | tMyD88 | CD28 | Myr |
| FRB | FKBP12wt | tMyD88 | 4-1BB | Myr |
| FRB | FKBP12wt | tMyD88 | OX40 | Myr |
| FRB | FKBP12wt | tMyD88 | ICOS | Myr |
| FRB | FKBP12wt | tMyD88 | BCMA | Myr |
| FRB | FKBP12wt | tMyD88 | CD27 | Myr |
| FRB | FKBP12wt | tMyD88 | CD30 | Myr |
| FRB | FKBP12wt | tMyD88 | CD122 | Myr |
| FRB | FKBP12wt | tMyD88 | GITR S180A | Myr |
| FRB | FKBP12wt | tMyD88 | GITR | Myr |
| | | | EEE191RVV | |
| FRB | FKBP12wt | tMyD88 | HVEM | Myr |
| FRB | FKBP12wt | tMyD88 | TWEAKR | Myr |
| FRB | FKBP12wt | tMyD88 | RANK | Myr |
| FRB | FKBP12wt | tMyD88 | RANK TRAF6 | Myr |
| FRB | FKBP12wt | tMyD88 | RANK TRAF | Myr |
| | | | 2/5 | |
| FRB | FKBP12wt | tMyD88 | RANK HCR- | Myr |
| | | | TRAF2/5 | |
| FRB | FKBP12wt | tMyD88 | CD40-HCR- | Myr |
| | | | CD40 | |
| FRB | FKBP12wt | tMyD88 | SOD1 | Myr |
| FRB | FKBP12wt | tMyD88 | SOD2 | Myr |
| FRB | FKBP12wt | tMyD88 | Costimulatory | Myr |
| | | | polypeptide | |
| | | | cytoplasmic | |
| | | | signaling region | |
| | | | selected from | |
| | | | the group | |
| | | | consisting of | |
| | | | TAC1, SOD3, | |
| | | | RANK88, | |
| | | | IL15Ra, | |
| | | | BTN3A1 (B30.2 | |
| | | | domain), CD86, | |
| | | | CD74Ra, | |
| | | | Dectin, ITA4, | |
| | | | ITGA5, TCL1A, | |
| | | | CTLA4, TIM1, | |
| | | | TREMBL2, | |
| | | | CD40-HCR- | |
| | | | CD40, and | |
| | | | DPP4 | |
| FRB_{L} | FKBP12v36 | tMyD88 | CD28 | Myr |
| FRB_{L} | FKBP12v36 | tMyD88 | 4-1BB | Myr |
| FRB_{L} | FKBP12v36 | tMyD88 | OX40 | Myr |
| FRB_{L} | FKBP12v36 | tMyD88 | ICOS | Myr |
| FRB_{L} | FKBP12v36 | tMyD88 | BCMA | Myr |
| FRB_{L} | FKBP12v36 | tMyD88 | CD27 | Myr |
| FRB_{L} | FKBP12v36 | tMyD88 | CD30 | Myr |
| FRB_{L} | FKBP12v36 | tMyD88 | CD122 | Myr |
| FRB_{L} | FKBP12v36 | tMyD88 | GITR S180A | Myr |
| FRB_{L} | FKBP12v36 | tMyD88 | GITR | Myr |
| | | | EEE191RVV | |
| FRB_{L} | FKBP12v36 | tMyD88 | HVEM | Myr |
| FRB_{L} | FKBP12v36 | tMyD88 | TWEAKR | Myr |
| FRB_{L} | FKBP12v36 | tMyD88 | RANK | Myr |
| FRB_{L} | FKBP12v36 | tMyD88 | RANK TRAF6 | Myr |
| FRB_{L} | FKBP12v36 | tMyD88 | RANK TRAF | Myr |
| | | | 2/5 | |
| FRB_{L} | FKBP12v36 | tMyD88 | RANK HCR- | Myr |
| | | | TRAF2/5 | |
| FRB_{L} | FKBP12v36 | tMyD88 | CD40-HCR- | Myr |
| | | | CD40 | |
| FRB_{L} | FKBP12v36 | tMyD88 | SOD1 | Myr |
| FRB_{L} | FKBP12v36 | tMyD88 | SOD2 | Myr |
| FRB_{L} | FKBP12v36 | tMyD88 | Costimulatory | Myr |
| | | | polypeptide | |
| | | | cytoplasmic | |
| | | | signaling region | |
| | | | selected from | |
| | | | the group | |
| | | | consisting of | |
| | | | TAC1, SOD3, | |
| | | | RANK88, | |
| | | | IL15Ra, | |
| | | | BTN3A1 (B30.2 | |
| | | | domain), CD86, | |
| | | | CD74Ra, | |
| | | | Dectin, ITA4, | |
| | | | ITGA5, TCL1A, | |
| | | | CTLA4, TIM1, | |
| | | | TREMBL2, | |
| | | | CD40-HCR- | |
| | | | CD40, and | |
| | | | DPP4 | |
| FRB_{L} | FKBP12wt | tMyD88 | CD28 | Myr |
| FRB_{L} | FKBP12wt | tMyD88 | 4-1BB | Myr |
| FRB_{L} | FKBP12wt | tMyD88 | OX40 | Myr |
| FRB_{L} | FKBP12wt | tMyD88 | ICOS | Myr |
| FRB_{L} | FKBP12wt | tMyD88 | BCMA | Myr |
| FRB_{L} | FKBP12wt | tMyD88 | CD27 | Myr |
| FRB_{L} | FKBP12wt | tMyD88 | CD30 | Myr |
| FRB_{L} | FKBP12wt | tMyD88 | CD122 | Myr |
| FRB_{L} | FKBP12wt | tMyD88 | GITR S180A | Myr |
| FRB_{L} | FKBP12wt | tMyD88 | GITR | Myr |
| | | | EEE191RVV | |
| FRB_{L} | FKBP12wt | tMyD88 | HVEM | Myr |
| FRB_{L} | FKBP12wt | tMyD88 | TWEAKR | Myr |
| FRB_{L} | FKBP12wt | tMyD88 | RANK | Myr |
| FRB_{L} | FKBP12wt | tMyD88 | RANK TRAF6 | Myr |
| FRB_{L} | FKBP12wt | tMyD88 | RANK TRAF | Myr |
| | | | 2/5 | |
| FRB_{L} | FKBP12wt | tMyD88 | RANK HCR- | Myr |
| | | | TRAF2/5 | |
| FRB_{L} | FKBP12wt | tMyD88 | CD40-HCR- | Myr |
| | | | CD40 | |
| FRB_{L} | FKBP12wt | tMyD88 | SOD1 | Myr |
| FRBL | FKBP12wt | tMyD88 | SOD2 | Myr |
| FRBL | FKBP12wt | tMyD88 | Costimulatory | |
| | | | polypeptide | |
| | | | cytoplasmic | |
| | | | signaling region | |
| | | | selected from | |
| | | | the group | |
| | | | consisting of | |
| | | | TAC1, SOD3, | |
| | | | RANK88, | |
| | | | IL15Ra, | |
| | | | BTN3A1 (B30.2 | |
| | | | domain), CD86, | |
| | | | CD74Ra, | |
| | | | Dectin, ITA4, | |
| | | | ITGA5, TCL1A, | |
| | | | CTLA4, TIM1, | |
| | | | TREMBL2, | |
| | | | CD40-HCR- | |
| | | | CD40, and | |
| | | | DPP4 | |

Chimeric signaling polypeptides provided in the present embodiments include, for example, chimeric signaling polypeptides comprising regions provided in Table 3. Costimulatory polypeptide cytoplasmic signaling regions include polypeptides and sequences provided herein, for purposes of the present application. Table 3 provides chimeric signaling polypeptides that include a constitutive signal, and are not inducible in the presence of a dimerizer. The chimeric signaling polypeptide of Table 3 do not comprise multimerizing regions such as FKBP12 or FRB polypeptides or variant polypeptides. In the following table, a MyD88 polypeptide region that is not truncated, such as, for example, a MyD88 polypeptide that comprises the TIR domain may be used in place of a truncated MyD88 polypeptide region lacking the TIR domain. The chimeric signling polypeptides of Table 3 may comprise a membrane-targeting region, such as, for example, a myristoylation region. A myristoylation region is indicated as “myr” in Table 3, however, it is understood that the chimeric signaling polypeptides may comprise any appropriate membrane targeting region, such as a membrane-targeting region provided herein. The order of the regions provided in the following table may vary; that is, the multimerizing region may be provided either at the amino terminal or carboxyl terminal portion of the polypeptide relative to the signaling region.

TABLE 3

Signaling region

| Truncated | | |
|---|---|---|
| MyD88 | | |
| polypeptide | Costimulatory | Membrane-targeting |
| region lacking | polypeptide | Region |
| the TIR domain | cytoplasmic | Membrane-targeting |
| (tMyD88) | signaling region | region |
| tMyD88 | CD28 | Myr |
| tMyD88 | 4-1BB | Myr |
| tMyD88 | OX40 | Myr |
| tMyD88 | ICOS | Myr |
| tMyD88 | BCMA | Myr |
| tMyD88 | CD27 | Myr |
| tMyD88 | CD30 | Myr |
| tMyD88 | CD122 | Myr |
| tMyD88 | GITR S180A | Myr |
| tMyD88 | GITR | Myr |
| | EEE191RVV | |
| tMyD88 | HVEM | Myr |
| tMyD88 | TWEAKR | Myr |
| tMyD88 | RANK | Myr |
| tMyD88 | RANK TRAF6 | Myr |
| tMyD88 | RANK TRAF | Myr |
| | 2/5 | |
| tMyD88 | RANK HCR- | Myr |
| | TRAF2/5 | |
| tMyD88 | CD40-HCR- | Myr |
| | CD40 | |
| tMyD88 | SOD1 | Myr |
| tMyD88 | SOD2 | Myr |
| | | Myr |
| tMyD88 | CD28 | Myr |
| tMyD88 | 4-1BB | Myr |
| tMyD88 | OX40 | Myr |
| tMyD88 | ICOS | Myr |
| tMyD88 | BCMA | Myr |
| tMyD88 | CD27 | Myr |
| tMyD88 | CD30 | Myr |
| tMyD88 | CD122 | Myr |
| tMyD88 | GITR S180A | Myr |
| tMyD88 | GITR | Myr |
| | EEE191RVV | |
| tMyD88 | HVEM | Myr |
| tMyD88 | TWEAKR | Myr |
| tMyD88 | RANK | Myr |
| tMyD88 | RANK TRAF6 | Myr |
| tMyD88 | RANK TRAF | Myr |
| | 2/5 | |
| tMyD88 | RANK HCR- | Myr |
| | TRAF2/5 | |
| tMyD88 | CD40-HCR- | Myr |
| | CD40 | |
| tMyD88 | SOD1 | Myr |
| tMyD88 | SOD2 | Myr |
| | | Myr |
| tMyD88 | CD28 | Myr |
| tMyD88 | 4-1BB | Myr |
| tMyD88 | OX40 | Myr |
| tMyD88 | ICOS | Myr |
| tMyD88 | BCMA | Myr |
| tMyD88 | CD27 | Myr |
| tMyD88 | CD30 | Myr |
| tMyD88 | CD122 | Myr |
| tMyD88 | GITR S180A | Myr |
| tMyD88 | GITR | Myr |
| | EEE191RVV | |
| tMyD88 | HVEM | Myr |
| tMyD88 | TWEAKR | Myr |
| tMyD88 | RANK | Myr |
| tMyD88 | RANK TRAF6 | Myr |
| tMyD88 | RANK TRAF | Myr |
| | 2/5 | |
| tMyD88 | RANK HCR- | Myr |
| | TRAF2/5 | |
| tMyD88 | CD40-HCR- | Myr |
| | CD40 | |
| tMyD88 | SOD1 | Myr |
| tMyD88 | SOD2 | Myr |
| | | Myr |
| tMyD88 | CD28 | Myr |
| tMyD88 | 4-1BB | Myr |
| tMyD88 | OX40 | Myr |
| tMyD88 | ICOS | Myr |
| tMyD88 | BCMA | Myr |
| tMyD88 | CD27 | Myr |
| tMyD88 | CD30 | Myr |
| tMyD88 | CD122 | Myr |
| tMyD88 | GITR S180A | Myr |
| tMyD88 | GITR | Myr |
| | EEE191RVV | |
| tMyD88 | HVEM | Myr |
| tMyD88 | TWEAKR | Myr |
| tMyD88 | RANK | Myr |
| tMyD88 | RANK TRAF6 | Myr |
| tMyD88 | RANK TRAF | Myr |
| | 2/5 | |
| tMyD88 | RANK HCR- | Myr |
| | TRAF2/5 | |
| tMyD88 | CD40-HCR- | Myr |
| | CD40 | |
| tMyD88 | SOD1 | Myr |
| tMyD88 | SOD2 | Myr |
| | | Myr |
| tMyD88 | CD28 | Myr |
| tMyD88 | 4-1BB | Myr |
| tMyD88 | OX40 | Myr |
| tMyD88 | ICOS | Myr |
| tMyD88 | BCMA | Myr |
| tMyD88 | CD27 | Myr |
| tMyD88 | CD30 | Myr |
| tMyD88 | CD122 | Myr |
| tMyD88 | GITR S180A | Myr |
| tMyD88 | GITR | Myr |
| | EEE191RVV | |
| tMyD88 | HVEM | Myr |
| tMyD88 | TWEAKR | Myr |
| tMyD88 | RANK | Myr |
| tMyD88 | RANK TRAF6 | Myr |
| tMyD88 | RANK TRAF | Myr |
| | 2/5 | |
| tMyD88 | RANK HCR- | Myr |
| | TRAF2/5 | |
| tMyD88 | CD40-HCR- | Myr |
| | CD40 | |
| tMyD88 | SOD1 | Myr |
| tMyD88 | SOD2 | Myr |
| | | Myr |
| tMyD88 | CD28 | Myr |
| tMyD88 | 4-1BB | Myr |
| tMyD88 | OX40 | Myr |
| tMyD88 | ICOS | Myr |
| tMyD88 | BCMA | Myr |
| tMyD88 | CD27 | Myr |
| tMyD88 | CD30 | Myr |
| tMyD88 | CD122 | Myr |
| tMyD88 | GITR S180A | Myr |
| tMyD88 | GITR | Myr |
| | EEE191RVV | |
| tMyD88 | HVEM | Myr |
| tMyD88 | TWEAKR | Myr |
| tMyD88 | RANK | Myr |
| tMyD88 | RANK TRAF6 | Myr |
| tMyD88 | RANK TRAF | Myr |
| | 2/5 | |
| tMyD88 | RANK HCR- | Myr |
| | TRAF2/5 | |
| tMyD88 | CD40-HCR- | Myr |
| | CD40 | |
| tMyD88 | SOD1 | Myr |
| tMyD88 | SOD2 | Myr |
| | | Myr |
| tMyD88 | CD28 | Myr |
| tMyD88 | 4-1BB | Myr |
| tMyD88 | OX40 | Myr |
| tMyD88 | ICOS | Myr |
| tMyD88 | BCMA | Myr |
| tMyD88 | CD27 | Myr |
| tMyD88 | CD30 | Myr |
| tMyD88 | CD122 | Myr |
| tMyD88 | GITR S180A | Myr |
| tMyD88 | GITR | Myr |
| | EEE191RVV | |
| tMyD88 | HVEM | Myr |
| tMyD88 | TWEAKR | Myr |
| tMyD88 | RANK | Myr |
| tMyD88 | RANK TRAF6 | Myr |
| tMyD88 | RANK TRAF | Myr |
| | 2/5 | |
| tMyD88 | RANK HCR- | Myr |
| | TRAF2/5 | |
| tMyD88 | CD40-HCR- | Myr |
| | CD40 | |
| tMyD88 | SOD1 | Myr |
| tMyD88 | SOD2 | Myr |

Chimeric signaling polypeptides provided in the present embodiments include, for example, chimeric signaling polypeptides comprising regions provided in Table 4. Costimulatory polypeptide cytoplasmic signaling regions include polypeptides and sequences provided herein, for purposes of the present application. Table 4 provides chimeric signaling polypeptides that include a constitutive signal, and are not inducible in the presence of a dimerizer. The chimeric signaling polypeptide of Table 4 do not comprise multimerizing regions such as FKBP12 or FRB polypeptides or variant polypeptides. In the following table, a MyD88 polypeptide region that is not truncated, such as, for example, a MyD88 polypeptide that comprises the TIR domain may be used in place of a truncated MyD88 polypeptide region lacking the TIR domain. The chimeric signling polypeptides of Table 4 may lack a membrane-targeting region, such as, for example, a myristoylation region. The order of the regions provided in the following table may vary; that is, the multimerizing region may be provided either at the amino terminal or carboxyl terminal portion of the polypeptide relative to the signaling region.

TABLE 4
Signaling region

| Truncated | |
|---|---|
| MyD88 | |
| polypeptide | Costimulatory |
| region lacking | polypeptide |
| the TIR domain | cytoplasmic |
| (tMyD88) | signaling region |
| tMyD88 | CD28 |
| tMyD88 | 4-1BB |
| tMyD88 | OX40 |
| tMyD88 | ICOS |
| tMyD88 | BCMA |
| tMyD88 | CD27 |
| tMyD88 | CD30 |
| tMyD88 | CD122 |
| tMyD88 | GITR S180A |
| tMyD88 | GITR |
| | EEE191RVV |
| tMyD88 | HVEM |
| tMyD88 | TWEAKR |
| tMyD88 | RANK |
| tMyD88 | RANK TRAF6 |
| tMyD88 | RANK TRAF |
| | 2/5 |
| tMyD88 | RANK HCR- |
| | TRAF2/5 |
| tMyD88 | CD40-HCR- |
| | CD40 |
| tMyD88 | SOD1 |
| tMyD88 | SOD2 |
| tMyD88 | CD28 |
| tMyD88 | 4-1BB |
| tMyD88 | OX40 |
| tMyD88 | ICOS |
| tMyD88 | BCMA |
| tMyD88 | CD27 |
| tMyD88 | CD30 |
| tMyD88 | CD122 |
| tMyD88 | GITR S180A |
| tMyD88 | GITR |
| | EEE191RVV |
| tMyD88 | HVEM |
| tMyD88 | TWEAKR |
| tMyD88 | RANK |
| tMyD88 | RANK TRAF6 |
| tMyD88 | RANK TRAF |
| | 2/5 |
| tMyD88 | RANK HCR- |
| | TRAF2/5 |
| tMyD88 | CD40-HCR- |
| | CD40 |
| tMyD88 | SOD1 |
| tMyD88 | SOD2 |
| tMyD88 | CD28 |
| tMyD88 | 4-1BB |
| tMyD88 | OX40 |
| tMyD88 | ICOS |
| tMyD88 | BCMA |
| tMyD88 | CD27 |
| tMyD88 | CD30 |
| tMyD88 | CD122 |
| tMyD88 | GITR S180A |
| tMyD88 | GITR |
| | EEE191RVV |
| tMyD88 | HVEM |
| tMyD88 | TWEAKR |
| tMyD88 | RANK |
| tMyD88 | RANK TRAF6 |
| tMyD88 | RANK TRAF |
| | 2/5 |
| tMyD88 | RANK HCR- |
| | TRAF2/5 |
| tMyD88 | CD40-HCR- |
| | CD40 |
| tMyD88 | SOD1 |
| tMyD88 | SOD2 |
| tMyD88 | CD28 |
| tMyD88 | 4-1BB |
| tMyD88 | OX40 |
| tMyD88 | ICOS |
| tMyD88 | BCMA |
| tMyD88 | CD27 |
| tMyD88 | CD30 |
| tMyD88 | CD122 |
| tMyD88 | GITR S180A |
| tMyD88 | GITR |
| | EEE191RVV |
| tMyD88 | HVEM |
| tMyD88 | TWEAKR |
| tMyD88 | RANK |
| tMyD88 | RANK TRAF6 |
| tMyD88 | RANK TRAF |
| | 2/5 |
| tMyD88 | RANK HCR- |
| | TRAF2/5 |
| tMyD88 | CD40-HCR- |
| | CD40 |
| tMyD88 | SOD1 |
| tMyD88 | SOD2 |
| tMyD88 | CD28 |
| tMyD88 | 4-1BB |
| tMyD88 | OX40 |
| tMyD88 | ICOS |
| tMyD88 | BCMA |
| tMyD88 | CD27 |
| tMyD88 | CD30 |
| tMyD88 | CD122 |
| tMyD88 | GITR S180A |
| tMyD88 | GITR |
| | EEE191RVV |
| tMyD88 | HVEM |
| tMyD88 | TWEAKR |
| tMyD88 | RANK |
| tMyD88 | RANK TRAF6 |
| tMyD88 | RANK TRAF |
| | 2/5 |
| tMyD88 | RANK HCR- |
| | TRAF2/5 |
| tMyD88 | CD40-HCR- |
| | CD40 |
| tMyD88 | SOD1 |
| tMyD88 | SOD2 |
| tMyD88 | CD28 |
| tMyD88 | 4-1BB |
| tMyD88 | OX40 |
| tMyD88 | ICOS |
| tMyD88 | BCMA |
| tMyD88 | CD27 |
| tMyD88 | CD30 |
| tMyD88 | CD122 |
| tMyD88 | GITR S180A |
| tMyD88 | GITR |
| | EEE191RVV |
| tMyD88 | HVEM |
| tMyD88 | TWEAKR |
| tMyD88 | RANK |
| tMyD88 | RANK TRAF6 |
| tMyD88 | RANK TRAF |
| | 2/5 |
| tMyD88 | RANK HCR- |
| | TRAF2/5 |
| tMyD88 | CD40-HCR- |
| | CD40 |
| tMyD88 | SOD1 |
| tMyD88 | SOD2 |
| tMyD88 | CD28 |
| tMyD88 | 4-1BB |
| tMyD88 | OX40 |
| tMyD88 | ICOS |
| tMyD88 | BCMA |
| tMyD88 | CD27 |
| tMyD88 | CD30 |
| tMyD88 | CD122 |
| tMyD88 | GITR S180A |
| tMyD88 | GITR |
| | EEE191RVV |
| tMyD88 | HVEM |
| tMyD88 | TWEAKR |
| tMyD88 | RANK |
| tMyD88 | RANK TRAF6 |
| tMyD88 | RANK TRAF |
| | 2/5 |
| tMyD88 | RANK HCR- |
| | TRAF2/5 |
| tMyD88 | CD40-HCR- |
| | CD40 |
| tMyD88 | SOD1 |
| tMyD88 | SOD2 |

It is understood that by “derived” is meant that the nucleotide sequence or amino acid sequence may be derived from the sequence of the molecule. The intracellular domain comprises at least one polypeptide which causes activation of the T cell, such as, for example, but not limited to, CD3 zeta, and, for example, costimulatory molecules, for example, but not limited to, CD28, OX40 and 4-1BB.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms.

The term “allogeneic” as used herein, refers to HLA or MHC loci that are antigenically distinct between the host and donor cells.

Thus, cells or tissue transferred from the same species can be antigenically distinct. Syngeneic mice can differ at one or more loci (congenics) and allogeneic mice can have the same background.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates. Exemplary organisms include but are not limited to, Helicobacters, Campylobacters, Clostridia, Corynebacterium diphtheriae, Bordetella pertussis, influenza virus, parainfluenza viruses, respiratory syncytial virus, Borrelia burgdorferi, Plasmodium, herpes simplex viruses, human immunodeficiency virus, papillomavirus, Vibrio cholera, E. coli, measles virus, rotavirus, shigella, Salmonella typhi, Neisseria gonorrhea. Therefore, any macromolecules, including virtually all proteins, polypeptides, or peptides, can serve as antigens. Furthermore, antigens can be derived from recombinant or genomic DNA. Any DNA that contains nucleotide sequences or partial nucleotide sequences of a pathogenic genome or a gene or a fragment of a gene for a protein that elicits an immune response results in synthesis of an antigen. Furthermore, the present methods are not limited to the use of the entire nucleic acid sequence of a gene or genome. It is readily inherent that the present embodiments include, but are not limited to, the use of partial nucleic acid sequences of more than one gene or genome and that these nucleic acid sequences are arranged in various combinations to elicit the desired immune response.

An “antigen recognition moiety” may be any polypeptide or fragment thereof, such as, for example, an antibody fragment variable domain, either naturally-derived, or synthetic, which binds to an antigen. Examples of antigen recognition moieties include, but are not limited to, polypeptides derived from antibodies, such as, for example, single chain variable fragments (scFv), Fab, Fab′, F(ab′)2, and Fv fragments; polypeptides derived from T Cell receptors, such as, for example, TCR variable domains; and any ligand or receptor fragment that binds to the extracellular cognate protein.

The term “antigen-presenting cell” is any of a variety of cells capable of displaying, acquiring, or presenting at least one antigen or antigenic fragment on (or at) its cell surface. In general, the term “cell” can be any cell that accomplishes the goal of aiding the enhancement of an immune response (i.e., from the T-cell or —B-cell arms of the immune system) against an antigen or antigenic composition. As discussed in Kuby, 2000, Immunology, 4.sup.th edition, W.H. Freeman and company, for example, (incorporated herein by reference), and used herein in certain embodiments, a cell that displays or presents an antigen normally or with a class II major histocompatibility molecule or complex to an immune cell is an “cell.” In certain aspects, a cell (e.g., an APC cell) may be fused with another cell, such as a recombinant cell or a tumor cell that expresses the desired antigen. Methods for preparing a fusion of two or more cells are discussed in, for example, Goding, J. W., Monoclonal Antibodies: Principles and Practice, pp. 65-66, 71-74 (Academic Press, 1986); Campbell, in: Monoclonal Antibody Technology, Laboratory Techniques in Biochemistry and Molecular Biology, Vol. 13, Burden & Von Knippenberg, Amsterdam, Elseview, pp. 75-83, 1984; Kohler & Milstein, Nature, 256:495-497, 1975; Kohler & Milstein, Eur. J. Immunol., 6:511-519, 1976, Gefter et al., Somatic Cell Genet., 3:231-236, 1977, each incorporated herein by reference. In some cases, the immune cell to which a cell displays or presents an antigen to is a CD4+TH cell. Additional molecules expressed on the APC or other immune cells may aid or improve the enhancement of an immune response. Secreted or soluble molecules, such as for example, cytokines and adjuvants, may also aid or enhance the immune response against an antigen. Various examples are discussed herein. In some cases, the immune cell to which a cell displays or presents an antigen to is a CD4+TH cell. Additional molecules expressed on the APC or other immune cells may aid or improve the enhancement of an immune response. Secreted or soluble molecules, such as for example, cytokines and adjuvants, may also aid or enhance the immune response against an antigen. Various examples are discussed herein.

The term “cancer” as used herein is defined as a hyperproliferation of cells whose unique trait-loss of normal controls—results in unregulated growth, lack of differentiation, local tissue invasion, and metastasis. Examples include but are not limited to, melanoma, non-small cell lung, small-cell lung, lung, hepatocarcinoma, leukemia, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

As used herein, the term “iCD40CD40 molecule” or “iCD40 polypeptide” is defined as an inducible CD40. This iCD40 can bypass mechanisms that extinguish endogenous CD40 signaling. The term “iCD40” embraces “iCD40 nucleic acids,” “iCD40 polypeptides” and/or iCD40 expression vectors.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There are times when the full or partial genomic sequence is used, such as where the non-coding regions are required for optimal expression or where non-coding regions such as introns are to be targeted in an antisense strategy.

The term “dendritic cell” (DC) is an cell existing in vivo, in vitro, ex vivo, or in a host or subject, or which can be derived from a hematopoietic stem cell or a monocyte. Dendritic cells and their precursors can be isolated from a variety of lymphoid organs, e.g., spleen, lymph nodes, as well as from bone marrow and peripheral blood. The DC has a characteristic morphology with thin sheets (lamellipodia) extending in multiple directions away from the dendritic cell body. Typically, dendritic cells express high levels of MHC and costimulatory (e.g., B7-1 and B7-2) molecules. Dendritic cells can induce antigen specific differentiation of T cells in vitro, and are able to initiate primary T cell responses in vitro and in vivo.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. The term “therapeutic construct” may also be used to refer to the expression construct or transgene. The expression construct or transgene may be used, for example, as a therapy to treat hyperproliferative diseases or disorders, such as cancer, thus the expression construct or transgene is a therapeutic construct or a prophylactic construct.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra.

As used herein, the term “ex vivo” refers to “outside” the body. The terms “ex vivo” and “in vitro” can be used interchangeably herein.

As used herein, the term “functionally equivalent,” as it relates to a MyD88 or truncated MyD88 polypeptide, or to a costimulatory polypeptide, for example, refers to a MyD88 polypeptide or truncated MyD88 polypeptide, or costimulatory polypeptide fragment, polypeptide variant, or analog, that stimulates an immune response to destroy tumors or hyperproliferative disease. Functionally equivalent truncated MyD88 polypeptides, for example, may include amino acid substitutions or deletions, including, for example, amino acid deletions at the N- or C-termini, that maintain at least 50, 60, 70, 80, 90, or 95% of the immune response stimulatory activity of the truncated MyD88 polypeptide, exemplified in SEQ ID NO: 2, when provided in a chimeric signaling polypeptide of the present embodiments. “Functionally equivalent” or “a functional fragment” of a costimulatory polypeptide cytoplasmic region refers, for example, to a costimulatory polypeptide that is lacking the extracellular domain, but is capable of costimulating the cell-mediated tumor killing response, such as, for example, the T cell-mediated, NK cell-mediated, invariant NK-T cell mediated, gamma delta T cell-mediated, or NK-T cell-mediated response. When the term “functionally equivalent” is applied to other nucleic acids or polypeptides, such as, for example, PSA polypeptide, PSMA polypeptide, it refers to fragments, variants, and the like that have the same or similar activity as the reference polypeptides of the methods herein. For example, a functional fragment of a tumor antigen polypeptide, such as, for example, PSMA, may be antigenic, allowing for antibodies to be produced that recognize the particular tumor antigen. A functional fragment of a ligand binding region, for example, Fvls, would include a sufficient portion of the ligand binding region polypeptide to bind the appropriate ligand. “Functionally equivalent” refers, for example, to a costimulatory polypeptide that is lacking the extracellular domain, but is capable of amplifying the T cell-mediated tumor killing response when expressed in T cells.

The term “hyperproliferative disease” is defined as a disease that results from a hyperproliferation of cells. Exemplary hyperproliferative diseases include, but are not limited to cancer or autoimmune diseases. Other hyperproliferative diseases may include vascular occlusion, restenosis, atherosclerosis, or inflammatory bowel disease.

As used herein, the term “gene” is defined as a functional protein, polypeptide, or peptide-encoding unit. As will be understood, this functional term includes genomic sequences, cDNA sequences, and smaller engineered gene segments that express, or are adapted to express, proteins, polypeptides, domains, peptides, fusion proteins, and mutants.

The term “immunogenic composition” or “immunogen” refers to a substance that is capable of provoking an immune response. Examples of immunogens include, e.g., antigens, autoantigens that play a role in induction of autoimmune diseases, and tumor-associated antigens expressed on cancer cells.

The term “immunocompromised” as used herein is defined as a subject that has reduced or weakened immune system. The immunocompromised condition may be due to a defect or dysfunction of the immune system or to other factors that heighten susceptibility to infection and/or disease. Although such a categorization allows a conceptual basis for evaluation, immunocompromised individuals often do not fit completely into one group or the other. More than one defect in the body's defense mechanisms may be affected. For example, individuals with a specific T-lymphocyte defect caused by HIV may also have neutropenia caused by drugs used for antiviral therapy or be immunocompromised because of a breach of the integrity of the skin and mucous membranes. An immunocompromised state can result from indwelling central lines or other types of impairment due to intravenous drug abuse; or be caused by secondary malignancy, malnutrition, or having been infected with other infectious agents such as tuberculosis or sexually transmitted diseases, e.g., syphilis or hepatitis.

As used herein, the term “pharmaceutically or pharmacologically acceptable” refers to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells presented herein, its use in therapeutic compositions is contemplated. Supplementary active ingredients also can be incorporated into the compositions. In some embodiments, the subject is a mammal. In some embodiments, the subject is a human.

As used herein, the term “polynucleotide” is defined as a chain of nucleotides. Furthermore, nucleic acids are polymers of nucleotides. Thus, nucleic acids and polynucleotides as used herein are interchangeable. Nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, polynucleotides include mutations of the polynucleotides, include but are not limited to, mutation of the nucleotides, or nucleosides by methods well known in the art. A nucleic acid may comprise one or more polynucleotides.

As used herein, the term “polypeptide” is defined as a chain of amino acid residues, usually having a defined sequence. As used herein the term polypeptide may be interchangeable with the term “proteins”.

As used herein, the term “promoter” is defined as a DNA sequence recognized by the synthetic machinery of the cell, or introduced synthetic machinery, required to initiate the specific transcription of a gene.

As used herein, the terms “regulate an immune response,” “modulate an immune response,” or “control an immune response,” refer to the ability to modify the immune response. For example, the composition is capable of enhancing and/or activating the immune response. Still further, the composition is also capable of inhibiting the immune response. The form of regulation is determined by the ligand that is used with the composition. For example, a dimeric analog of the chemical results in dimerization of the costimulatory polypeptide leading to activation of the T cell, however, a monomeric analog of the chemical does not result in dimerization of the costimulatory polypeptide, which would not activate the T cells.

The term “transfection” and “transduction” are interchangeable and refer to the process by which an exogenous DNA sequence is introduced into a eukaryotic host cell. Transfection (or transduction) can be achieved by any one of a number of means including electroporation, microinjection, gene gun delivery, retroviral infection, lipofection, superfection and the like.

As used herein, the term “syngeneic” refers to cells, tissues or animals that have genotypes that are identical or closely related enough to allow tissue transplant, or are immunologically compatible. For example, identical twins or animals of the same inbred strain. Syngeneic and isogeneic can be used interchangeably.

The term “patient” or “subject” are interchangeable, and, as used herein include, but are not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

By “T cell activation molecule” is meant a polypeptide that, when incorporated into a T cell expressing a chimeric antigen receptor, enhances activation of the T cell. Examples include, but are not limited to, ITAM-containing, Signal 1 conferring molecules such as, for example, CD3ζ polypeptide, and Fc receptor gamma, such as, for example, Fc epsilon receptor gamma (FcεR1γ) subunit (Haynes, N. M., et al. J. Immunol. 166:182-7 (2001).

As used herein, the term “vaccine” refers to a formulation that contains a composition presented herein which is in a form that is capable of being administered to an animal. Typically, the vaccine comprises a conventional saline or buffered aqueous solution medium in which the composition is suspended or dissolved. In this form, the composition can be used conveniently to prevent, ameliorate, or otherwise treat a condition. Upon introduction into a subject, the vaccine is able to provoke an immune response including, but not limited to, the production of antibodies, cytokines and/or other cellular responses.

As used herein, the term “under transcriptional control” or “operatively linked” is defined as the promoter is in the correct location and orientation in relation to the nucleic acid to control RNA polymerase initiation and expression of the gene.

As used herein, the terms “treatment”, “treat”, “treated”, or “treating” refer to prophylaxis and/or therapy. When used with respect to a solid tumor, such as a cancerous solid tumor, for example, the term refers to prevention by prophylactic treatment, which increases the subject's resistance to solid tumors or cancer. In some examples, the subject may be treated to prevent cancer, where the cancer is familial, or is genetically associated. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e. g., reduce or eliminate the infection or prevent it from becoming worse.

As used herein, the term “vaccine” refers to a formulation which contains a composition presented herein which is in a form that is capable of being administered to an animal. Typically, the vaccine comprises a conventional saline or buffered aqueous solution medium in which the composition is suspended or dissolved. In this form, the composition can be used conveniently to prevent, ameliorate, or otherwise treat a condition. Upon introduction into a subject, the vaccine is able to provoke an immune response including, but not limited to, the production of antibodies, cytokines and/or other cellular responses.

Blood disease: The terms “blood disease”, “blood disease” and/or “diseases of the blood” as used herein, refers to conditions that affect the production of blood and its components, including but not limited to, blood cells, hemoglobin, blood proteins, the mechanism of coagulation, production of blood, production of blood proteins, the like and combinations thereof. Non-limiting examples of blood diseases include anemias, leukemias, lymphomas, hematological neoplasms, albuminemias, haemophilias and the like.

Bone marrow disease: The term “bone marrow disease” as used herein, refers to conditions leading to a decrease in the production of blood cells and blood platelets. In some bone marrow diseases, normal bone marrow architecture can be displaced by infections (e.g., tuberculosis) or malignancies, which in turn can lead to the decrease in production of blood cells and blood platelets. Non-limiting examples of bone marrow diseases include leukemias, bacterial infections (e.g., tuberculosis), radiation sickness or poisoning, apnocytopenia, anemia, multiple myeloma and the like.

T cells (also referred to as T lymphocytes) belong to a group of white blood cells referred to as lymphocytes. Lymphocytes generally are involved in cell-mediated immunity. The “T” in “T cells” refers to cells derived from or whose maturation is influenced by the thymus. T cells can be distinguished from other lymphocytes types such as B cells and Natural Killer (NK) cells by the presence of cell surface proteins known as T cell receptors. The term “activated T cells” as used herein, refers to T cells that have been stimulated to produce an immune response (e.g., clonal expansion of activated T cells) by recognition of an antigenic determinant presented in the context of a Class II major histo-compatibility (MHC) marker. T-cells are activated by the presence of an antigenic determinant, cytokines and/or lymphokines and cluster of differentiation cell surface proteins (e.g., CD3, CD4, CD8, the like and combinations thereof). Cells that express a cluster of differential protein often are said to be “positive” for expression of that protein on the surface of T-cells (e.g., cells positive for CD3 or CD 4 expression are referred to as CD3⁺ or CD4⁺). CD3 and CD4 proteins are cell surface receptors or co-receptors that may be directly and/or indirectly involved in signal transduction in T cells. Other cells discussed herein may also be involved in cell-mediated immune response, and cell-mediated immune responses are not limited to those induced by the activation of T cells, but may, in some embodiments, be related to the activation of other immune cells discussed herein, for example, NK cells, NK-T cells, for example, invariant NK-T cells, and gamma delta T cells.

Natural killer cells (NK cells) are cytotoxic lymphocytes that are part of the innate immune system. NK cells, in general, do not express markers indicative of T or B cells. NK cell markers include CD16 and/or CD56. NK cells act by killing target cells, such as tumor cells, infected cells, and antibody-targeted cells. NK cells may be obtained or isolated from, for example, peripheral blood, bone marrow, and umbilical cord blood, or they can be derived from stem cell populations including CD34 positive hematopoeitic stem cells themselves derived from Induced Pluripotent Stem (IPS) cells that can be genetically modified.

The embodiments provided herein to the extent that they refer to natural killer cells may, in some embodiments, refer to cells having reduced alloreactivity, such as, for example, invariant NK-T cells, and gamma delta T cells. Thus, in some embodiments are provided invariant NK-T cells and gamma delta T cells modified as provided in the present application.

Peripheral blood: The term “peripheral blood” as used herein, refers to cellular components of blood (e.g., red blood cells, white blood cells and platelets), which are obtained or prepared from the circulating pool of blood and not sequestered within the lymphatic system, spleen, liver or bone marrow.

Umbilical cord blood: Umbilical cord blood is distinct from peripheral blood and blood sequestered within the lymphatic system, spleen, liver or bone marrow. The terms “umbilical cord blood”, “umbilical blood” or “cord blood”, which can be used interchangeably, refers to blood that remains in the placenta and in the attached umbilical cord after child birth. Cord blood often contains stem cells including hematopoietic cells.

By “obtained or prepared” as, for example, in the case of cells, is meant that the cells or cell culture are isolated, purified, or partially purified from the source, where the source may be, for example, umbilical cord blood, bone marrow, or peripheral blood. The terms may also apply to the case where the original source, or a cell culture, has been cultured and the cells have replicated, and where the progeny cells are now derived from the original source.

By “kill” or “killing” as in a percent of cells killed, is meant the death of a cell through apoptosis, as measured using any method known for measuring apoptosis. The term may also refer to cell ablation.

Allodepletion: The term “allodepletion” as used herein, refers to the selective depletion of alloreactive T cells. The term “alloreactive T cells” as used herein, refers to T cells activated to produce an immune response in reaction to exposure to foreign cells, such as, for example, in a transplanted allograft. The selective depletion generally involves targeting various cell surface expressed markers or proteins, (e.g., sometimes cluster of differentiation proteins (CD proteins), CD19, or the like), for removal using immunomagnets, immunotoxins, flow sorting, induction of apoptosis, photodepletion techniques, the like or combinations thereof. In the present methods, the cells may be transduced or transfected with the chimeric protein-encoding vector before or after allodepletion. Also, the cells may be transduced or transfected with the chimeric protein-encoding vector without an allodepletion step, and the non-allodepleted cells may be administered to the patient. Because of the added “safety switch” in certain embodiments, in cells that express the inducible chimeric Caspase-9 polypeptide, it is, for example, possible to administer the non-allo-depleted (or only partially allo-depleted) T cells because an adverse event such as, for example, graft versus host disease, may be alleviated upon the administration of the multimeric ligand.

Donor T cell: The term “donor T cell” as used here refers to T cells that often are administered to a recipient to confer anti-viral and/or anti-tumor immunity following allogeneic stem cell transplantation. Donor T cells often are utilized to inhibit marrow graft rejection and increase the success of alloengraftment, however the same donor T cells can cause an alloaggressive response against host antigens, which in turn can result in graft versus host disease (GVHD). Certain activated donor T cells can cause a higher or lower GvHD response than other activated T cells. Donor T cells may also be reactive against recipient tumor cells, causing a beneficial graft vs. tumor effect.

Function-conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

Amino acids other than those indicated as conserved may differ in a protein or enzyme so that the percent protein or amino acid sequence similarity between any two proteins of similar function may vary and can be, for example, at least 70%, at least 80%, at least 90%, and most at least 95%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA. When using any of these programs, the settings usually selected are those that results in the highest sequence similarity.

Mesenchymal stromal cell: The terms “mesenchymal stromal cell” or “bone marrow derived mesenchymal stromal cell” as used herein, refer to multipotent stem cells that can differentiate ex vivo, in vitro and in vivo into adipocytes, osteoblasts and chondroblasts, and may be further defined as a fraction of mononuclear bone marrow cells that adhere to plastic culture dishes in standard culture conditions, are negative for hematopoietic lineage markers and are positive for CD73, CD90 and CD105.

Embryonic stem cell: The term “embryonic stem cell” as used herein, refers to pluripotent stem cells derived from the inner cell mass of the blastocyst, an early-stage embryo of between 50 to 150 cells. Embryonic stem cells are characterized by their ability to renew themselves indefinitely and by their ability to differentiate into derivatives of all three primary germ layers, ectoderm, endoderm and mesoderm. Pluripotent is distinguished from mutipotent in that pluripotent cells can generate all cell types, while multipotent cells (e.g., adult stem cells) can only produce a limited number of cell types.

Inducible pluripotent stem cell: The terms “inducible pluripotent stem cell” or “induced pluripotent stem cell” as used herein refers to adult, or differentiated cells, that are “reprogrammed” or induced by genetic (e.g., expression of genes that in turn activates pluripotency), biological (e.g., treatment viruses or retroviruses) and/or chemical (e.g., small molecules, peptides and the like) manipulation to generate cells that are capable of differentiating into many if not all cell types, like embryonic stem cells. Inducible pluripotent stem cells are distinguished from embryonic stem cells in that they achieve an intermediate or terminally differentiated state (e.g., skin cells, bone cells, fibroblasts, and the like) and then are induced to dedifferentiate, thereby regaining some or all of the ability to generate multipotent or pluripotent cells.

CD34⁺ cell: The term “CD34⁺ cell” as used herein refers to a cell expressing the CD34 protein on its cell surface. “CD34” as used herein refers to a cell surface glycoprotein (e.g., sialomucin protein) that often acts as a cell-cell adhesion factor and is involved in T cell entrance into lymph nodes, and is a member of the “cluster of differentiation” gene family. CD34 also may mediate the attachment of stem cells to bone marrow, extracellular matrix or directly to stromal cells. CD34⁺ cells often are found in the umbilical cord and bone marrow as hematopoietic cells, a subset of mesenchymal stem cells, endothelial progenitor cells, endothelial cells of blood vessels but not lymphatics (except pleural lymphatics), mast cells, a sub-population of dendritic cells (which are factor XIIIa negative) in the interstitium and around the adnexa of dermis of skin, as well as cells in certain soft tissue tumors (e.g., alveolar soft part sarcoma, pre-B acute lymphoblastic leukemia (Pre-B-ALL), acute myelogenous leukemia (AML), AML-M7, dermatofibrosarcoma protuberans, gastrointestinal stromal tumors, giant cell fibroblastoma, granulocytic sarcoma, Kaposi's sarcoma, liposarcoma, malignant fibrous histiocytoma, malignant peripheral nerve sheath tumors, mengingeal hemangiopericytomas, meningiomas, neurofibromas, schwannomas, and papillary thyroid carcinoma).

Tumor infiltrating lymphocytes (TILs) refer to T cells having various receptors which infiltrate tumors and kill tumor cells in a targeted manor. Regulating the activity of the TILs using the methods of the present application would allow for more direct control of the elimination of tumor cells.

Gene expression vector: The terms “gene expression vector”, “nucleic acid expression vector”, or “expression vector” as used herein, which can be used interchangeably throughout the document, generally refers to a nucleic acid molecule (e.g., a plasmid, phage, autonomously replicating sequence (ARS), artificial chromosome, yeast artificial chromosome (e.g., YAC) that can be replicated in a host cell and be utilized to introduce a gene or genes into a host cell. The genes introduced on the expression vector can be endogenous genes (e.g., a gene normally found in the host cell or organism) or heterologous genes (e.g., genes not normally found in the genome or on extra-chromosomal nucleic acids of the host cell or organism). The genes introduced into a cell by an expression vector can be native genes or genes that have been modified or engineered. The gene expression vector also can be engineered to contain 5′ and 3′ untranslated regulatory sequences that sometimes can function as enhancer sequences, promoter regions and/or terminator sequences that can facilitate or enhance efficient transcription of the gene or genes carried on the expression vector. A gene expression vector sometimes also is engineered for replication and/or expression functionality (e.g., transcription and translation) in a particular cell type, cell location, or tissue type. Expression vectors sometimes include a selectable marker for maintenance of the vector in the host or recipient cell.

Developmentally regulated promoter: The term “developmentally regulated promoter” as used herein refers to a promoter that acts as the initial binding site for RNA polymerase to transcribe a gene which is expressed under certain conditions that are controlled, initiated by or influenced by a developmental program or pathway. Developmentally regulated promoters often have additional control regions at or near the promoter region for binding activators or repressors of transcription that can influence transcription of a gene that is part of a development program or pathway. Developmentally regulated promoters sometimes are involved in transcribing genes whose gene products influence the developmental differentiation of cells.

Developmentally differentiated cells: The term “developmentally differentiated cells”, as used herein refers to cells that have undergone a process, often involving expression of specific developmentally regulated genes, by which the cell evolves from a less specialized form to a more specialized form in order to perform a specific function. Non-limiting examples of developmentally differentiated cells are liver cells, lung cells, skin cells, nerve cells, blood cells, and the like. Changes in developmental differentiation generally involve changes in gene expression (e.g., changes in patterns of gene expression), genetic re-organization (e.g., remodeling or chromatin to hide or expose genes that will be silenced or expressed, respectively), and occasionally involve changes in DNA sequences (e.g., immune diversity differentiation). Cellular differentiation during development can be understood as the result of a gene regulatory network. A regulatory gene and its cis-regulatory modules are nodes in a gene regulatory network that receive input (e.g., protein expressed upstream in a development pathway or program) and create output elsewhere in the network (e.g., the expressed gene product acts on other genes downstream in the developmental pathway or program).

The term “hyperproliferative disease” is defined as a disease that results from a hyperproliferation of cells. Exemplary hyperproliferative diseases include, but are not limited to cancer or autoimmune diseases. Other hyperproliferative diseases may include vascular occlusion, restenosis, atherosclerosis, or inflammatory bowel disease.

In some embodiments, the nucleic acid is contained within a viral vector. In certain embodiments, the viral vector is an adenoviral vector, or a retroviral or lentiviral vector. It is understood that in some embodiments, the cell is contacted with the viral vector ex vivo, and in some embodiments, the cell is contacted with the viral vector in vivo.

In some embodiments, the cell is a dendritic cell, for example, a mammalian dendritic cell. Often, the cell is a human dendritic cell.

In certain embodiments, the cell is also contacted with an antigen. Often, the cell is contacted with the antigen ex vivo. Sometimes, the cell is contacted with the antigen in vivo. In some embodiments, the cell is in a subject and an immune response is generated against the antigen. Sometimes, the immune response is a cytotoxic T-lymphocyte (CTL) immune response. Sometimes, the immune response is generated against a tumor antigen. In certain embodiments, the cell is activated without the addition of an adjuvant.

In some embodiments, the cell is transduced with the nucleic acid ex vivo and administered to the subject by intradermal administration. In some embodiments, the cell is transduced with the nucleic acid ex vivo and administered to the subject by subcutaneous administration. Sometimes, the cell is transduced with the nucleic acid ex vivo. Sometimes, the cell is transduced with the nucleic acid in vivo.

By MyD88 is meant the myeloid differentiation primary response gene 88, for example, but not limited to the human version, cited as ncbi Gene ID 4615. By “truncated,” is meant that the protein is not full length and may lack, for example, a domain. For example, a truncated MyD88 is not full length and may, for example, be missing the Toll/Interleukin-1 receptor domain (TIR domain). In some examples, the truncated MyD88 polypeptide includes a non-significant portion of the TIR domain, but the TIR domain is not functional. One example of a truncated MyD88 is indicated as MyD88L herein, and is also presented as SEQ ID NO: 2. By a nucleic acid sequence coding for “truncated MyD88” is meant the nucleic acid sequence coding for the truncated MyD88 polypeptide, the term may also refer to the nucleic acid sequence including the portion coding for any amino acids added as an artifact of cloning, including any amino acids coded for by the linkers. The inducible MyD88/CD40 polypeptide may also include full length MyD88 polypeptide, for example, having the nucleotide or amino acid sequence provided in SEQ ID NOs: 906 or 907. The nucleic acid sequence coding for MyD88 or other polypeptides of the present application may be, for example, codon-optimized, comprising preferred codons in modified cells, or wobbled codons as provided herein.

Truncated MyD88 polypeptides include non-full length MyD88 polypeptides that are functionally equivalent to truncated MyD88L polypeptide discussed and exemplified herein. As used herein, the term “functionally equivalent,” as it relates to truncated MyD88, for example, refers to a MyD88 polypeptide that lacks the TIR domain that stimulates an immune response to destroy tumors or hyperproliferative disease. “Functionally equivalent” or “a functional fragment” of a MyD88 polypeptide refers, for example, to a truncated MyD88 polypeptide whether lacking the TIR domain or not that is capable of amplifying the cell-mediated tumor killing response when expressed in cells, for example, T cells, NK cells, or NK-T cells, such as, for example, the T cell-mediated, NK cell-mediated, NK-T cell-mediated, for example invariant NK-T cell-mediated, or gamma delta T cell-mediated response, by, for example, activating the NFκB pathway.

In the methods herein, the costimulatory fragment (e.g., OX40, ICOS, MyD88, CD28, 4-1BB, and the like) portion of the chimeric signaling polypeptide may be located either upstream or downstream from the inducible MyD88 or truncated MyD88 polypeptide portion. Also, the inducible CD40 portion and the inducible MyD88 or truncated MyD88 adapter protein portions may be transfected or transduced into the cells either on the same vector, in cis, or on separate vectors, in trans.

Truncated MyD88 polypeptides may, for example, comprise amino acid residues 1-172 of the full length MyD88 amino acid sequence, for example, residues 1-172 of SEQ ID NO: 907. In some embodiments, Truncated MyD88 polypeptides may, for example, comprise amino acid residues 1-151 or 1-155 of the full length MyD88 amino acid sequence, for example, residues 1-151 or 1-155 of SEQ ID NO: 907. In some embodiments, truncated MyD88 polypeptides may, for example, comprise amino acid residues 1-152, 153, 154, 155, 156, 157, 158, 159, 160, 161, 162, 163, 164, 165, 166, 167, 168, 169, 170, or 171 of the full length MyD88 amino acid sequence; an example of a full length MyD88 amino acid sequence is provided as SEQ ID NO: 907.

Truncated MyD88 polypeptides may, for example, consist of amino acid residues 1-172 of the full length MyD88 amino acid sequence, for example, residues 1-172 of SEQ ID NO: 907. In some embodiments, Truncated MyD88 polypeptides may, for example, consist of amino acid residues 1-151 or 1-155 of the full length MyD88 amino acid sequence, for example, residues 1-151 or 1-155 of SEQ ID NO: 907. In some embodiments, truncated MyD88 polypeptides may, for example, consist of amino acid residues 1-152, 153, 154, 155, 156, 157, 158, 159, 160, 161, 162, 163, 164, 165, 166, 167, 168, 169, 170, or 171 of the full length MyD88 amino acid sequence; an example of a full length MyD88 amino acid sequence is provided as SEQ ID NO: 907.

In some embodiments, the truncated MyD88 amino acid sequence does not include contiguous amino acid residues 173-296 of the full length MyD88 amino acid sequence. In some embodiments, the truncated MyD88 amino acid sequence does not include contiguous amino acid residues 152-296 of the full length MyD88 amino acid sequence. In some embodiments, the truncated MyD88 amino acid sequence does not include contiguous amino acid residues 156-296 of the full length MyD88 amino acid sequence. In some embodiments, the truncated MyD88 amino acid sequence does not include contiguous amino acid residues 152, 153, 154, 155, 156, 157, 158, 159, 160, 161, 162, 163, 164, 165, 166, 167, 168, 169, 170, 171, or 172-296 of the full length MyD88 amino acid sequence. By “full length MyD88 amino acid sequence” is meant a full length MyD88 amino acid sequence that corresponds to, for example, SEQ ID NO: 907.

The cell in some embodiments is contacted with an antigen, sometimes ex vivo. In certain embodiments the cell is in a subject and an immune response is generated against the antigen, such as a cytotoxic T-lymphocyte (CTL) immune response. In certain embodiments, an immune response is generated against a tumor antigen (e.g., PSMA). In some embodiments, the nucleic acid is prepared ex vivo and administered to the subject by intradermal administration or by subcutaneous administration, for example. Sometimes the cell is transduced or transfected with the nucleic acid ex vivo or in vivo.

In some embodiments, the nucleic acid comprises a promoter sequence operably linked to the polynucleotide sequence. Alternatively, the nucleic acid comprises an ex vivo-transcribed RNA, containing the protein-coding region of the chimeric protein.

By “reducing tumor size” or “inhibiting tumor growth” of a solid tumor is meant a response to treatment, or stabilization of disease, according to standard guidelines, such as, for example, the Response Evaluation Criteria in Solid Tumors (RECIST) criteria. For example, this may include a reduction in the diameter of a solid tumor of about 5%, 10%, 20%, 30%, 40%, 50%, 60%, 70%, 80%, 90%, or 100%, or the reduction in the number of tumors, circulating tumor cells, or tumor markers, of about 5%, 10%, 20%, 30%, 40%, 50%, 60%, 70%, 80%, 90%, or 100%. The size of tumors may be analyzed by any method, including, for example, CT scan, MRI, for example, CT-MRI, chest X-ray (for tumors of the lung), or molecular imaging, for example, PET scan, such as, for example, a PET scan after administering an iodine 123-labelled PSA, for example, PSMA ligand, such as, for example, where the inhibitor is TROFEX™/MIP-1072/1095, or molecular imaging, for example, SPECT, or a PET scan using PSA, for example, PSMA antibody, such as, for example, capromad pendetide (Prostascint), a 111-iridium labeled PSMA antibody.

By “reducing, slowing, or inhibiting tumor vascularization is meant a reduction in tumor vascularization of about 5%, 10%, 20%, 30%, 40%, 50%, 60%, 70%, 80%, 90%, or 100%, or a reduction in the appearance of new vasculature of about 5%, 10%, 20%, 30%, 40%, 50%, 60%, 70%, 80%, 90%, or 100%, when compared to the amount of tumor vascularization before treatment. The reduction may refer to one tumor, or may be a sum or an average of the vascularization in more than one tumor. Methods of measuring tumor vascularization include, for example, CAT scan, MRI, for example, CT-MRI, or molecular imaging, for example, SPECT, or a PET scan, such as, for example, a PET scan after administering an iodine 123-labelled PSA, for example, PSMA ligand, such as, for example, where the inhibitor is TROFEX™/MIP-1072/1095, or a PET scan using PSA, for example, PSMA antibody, such as, for example, capromad pendetide (Prostascint), a 111-iridium labeled PSMA antibody.

A tumor is classified as a prostate cancer tumor when, for example, the tumor is present in the prostate gland, or has derived from or metastasized from a tumor in the prostate gland, or produces PSA. A tumor has metastasized from a tumor in the prostate gland, when, for example, it is determined that the tumor has chromosomal breakpoints that are the same as, or similar to, a tumor in the prostate gland of the subject.

Incorporated by reference in their entirety are U.S. Pat. No. 7,404,950, titled Induced Activation in Dendritic Cells, issued Jun. 29, 2008, to Spencer, D. et al.; U.S. Pat. No. 8,691,210, titled Methods and Compositions for Generating an Immune Response by Inducing CD40 and Pattern Recognition Receptors and Adaptors Thereof, to Spencer, D., et al., issued Apr. 8, 2014; and U.S. Pat. No. 9,315,559, titled Methods and Compositions for Generating an Immune Response by Inducing Pattern Recognition Adapters, to Spencer, D. et al., issued Apr. 19, 2016. Also incorporated by reference in their entirety are U.S. patent application Ser. No. 13/087,329, titled Methods for Treating Prostate Cancer, by Slawin, K., et al., filed Apr. 14, 2011; U.S. patent application Ser. No. 13/763,591 by Spencer, D., et al., filed Feb. 8, 2013; International Patent Application PCT/US2009/057738, filed Sep. 21, 2009, published Mar. 28, 2010 as WO2010/033949; International Patent Application PCT/US2011/032572, titled Method for Treating Solid Tumors, by Slawin, K., et al., filed Apr. 14, 2011, published as WO2011/130566, Oct. 20, 2011; U.S. patent application Ser. No. 14/210,034, titled Methods for Controlling T Cell Proliferation, by Spencer, D., et al., filed Mar. 13, 2014, published as US-2014-0286987A1 on Sep. 25, 2014; International Patent Application PCT/US2014/026734, filed Mar. 13, 2014, published as WO/2014/151960 on Sep. 25, 2014; U.S. patent application Ser. No. 14/622,018, titled Methods for Activating T Cells Using an Inducible Chimeric Polypeptide, by Foster, A. E., et al., filed Feb. 13, 2015, published as US-2016-0046700 on Feb. 18, 2016; International Patent Application PCT/US2015/015829, filed Feb. 13, 2015, published as WO/2015/123527 on Aug. 20, 2015; U.S. patent application Ser. No. 14/842,710, titled Costimulation of Chimeric Antigen Receptors by MyD88 and CD40 polypeptide, by Spencer, D., et al, filed Sep. 1, 2015, published as US-2016-0058857A1 on Mar. 3, 2016; and International Patent Application PCT/US2015/047957, filed Dec. 14, 2015, published as WO/2016/036746 on Mar. 10, 2016.

Chimeric Antigen Receptors

By “chimeric antigen receptor” or “CAR” is meant, for example, a chimeric polypeptide which comprises a polypeptide sequence that recognizes a target antigen (an antigen-recognition domain) linked to a transmembrane polypeptide and intracellular domain polypeptide selected to activate the T cell and provide specific immunity. The antigen-recognition domain may be a single-chain variable fragment (scFv), or may, for example, be derived from other molecules such as, for example, a T cell receptor or Pattern Recognition Receptor. The intracellular domain comprises at least one polypeptide which causes activation of the T cell, such as, for example, but not limited to, CD3 zeta, and, for example, costimulatory molecules, for example, but not limited to, CD28, OX40 and 4-1BB. The term “chimeric antigen receptor” may also refer to chimeric receptors that are not derived from antibodies, but are chimeric T cell receptors. These chimeric T cell receptors may comprise a polypeptide sequence that recognizes a target antigen, where the recognition sequence may be, for example, but not limited to, the recognition sequence derived from a T cell receptor or a scFv. The intracellular domain polypeptides are those that act to activate the T cell. Chimeric T cell receptors are discussed in, for example, Gross, G., and Eshar, Z., FASEB Journal 6:3370-3378 (1992), and Zhang, Y., et al., PLOS Pathogens 6:1-13 (2010).

In one type of chimeric antigen receptor (CAR), the variable heavy (VH) and light (VL) chains for a tumor-specific monoclonal antibody are fused in-frame with the CD3 zeta chain (ζ) from the T cell receptor complex. The VH and VL are generally connected together using a flexible glycine-serine linker, and then attached to the transmembrane domain by a spacer (CH2CH3) to extend the scFv away from the cell surface so that it can interact with tumor antigens. Following transduction, T cells now express the CAR on their surface, and upon contact and ligation with a tumor antigen, signal through the CD3 zeta chain inducing cytotoxicity and cellular activation.

Investigators have noted that activation of T cells through CD3 zeta is sufficient to induce a tumor-specific killing, but is insufficient to induce T cell proliferation and survival. Early clinical trials using T cells modified with first generation CARs expressing only the zeta chain showed that gene-modified T cells exhibited poor survival and proliferation in vivo.

As costimulation through the B7 axis is necessary for complete T cell activation, investigators added the costimulating polypeptide CD28 signaling domain to the CAR construct. This region generally contains the transmembrane region (in place of the CD3 zeta version) and the YMNM motif for binding PI3K and Lck. In vivo comparisons between T cells expressing CARs with only zeta or CARs with both zeta and CD28 demonstrated that CD28 enhanced expansion in vivo, in part due to increased IL-2 production following activation. The inclusion of CD28 is called a 2nd generation CAR. The most commonly used costimulating molecules include CD28 and 4-1BB, which, following tumor recognition, can initiate a signaling cascade resulting in NF-κB activation, which promotes both T cell proliferation and cell survival.

T Cell Receptors

T cell receptors are molecules composed of two different polypeptides that are on the surface of T cells. They recognize antigens bound to major histocompatibility complex molecules; upon recognition with the antigen, the T cell is activated. By “recognize” is meant, for example, that the T cell receptor, or fragment or fragments thereof, such as TCRα polypeptide and TCRβ together, is capable of contacting the antigen and identifying it as a target. TCRs may comprise α and β polypeptides, or chains. The α and β polypeptides include two extracellular domains, the variable and the constant domains. The variable domain of the α and β polypeptides has three complementarity determining regions (CDRs); CDR3 is considered to be the main CDR responsible for recognizing the epitope. The a polypeptide includes the V and J regions, generated by VJ recombination, and the β polypeptide includes the V, D, and J regions, generated by VDJ recombination. The intersection of the VJ regions and VDJ regions corresponds to the CDR3 region. TCRs are often named using the International Immunogenetics (IMGT) TCR nomenclature (IMGT Database, www.IMGT.org; Giudicelli, V., et al., IMGT/LIGM-DB, the IMGT® comprehensive database of immunoglobulin and T cell receptor nucleotide sequences, Nucl. Acids Res., 34, D781-D784 (2006). PMID: 16381979; T cell Receptor Factsbook, LeFranc and LeFranc, Academic Press ISBN 0-12-441352-8).

Recombinant T cell receptors may bind to, for example, antigenic polypeptides such as Bob-1, PRAME, and NY-ESO-1. (U.S. patent application Ser. No. 14/930,572, filed Nov. 2, 2015, titled T Cell Receptors Directed Against Bob1 and Uses Thereof, published as US2016-0129094A1 on May 12, 2016; International Patent Application PCT/IB2015/002191, filed Nov. 2, 2015, published as WO2016/071758 on May 12, 2016; U.S. patent application Ser. No. 15/065,567, filed Mar. 9, 2016, titled T Cell Receptors Directed Against the Preferentially-Expressed Antigen of Melanoma and Uses Thereof, published as US20160263155A1 on Sep. 15, 2016; and International Patent Application PCT/IB2016/000399, filed Mar. 9, 2016, each of which is incorporated by reference in its entirety herein).

The present application includes, in some embodiments, inducible chimeric signaling polypeptides, or cells that express inducible chimeric signaling polypeptides that also express heterologous polypeptides. For example, in another example of cell therapy, T cells are modified so that they express a non-functional TGF-beta receptor, rendering them resistant to TGF-beta. This allows the modified T cells to avoid the cytotoxicity caused by TGF-beta, and allows the cells to be used in cellular therapy (Bollard, C. J., et al., (2002) Blood 99:3179-3187; Bollard, C. M., et al., (2004) J. Exptl. Med. 200:1623-1633). However, it also could result in a T cell lymphoma, or other adverse effect, as the modified T cells now lack part of the normal cellular control; these therapeutic T cells could themselves become malignant. Transducing these modified T cells with a chimeric Caspase-9-based safety switch as presented herein, would provide a safety switch that could avoid this result.

In other examples of the expression of a heterologous polypeptide in the compositions and methods of the present application, natural Killer cells are modified to express the membrane-targeting polypeptide. Instead of a chimeric antigen receptor, in certain embodiments, the heterologous membrane bound polypeptide is a NKG2D receptor. NKG2D receptors can bind to stress proteins (e.g. MICA/B) on tumor cells and can thereby activate NK cells. The extracellular binding domain can also be fused to signaling domains (Barber, A., et al., Cancer Res 2007; 67: 5003-8; Barber A, et al., Exp Hematol. 2008; 36:1318-28; Zhang T., et al., Cancer Res. 2007; 67:11029-36., and this could, in turn, be linked to FRB domains, analogous to FRB-linkered CARs.

Moreover, other cell surface receptors, such as VEGF-R could be used as a docking site for FRB domains to enhance tumor-dependent clustering in the presence of hypoxia-triggered VEGF, found at high levels within many tumors.

Cells used in cellular therapy, that express a heterologous gene, such as a modified receptor, or a chimeric receptor, may be transduced with nucleic acid that encodes the inducible chimeric signaling polypeptides or the chimeric signaling polypeptides, and/or with nucleic acid that encodes a chimeric Caspase-9-based safety switch before, after, or at the same time, as the cells are transduced with the heterologous gene.

Immune Cell Activation

A cell is “activated,” when one or more activities associated with activated cells may be observed and/or measured. For example, an cell is activated when following contact with an expression vector presented herein, an activity associated with activation may be measured in the expression vector-contacted cell as compared to an cell that has either not been contacted with the expression vector, or has been contacted with a negative control vector. In one example, the increased activity may be at a level of two, three, four, five, six, seven, eight, nine, or ten fold, or more, than that of the non-contacted cell, or the cell contacted with the negative control. For example, one of the following activities may be enhanced in an cell that has been contacted with the expression vector: costimulatory molecule expression on the cell, nuclear translocation of NF-kappaB in cells, DC maturation marker expression, such as, for example, toll-like receptor expression or CCR7 expression, specific cytotoxic T lymphocyte responses, such as, for example, specific lytic activity directed against tumor cells, or cytokine (for example, IL-2) or chemokine expression.

An amount of a composition that activates cells or that “enhances” an immune response refers to an amount in which an immune response is observed that is greater or intensified or deviated in any way with the addition of the composition when compared to the same immune response measured without the addition of the composition. For example, the lytic activity of cytotoxic T cells can be measured, for example, using a ⁵¹Cr release assay, with and without the composition. The amount of the substance at which the CTL lytic activity is enhanced as compared to the CTL lytic activity without the composition is said to be an amount sufficient to enhance the immune response of the animal to the antigen. For example, the immune response may be enhanced by a factor of at least about 2, or, for example, by a factor of about 3 or more. The amount of cytokines secreted may also be altered.

The enhanced immune response may be an active or a passive immune response. Alternatively, the response may be part of an adaptive immunotherapy approach in which cells are obtained with from a subject (e.g., a patient), then transduced or transfected with a composition comprising the expression vector or construct presented herein. The cells may be obtained from, for example, the blood of the subject or bone marrow of the subject. The cells may then be administered to the same or different animal, or same or different subject (e.g., same or different donors). In certain embodiments the subject (for example, a patient) has or is suspected of having a cancer, such as for example, prostate cancer, or has or is suspected of having an infectious disease. In other embodiments the method of enhancing the immune response is practiced in conjunction with a known cancer therapy or any known therapy to treat the infectious disease.

Dendritic Cells

Antigen presenting cells (APCs) are cells that can prime T-cells against a foreign antigen by displaying the foreign antigen with major histocompatibility complex (MHC) molecules on their surface. There are two types of APCs, professional and non-professional. The professional APCs express both MHC class I molecules and MHC class II molecules, the non-professional APCs do not constitutively express MHC class II molecules. In particular embodiments, professional APCs are used in the methods herein. Professional APCs include, for example, B-cells, macrophages, and dendritic cells.

The innate immune system uses a set of germline-encoded receptors for the recognition of conserved molecular patterns present in microorganisms. These molecular patterns occur in certain constituents of microorganisms including: lipopolysaccharides, peptidoglycans, lipoteichoic acids, phosphatidyl cholines, bacteria-specific proteins, including lipoproteins, bacterial DNAs, viral single and double-stranded RNAs, unmethylated CpG-DNAs, mannans and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism (Janeway et al. (1989) Cold Spring Harb. Symp. Quant. Biol., 54: 1-13; Medzhitov et al., Nature, 388:394-397, 1997).

The receptors of the innate immune system that recognize PAMPs are called Pattern Recognition Receptors (PRRs) (Janeway et al., 1989; Medzhitov et al., 1997). These receptors vary in structure and belong to several different protein families. Some of these receptors recognize PAMPs directly (e.g., CD14, DEC205, collectins), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly (Medzhitov et al., 1997; Fearon et al. (1996) Science 272: 50-3).

Cellular PRRs are expressed on effector cells of the innate immune system, including cells that function as professional cells (APC) in adaptive immunity. Such effector cells include, but are not limited to, macrophages, dendritic cells, B lymphocytes and surface epithelia. This expression profile allows PRRs to directly induce innate effector mechanisms, and also to alert the host organism to the presence of infectious agents by inducing the expression of a set of endogenous signals, such as inflammatory cytokines and chemokines, as discussed below. This latter function allows efficient mobilization of effector forces to combat the invaders.

The primary function of dendritic cells (DCs) is to acquire antigen in the peripheral tissues, travel to secondary lymphoid tissue, and present antigen to effector T cells of the immune system (Banchereau, J., et al., Annu Rev Immunol, 2000, 18: p. 767-811; Banchereau, J., & Steinman, R. M., Nature 392, 245-252 (1998)). As DCs carry out their crucial role in the immune response, they undergo maturational changes allowing them to perform the appropriate function for each environment (Termeer, C. C., et al., J Immunol, 2000, Aug. 15. 165: p. 1863-70). During DC maturation, antigen uptake potential is lost, the surface density of major histocompatibility complex (MHC) class I and class II molecules increases by 10-100 fold, and CD40, costimulatory and adhesion molecule expression also greatly increases (Lanzavecchia, A. and F. Sallusto, Science, 2000. 290: p. 92-96). In addition, other genetic alterations permit the DCs to home to the T cell-rich paracortex of draining lymph nodes and to express T-cell chemokines that attract naïve and memory T cells and prime antigen-specific naïve TH0 cells (Adema, G. J., et al., Nature, 1997, June 12. 387: p. 713-7). During this stage, mature DCs present antigen via their MHC II molecules to CD4⁺ T helper cells, inducing the upregulation of T cell CD40 ligand (CD40L) that, in turn, engages the DC CD40 receptor. This DC:T cell interaction induces rapid expression of additional DC molecules that are crucial for the initiation of a potent CD8⁺ cytotoxic T lymphocyte (CTL) response, including further upregulation of MHC I and II molecules, adhesion molecules, costimulatory molecules (e.g., B7.1,B7.2), cytokines (e.g., IL-12) and anti-apoptotic proteins (e.g., Bcl-2) (Anderson, D. M., et al., Nature, 1997, Nov. 13. 390: p. 175-9; Ohshima, Y., et al., J Immunol, 1997, Oct. 15. 159: p. 3838-48; Sallusto, F., et al., Eur J Immunol, 1998, Sep. 28: p. 2760-9; Caux, C. Adv Exp Med Biol. 1997, 417:21-5). CD8⁺ T cells exit lymph nodes, reenter circulation and home to the original site of inflammation to destroy pathogens or malignant cells.

One key parameter influencing the function of DCs is the CD40 receptor, serving as the “on switch” for DCs (Bennett, S. R., et all, Nature, 1998, Jun. 4. 393: p. 478-80; Clarke, S. R., J Leukoc Biol, 2000, May. 67: p. 607-14; Fernandez, N. C., et al., Nat Med, 1999, Apr. 5: p. 405-11; Ridge, J. P., D. R. F, and P. Nature, 1998, Jun. 4. 393: p. 474-8; Schoenberger, S. P., et al., Nature, 1998, Jun. 4. 393: p. 480-3). CD40 is a 48-kDa transmembrane member of the TNF receptor superfamily (McWhirter, S. M., et al., Proc Natl Acad Sci USA, 1999, Jul. 20. 96: p. 8408-13). CD40L interaction induces CD40 trimerization, necessary for initiating signaling cascades involving TNF receptor associated factors (TRAFs) (Ni, C., et al., PNAS, 2000, 97(19): 10395-10399; Pullen, S. S., et al., J Biol Chem, 1999, May 14.274: p. 14246-54). CD40 uses these signaling molecules to activate several transcription factors in DCs, including NF-kappa B, AP-1, STAT3, and p38MAPK (McWhirter, S. M., et al., 1999).

Due to their unique method of processing and presenting antigens and the potential for high-level expression of costimulatory and cytokine molecules, dendritic cells (DC) are effective cells (APCs) for priming and activating naïve T cells (Banchereau J, et al., Ann N Y Acad Sci. 2003; 987:180-187). This property has led to their widespread use as a cellular platform for vaccination in a number of clinical trials with encouraging results (O'Neill D W, et al., Blood. 2004; 104:2235-2246; Rosenberg S A, Immunity. 1999; 10:281-287). However, the clinical efficacy of DC vaccines in cancer patients has been unsatisfactory, probably due to a number of key deficiencies, including suboptimal activation, limited migration to draining lymph nodes, and an insufficient life span for optimal T cell activation in the lymph node environment.

A parameter in the optimization of DC-based cancer vaccines is the interaction of DCs with immune effector cells, such as CD4⁺, CD8⁺ T cells and T regulatory (Treg) cells. In these interactions, the maturation state of the DCs is a key factor in determining the resulting effector functions (Steinman R M, Annu Rev Immunol. 2003; 21:685-711). To maximize CD4⁺ and CD8⁺ T cell priming while minimizing Treg expansion, DCs need to be fully mature, expressing high levels of costimulatory molecules, (like CD40, CD80, and CD86), and pro-inflammatory cytokines, like IL-12p70 and IL-6. Equally important, the DCs must be able to migrate efficiently from the site of vaccination to draining lymph nodes to initiate T cell interactions (Vieweg J, et al., Springer Semin Immunopathol. 2005; 26:329-341).

For the ex vivo maturation of monocyte-derived immature DCs, the majority of DC-based trials have used a standard maturation cytokine cocktail (MC), comprised of TNF-alpha, IL-1beta, IL-6, and PGE2. The principal function of prostaglandin E2 (PGE2) in the standard maturation cocktail is to sensitize the CC chemokine receptor 7 (CCR7) to its ligands, CC chemokine ligand 19 (CCL19) and CCL21 and thereby enhance the migratory capacity of DCs to the draining lymph nodes (Scandella E, et al., Blood. 2002; 100:1354-1361; Luft T, et al., Blood. 2002; 100:1362-1372). However, PGE2 has also been reported to have numerous properties that are potentially deleterious to the stimulation of an immune response, including suppression of T-cell proliferation, (Goodwin J S, et al., J Exp Med. 1977; 146:1719-1734; Goodwin J S, Curr Opin Immunol. 1989; 2:264-268) inhibition of pro-inflammatory cytokine production (e.g., IL-12p70 and TNF-alpha (Kalinski P, Blood. 2001; 97:3466-3469; van der Pouw Kraan T C, et al., J Exp Med. 1995; 181:775-779)), and down-regulation of major histocompatibility complex (MHC) II surface expression (Snyder D S, Nature. 1982; 299:163-165). Therefore, maturation protocols that can avoid PGE2 while promoting migration are likely to improve the therapeutic efficacy of DC-based vaccines.

A DC activation system based on targeted temporal control of the CD40 signaling pathway has been developed to extend the pro-stimulatory state of DCs within lymphoid tissues. DC functionality was improved by increasing both the amplitude and the duration of CD40 signaling (Hanks B A, et al., Nat Med. 2005; 11:130-137). To accomplish this, the CD40 receptor was re-engineered so that the cytoplasmic domain of CD40 was fused to synthetic ligand-binding domains along with a membrane-targeting sequence. Administration of a lipid-permeable, dimerizing drug, AP20187 (AP), called a chemical inducer of dimerization (CID) (Spencer D M, et al., Science. 1993; 262:1019-1024), led to the in vivo induction of CD40-dependent signaling cascades in murine DCs. This induction strategy significantly enhanced the immunogenicity against both defined antigens and tumors in vivo beyond that achieved with other activation modalities (Hanks B A, et al., Nat Med. 2005; 11:130-137).

Pattern recognition receptor (PRR) signaling, an example of which is Toll-like receptor (TLR) signaling also plays a critical role in the induction of DC maturation and activation; human DCs express, multiple distinct TLRs (Kadowaki N, et al., J Exp Med. 2001; 194:863-869). The eleven mammalian TLRs respond to various pathogen-derived macromolecules, contributing to the activation of innate immune responses along with initiation of adaptive immunity.

Lipopolysaccharide (LPS) and a clinically relevant derivative, monophosphoryl lipid A (MPL), bind to cell surface TLR-4 complexes (Kadowaki N, et al., J Exp Med. 2001; 194:863-869), leading to various signaling pathways that culminate in the induction of transcription factors, such as NF-kappaB and IRF3, along with mitogen-activated protein kinases (MAPK) p38 and c-Jun kinase (JNK) (Ardeshna K M, et al., Blood. 2000; 96:1039-1046; Ismaili J, et al., J Immunol. 2002; 168:926-932). During this process DCs mature, and partially upregulate pro-inflammatory cytokines, like IL-6, IL-12, and Type I interferons (Rescigno M, et al., J Exp Med. 1998; 188:2175-2180). LPS-induced maturation has been shown to enhance the ability of DCs to stimulate antigen-specific T cell responses in vitro and in vivo (Lapointe R, et al., Eur J Immunol. 2000; 30:3291-3298). Methods for activating an cell, comprising transducing the cell with a nucleic acid coding for a CD40 polypeptide have been discussed in U.S. Pat. No. 7,404,950, and methods for activating an cell, comprising transfecting the cell with a nucleic acid coding for a chimeric protein including an inducible CD40 polypeptide and a Pattern Recognition Receptor, or other downstream proteins in the pathway have been discussed in International Patent Application No. PCT/US2007/081963, filed Oct. 19, 2007, published as WO 2008/049113, which are hereby incorporated by reference herein.

An inducible CD40 (iCD40) system has been applied to human dendritic cells (DCs) and it has been demonstrated that combining iCD40 signaling with Pattern recognition receptor (PRR) adapter ligation causes persistent and robust activation of human DCs. (Spencer, et al., U.S. Ser. No. 12/563,991, filed Sep. 21, 2009, related international application published on Mar. 25, 2010 as WO 2010/033949, hereby incorporated by reference herein).

Engineering Expression Constructs

Expression constructs encode a truncated MyD88 polypeptide, costimulatory polypeptide cytoplasmic signaling region and optionally a ligand-binding domain, all operatively linked; the expression constructs may also encode an antigen recognition polypeptide of a CAR, as well as a transmembrane region, also operatively linked. In some embodiments, the inducible or constitutive chimeric signaling polypeptide is expressed as a separate polypeptide from a CAR or a recombinant TCR, where the expression of the two polypeptides is operatively linked. In yet other embodiments, the expression constructs may comprise inducible Caspase-9 polypeptides, also operatively linked to the inducible chimeric signaling polypeptides. In general, the term “operably linked” is meant to indicate that the promoter sequence is functionally linked to a second sequence, wherein the promoter sequence initiates and mediates transcription of the DNA corresponding to the second sequence.

Expression constructs may comprise one or more isolated nucleic acids. The term “isolation” as applied to a nucleic acid refers to the separation of one region of a nucleotide sequence from other regions of the nucleotide sequence. Thus, isolated nucleic acids are isolated from chromosomes. Isolation may, for example, be performed using an amplification reaction, such as, for example, PCR; in other examples, nucleic acids may be isolated from the cells from which they naturally are found. A pool of isolated nucleic acids may be enriched in nucleic acid segments containing only sequences for a particular region of interest. In some embodiments, isolated nucleic acids are shorter than full length sequences encoding an entire protein.

For expression constructs encoding inducible chimeric signaling polypeptides, more than one ligand-binding domain may be used in the expression construct. Yet further, the expression construct may contain a membrane-targeting sequence for either the inducible or the constitutive chimeric signaling polypeptides. Appropriate expression constructs may include the MyD88/costimulatory polypeptide element on either side, that is, for the polynucleotide, 5′ or 3′, of the above FKBP ligand-binding elements. The expression construct may be inserted into a vector, for example a viral vector or plasmid. The steps of the methods provided may be performed using any suitable method; these methods include, without limitation, methods of transfecting, transducing, or otherwise providing nucleic acid to the cell, presented herein. In some embodiments, the truncated MyD88 polypeptide is encoded by the nucleotide sequence of SEQ ID NO: 1 (with or without DNA linkers or has the amino acid sequence of SEQ ID NO: 2).

In some embodiments, the polynucleotide may encode the inducible or constitutive chimeric signaling polypeptide and heterologous polypeptide, which may be, for example a marker polypeptide and may be, for example, a chimeric antigen receptor. The heterologous polypeptide, for example, the chimeric antigen receptor, may be linked to the inducible chimeric signaling polypeptide via a polypeptide sequence, such as, for example, a 2A-like linker polypeptide.

In certain examples, a nucleic acid comprising a polynucleotide coding for an inducible chimeric signaling polypeptide is included in the same vector, such as, for example, a viral or plasmid vector, as a polynucleotide coding for a second polypeptide. This second polypeptide may be, for example, a chimeric antigen receptor polypeptide, a recombinant T cell receptor, as discussed herein, or a marker polypeptide. In these examples, the construct may be designed with one promoter operably linked to a nucleic acid comprising a polynucleotide coding for the two polypeptides, linked by a 2A polypeptide. In this example, the first and second polypeptides are separated during translation, resulting in a chimeric signaling polypeptide, and the second polypeptide. In other examples, the two polypeptides may be expressed separately from the same vector, where each nucleic acid comprising a polynucleotide coding for one of the polypeptides is operably linked to a separate promoter. In yet other examples, one promoter may be operably linked to the two nucleic acids, directing the production of two separate RNA transcripts, and thus two polypeptides. Therefore, the expression constructs discussed herein may comprise at least one, or at least two promoters.

The expression constructs may further comprise a polynucleotide sequence that encodes a heterologous polypeptide. The terms heterologous protein and heterologous polypeptide may be interchangeable. By “heterologous polypeptide” or “heterologous protein” s meant a polypeptide protein that is not a functional domain of the chimeric signaling polypeptide. Examples of heterologous polypeptides include, for example, marker polypeptides, chimeric antigen receptors, recombinant T cell receptors, polypeptides that may comprise an antigen recognition moiety, polypeptides having enzymatic activity. In certain embodiments, the heterologous polypeptide is linked to the inducible chimeric signaling polypeptide. For example, the heterologous polypeptide may be linked to the inducible chimeric signaling polypeptide via a polypeptide sequence, such as, for example, a cleavable 2A-like sequence. Or, the heterologous polypeptide may be expressed as a separate polypeptide, under the control of a separate promoter.

The expression constructs may further comprise a polynucleotide sequence that encodes a marker polypeptide. In certain embodiments, the marker polypeptide is linked to the inducible chimeric signaling polypeptide. For example, the marker polypeptide may be linked to the inducible chimeric signaling polypeptide via a polypeptide sequence, such as, for example, a cleavable 2A-like sequence. Or, the marker polypeptide may be expressed as a separate polypeptide, under the control of a separate promoter. The marker polypeptide may be, for example, CD19, ΔCD19, or may be any polypeptide that can be detected and used as a marker to determine if a cell expresses the nucleic acid coding for the chimeric signaling polypeptide. Markers may be detected using, for example, immunological, biochemical, or other functional assays.

Cleavable Linker Sequences

In some embodiments, vectors, such as plasmid or viral vectors, are provided that comprise a nucleic acid that encodes two or more polypeptides under the control of a single promoter. In some examples, the nucleic acid encodes a cleavable linker polypeptide, or cleavable linker sequence, between polypeptides. The polypeptides are then separated during translation of the polypeptide.

2A-like sequences, or “cleavable” 2A sequences, are derived from, for example, many different viruses, including, for example, from Thosea asigna virus. These sequences are sometimes also known as “peptide skipping sequences.” When this type of sequence is placed within a cistron, between two peptides that are intended to be separated, the ribosome appears to skip a peptide bond, in the case of Thosea asigna sequence; the bond between the Gly and Pro amino acids is omitted. This leaves two polypeptides, in for example, a costimulatory polypeptide and a chimeric antigen receptor, or in some cases, a chimeric inducible caspase-9 polypeptide and a marker polypeptide. When this sequence is used, the polypeptide that is encoded 5′ of the 2A sequence may end up with additional amino acids at the carboxy terminus, including the Gly residue and any upstream in the 2A sequence. The polypeptide that is encoded 3′ of the 2A sequence may end up with additional amino acids at the amino terminus, generally the Pro residue at the new amino terminus of the polypeptide. “2A” or “2A-like” sequences are part of a large family of polypeptides that can cause peptide bond-skipping. (Donnelly, M L 2001, J. Gen. Virol. 82:1013-25). Various 2A sequences have been characterized (e.g., F2A, P2A, T2A), and are examples of 2A-like sequences that may be used in the polypeptides of the present application.

The 2A-like sequences are sometimes “leaky” in that some of the polypeptides are not separated during translation, and instead, remain as one long polypeptide following translation. One theory as to the cause of the leaky linker, is that the short 2A sequence occasionally may not fold into the required structure that promotes ribosome skipping (a “2A fold”). In these instances, ribosomes may not miss the proline, which then results in a fusion protein. To reduce the level of leakiness, and thus reduce the number of fusion proteins that form, a GSG (or similar) linker may be added to the amino terminal side of the 2A polypeptide; the GSG linker blocks secondary structures of newly-translated polypeptides from spontaneously folding and disrupting the ‘2A fold’.

Costimulatory and Co-Activation Polypeptides

Costimulatory polypeptide molecules are capable of amplifying the cell-mediated immune response through activation of signaling pathways involved in cell survival and proliferation. Costimulatory polypeptides may also be referred to herein as co-activation polypeptides in the embodiments provided herein. In some embodiments, costimulation refers to signaling domains that complement a CD3 zeta chain of a chimeric antigen receptor, such as, for example, the cytoplasmic signaling domains of CD28 and 4-1BB. For purposes of the present embodiments, costimulation, or co-activation may also refer to cytoplasmic signaling regions that are members of the CD28 and TNF families of proteins. In some embodiments, costimulatory polypeptides include any molecule or polypeptide that activates the NF-kappaB pathway, Akt pathway, and/or p38 pathway. The cellular activation system is based upon utilizing a recombinant signaling molecule fused to one or more ligand-binding domains (i.e., a small molecule binding domain) in which the costimulatory polypeptide is activated and/or regulated with a ligand resulting in oligomerization (i.e., a lipid-permeable, organic, dimerizing drug). Other systems that may be used for crosslinking, or for oligomerization, of costimulatory polypeptides include antibodies, natural ligands, and/or artificial cross-reacting or synthetic ligands. Yet further, another dimerization systems contemplated include the coumermycin/DNA gyrase B system.

Costimulatory polypeptides that may be contemplated as part of the chimeric signaling polypeptides herein include those that activate NF-kappaB and other variable signaling cascades for example the p38 pathway and/or Akt pathway. Co-activation or costimulatory polypeptides that may be contemplated also include chimeric polypeptides or fusion polypeptides between, for example, one or more costimulatory polypeptides, such as, for example a fusiong of regions of RANK polypeptide and CD40 polypeptide. In some embodiments, the chimeric signaling polypeptides provided herein, and the inducible chimeric signaling polypeptides provided herein lack a CD40 cytoplasmic region, or comprise no CD40 polypeptide region. In some embodiments, the modified cells and nucleic acids provided herein encode chimeric signaling polylpeptides or inducible chimeric signaling polypeptides that lack a CD40 cytoplasmic region, or comprise no CD40 cytoplasmic region. In some embodiments provided herein are modified cells or nucleic acids that comprise polynucleotides that encode costimulatory polypeptides that comprise or consist of fusions between CD40 polypeptide regions and RANK cytoplasmic regions. In some embodiments provided herein are costimulatory polypeptides that comprise or consist of fusions between CD40 polypeptide regions and RANK cytoplasmic regions. In some embodiments provided herein are modified cells or nucleic acids that comprise polynucleotides that encode costimulatory polypeptides that comprise or consist of fusions between CD40 polypeptide regions and RANK cytoplasmic regions, that lack a functional CD40 cytoplasmic region. In some embodiments provided herein are costimulatory polypeptides that comprise or consist of fusions between CD40 polypeptide regions and RANK cytoplasmic regions, that lack a functional CD40 cytoplasmic region. By lacking a functional CD40 cytoplasmic region is meant that the fusion polypeptide does not comprise a sufficient portion of the CD40 cytoplasmic region for CD40 cytoplasmic polypeptide activity, either as part of a constitutive or an inducible chimeric signaling polypeptide. For example, for a chimeric signaling polypeptide comprising the MyD88-CD40-HCR-CD40 polypeptide provided herein, lacking a functional CD40 cytoplasmic polypeptide refers to removing the HCR portion (of the RANK polypeptide) from the chimeric signaling polypeptide, resulting in a chimeric signaling polypeptide comprising MyD88 and non-functional portions of CD40.

The expression constructs comprise the cytoplasmic signaling regions of the costimulatory polypeptides. Thus, in the present embodiments, the cytoplasmic signaling regions of the costimulatory polypeptides do not include the costimulatory polypeptide extracellular domain, and in some embodiments, the cytoplasmic signaling region of the costimulatory polypeptide does not include the transmembrane domain. In some embodiments, a chimeric antigen receptor, such as, for example, an inducible chimeric antigen receptor, is provided, that comprises a costimulatory polypeptide signaling region. In some embodiments, the chimeric antigen receptor polypeptide or inducible chimeric antigen receptor polypeptide comprises, for example, a MyD88 polypeptide or truncated MyD88 polypeptide, and a costimulatory polypeptide cytoplasmic region. In these embodiments, when referring to a costimulatory polypeptide cytoplasmic signaling region, it is understood that the costimulatory polypeptide cytoplasmic signaling region does not have a transmembrane or does not have an extracellular region. However, the chimeric antigen receptor may comprise an additional polypeptide, such as a transmembrane region of a costimulatory polypeptide. Thus, in one illustrative embodiment, an inducible chimeric antigen receptor may comprise a) a multimeric ligand binding region that binds to a multimeric ligand; b) a MyD88 polypeptide or a truncated MyD88 polypeptide lacking a TIR domain; c) a costimulatory polypeptide cytoplasmic signaling region selected from the group consisting of CD27, CD28, ICOS, 4-1BB, RANK/TRANCE-R, and OX40 cytoplasmic signaling regions; d) a transmembrane region; e) a T cell activation molecule, and f) an antigen recognition moiety. In this example, inducible chimeric antigen receptor may comprise as (c), a CD28 polypeptide cytoplasmic signaling region. This polypeptide includes the cytoplasmic signaling region, and not the CD28 transmembrane region; however, as part (d), the chimeric antigen receptor may comprise a CD28 transmembrane region. In some embodiments, the transmembrane region is not directly contiguous with the costimulatory polypeptide cytoplasmic signaling region, that is, another region or domain, such as, for example, a MyD88 or truncated MyD88 polypeptide, or the multimeric ligand binding region, is located between the transmembrane region and the costimulatory polypeptide cytoplasmic signaling region. In some embodiments, the chimeric antigen receptor does not include a multimeric ligand binding region.

Such costimulatory polypeptides include, but are not limited to CD28 family members (e.g. CD28, ICOS), and TNF receptor family member e.g., RANK/TRANCE-R (TNFRSF11A), OX40, 4-1BB). Therefore, in some embodiments, the chimeric signaling polypeptides may comprise a costimulatory polypeptide selected from the group consisting of TNFR family members, for example, TNFR family members that lack a death domain. In some embodiments, the costimulatory polypeptide is selected from the group consisting of CD27, CD30, TweakR, TAC1, BCMA and HVEM; in some embodiments, the costimulatory polypeptide is selected from the group consisting of RANK, HVEM, CD27, CD30, BCMA, and TweakR; in some embodiments, the costimulatory polypeptide is selected from the group consisting of CD28, 4-1BB, OX40, and ICOS; in some embodiments the costimulatory polypeptide is selected from the group consisting of CD27, CD28, ICOS, 4-1BB, RANK/TRANCE-R, and OX40; in some embodiments the costimulatory polypeptide is selected from the group consisting of CD27, CD28, ICOS, 4-1BB, and OX40.

Polypeptides comprising CD40 cytoplasmic region polypeptides, a TNF receptor family member and truncated MyD88 polypeptides are discussed in U.S. patent application Ser. No. 12/563,991, filed Sep. 21, 2009, entitled METHODS AND COMPOSITIONS FOR GENERATING AN IMMUNE RESPONSE BY INDUCING CD40 AND PATTERN RECOGNITION RECEPTOR ADAPTERS, which is hereby incorporated by reference herein in its entirety.

Costimulatory polypeptides provided herein, such as, for example, the OX40, 4-1BB, ICOS, and CD28 polypeptides include the cytoplasmic costimulatory signaling region or domain of the polypeptide, and, in some embodiments, do not comprise a functional extracellular region or domain. In some embodiments, the costimulatory polypeptides do not comprise a transmembrane domain. The costimulatory polypeptide cytoplasmic costimulatory signaling regions may comprise, but are not limited to, the amino acid sequences provided herein, and may include functional conservative mutations, including deletions or truncations, and may comprise amino acid sequences that are 70%, 75%, 80%, 85%, 90%, 95% or 100% identical to the amino acid sequences provided herein.

Costimulatory polypeptide expression in cells, such as T cells, is discussed, for example, in U.S. patent application Ser. No. 14/210,034, titled METHODS FOR CONTROLLING T CELL PROLIFERATION, filed Mar. 13, 2014, and International Patent Application No: PCT/US2014/026734, published on Sep. 25, 2014 as WO 2014/151960 which are hereby incorporated by reference herein in their entirety.

Ligand Binding Regions

The ligand-binding (“dimerization”) domain of the expression construct can be any convenient domain that will allow for induction using a natural or unnatural ligand, for example, an unnatural synthetic ligand. The multimerizing region or ligand-binding domain can be internal or external to the cellular membrane, depending upon the nature of the construct and the choice of ligand. A wide variety of ligand-binding proteins, including receptors, are known, including ligand-binding proteins associated with the cytoplasmic regions indicated above. As used herein the term “ligand-binding domain can be interchangeable with the term “receptor”. Of particular interest are ligand-binding proteins for which ligands (for example, small organic ligands) are known or may be readily produced. These ligand-binding domains or receptors include the FKBPs and cyclophilin receptors, the steroid receptors, the tetracycline receptor, the other receptors indicated above, and the like, as well as “unnatural” receptors, which can be obtained from antibodies, particularly the heavy or light chain subunit, mutated sequences thereof, random amino acid sequences obtained by stochastic procedures, combinatorial syntheses, and the like. In certain embodiments, the ligand-binding region is selected from the group consisting of FKBP ligand-binding region, cyclophilin receptor ligand-binding region, steroid receptor ligand-binding region, cyclophilin receptors ligand-binding region, and tetracycline receptor ligand-binding region. Often, the ligand-binding region comprises an FvFvls sequence. Sometimes, the Fv′Fvls sequence further comprises an additional Fv′ sequence. Examples include, for example, those discussed in Kopytek, S. J., et al., Chemistry & Biology 7:313-321 (2000) and in Gestwicki, J. E., et al., Combinatorial Chem. & High Throughput Screening 10:667-675 (2007); Clackson T (2006) Chem Biol Drug Des 67:440-2; Clackson, T., in Chemical Biology: From Small Molecules to Systems Biology and Drug Design (Schreiber, s., et al., eds., Wley, 2007)).

For the most part, the ligand-binding domains or receptor domains will be at least about 50 amino acids, and fewer than about 350 amino acids, usually fewer than 200 amino acids, either as the natural domain or truncated active portion thereof. The binding domain may, for example, be small (<25 kDa, to allow efficient transfection in viral vectors), monomeric, nonimmunogenic, have synthetically accessible, cell permeable, nontoxic ligands that can be configured for dimerization.

The receptor domain can be intracellular or extracellular depending upon the design of the expression construct and the availability of an appropriate ligand. For hydrophobic ligands, the binding domain can be on either side of the membrane, but for hydrophilic ligands, particularly protein ligands, the binding domain will usually be external to the cell membrane, unless there is a transport system for internalizing the ligand in a form in which it is available for binding. For an intracellular receptor, the construct can encode a signal peptide and transmembrane domain 5′ or 3′ of the receptor domain sequence or may have a lipid attachment signal sequence 5′ of the receptor domain sequence. Where the receptor domain is between the signal peptide and the transmembrane domain, the receptor domain will be extracellular.

The portion of the expression construct encoding the receptor can be subjected to mutagenesis for a variety of reasons. The mutagenized protein can provide for higher binding affinity, allow for discrimination by the ligand of the naturally occurring receptor and the mutagenized receptor, provide opportunities to design a receptor-ligand pair, or the like. The change in the receptor can involve changes in amino acids known to be at the binding site, random mutagenesis using combinatorial techniques, where the codons for the amino acids associated with the binding site or other amino acids associated with conformational changes can be subject to mutagenesis by changing the codon(s) for the particular amino acid, either with known changes or randomly, expressing the resulting proteins in an appropriate prokaryotic host and then screening the resulting proteins for binding.

Antibodies and antibody subunits, e.g., heavy or light chain, particularly fragments, more particularly all or part of the variable region, or fusions of heavy and light chain to create high-affinity binding, can be used as the binding domain. Antibodies that are contemplated include ones that are an ectopically expressed human product, such as an extracellular domain that would not trigger an immune response and generally not expressed in the periphery (i.e., outside the CNS/brain area). Such examples, include, but are not limited to low affinity nerve growth factor receptor (LNGFR), and embryonic surface proteins (i.e., carcinoembryonic antigen). Yet further, antibodies can be prepared against haptenic molecules, which are physiologically acceptable, and the individual antibody subunits screened for binding affinity. The cDNA encoding the subunits can be isolated and modified by deletion of the constant region, portions of the variable region, mutagenesis of the variable region, or the like, to obtain a binding protein domain that has the appropriate affinity for the ligand. In this way, almost any physiologically acceptable haptenic compound can be employed as the ligand or to provide an epitope for the ligand. Instead of antibody units, natural receptors can be employed, where the binding domain is known and there is a useful ligand for binding.

Oligomerization

The transduced signal will normally result from ligand-mediated oligomerization of the chimeric protein molecules, i.e., as a result of oligomerization following ligand-binding, although other binding events, for example allosteric activation, can be employed to initiate a signal. The construct of the chimeric protein will vary as to the order of the various domains and the number of repeats of an individual domain.

For multimerizing the receptor, the ligand for the ligand-binding domains/receptor domains of the chimeric surface membrane proteins will usually be multimeric in the sense that it will have at least two binding sites, with each of the binding sites capable of binding to the ligand receptor domain. By “multimeric ligand binding region” is meant a ligand binding region that binds to a multimeric ligand. The term “multimeric ligands” include dimeric ligands. A dimeric ligand will have two binding sites capable of binding to the ligand receptor domain. Desirably, the subject ligands will be a dimer or higher order oligomer, usually not greater than about tetrameric, of small synthetic organic molecules, the individual molecules typically being at least about 150 Da and less than about 5 kDa, usually less than about 3 kDa. A variety of pairs of synthetic ligands and receptors can be employed. For example, in embodiments involving natural receptors, dimeric FK506 can be used with an FKBP12 receptor, dimerized cyclosporin A can be used with the cyclophilin receptor, dimerized estrogen with an estrogen receptor, dimerized glucocorticoids with a glucocorticoid receptor, dimerized tetracycline with the tetracycline receptor, dimerized vitamin D with the vitamin D receptor, and the like. Alternatively higher orders of the ligands, e.g., trimeric can be used. For embodiments involving unnatural receptors, e.g., antibody subunits, modified antibody subunits, single chain antibodies comprised of heavy and light chain variable regions in tandem, separated by a flexible linker domain, or modified receptors, and mutated sequences thereof, and the like, any of a large variety of compounds can be used. A significant characteristic of these ligand units is that each binding site is able to bind the receptor with high affinity and they are able to be dimerized chemically. Also, methods are available to balance the hydrophobicity/hydrophilicity of the ligands so that they are able to dissolve in serum at functional levels, yet diffuse across plasma membranes for most applications.

In certain embodiments, the present methods utilize the technique of chemically induced dimerization (CID) to produce a conditionally controlled protein or polypeptide. In addition to this technique being inducible, it also is reversible, due to the degradation of the labile dimerizing agent or administration of a monomeric competitive inhibitor.

The CID system uses synthetic bivalent ligands to rapidly crosslink signaling molecules that are fused to ligand-binding domains. This system has been used to trigger the oligomerization and activation of cell surface (Spencer, D. M., et al., Science, 1993. 262: p. 1019-1024; Spencer D. M. et al., Curr Biol 1996, 6:839-847; Blau, C. A. et al., Proc Natl Acad. Sci. USA 1997, 94:3076-3081), or cytosolic proteins (Luo, Z. et al., Nature 1996, 383:181-185; MacCorkle, R. A. et al., Proc Natl Acad Sci USA 1998, 95:3655-3660), the recruitment of transcription factors to DNA elements to modulate transcription (Ho, S. N. et al., Nature 1996, 382:822-826; Rivera, V. M. et al., Nat. Med. 1996, 2:1028-1032) or the recruitment of signaling molecules to the plasma membrane to stimulate signaling (Spencer D. M. et al., Proc. Natl. Acad. Sci. USA 1995, 92:9805-9809; Holsinger, L. J. et al., Proc. Natl. Acad. Sci. USA 1995, 95:9810-9814).

The CID system is based upon the notion that surface receptor aggregation effectively activates downstream signaling cascades. In the simplest embodiment, the CID system uses a dimeric analog of the lipid permeable immunosuppressant drug, FK506, which loses its normal bioactivity while gaining the ability to crosslink molecules genetically fused to the FK506-binding protein, FKBP12. By fusing one or more FKBPs and a myristoylation sequence to the cytoplasmic signaling domain of a target receptor, one can stimulate signaling in a dimerizer drug-dependent, but ligand and ectodomain-independent manner. This provides the system with temporal control, reversibility using monomeric drug analogs, and enhanced specificity. The high affinity of third-generation AP20187/AP1903 CIDs for their binding domain, FKBP12 permits specific activation of the recombinant receptor in vivo without the induction of non-specific side effects through endogenous FKBP12. FKBP12 variants having amino acid substitutions and deletions, such as FKBP12v36, that bind to a dimerizer drug, may also be used. In addition, the synthetic ligands are resistant to protease degradation, making them more efficient at activating receptors in vivo than most delivered protein agents.

The ligands used are capable of binding to two or more of the ligand-binding domains. The chimeric proteins may be able to bind to more than one ligand when they contain more than one ligand-binding domain. The ligand is typically a non-protein or a chemical. Exemplary ligands include, but are not limited to dimeric FK506 (e.g., FK1012).

In some embodiments, the multimeric ligand binding region comprises an FKBP12 variant region that is optimized to bind a chemical inducer of dimerization (CID). Variants may include, for example, an FKBP region that has an amino acid substitution at position 36 selected from the group consisting of valine, leucine, isoleuceine and alanine (Clackson T, et al., Proc Natl Acad Sci USA. 1998, 95:10437-10442). Rimiducid is a synthetic molecule that has proven safe in healthy volunteers (luliucci J D, et al., J Clin Pharmacol. 2001, 41:870-879). Administration of this small molecule results in cross-linking and activation of the proapoptotic target molecules. The application of this inducible system in human T lymphocytes has been explored using Fas or the death effector domain (DED) of the Fas-associated death domain—containing protein (FADD) as proapoptotic molecules. Up to 90% of T cells transduced with these inducible death molecules underwent apoptosis after administration of CID (Thomis D C, et al., Blood. 2001, 97:1249-1257; Spencer D M, et al., Curr Biol. 1996, 6: 839-847; Fan L, et al., Hum Gene Ther. 1999, 10: 2273-2285; Berger C, et al., Blood. 2004, 103:1261-1269; Junker K, et al., Gene Ther. 2003, 10:1189-197). CID-based activation strategy may be used in any appropriate cell used for cell therapy including, for example, hematopoietic stem cells, and other progenitor cells, including, for example, mesenchymal stromal cells, embryonic stem cells, and inducible pluripotent stem cells. AP20187 and AP1950, a synthetic version of rimiducid, may also be used as the ligand inducer. (Amara J F (97) PNAS 94:10618-23, Clontech Laboratories-Takara Bio).

Other ligand binding regions may be, for example, dimeric regions, or modified ligand binding regions with a wobble substitution, such as, for example, FKBP12(V36): The human 12 kDa FK506-binding protein with an F36 to V substitution, the complete mature coding sequence (amino acids 1-107), provides a binding site for synthetic dimerizer drug rimiducid (Jemal, A. et al., CA Cancer J. Clinic. 58, 71-96 (2008); Scher, H. I. and Kelly, W. K., Journal of Clinical Oncology 11, 1566-72 (1993)). Two tandem copies of the protein may also be used in the construct so that higher-order oligomers are induced upon cross-linking by rimiducid.

F36V′-FKBP: F36V′-FKBP is a codon—wobbled version of F36V-FKBP. It encodes the identical polypeptide sequence as F36V-FKPB but has only 62% homology at the nucleotide level. F36V′-FKBP was designed to reduce recombination in retroviral vectors (Schellhammer, P. F. et al., J. Urol. 157, 1731-5 (1997)). F36V′-FKBP was constructed by a PCR assembly procedure. The transgene contains one copy of F36V′-FKBP linked directly to one copy of F36V-FKBP.

In some embodiments, the inducible chimeric signaling polypeptides and the inducible chimeric antigen receptors comprise a multimeric ligand binding region comprising at least two FKBP12 or FKBP12 variant regions. In some embodiments, the nucleic acids or cells express an inducible Caspase 9 polypeptide; in these embodiments the multimeric ligand binding region comprises at least one FKBP12 or FKBP12 variant region.

In some embodiments, the ligand is a small molecule. The appropriate ligand for the selected ligand-binding region may be selected. Often, the ligand is dimeric, sometimes, the ligand is a dimeric FK506 or a dimeric FK506 analog. In certain embodiments, the ligand is rimiducid (CAS Index Name: 2-Piperidinecarboxylic acid, 1-[(2S)-1-oxo-2-(3, 4,5-trimethoxyphenyl)butyl]-, 1,2-ethanediylbis [imino(2-oxo-2,1-ethanediyl)oxy-3,1-phenylene[(1R)-3-(3,4-Dimethoxyphenyl)propylidene]] ester, [2S-[1(R*),2R*[S*[S*[1(R*),2R1]]]]]-(9C1) CAS Registry Number: 195514-63-7; Molecular Formula: C78H98N4020 Molecular Weight: 1411.65). In certain embodiments, the ligand is AP20187. In certain embodiments, the ligand is an AP20187 analog, such as, for example, AP1510. In some embodiments, certain analogs will be appropriate for the FKBP12, and certain analogs appropriate for the wobbled version of FKBP12. In certain embodiments, one ligand binding region is included in the chimeric protein. In other embodiments, two or more ligand binding regions are included. Where, for example, the ligand binding region is FKBP12, where two of these regions are included, one may, for example, be the wobbled version.

In such methods, the multimeric molecule can be an antibody that binds to an epitope in the CD40 extracellular domain (e.g., humanized anti-CD40 antibody; Tai et al., Cancer Research 64, 2846-2852 (2004)), can be a CD40 ligand (e.g., U.S. Pat. No. 6,497,876 (Maraskovsky et al.)) or may be another costimulatory molecule (e.g., B7/CD28). It is understood that conservative variations in sequence, that do not affect the function, as assayed herein, are within the scope of the present claims.

Since the mechanism of CD40 activation is fundamentally based on trimerization, this receptor is particularly amenable to the CID system. CID regulation provides the system with 1) temporal control, 2) reversibility by addition of a non-active monomer upon signs of an autoimmune reaction, and 3) limited potential for non-specific side effects. In addition, inducible in vivo DC CD40 activation would circumvent the requirement of a second “danger” signal normally required for complete induction of CD40 signaling and would potentially promote DC survival in situ allowing for enhanced T cell priming. Thus, engineering DC vaccines to express iCD40 amplifies the T cell-mediated killing response by upregulating DC expression of antigen presentation molecules, adhesion molecules, TH1 promoting cytokines, and pro-survival factors.

Other dimerization systems contemplated include the coumermycin/DNA gyrase B system. Coumermycin-induced dimerization activates a modified Raf protein and stimulates the MAP kinase cascade. See Farrar et al., 1996.

Membrane-Targeting

A membrane-targeting sequence provides for transport of the chimeric protein to the cell surface membrane, where the same or other sequences can encode binding of the chimeric protein to the cell surface membrane. Molecules in association with cell membranes contain certain regions that facilitate the membrane association, and such regions can be incorporated into a chimeric protein molecule to generate membrane-targeted molecules. For example, some proteins contain sequences at the N-terminus or C-terminus that are acylated, and these acyl moieties facilitate membrane association. Such sequences are recognized by acyltransferases and often conform to a particular sequence motif. Certain acylation motifs are capable of being modified with a single acyl moiety (often followed by several positively charged residues (e.g. human c-Src: M-G-S-N-K-S-K-P-K-D-A-S-Q-R-R-R) to improve association with anionic lipid head groups) and others are capable of being modified with multiple acyl moieties. For example the N-terminal sequence of the protein tyrosine kinase Src can comprise a single myristoyl moiety. Dual acylation regions are located within the N-terminal regions of certain protein kinases, such as a subset of Src family members (e.g., Yes, Fyn, Lck) and G-protein alpha subunits. Such dual acylation regions often are located within the first eighteen amino acids of such proteins, and conform to the sequence motif Met-Gly-Cys-Xaa-Cys, where the Met is cleaved, the Gly is N-acylated and one of the Cys residues is S-acylated. The Gly often is myristoylated and a Cys can be palmitoylated. Acylation regions conforming to the sequence motif Cys-Ala-Ala-Xaa (so called “CAAX boxes”), which can modified with C15 or 010 isoprenyl moieties, from the C-terminus of G-protein gamma subunits and other proteins (e.g., World Wde Web address ebi.ac.uk/interpro/DisplaylproEntry?ac=1PR001230) also can be utilized. These and other acylation motifs include, for example, those discussed in Gauthier-Campbell et al., Molecular Biology of the Cell 15: 2205-2217 (2004); Glabati et al., Biochem. J. 303: 697-700 (1994) and Zlakine et al., J. Cell Science 110: 673-679 (1997), and can be incorporated in chimeric molecules to induce membrane localization. In certain embodiments, a native sequence from a protein containing an acylation motif is incorporated into a chimeric protein. For example, in some embodiments, an N-terminal portion of Lck, Fyn or Yes or a G-protein alpha subunit, such as the first twenty-five N-terminal amino acids or fewer from such proteins (e.g., about 5 to about 20 amino acids, about 10 to about 19 amino acids, or about 15 to about 19 amino acids of the native sequence with optional mutations), may be incorporated within the N-terminus of a chimeric protein. In certain embodiments, a C-terminal sequence of about 25 amino acids or less from a G-protein gamma subunit containing a CAAX box motif sequence (e.g., about 5 to about 20 amino acids, about 10 to about 18 amino acids, or about 15 to about 18 amino acids of the native sequence with optional mutations) can be linked to the C-terminus of a chimeric protein.

In some embodiments, an acyl moiety has a log p value of +1 to +6, and sometimes has a log p value of +3 to +4.5. Log p values are a measure of hydrophobicity and often are derived from octanol/water partitioning studies, in which molecules with higher hydrophobicity partition into octanol with higher frequency and are characterized as having a higher log p value. Log p values are published for a number of lipophilic molecules and log p values can be calculated using known partitioning processes (e.g., Chemical Reviews, Vol. 71, Issue 6, page 599, where entry 4493 shows lauric acid having a log p value of 4.2). Any acyl moiety can be linked to a polypeptide composition discussed above and tested for antimicrobial activity using known methods and those discussed hereafter. The acyl moiety sometimes is a C1-C20 alkyl, C2-C20 alkenyl, C2-C20 alkynyl, C3-C6 cycloalkyl, C1-C4 haloalkyl, C4-C12 cyclalkylalkyl, aryl, substituted aryl, or aryl (C1-C4) alkyl, for example. Any acyl-containing moiety sometimes is a fatty acid, and examples of fatty acid moieties are propyl (C3), butyl (C4), pentyl (C5), hexyl (C6), heptyl (C7), octyl (C8), nonyl (C9), decyl (C10), undecyl (C11), lauryl (C12), myristyl (C14), palmityl (C16), stearyl (C18), arachidyl (C20), behenyl (C22) and lignoceryl moieties (C24), and each moiety can contain 0, 1, 2, 3, 4, 5, 6, 7 or 8 unsaturations (i.e., double bonds). An acyl moiety sometimes is a lipid molecule, such as a phosphatidyl lipid (e.g., phosphatidyl serine, phosphatidyl inositol, phosphatidyl ethanolamine, phosphatidyl choline), sphingolipid (e.g., shingomyelin, sphingosine, ceramide, ganglioside, cerebroside), or modified versions thereof. In certain embodiments, one, two, three, four or five or more acyl moieties are linked to a membrane association region. A chimeric protein herein also may include a single-pass or multiple pass transmembrane sequence (e.g., at the N-terminus or C-terminus of the chimeric protein). Single pass transmembrane regions are found in certain CD molecules, tyrosine kinase receptors, serine/threonine kinase receptors, TGFbeta, BMP, activin and phosphatases. Single pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane. Multiple pass proteins include ion pumps, ion channels, and transporters, and include two or more helices that span the membrane multiple times. All or substantially all of a multiple pass protein sometimes is incorporated in a chimeric protein. Sequences for single pass and multiple pass transmembrane regions are known and can be selected for incorporation into a chimeric protein molecule.

Any membrane-targeting sequence can be employed that is functional in the host and may, or may not, be associated with one of the other domains of the chimeric protein. In some embodiments, such sequences include, but are not limited to myristoylation-targeting sequence, palmitoylation-targeting sequence, prenylation sequences (i.e., farnesylation, geranyl-geranylation, CAAX Box), protein-protein interaction motifs or transmembrane sequences (utilizing signal peptides) from receptors. Examples include those discussed in, for example, ten Klooster J P et al, Biology of the Cell (2007) 99, 1-12, Vincent, S., et al., Nature Biotechnology 21:936-40, 1098 (2003).

Additional protein domains exist that can increase protein retention at various membranes. For example, an ˜120 amino acid pleckstrin homology (PH) domain is found in over 200 human proteins that are typically involved in intracellular signaling. PH domains can bind various phosphatidylinositol (PI) lipids within membranes (e.g. PI (3, 4,5)-P3, PI (3,4)-P2, PI (4,5)-P2) and thus play a key role in recruiting proteins to different membrane or cellular compartments. Often the phosphorylation state of PI lipids is regulated, such as by PI-3 kinase or PTEN, and thus, interaction of membranes with PH domains are not as stable as by acyl lipids.

AP1903 API, rimiducid, is manufactured by Alphora Research Inc. and AP1903 Drug Product for Injection is made by AAI Pharma Services Corp. It is formulated as a 5 mg/mL solution of rimiducid in a 25% solution of the non-ionic solubilizer Solutol HS 15 (250 mg/mL, BASF). At room temperature, this formulation is a clear solution. Upon refrigeration, this formulation undergoes a reversible phase transition on extended storage, resulting in a milky solution. This phase transition is reversed upon re-warming to room temperature. The fill is 8 mL in a 10 mL glass vial (˜40 mg rimiducid for Injection total per vial).

For use, the rimiducid will be warmed to room temperature and diluted prior to administration. For subjects over 50 kg, the rimiducid is administered via i.v. infusion at a dose of 40 mg diluted in 100 mL physiological saline over 2 hours at a rate of 50 mL per hour using a DEHP-free saline bag and solution set. Subjects less than 50 kg receive 0.4 mg/kg AP1903.

All study medication is maintained at a temperature between 2 degrees C. and 8 degrees C., protected from excessive light and heat, and stored in a locked area with restricted access.

Upon determining a need to administer rimiducid and activate the therapeutic T cells, for example the chimeric antigen-receptor and inducible chimeric costimulatory polypeptide-expressing T cells, patients may be, for example, administered a single fixed dose of rimiducid for Injection (0.4 mg/kg) via IV infusion over 2 hours, using a non-DEHP, non-ethylene oxide sterilized infusion set. The dose of rimiducid is calculated individually for all patients, and is not be recalculated unless body weight fluctuates by 0%. The calculated dose is diluted in 100 mL in 0.9% normal saline before infusion.

Upon determining a need to administer rimiducid and activate Caspase-9 in order to induce apoptosis of the modified cells, patients may be, for example, administered a single fixed dose of rimiducid for Injection (0.4 mg/kg) via IV infusion over 2 hours, using a non-DEHP, non-ethylene oxide sterilized infusion set. The dose of rimiducid is calculated individually for all patients, and is not be recalculated unless body weight fluctuates by 10%. The calculated dose is diluted in 100 mL in 0.9% normal saline before infusion.

In a previous Phase I study of rimiducid, 24 healthy volunteers were treated with single doses of rimiducid for Injection at dose levels of 0.01, 0.05, 0.1, 0.5 and 1.0 mg/kg infused IV over 2 hours. Rimiducid plasma levels were directly proportional to dose, with mean Cmax values ranging from approximately 10-1275 ng/mL over the 0.01-1.0 mg/kg dose range. Following the initial infusion period, blood concentrations demonstrated a rapid distribution phase, with plasma levels reduced to approximately 18, 7, and 1% of maximal concentration at 0.5, 2 and 10 hours post-dose, respectively. Rimiducid for Injection was shown to be safe and well tolerated at all dose levels and demonstrated a favorable pharmacokinetic profile. luliucci J D, et al., J Clin Pharmacol. 41: 870-9, 2001.

The fixed dose of rimiducid for injection used, for example, may be 0.4 mg/kg intravenously infused over 2 hours. The amount of rimiducid needed in vitro for effective signaling of cells is about 10-100 nM (MW: 1412 Da). This equates to 14-140 μg/L or ˜0.014-0.14 mg/kg (1.4-140 μg/kg). The dosage may vary according to the application, and may, in certain examples, be more in the range of 0.1-10 nM, or in the range of 50-150 nM, 10-200 nM, 75-125 nM, 100-500 nM, 100-600 nM, 100-700 nM, 100-800 nM, or 100-900 nM. Doses up to 1 mg/kg were well-tolerated in the Phase I study of rimiducid described above.

Dual Controls for Selective Activation or Elimination

In some embodiments, a molecular switch is provided that is controlled by a distinct dimerizer ligand, based on the heterodimerizing small molecule, rapamycin, or rapamycin analogs (“rapalogs”). Rapamycin binds to FKBP12, and its variants, and can induce heterodimerization of signaling domains that are fused to FKBP12 by binding to both FKBP12 and to polypeptides that contain the FKBP-rapamycin-binding (FRB) domain of mTOR. Provided in some embodiments of the present application are molecular switches that greatly augment the use of rapamycin, rapalogs and rimiducid as agents for therapeutic applications. In certain embodiments, the allele specificity of rimiducid is used to allow selective dimerization of Fᵥ-fusions. In other embodiments, a rapamycin or rapalog-inducible pro-apoptotic polypeptide, such as, for example, Caspase-9 or a rapamycin or rapalog-inducible costimulatory polypeptide, such as, for example, MyD88/4-1BB, or an inducible chimeric MyD88-costimulatory polypeptide cytoplasmic region other than 4-1BB (iM-X), is used in combination with a rimiducid-inducible pro-apoptotic polypeptide, such as, for example, Caspase-9, or a rimiducid-inducible chimeric stimulating polypeptide, such as, for example, iMC to produce dual-switches. These dual-switches can be used to control both cell proliferation and apoptosis selectively by administration of either of two distinct ligand inducers.

In other embodiments, a molecular switch is provided that provides the option to activate a pro-apoptotic polypeptide, such as, for example, Caspase-9, with either rimiducid, or rapamycin or a rapalog, wherein the chimeric pro-apoptotic polypeptide comprises both a rimiducid-induced switch and a rapamycin-, or rapalog-, induced switch. Including both molecular switches on the same chimeric pro-apoptotic polypeptide provides flexibility in a clinical setting, where the clinician can choose to administer the appropriate drug based on its specific pharmacological properties, or for other considerations, such as, for example, availability. These chimeric pro-apoptotic polypeptides may comprise, for example, both a FKBP12-Rapamycin-binding domain of mTOR (FRB), or an FRB variant, and an FKBP12 variant polypeptide, such as, for example, FKBP12v36. By FRB variant polypeptide is meant an FRB polypeptide that binds to a rapamycin analog (rapalog), for example, a rapalog provided in the present application. FRB variant polypeptides comprise one or more amino acid substitutions, bind to a rapalog, and may bind, or may not bind to rapamycin. U.S. patent application Ser. No. 15/377,776, filed Dec. 13, 2016, by Joseph Henri Bayle, titled “Dual Controls for Therapeutic Cell Activation or Elimination” is hereby incorporated by reference herein in its entirety.

In one embodiment of the dual-switch technology, (Fwt.FRBΔC9/M-X.FvFv) a homodimerizer, such as AP1903 (rimiducid), induces activation of a modified cell, and a heterodimerizer, such as rapamycin or a rapalog, activates a safety switch, causing apoptosis of the modified cell. In this embodiment, for example, a chimeric pro-apoptotic polypeptide, such as, for example, Caspase-9, comprising both an FKBP12 and an FRB, or FRB variant region (iFwtFRBC9) is expressed in a cell along with an inducible chimeric MyD88/costimulating polypeptide, that comprises MyD88 and a costimulatory polypeptide cytoplasmic region and at least two copies of FKBP12v36 (M-X.FvFv). Upon contacting the cell with a dimerizer that binds to the Fv regions, the M-X.FvFv dimerizes or multimerizes, and activates the cell. The cell may, for example, be a T cell that expresses a chimeric antigen receptor directed against a target antigen (CARζ). As a safety switch, the cell may be contacted with a heterodimerizer, such as, for example, rapamycin, or a rapalog, that binds to the FRB region on the iFwtFRBC9 polypeptide, as well as the FKBP12 region on the iFwtFRBC9 polypeptide, causing direct dimerization of the Caspase-9 polypeptide, and inducing apoptosis. In another mechanism, the heterodimerizer binds to the FRB region on the iFwtFRBC9 polypeptide, and the Fv region on the M-X.FvFv polypeptide, causing scaffold-induced dimerization, due to the scaffold of two FKBP12v36 polypeptides on each M-X.FvFv polypeptide, and inducing apoptosis. By FKBP12 variant polypeptide is meant an FKBP12 polypeptide that comprises one or more amino acid substitutions and that binds to a ligand such as, for example, rimiducid, with at least 100 times, 500 times, or 1000 times more affinity than the ligand binds to the FKBP12 polypeptide region.

In another embodiment of the dual-switch technology, (FRBFwtM-X/FvC9) a heterodimerizer, such as rapamycin or a rapalog, induces activation of a modified cell, and a homodimerizer, such as AP1903 activates a safety switch, causing apoptosis of the modified cell. In this embodiment, for example, a chimeric pro-apoptotic polypeptide, such as, for example, Caspase-9, comprising an Fv region (iFvC9) is expressed in a cell along with an inducible chimeric M-X costimulating polypeptide, that comprises MyD88 and a costimulatory polypeptide cytoplasmic region and both an FKBP12 and an FRB or FRB variant region (iFRBFwtM-X) (M-X.FvFv). Upon contacting the cell with rapamycin or a rapalog that heterodimerizes the FKBP12 and FRB regions, the iFRBFwtM-X dimerizes or multimerizes, and activates the cell. The cell may, for example, be a T cell that expresses a chimeric antigen receptor directed against a target antigen (CARζ). As a safety switch, the cell may be contacted with a homodimerizer, such as, for example, AP1903, which binds to the iFvC9 polypeptide, causing direct dimerization of the Caspase-9 polypeptide, and inducing apoptosis.

In some embodiments, a homodimer-based switch is used to activate the chimeric polypeptides expressed in the modified cells. Thus, the rimiducid-based switch discussed herein, and including as discussed for the dual switch technology, may be used as part of a single switch, in the absence of a chimeric caspase polypeptide. In such embodiments, the chimeric polypeptides comprise a first and a second ligand binding region, where each ligand binding domain comprises an FKBP12 polypeptide region, such as, for example, a wild type FKBP12 polypeptide, or, for example, a FKBP12 variant polypeptide region, where the FKBP12 variant polypeptide binds, for example, to AP1903 or AP20187. In the presence of a homodimeric ligand, such as, for example rimiducid or a rimiducid variant, at least two chimeric polypeptides dimerize, and the chimeric polypeptides are activated in the cell.

In some embodiments, a heterodimer-based switch is used to activate the chimeric polypeptides expressed in the modified cells. Thus, the rapamycin, or rapalog based switch discussed for the dual switch technology, may be used as part of a single switch, in the absence of a chimeric caspase polypeptide. In such embodiments, the chimeric polypeptides comprise a first and a second ligand binding region, where one ligand binding region comprises, for example, an FKBP12 polypeptide region, such as, for example, a wild type FKBP12 polypeptide, or, for example, a FKBP12 variant polypeptide region, where the FKBP12 variant polypeptide binds, for example, to AP1903 or AP20187, and the second ligand binding region comprises, for example, an FRB polypeptide region, such as for example, a wild type FRB polypeptide or an FRB variant that binds to a rapalog. In the presence of a heterodimeric ligand, such as, for example rapamycin, or a rapalog, at least two chimeric polypeptides dimerize, and the chimeric polypeptides are activated in the cell.

As used here, the term “rapalog” is meant as an analog of the natural antibiotic rapamycin. Certain rapalogs in the present embodiments have properties such as stability in serum, a poor affinity to wildtype FRB (and hence the parent protein, mTOR, leading to reduction or elimination of immunosuppressive properties), and a relatively high affinity to a mutant FRB domain. For commercial purposes, in certain embodiments, the rapalogs have useful scaling and production properties. Examples of rapalogs include, but are not limited to, S-o,p-dimethoxyphenyl (DMOP)-rapamycin: EC₅₀ (wt FRB (K2095 T2098 W2101)˜1000 nM), EC₅₀ (FRB-KLW˜5 nM) Luengo J I (95) Chem & Biol 2:471-81; Luengo J I (94) J. Org Chem 59:6512-6513; U.S. Pat. No. 6,187,757; R-Isopropoxyrapamycin: EC₅₀ (wt FRB (K2095 T2098 W2101)˜300 nM), EC50 (FRB-PLF˜8.5 nM); Liberles S (97) PNAS 94: 7825-30; and S-Butanesulfonamidorap (AP23050): EC₅₀ (wt FRB (K2095 T2098 W2101)˜2.7 nM), EC₅₀ (FRB-KTF˜>200 nM) Bayle (06) Chem & Bio. 13: 99-107, C7-1 sobutyloxyrapamycin; 40-(S)-Fluoro-Rapamycin; 40-(S)-Chloro-Rapamycin; 40-(S)-Bromo-Rapamycin; 40-(S)-lodo-Rapamycin; 40-(S)-Amino-Rapamycin; 40-(S)-Fluoro-7-(S)-DMOP-Rapamycin; 40-(S)-Chloro-7-(S)-DMOP-Rapamycin; 40-(S)-lodo-7-(S)-DMOP-Rapamycin; 40-(S)-Azide-7-(S)-DMOP-Rapamycin; 40-(R)-p-Bromomethylbenzoyl-Rapamycin; 40-(R)-p-Chloromethylbenzoyl-Rapamycin; 40-(R)-((4-methylpiperazin-1-yl)p-methylbenzoyl)-Rapamycin; di-p-Bromomethylbenzoyl-Rapamycin; and 40-(S)—N-(3-(4-methylpiperazin-1-yl)propyl)-Rapamycinamine (see, e.g., U.S. Patent Application Publication No. US2017/0166877, which is incorporated by reference herein), R and S C7-ethyloxyrapamycin, R and S C7-isopropyloxyrapamycin, R and S C7-isobutylrapamycin, R and S ethylcarbamaterapamycin, R and S C7-phenylcarbamaterapamycin, R and S C7-(3-methyl)indole rapamycin, temsirolimus, everolimus, zotarolimus, and R and S C7-(7-methyl)indole rapamycin.

The term “FRB” refers to the FKBP12-Rapamycin-Binding (FRB) domain (residues 2015-2114 encoded within mTOR), and analogs thereof. In certain embodiments, FRB analogs or variants are provided. The properties of an FRB analog or variant are stability (some variants are more labile than others) and ability to bind to various rapalogs. In certain embodiments, the FRB analog or variant binds to a C7 rapalog, such as, for example, those provided in the present application, and those referred to in publications that are incorporated by reference herein. In certain embodiments, the FRB analog or variant comprises an amino acid substitution at position T2098. Based on the crystal structure conjugated to rapamycin, there are 3 key rapamycin-interacting residues that have been most analyzed, K2095, T2098, and W2101. Mutation of all three leads to an unstable protein that can be stabilized in the presence of rapamycin or some rapalogs. This feature can be used to further increase the signal:noise ratio in some applications. Examples of mutants are discussed in Bayle et al (06) Chem & Bio 13: 99-107; Stankunas et al (07) Chembiochem 8:1162-1169; and Liberles S (97) PNAS 94:7825-30). Examples of FRB variant polypeptide regions of the present embodiments include, but are not limited to, KLW (with L2098); KTF (with F2101); and KLF (L2098, F2101). FRB variant KLW corresponds to the FRBL polypeptide, for example, consisting of the amino acid of SEQ ID NO: 903, and has a substitution of an L residue at position 2098. By comparing the KLW variant of SEQ ID NO: 903 with the wild type FRB polypeptide, for example, the polypeptide consisting of the amino acid sequence of SEQ ID NO: 905, one can determine the sequence of the other FRB variants listed herein.

Rapamycin is a natural product macrolide that binds with high affinity (<1 nM) to FKBP12 and together initiates the high-affinity, inhibitory interaction with the FKBP-Rapamycin-Binding (FRB) domain of mTOR (8). FRB is small (89 amino acids) and can thereby be used as a protein “tag” or “handle” when appended to many proteins (9-11). Coexpression of a FRB-fused protein with a FKBP12-fused protein renders their approximation rapamycin-inducible (12-16). This and the examples that follow provide experiments and results designed to test whether expression of Caspase-9 bound with FKBP and FRB in tandem can also direct apoptosis and serve as the basis for a cell safety switch regulated by the orally available ligand, rapamycin. Further, an inducible M-X rapamycin-sensitive costimulatory polypeptide was developed by fusing FKBP and FRB in tandem with the M-X polypeptide. For this tandem fusion of FKBP and FRB, derivatives of rapamycin (rapalogs) may also be used that do not inhibit mTOR at a low, therapeutic dose. For example, rapamycin, or these rapamycin analogs may bind with selected, M-X-FKBP-fused mutant FRB domains, using a heterdimerizer to homodimerize two M-X-FKBP-FRB polypeptides.

The following references are referred to in this section, and are hereby incorporated by reference herein in their entireties.
1. Straathof K C, Pule M A, Yotnda P, Dotti G, Vanin E F, Brenner M K, Heslop H E, Spencer D M, and Rooney C M. An inducible caspase 9 safety switch for T-cell therapy. Blood. 2005; 105(11):4247-54.
2. Fan L, Freeman K W, Khan T, Pham E, and Spencer D M. Improved artificial death switches based on caspases and FADD. Hum Gene Ther. 1999; 10(14):2273-85.
3. Spencer D M, Wandless T J, Schreiber S L, and Crabtree G R. Controlling signal transduction with synthetic ligands. Science. 1993; 262(5136):1019-24.
4. Acevedo V D, Gangula R D, Freeman K W, Li R, Zhang Y, Wang F, Ayala G E, Peterson L E, lttmann M, and Spencer D M. Inducible FGFR-1 activation leads to irreversible prostate adenocarcinoma and an epithelial-to-mesenchymal transition. Cancer Cell. 2007; 12(6):559-71.
5. Spencer D M, Belshaw P J, Chen L, Ho S N, Randazzo F, Crabtree G R, and Schreiber S L. Functional analysis of Fas signaling in vivo using synthetic inducers of dimerization. Curr Biol. 1996; 6(7):839-47.
6. Strasser A, Cory S, and Adams J M. Deciphering the rules of programmed cell death to improve therapy of cancer and other diseases. EMBO J. 2011; 30(18):3667-83.
7. Narayanan P, Lapteva N, Seethammagari M, Levitt J M, Slawin K M, and Spencer D M. A composite MyD88/CD40 switch synergistically activates mouse and human dendritic cells for enhanced antitumor efficacy. J Clin Invest. 2011; 121(4):1524-34.
8. Sabatini D M, Erdjument-Bromage H, Lui M, Tempst P, and Snyder S H. RAFT1: a mammalian protein that binds to FKBP12 in a rapamycin-dependent fashion and is homologous to yeast TORs. Cell. 1994; 78(1):35-43.
9. Brown E J, Albers M W, Shin T B, Ichikawa K, Keith C T, Lane W S, and Schreiber S L. A mammalian protein targeted by G1-arresting rapamycin-receptor complex. Nature. 1994; 369(6483):756-8.
10. Chen J, Zheng X F, Brown E J, and Schreiber S L. Identification of an 11-kDa FKBP12-rapamycin-binding domain within the 289-kDa FKBP12-rapamycin-associated protein and characterization of a critical serine residue. Proc Natl Acad Sci USA. 1995; 92(11):4947-51.
11. Choi J, Chen J, Schreiber S L, and Clardy J. Structure of the FKBP12-rapamycin complex interacting with the binding domain of human FRAP. Science. 1996; 273(5272):239-42.
12. Ho S N, Biggar S R, Spencer D M, Schreiber S L, and Crabtree G R. Dimeric ligands define a role for transcriptional activation domains in reinitiation. Nature. 1996; 382(6594):822-6.
13. Klemm J D, Beals C R, and Crabtree G R. Rapid targeting of nuclear proteins to the cytoplasm. Curr Biol. 1997; 7(9):638-44.
14. Bayle J H, Grimley J S, Stankunas K, Gestwicki J E, Wandless T J, and Crabtree G R. Rapamycin analogs with differential binding specificity permit orthogonal control of protein activity. Chem Biol. 2006; 13(1):99-107.
15. Stankunas K, Bayle J H, Gestwicki J E, Lin Y M, Wandless T J, and Crabtree G R. Conditional protein alleles using knockin mice and a chemical inducer of dimerization. Mol Cell. 2003; 12(6):1615-24.
16. Stankunas K, Bayle J H, Havranek J J, Wandless T J, Baker D, Crabtree G R, and Gestwicki J E. Rescue of Degradation-Prone Mutants of the FK506-Rapamycin Binding (FRB) Protein with Chemical Ligands. Chembiochem. 2007.

Dual-Switch, Chimeric Pro-Apoptotic Polypeptides

Chemical Induction of Dimerization (CID) with small molecules is an effective technology used to generate switches of protein function to alter cell physiology. Rimiducid or AP1903 is a highly specific and efficient dimerizer composed of two identical protein-binding surfaces (based on FK506) arranged tail-to-tail, each with high affinity and specificity for an FKBP mutant, FKBP12v36 or FKBPᵥ. FKBP12v36 is a modified version of FKBP12, in which phenylalanine 36, is replaced with the smaller hydrophobic residue, valine, which accommodates the bulky modification on the FKBP12-binding site of AP1903 [1]. This change increases binding of AP1903 to FKBP12v36 (˜j 0.1 nM), while binding of AP1903 to native FKBP12 is reduced around 100-fold relative to FK506 [1, 2]. Attachment of one or more Fᵥ domains onto one or more cell signaling molecules that normally rely on homodimerization can convert that protein to rimiducid-induced signaling control. Homodimerization with rimiducid is the basis of both the inducible Caspase-9 (iCaspase-9) “safety switch” and the inducible MyD88/CD40 (iMC) “activation switch” for cellular therapy.

Rapamycin binds to FKBP12, but unlike rimiducid, rapamycin also binds to the FKBP12-Rapamycin-Binding (FRB) domain of mTOR and can induce heterodimerization of signaling domains that are fused to FKBP12 with fusions containing FRB. Expression of Caspase-9 fused with FKBP and FRB in tandem (in both orientations: FKBP.FRB.ΔC9 or FRB.FKBP.ΔC9) can direct apoptosis and serve as the basis for a cell safety switch regulated by the orally available ligand, rapamycin. Importantly, since rimiducid contains a bulky modification on the FKBP12-binding site, this dimerizer is not able to bind to wild type FKBP12.

The FRB.FKBPᵥ.ΔC9 switch provides the option to activate caspase-9 with either rimiducid or rapamycin by mutating the FKBP domain to FKBPᵥ. This flexibility in terms of choice of activating drug may be important in a clinical setting where the clinician can choose to administer the drug based on its specific pharmacological properties. Additionally, this switch provides a molecule to allow for direct comparison between the drug-activating kinetics of rimiducid and rapamycin where the effector is contained within a single molecule.
1. D. Spencer, et al., Science, vol. 262, pp. 1019-1024, 1993.
2. T. Clackson, et al., Proc Natl Acad Sci USA, vol. 95, pp. 10437-10442, 1998.

Selectable Markers

In certain embodiments, the expression constructs contain nucleic acid constructs whose expression is identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as Herpes Simplex Virus thymidine kinase (tk) are employed. Immunologic surface markers containing the extracellular, non-signaling domains or various proteins (e.g. CD34, CD19, LNGFR) also can be employed, permitting a straightforward method for magnetic or fluorescence antibody-mediated sorting. The selectable marker employed is not believed to be important, so long as it is capable of being expressed simultaneously with the nucleic acid encoding a gene product. Further examples of selectable markers include, for example, reporters such as GFP, EGFP, beta-gal or chloramphenicol acetyltransferase (CAT). In certain embodiments, the marker protein, such as, for example, CD19 is used for selection of the cells for transfusion, such as, for example, in immunomagnetic selection.

As discussed herein, a CD19 marker is distinguished from an anti-CD19 antibody, or, for example, a scFv, TCR, or other antigen recognition moiety that binds to CD19.

In some embodiments, a polypeptide may be included in the expression vector to aid in sorting cells. For example, the CD34 minimal epitope may be incorporated into the vector. In some embodiments, the expression vectors used to express the chimeric antigen receptors or chimeric signaling or inducible chimeric signaling polypeptides provided herein further comprise a polynucleotide that encodes the 16 amino acid CD34 minimal epitope. In some embodiments, such as certain embodiments provided in the examples herein, the CD34 minimal epitope is incorporated at the amino terminal position of the CD8 stalk.

Transmembrane Regions

A chimeric antigen receptor herein may include a single-pass or multiple pass transmembrane sequence (e.g., at the N-terminus or C-terminus of the chimeric protein). Single pass transmembrane regions are found in certain CD molecules, tyrosine kinase receptors, serine/threonine kinase receptors, TGFβ, BMP, activin and phosphatases. Single pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane. Multiple pass proteins include ion pumps, ion channels, and transporters, and include two or more helices that span the membrane multiple times. All or substantially all of a multiple pass protein sometimes is incorporated in a chimeric protein. Sequences for single pass and multiple pass transmembrane regions are known and can be selected for incorporation into a chimeric protein molecule.

In some embodiments, the transmembrane domain is fused to the extracellular domain of the CAR. In one embodiment, the transmembrane domain that naturally is associated with one of the domains in the CAR is used. In other embodiments, a transmembrane domain that is not naturally associated with one of the domains in the CAR is used. In some instances, the transmembrane domain can be selected or modified by amino acid substitution (e.g., typically charged to a hydrophobic residue) to avoid binding of such domains to the transmembrane domains of the same or different surface membrane proteins to minimize interactions with other members of the receptor complex.

Transmembrane domains may, for example, be derived from the alpha, beta, or zeta chain of the T cell receptor, CD3-ε, CD3ζ, CD4, CD5, CD8, CD8α, CD9, CD16, CD22, CD28, CD33, CD38, CD64, CD80, CD86, CD134, CD137, or CD154. Or, in some examples, the transmembrane domain may be synthesized de novo, comprising mostly hydrophobic residues, such as, for example, leucine and valine. In certain embodiments a short polypeptide linker may form the linkage between the transmembrane domain and the intracellular domain of the chimeric antigen receptor. The chimeric antigen receptors may further comprise a stalk, that is, an extracellular region of amino acids between the extracellular domain and the transmembrane domain. For example, the stalk may be a sequence of amino acids naturally associated with the selected transmembrane domain. In some embodiments, the chimeric antigen receptor comprises a CD8 transmembrane domain, in certain embodiments, the chimeric antigen receptor comprises a CD8 transmembrane domain, and additional amino acids on the extracellular portion of the transmembrane domain, in certain embodiments, the chimeric antigen receptor comprises a CD8 transmembrane domain and a CD8 stalk. The chimeric antigen receptor may further comprise a region of amino acids between the transmembrane domain and the cytoplasmic domain, which are naturally associated with the polypeptide from which the transmembrane domain is derived.

Control Regions

1 Promoters

The particular promoter employed to control the expression of a polynucleotide sequence of interest is not believed to be important, so long as it is capable of directing the expression of the polynucleotide in the targeted cell. Thus, where a human cell is targeted the polynucleotide sequence-coding region may, for example, be placed adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, ß-actin, rat insulin promoter and glyceraldehyde-3-phosphate dehydrogenase can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it is desirable to prohibit or reduce expression of one or more of the transgenes. Examples of transgenes that are toxic to the producer cell line are pro-apoptotic and cytokine genes. Several inducible promoter systems are available for production of viral vectors where the transgene products are toxic (add in more inducible promoters).

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter, which drives expression of the gene of interest, is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that may be useful is the Tet-Off™ or Tet-On™ system (Clontech, Palo Alto, Calif.) originally developed by Gossen and Bujard (Gossen and Bujard, Proc. Natl. Acad. Sci. USA, 89:5547-5551, 1992; Gossen et al., Science, 268:1766-1769, 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tetracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system may be used so that the producer cells could be grown in the presence of tetracycline or doxycycline and prevent expression of a potentially toxic transgene, but when the vector is introduced to the patient, the gene expression would be constitutively on.

In some circumstances, it is desirable to regulate expression of a transgene in a gene therapy vector. For example, different viral promoters with varying strengths of activity are utilized depending on the level of expression desired. In mammalian cells, the CMV immediate early promoter is often used to provide strong transcriptional activation. The CMV promoter is reviewed in Donnelly, J. J., et al., 1997. Annu. Rev. Immunol. 15:617-48. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus.

Similarly tissue specific promoters are used to effect transcription in specific tissues or cells so as to reduce potential toxicity or undesirable effects to non-targeted tissues. These promoters may result in reduced expression compared to a stronger promoter such as the CMV promoter, but may also result in more limited expression, and immunogenicity. (Bojak, A., et al., 2002. Vaccine. 20:1975-79; Cazeaux, N., et al., 2002. Vaccine 20:3322-31). For example, tissue specific promoters such as the PSA associated promoter or prostate-specific glandular kallikrein, or the muscle creatine kinase gene may be used where appropriate.

Examples of tissue specific or differentiation specific promoters include, but are not limited to, the following: B29 (B cells); CD14 (monocytic cells); CD43 (leukocytes and platelets); CD45 (hematopoietic cells); CD68 (macrophages); desmin (muscle); elastase-1 (pancreatic acinar cells); endoglin (endothelial cells); fibronectin (differentiating cells, healing tissues); and Flt-1 (endothelial cells); GFAP (astrocytes).

In certain indications, it is desirable to activate transcription at specific times after administration of the gene therapy vector. This is done with such promoters as those that are hormone or cytokine regulatable. Cytokine and inflammatory protein responsive promoters that can be used include K and T kininogen (Kageyama et al., (1987) J. Biol. Chem., 262, 2345-2351), c-fos, TNF-alpha, C-reactive protein (Arcone, et al., (1988) Nucl. Acids Res., 16(8), 3195-3207), haptoglobin (Oliviero et al., (1987) EMBO J., 6, 1905-1912), serum amyloid A2, C/EBP alpha, IL-1, IL-6 (Poli and Cortese, (1989) Proc. Nat'l Acad. Sci. USA, 86, 8202-8206), Complement C3 (Wilson et al., (1990) Mol. Cell. Biol., 6181-6191), IL-8, alpha-1 acid glycoprotein (Prowse and Baumann, (1988) Mol Cell Biol, 8,42-51), alpha-1 antitrypsin, lipoprotein lipase (Zechner et al., Mol. Cell. Biol., 2394-2401, 1988), angiotensinogen (Ron, et al., (1991) Mol. Cell. Biol., 2887-2895), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 anti-chymotrypsin. Other promoters include, for example, SV40, MMTV, Human Immunodeficiency Virus (MV), Moloney virus, ALV, Epstein Barr virus, Rous Sarcoma virus, human actin, myosin, hemoglobin, and creatine.

It is envisioned that any of the above promoters alone or in combination with another can be useful depending on the action desired. Promoters, and other regulatory elements, are selected such that they are functional in the desired cells or tissue. In addition, this list of promoters should not be construed to be exhaustive or limiting; other promoters that are used in conjunction with the promoters and methods disclosed herein.

2. Enhancers

Enhancers are genetic elements that increase transcription from a promoter located at a distant position on the same molecule of DNA. Early examples include the enhancers associated with immunoglobulin and T cell receptors that both flank the coding sequence and occur within several introns. Many viral promoters, such as CMV, SV40, and retroviral LTRs are closely associated with enhancer activity and are often treated like single elements. Enhancers are organized much like promoters. That is, they are composed of many individual elements, each of which binds to one or more transcriptional proteins. The basic distinction between enhancers and promoters is operational. An enhancer region as a whole stimulates transcription at a distance and often independent of orientation; this need not be true of a promoter region or its component elements. On the other hand, a promoter has one or more elements that direct initiation of RNA synthesis at a particular site and in a particular orientation, whereas enhancers lack these specificities.

Promoters and enhancers are often overlapping and contiguous, often seeming to have a very similar modular organization. A subset of enhancers includes locus-control regions (LCRs) that can not only increase transcriptional activity, but (along with insulator elements) can also help to insulate the transcriptional element from adjacent sequences when integrated into the genome. Any promoter/enhancer combination (as per the Eukaryotic Promoter Data Base EPDB) can be used to drive expression of the gene, although many will restrict expression to a particular tissue type or subset of tissues. (reviewed in, for example, Kutzler, M. A., and Weiner, D. B., 2008. Nature Reviews Genetics 9:776-88). Examples include, but are not limited to, enhancers from the human actin, myosin, hemoglobin, muscle creatine kinase, sequences, and from viruses CMV, RSV, and EBV. Appropriate enhancers may be selected for particular applications. Eukaryotic cells can support cytoplasmic transcription from certain bacterial promoters if the appropriate bacterial polymerase is provided, either as part of the delivery complex or as an additional genetic expression construct.

3. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the present methods, and any such sequence is employed such as human or bovine growth hormone and SV40 polyadenylation signals and LTR polyadenylation signals. One non-limiting example is the SV40 polyadenylation signal present in the pCEP3 plasmid (Invitrogen, Carlsbad, Calif.). Also contemplated as an element of the expression cassette is a terminator. These elements can serve to enhance message levels and to minimize read through from the cassette into other sequences. Termination or poly(A) signal sequences may be, for example, positioned about 11-30 nucleotides downstream from a conserved sequence (AAUAAA) at the 3′ end of the mRNA. (Montgomery, D. L., et al., 1993. DNA Cell Biol. 12:777-83; Kutzler, M. A., and Weiner, D. B., 2008. Nature Rev. Gen. 9:776-88).

4. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. The initiation codon is placed in-frame with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements is used to create multigene, or polycistronic messages. IRES elements are able to bypass the ribosome-scanning model of 5′ methylated cap-dependent translation and begin translation at internal sites (Pelletier and Sonenberg, Nature, 334:320-325, 1988). IRES elements from two members of the picornavirus family (polio and encephalomyocarditis) have been discussed (Pelletier and Sonenberg, 1988), as well as an IRES from a mammalian message (Macejak and Sarnow, Nature, 353:90-94, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Sequence Optimization

Protein production may also be increased by optimizing the codons in the transgene. Species specific codon changes may be used to increase protein production. Also, codons may be optimized to produce an optimized RNA, which may result in more efficient translation. By optimizing the codons to be incorporated in the RNA, elements such as those that result in a secondary structure that causes instability, secondary mRNA structures that can, for example, inhibit ribosomal binding, or cryptic sequences that can inhibit nuclear export of mRNA can be removed. (Kutzler, M. A., and Weiner, D. B., 2008. Nature Rev. Gen. 9:776-88; Yan, J. et al., 2007. Mol. Ther. 15:411-21; Cheung, Y. K., et al., 2004. Vaccine 23:629-38; Narum, D. L., et al., 2001. 69:7250-55; Yadava, A., and Ockenhouse, C. F., 2003. Infect. Immun. 71:4962-69; Smith, J. M., et al., 2004. AIDS Res. Hum. Retroviruses 20:1335-47; Zhou, W., et al., 2002. Vet. Microbiol. 88:127-51; Wu, X., et al., 2004. Biochem. Biophys. Res. Commun. 313:89-96; Zhang, W., et al., 2006. Biochem. Biophys. Res. Commun. 349:69-78; Deml, L. A., et al., 2001. J. Virol. 75:1099-11001; Schneider, R. M., et al., 1997. J. Virol. 71:4892-4903; Wang, S. D., et al., 2006. Vaccine 24:4531-40; zur Megede, J., et al., 2000. J. Virol. 74:2628-2635). For example, the FKBP12 or other multimerizing region polypeptide, the costimulatory polypeptide cytoplasmic signaling region, and the CD19 sequences may be optimized by changes in the codons.

Leader Sequences

Leader sequences may be added to enhance the stability of mRNA and result in more efficient translation. The leader sequence is usually involved in targeting the mRNA to the endoplasmic reticulum. Examples include the signal sequence for the HIV-1 envelope glycoprotein (Env), which delays its own cleavage, and the IgE gene leader sequence (Kutzler, M. A., and Weiner, D. B., 2008. Nature Rev. Gen. 9:776-88; Li, V., et al., 2000. Virology 272:417-28; Xu, Z. L., et al. 2001. Gene 272:149-56; Malin, A. S., et al., 2000. Microbes Infect. 2:1677-85; Kutzler, M. A., et al., 2005. J. Immunol. 175:112-125; Yang, J. S., et al., 2002. Emerg. Infect. Dis. 8:1379-84; Kumar, S., et al., 2006. DNA Cell Biol. 25:383-92; Wang, S., et al., 2006. Vaccine 24:4531-40). The IgE leader may be used to enhance insertion into the endoplasmic reticulum (Tepler, I, et al. (1989) J. Biol. Chem. 264:5912).

Expression of the transgenes may be optimized and/or controlled by the selection of appropriate methods for optimizing expression. These methods include, for example, optimizing promoters, delivery methods, and gene sequences, (for example, as presented in Laddy, D. J., et al., 2008. PLoS.ONE 3 e2517; Kutzler, M. A., and Weiner, D. B., 2008. Nature Rev. Gen. 9:776-88).

Nucleic Acids

A “nucleic acid” as used herein generally refers to a molecule (one, two or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.” Nucleic acids may be, be at least, be at most, or be about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length.

Nucleic acids herein provided may have regions of identity or complementarity to another nucleic acid. It is contemplated that the region of complementarity or identity can be at least 5 contiguous residues, though it is specifically contemplated that the region is, is at least, is at most, or is about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides.

As used herein, “hybridization”, “hybridizes” or “capable of hybridizing” is understood to mean forming a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are known, and are often used for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42 degrees C. to about 70 degrees C. It is understood that the temperature and ionic strength of a desired stringency are determined in part by the length of the particular nucleic acid(s), the length and nucleobase content of the target sequence(s), the charge composition of the nucleic acid(s), and the presence or concentration of formamide, tetramethylammonium chloride or other solvent(s) in a hybridization mixture.

It is understood that these ranges, compositions and conditions for hybridization are mentioned by way of non-limiting examples only, and that the desired stringency for a particular hybridization reaction is often determined empirically by comparison to one or more positive or negative controls. Depending on the application envisioned varying conditions of hybridization may be employed to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20 degrees C. to about 50 degrees C. The low or high stringency conditions may be further modified to suit a particular application.

“Function-conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

Amino acids other than those indicated as conserved may differ in a protein or enzyme so that the percent protein or amino acid sequence similarity between any two proteins of similar function may vary and can be, for example, at least 70%, at least 80%, at least 90%, or at least 95%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA. When using any of these programs, the preferred settings are those that results in the highest sequence similarity.

Nucleic Acid Modification

Any of the modifications discussed below may be applied to a nucleic acid. Examples of modifications include alterations to the RNA or DNA backbone, sugar or base, and various combinations thereof. Any suitable number of backbone linkages, sugars and/or bases in a nucleic acid can be modified (e.g., independently about 5%, 10%, 15%, 20%, 25%, 30%, 35%, 40%, 45%, 50%, 55%, 60%, 65%, 70%, 75%, 80%, 85%, 90%, 95%, up to 100%). An unmodified nucleoside is any one of the bases adenine, cytosine, guanine, thymine, or uracil joined to the 1′ carbon of beta-D-ribo-furanose.

A modified base is a nucleotide base other than adenine, guanine, cytosine and uracil at a 1′ position. Non-limiting examples of modified bases include inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e. g., 5-methylcytidine), 5-alkyluridines (e. g., ribothymidine), 5-halouridine (e. g., 5-bromouridine) or 6-azapyrimidines or 6-alkylpyrimidines (e. g. 6-methyluridine), propyne, and the like. Other non-limiting examples of modified bases include nitropyrrolyl (e.g., 3-nitropyrrolyl), nitroindolyl (e.g., 4-, 5-, 6-nitroindolyl), hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, difluorotolyl, 4-fluoro-6-methylbenzimidazole, 4-methylbenzimidazole, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenyl, tetracenyl, pentacenyl and the like.

In some embodiments, for example, a nucleid acid may comprise modified nucleic acid molecules, with phosphate backbone modifications. Non-limiting examples of backbone modifications include phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl modifications. In certain instances, a ribose sugar moiety that naturally occurs in a nucleoside is replaced with a hexose sugar, polycyclic heteroalkyl ring, or cyclohexenyl group. In certain instances, the hexose sugar is an allose, altrose, glucose, mannose, gulose, idose, galactose, talose, or a derivative thereof. The hexose may be a D-hexose, glucose, or mannose. In certain instances, the polycyclic heteroalkyl group may be a bicyclic ring containing one oxygen atom in the ring. In certain instances, the polycyclic heteroalkyl group is a bicyclo[2.2.1]heptane, a bicyclo[3.2.1]octane, or a bicyclo[3.3.1]nonane.

Nitropyrrolyl and nitroindolyl nucleobases are members of a class of compounds known as universal bases. Universal bases are those compounds that can replace any of the four naturally occurring bases without substantially affecting the melting behavior or activity of the oligonucleotide duplex. In contrast to the stabilizing, hydrogen-bonding interactions associated with naturally occurring nucleobases, oligonucleotide duplexes containing 3-nitropyrrolyl nucleobases may be stabilized solely by stacking interactions. The absence of significant hydrogen-bonding interactions with nitropyrrolyl nucleobases obviates the specificity for a specific complementary base. In addition, 4-, 5- and 6-nitroindolyl display very little specificity for the four natural bases. Procedures for the preparation of 1-(2′-O-methyl-.beta.-D-ribofuranosyl)-5-nitroindole are discussed in Gaubert, G.; Wengel, J. Tetrahedron Letters 2004, 45, 5629. Other universal bases include hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, and structural derivatives thereof.

Difluorotolyl is a non-natural nucleobase that functions as a universal base. Difluorotolyl is an isostere of the natural nucleobase thymine. But unlike thymine, difluorotolyl shows no appreciable selectivity for any of the natural bases. Other aromatic compounds that function as universal bases are 4-fluoro-6-methylbenzimidazole and 4-methylbenzimidazole. In addition, the relatively hydrophobic isocarbostyrilyl derivatives 3-methyl isocarbostyrilyl, 5-methyl isocarbostyrilyl, and 3-methyl-7-propynyl isocarbostyrilyl are universal bases which cause only slight destabilization of oligonucleotide duplexes compared to the oligonucleotide sequence containing only natural bases. Other non-natural nucleobases include 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenyl, tetracenyl, pentacenyl, and structural derivates thereof. For a more detailed discussion, including synthetic procedures, of difluorotolyl, 4-fluoro-6-methylbenzimidazole, 4-methylbenzimidazole, and other non-natural bases mentioned above, see: Schweitzer et al., J. Org. Chem., 59:7238-7242 (1994);

In addition, chemical substituents, for example cross-linking agents, may be used to add further stability or irreversibility to the reaction. Non-limiting examples of cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl) dithio]propioimidate.

A nucleotide analog may also include a “locked” nucleic acid. Certain compositions can be used to essentially “anchor” or “lock” an endogenous nucleic acid into a particular structure. Anchoring sequences serve to prevent disassociation of a nucleic acid complex, and thus not only can prevent copying but may also enable labeling, modification, and/or cloning of the endogenous sequence. The locked structure may regulate gene expression (i.e. inhibit or enhance transcription or replication), or can be used as a stable structure that can be used to label or otherwise modify the endogenous nucleic acid sequence, or can be used to isolate the endogenous sequence, i.e. for cloning.

Nucleic acid molecules need not be limited to those molecules containing only RNA or DNA, but further encompass chemically-modified nucleotides and non-nucleotides. The percent of non-nucleotides or modified nucleotides may be from 1% to 100% (e.g., about 5, 10, 15, 20, 25, 30, 35, 40, 45, 50, 55, 60, 65, 70, 75, 80, 85, 90 or 95%).

Nucleic Acid Preparation

In some embodiments, a nucleic acid is provided for use as a control or standard in an assay, or therapeutic, for example. A nucleic acid may be made by any technique known in the art, such as for example, chemical synthesis, enzymatic production or biological production. Nucleic acids may be recovered or isolated from a biological sample. The nucleic acid may be recombinant or it may be natural or endogenous to the cell (produced from the cell's genome). It is contemplated that a biological sample may be treated in a way so as to enhance the recovery of small nucleic acid molecules. Generally, methods may involve lysing cells with a solution having guanidinium and a detergent.

Nucleic acid synthesis may also be performed according to standard methods. Non-limiting examples of a synthetic nucleic acid (e.g., a synthetic oligonucleotide), include a nucleic acid made by in vitro chemical synthesis using phosphotriester, phosphite, or phosphoramidite chemistry and solid phase techniques or via deoxynucleoside H-phosphonate intermediates. Various different mechanisms of oligonucleotide synthesis have been disclosed elsewhere.

Nucleic acids may be isolated using known techniques. In particular embodiments, methods for isolating small nucleic acid molecules, and/or isolating RNA molecules can be employed. Chromatography is a process used to separate or isolate nucleic acids from protein or from other nucleic acids. Such methods can involve electrophoresis with a gel matrix, filter columns, alcohol precipitation, and/or other chromatography. If a nucleic acid from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

Methods may involve the use of organic solvents and/or alcohol to isolate nucleic acids. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column is effective for such isolation procedures.

A nucleic acid isolation processes may sometimes include: a) lysing cells in the sample with a lysing solution comprising guanidinium, where a lysate with a concentration of at least about 1 M guanidinium is produced; b) extracting nucleic acid molecules from the lysate with an extraction solution comprising phenol; c) adding to the lysate an alcohol solution for form a lysate/alcohol mixture, wherein the concentration of alcohol in the mixture is between about 35% to about 70%; d) applying the lysate/alcohol mixture to a solid support; e) eluting the nucleic acid molecules from the solid support with an ionic solution; and, f) capturing the nucleic acid molecules. The sample may be dried down and resuspended in a liquid and volume appropriate for subsequent manipulation.

Methods of Gene Transfer

In order to mediate the effect of the transgene expression in a cell, it will be necessary to transfer the expression constructs into a cell. Such transfer may employ viral or non-viral methods of gene transfer. This section provides a discussion of methods and compositions of gene transfer. A transformed cell comprising an expression vector is generated by introducing into the cell the expression vector. Suitable methods for polynucleotide delivery for transformation of an organelle, a cell, a tissue or an organism for use with the current methods include virtually any method by which a polynucleotide (e.g., DNA) can be introduced into an organelle, a cell, a tissue or an organism.

A host cell can, and has been, used as a recipient for vectors. Host cells may be derived from prokaryotes or eukaryotes, depending upon whether the desired result is replication of the vector or expression of part or all of the vector-encoded polynucleotide sequences. Numerous cell lines and cultures are available for use as a host cell, and they can be obtained through the American Type Culture Collection (ATCC), which is an organization that serves as an archive for living cultures and genetic materials. In specific embodiments, the host cell is a T cell, a tumor-infiltrating lymphocyte, a natural killer cell, or a natural killer T cell.

An appropriate host may be determined. Generally this is based on the vector backbone and the desired result. A plasmid or cosmid, for example, can be introduced into a prokaryote host cell for replication of many vectors. Bacterial cells used as host cells for vector replication and/or expression include DH5alpha, JM109, and KC8, as well as a number of commercially available bacterial hosts such as SURE® Competent Cells and SOLOPACK Gold Cells (STRATAGENE®, La Jolla, Calif.). Alternatively, bacterial cells such as E. coli LE392 could be used as host cells for phage viruses. Eukaryotic cells that can be used as host cells include, but are not limited to yeast, insects and mammals. Examples of mammalian eukaryotic host cells for replication and/or expression of a vector include, but are not limited to, HeLa, NIH3T3, Jurkat, 293, COS, CHO, Saos, and PC12. Examples of yeast strains include, but are not limited to, YPH499, YPH500 and YPH501.

Nucleic acid vaccines may include, for example, non-viral DNA vectors, “naked” DNA and RNA, and viral vectors. Methods of transforming cells with these vaccines, and for optimizing the expression of genes included in these vaccines are known and are also discussed herein.

Examples of Methods of Nucleic Acid or Viral Vector Transfer

Any appropriate method may be used to transfect or transduce the cells, for example, the T cells, or to administer the nucleotide sequences or compositions of the present methods. Certain examples are presented herein, and further include methods such as delivery using cationic polymers, lipid like molecules, and certain commercial products such as, for example, IN-VIVO-JET PEI.

1. Ex vivo Transformation

Various methods are available for transfecting vascular cells and tissues removed from an organism in an ex vivo setting. For example, canine endothelial cells have been genetically altered by retroviral gene transfer in vitro and transplanted into a canine (Wilson et al., Science, 244:1344-1346, 1989). In another example, Yucatan minipig endothelial cells were transfected by retrovirus in vitro and transplanted into an artery using a double-balloon catheter (Nabel et al., Science, 244(4910):1342-1344, 1989). Thus, it is contemplated that cells or tissues may be removed and transfected ex vivo using the polynucleotides presented herein. In particular aspects, the transplanted cells or tissues may be placed into an organism. For example, dendritic cells from an animal, transfect the cells with the expression vector and then administer the transfected or transduced cells back to the animal.

2. Injection

In certain embodiments, a cell or a nucleic acid or viral vector may be delivered to an organelle, a cell, a tissue or an organism via one or more injections (i.e., a needle injection), such as, for example, subcutaneous, intradermal, intramuscular, intravenous, intraprotatic, intratumor, intrintraperitoneal, etc. Methods of injection include, for example, injection of a composition comprising a saline solution. Further embodiments include the introduction of a polynucleotide by direct microinjection. The amount of the expression vector used may vary upon the nature of the antigen as well as the organelle, cell, tissue or organism used.

Intradermal, intranodal, or intralymphatic injections are some of the more commonly used methods of DC administration. Intradermal injection is characterized by a low rate of absorption into the bloodstream but rapid uptake into the lymphatic system. The presence of large numbers of Langerhans dendritic cells in the dermis will transport intact as well as processed antigen to draining lymph nodes. Proper site preparation is necessary to perform this correctly (i.e., hair is clipped in order to observe proper needle placement). Intranodal injection allows for direct delivery of antigen to lymphoid tissues. Intralymphatic injection allows direct administration of DCs.

3. Electroporation

In certain embodiments, a polynucleotide is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference).

Transfection of eukaryotic cells using electroporation has been quite successful. Mouse pre-B lymphocytes have been transfected with human kappa-immunoglobulin genes (Potter et al., (1984) Proc. Nat'l Acad. Sci. USA, 81, 7161-7165), and rat hepatocytes have been transfected with the chloramphenicol acetyltransferase gene (Tur-Kaspa et al., (1986) Mol. Cell Biol., 6, 716-718) in this manner.

In vivo electroporation for vaccines, or eVac, is clinically implemented through a simple injection technique. A DNA vector encoding tumor antigen is injected intradermally in a patient. Then electrodes apply electrical pulses to the intradermal space causing the cells localized there, especially resident dermal dendritic cells, to take up the DNA vector and express the encoded tumor antigen. These tumor antigen-expressing dendritic cells activated by local inflammation can then migrate to lymph-nodes, presenting tumor antigens and priming tumor antigen-specific T cells. A nucleic acid is electroporetically administered when it is administered using electroporation, following, for example, but not limited to, injection of the nucleic acid or any other means of administration where the nucleic acid may be delivered to the cells by electroporation Methods of electroporation are discussed in, for example, Sardesai, N.Y., and Weiner, D. B., Current Opinion in Immunotherapy 23:421-9 (2011) and Ferraro, B. et al., Human Vaccines 7:120-127 (2011), which are hereby incorporated by reference herein in their entirety.

4. Calcium Phosphate

In other embodiments, a polynucleotide is introduced to the cells using calcium phosphate precipitation. Human KB cells have been transfected with adenovirus 5 DNA (Graham and van der Eb, (1973) Virology, 52, 456-467) using this technique. Also in this manner, mouse L(A9), mouse C127, CHO, CV-1, BHK, NIH3T3 and HeLa cells were transfected with a neomycin marker gene (Chen and Okayama, Mol. Cell Biol., 7(8):2745-2752, 1987), and rat hepatocytes were transfected with a variety of marker genes (Rippe et al., Mol. Cell Biol., 10:689-695, 1990).

5. DEAE-Dextran

In another embodiment, a polynucleotide is delivered into a cell using DEAE-dextran followed by polyethylene glycol. In this manner, reporter plasmids were introduced into mouse myeloma and erythroleukemia cells (Gopal, T. V., Mol Cell Biol. 1985 May; 5(5):1188-90).

6. Sonication Loading

Additional embodiments include the introduction of a polynucleotide by direct sonic loading. LTK-fibroblasts have been transfected with the thymidine kinase gene by sonication loading (Fechheimer et al., (1987) Proc. Nat'l Acad. Sci. USA, 84, 8463-8467).

7. Liposome-Mediated Transfection

In a further embodiment, a polynucleotide may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine (Gibco BRL) or Superfect (Qiagen).

8. Receptor Mediated Transfection

Still further, a polynucleotide may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity. Certain receptor-mediated gene targeting vehicles comprise a cell receptor-specific ligand and a polynucleotide-binding agent. Others comprise a cell receptor-specific ligand to which the polynucleotide to be delivered has been operatively attached. Several ligands have been used for receptor-mediated gene transfer (Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085), which establishes the operability of the technique. Specific delivery in the context of another mammalian cell type has been discussed (Wu and Wu, Adv. Drug Delivery Rev., 12:159-167, 1993; incorporated herein by reference). In certain aspects, a ligand is chosen to correspond to a receptor specifically expressed on the target cell population. In other embodiments, a polynucleotide delivery vehicle component of a cell-specific polynucleotide-targeting vehicle may comprise a specific binding ligand in combination with a liposome. The polynucleotide(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a polynucleotide to cells that exhibit upregulation of the EGF receptor.

In still further embodiments, the polynucleotide delivery vehicle component of a targeted delivery vehicle may be a liposome itself, which may, for example, comprise one or more lipids or glycoproteins that direct cell-specific binding. For example, lactosyl-ceramide, a galactose-terminal asialoganglioside, have been incorporated into liposomes and observed an increase in the uptake of the insulin gene by hepatocytes (Nicolau et al., (1987) Methods Enzymol., 149, 157-176). It is contemplated that the tissue-specific transforming constructs may be specifically delivered into a target cell in a similar manner.

9. Microprojectile Bombardment

Microprojectile bombardment techniques can be used to introduce a polynucleotide into at least one, organelle, cell, tissue or organism (U.S. Pat. Nos. 5,550,318; 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., (1987) Nature, 327, 70-73). There are a wide variety of microprojectile bombardment techniques known in the art, many of which are applicable to the present methods. In this microprojectile bombardment, one or more particles may be coated with at least one polynucleotide and delivered into cells by a propelling force. Several devices for accelerating small particles have been developed. One such device relies on a high voltage discharge to generate an electrical current, which in turn provides the motive force (Yang et al., (1990) Proc. Nat'l Acad. Sci. USA, 87, 9568-9572). The microprojectiles used have consisted of biologically inert substances such as tungsten or gold particles or beads. Exemplary particles include those comprised of tungsten, platinum, and, in certain examples, gold, including, for example, nanoparticles. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

Examples of Methods of Viral Vector-Mediated Transfer

Any viral vector suitable for administering nucleotide sequences, or compositions comprising nucleotide sequences, to a cell or to a subject, such that the cell or cells in the subject may express the genes encoded by the nucleotide sequences may be employed in the present methods. In certain embodiments, a transgene is incorporated into a viral particle to mediate gene transfer to a cell. Typically, the virus simply will be exposed to the appropriate host cell under physiologic conditions, permitting uptake of the virus. The present methods are advantageously employed using a variety of viral vectors, as discussed below.

1. Adenovirus

Adenovirus is particularly suitable for use as a gene transfer vector because of its mid-sized DNA genome, ease of manipulation, high titer, wide target-cell range, and high infectivity. The roughly 36 kb viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, M. J. (1990) Radiother Oncol., 19, 197-218). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence, which makes them useful for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present methods, it is possible to achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay, R. T., et al., J Mol Biol. 1984 Jun. 5; 175(4):493-510). Therefore, inclusion of these elements in an adenoviral vector may permits replication.

In addition, the packaging signal for viral encapsulation is localized between 194-385 bp (0.5-1.1 map units) at the left end of the viral genome (Hearing et al., J. (1987) Virol., 67, 2555-2558). This signal mimics the protein recognition site in bacteriophage lambda DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., Gene, 101:195-202, 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element derives from the packaging function of adenovirus.

It has been shown that a packaging signal for adenovirus exists in the left end of the conventional adenovirus map (Tibbetts et. al. (1977) Cell, 12, 243-249). Later studies showed that a mutant with a deletion in the E1A (194-358 bp) region of the genome grew poorly even in a cell line that complemented the early (E1A) function (Hearing and Shenk, (1983) J. Mol. Biol. 167, 809-822). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved toward the interior of the Ad5 DNA molecule (Hearing et al., J. (1987) Virol., 67, 2555-2558).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals is packaged selectively when compared to the helpers. If the preference is great enough, stocks approaching homogeneity may be achieved.

To improve the tropism of ADV constructs for particular tissues or species, the receptor-binding fiber sequences can often be substituted between adenoviral isolates. For example the Coxsackie-adenovirus receptor (CAR) ligand found in adenovirus 5 can be substituted for the CD46-binding fiber sequence from adenovirus 35, making a virus with greatly improved binding affinity for human hematopoietic cells. The resulting “pseudotyped” virus, Ad5f35, has been the basis for several clinically developed viral isolates. Moreover, various biochemical methods exist to modify the fiber to allow re-targeting of the virus to target cells, such as, for example, T cells. Methods include use of bifunctional antibodies (with one end binding the CAR ligand and one end binding the target sequence), and metabolic biotinylation of the fiber to permit association with customized avidin-based chimeric ligands. Alternatively, one could attach ligands (e.g. anti-CD205 by heterobifunctional linkers (e.g. PEG-containing), to the adenovirus particle.

2. Retrovirus

The retroviruses are a group of single-stranded RNA viruses characterized by an ability to convert their RNA to double-stranded DNA in infected cells by a process of reverse-transcription (Coffin, (1990) In: Virology, ed., New York: Raven Press, pp. 1437-1500). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990). Thus, for example, the present technology includes, for example, cells whereby the polynucleotide used to transduce the cell is integrated into the genome of the cell.

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and psi components is constructed (Mann et al., (1983) Cell, 33, 153-159). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas, J. F., and Rubenstein, J. L. R., (1988) In: Vectors: a Survey of Molecular Cloning Vectors and Their Uses, Rodriquez and Denhardt, Eds.). Nicolas and Rubenstein; Temin et al., (1986) In: Gene Transfer, Kucherlapati (ed.), New York: Plenum Press, pp. 149-188; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., (1975) Virology, 67, 242-248). An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, may this be desired.

A different approach to targeting of recombinant retroviruses was designed which used biotinylated antibodies against a retroviral envelope protein and against a specific cell receptor. The antibodies were coupled via the biotin components by using streptavidin (Roux et al., (1989) Proc. Nat'l Acad. Sci. USA, 86, 9079-9083). Using antibodies against major histocompatibility complex class I and class II antigens, the infection of a variety of human cells that bore those surface antigens was demonstrated with an ecotropic virus in vitro (Roux et al., 1989).

3. Adeno-associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low-level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., J. Virol., 61:3096-3101 (1987)), or by other methods, including but not limited to chemical or enzymatic synthesis of the terminal repeats based upon the published sequence of AAV. It can be determined, for example, by deletion analysis, the minimum sequence or part of the AAV ITRs which is required to allow function, i.e., stable and site-specific integration. It can also be determined which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, (1995) Ann. N.Y. Acad. Sci., 770; 79-90; Chatteijee, et al., (1995) Ann. N.Y. Acad. Sci., 770, 79-90; Ferrari et al., (1996) J. Virol., 70, 3227-3234; Fisher et al., (1996) J. Virol., 70, 520-532; Flotte et al., Proc. Nat'l Acad. Sci. USA, 90, 10613-10617, (1993); Goodman et al. (1994), Blood, 84, 1492-1500; Kaplitt et al., (1994) Nat'l Genet., 8, 148-153; Kaplitt, M. G., et al., Ann Thorac Surg. 1996 December; 62(6):1669-76; Kessler et al., (1996) Proc. Nat'l Acad. Sci. USA, 93, 14082-14087; Koeberl et al., (1997) Proc. Nat'l Acad. Sci. USA, 94, 1426-1431; Mizukami et al., (1996) Virology, 217, 124-130).

AAV-mediated efficient gene transfer and expression in the lung has led to clinical trials for the treatment of cystic fibrosis (Carter and Flotte, 1995; Flotte et al., Proc. Nat'l Acad. Sci. USA, 90, 10613-10617, (1993)). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., (1996) J. Virol., 70, 520-532; Flotte et al., 1993; Kaplitt et al., 1994; 1996; Koeberl et al., 1997; McCown et al., (1996) Brain Res., 713, 99-107; Ping et al., (1996) Microcirculation, 3, 225-228; Xiao et al., (1996) J. Virol., 70, 8098-8108).

4. Other Viral Vectors

Other viral vectors are employed as expression constructs in the present methods and compositions. Vectors derived from viruses such as vaccinia virus (Ridgeway, (1988) In: Vectors: A survey of molecular cloning vectors and their uses, pp. 467-492; Baichwal and Sugden, (1986) In, Gene Transfer, pp. 117-148; Coupar et al., Gene, 68:1-10, 1988) canary poxvirus, and herpes viruses are employed. These viruses offer several features for use in gene transfer into various mammalian cells.

Once the construct has been delivered into the cell, the nucleic acid encoding the transgene are positioned and expressed at different sites. In certain embodiments, the nucleic acid encoding the transgene is stably integrated into the genome of the cell. This integration is in the cognate location and orientation via homologous recombination (gene replacement) or it is integrated in a random, non-specific location (gene augmentation). In yet further embodiments, the nucleic acid is stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

Methods for Treating a Disease

The present methods also encompass methods of treatment or prevention of a disease where administration of cells by, for example, infusion, may be beneficial.

Cells, such as, for example, T cells, tumor infiltrating lymphocytes, natural killer cells, TCR-expressing cells, natural killer T cells, or progenitor cells, such as, for example, hematopoietic stem cells, mesenchymal stromal cells, stem cells, pluripotent stem cells, and embryonic stem cells may be used for cell therapy. The cells may be from a donor, or may be cells obtained from the patient. The cells may, for example, be used in regeneration, for example, to replace the function of diseased cells. The cells may also be modified to express a heterologous gene so that biological agents may be delivered to specific microenvironments such as, for example, diseased bone marrow or metastatic deposits. Mesenchymal stromal cells have also, for example, been used to provide immunosuppressive activity, and may be used in the treatment of graft versus host disease and autoimmune disorders. The cells provided in the present application contain a safety switch that may be valuable in a situation where following cell therapy, the activity of the therapeutic cells needs to be removed. increased, or decreased. For example, where progenitor T cells that express a chimeric antigen receptor are provided to the patient, in some situations there may be an adverse event, such as inappropriate differentiation of the cell into a more mature cell type, or an undesired invitation into another tissue off-target toxicity. Ceasing the administration of the ligand would return the therapeutic T cells to a non-activated state, remaining at a low, non-toxic, level of expression. Or, for example, where it is necessary to remove the therapeutic cells. The therapeutic cell may work to decrease the tumor cell, or tumor size, and may no longer be needed. In this situation, administration of the ligand may cease, and the therapeutic cells would no longer be activated. If the tumor cells return, or the tumor size increases following the initial therapy, the ligand may be administered again, in order to activate the chimeric antigen receptor-expressing T cells, and re-treat the patient. In some embodiments, cells are transfected or transduced with nucleic acids that encode the chimeric signaling polypeptides and inducible chimeric signaling polypeptides of the present application. In some embodiments, the transfected or transduced cells are selected from the group consisting of T cells, NK cells, NK-T cells, for example invariant NK-T cells, gamma delta T cells, and tumor infiltrating lymphocytes. In some embodiments, the transfected or transduced cells are selected from the group consisting of T cells, NK cells, NK-T cells and tumor infiltrating lymphocytes, wherein the lymphocytes are not antigen-presenting cells. In some embodiments, the tumor infiltrating lymphocytes are not B cells or macrophages. In some embodiments, the cells are T cells; in other embodiments the cells are NK-T cells. In some embodiments, the cells are invariant NK-T cells. In some embodiments, the cells are gamma delta T cells. In other embodiments, the cells are NK cells.

By “therapeutic cell” is meant a cell used for cell therapy, that is, a cell administered to a subject to treat or prevent a condition or disease. Therapeutic cells may, for example, be immune cells. The therapeutic cells may be, for example, any cell administered to a patient for a desired therapeutic result. The therapeutic cells may be, for example, immune cells such as, for example, T cells, natural killer cells, B cells, tumor infiltrating lymphocytes, or macrophages; the therapeutic cells may be, for example, peripheral blood cells, hematopoietic progenitor cells, bone marrow cells, or tumor cells. To further improve the tumor microenvironment to be more immunogenic, the treatment may be combined with one or more adjuvants (e.g., IL-12, TLRs, IDO inhibitors, etc.). In some embodiments, the cells may be delivered to treat a solid tumor, such as, for example, delivery of the cells to a tumor bed.

Also provided in some embodiments are nucleic acid vaccines, such as DNA vaccines, wherein the vaccine comprises a nucleic acid comprising a polynucleotide that encodes an inducible, or constitutive chimeric signaling polypeptide of the present application. The vaccine may be administered to a subject, thereby transforming or transducing target cells in vivo.

The term “unit dose” as it pertains to the inoculum refers to physically discrete units suitable as unitary dosages for mammals, each unit containing a predetermined quantity of pharmaceutical composition calculated to produce the desired immune-stimulating effect in association with the required diluent. The specifications for the unit dose of an inoculum are dictated by and are dependent upon the unique characteristics of the pharmaceutical composition and the particular immunologic effect to be achieved.

An effective amount of the pharmaceutical composition, such as the modified cell or multimeric ligand presented herein, would be the amount that achieves this selected result of activating the inducible chimeric signaling polypeptide—expressing cells, such that over 60%, 70%, 80%, 85%, 90%, 95%, or 97%, or that under 80%, 70%, 60%, 50%, 40%, 30%, 20%, or 10% of the therapeutic cells are activated. The term is also synonymous with “sufficient amount.” The effective amount may also be the amount that achieves the desired therapeutic response, such as, the reduction of tumor size, the decrease in the level of tumor cells, or the decrease in the level of target antigen-expressing, such as, for example, CD19-expressing leukemic cells, compared to the time before the ligand inducer, or cells, are administered.

By administering a modified cell, it is understood that an effective amount of modified cells are administered.

To determine if an effective amount of ligand or modified cells is administered, any means of assaying or measuring the number of target cells, or amount of target antigen, or size of a tumor may be used to determine whether the number of target cells, amount of target antigen or size of a tumor has increased, decreased, or remained the same. Samples, images, or other means of measurement taken before administration of the modified cells or ligand may be used to compare with samples, images, or other means of measurement taken after administration of the modified cells or ligand. Thus, for example, to determine whether the the amount or concentration of cells expressing a target antigen has increased, decreased, or remained the same, a first sample may be obtained from a subject before administration of the ligand or modified cells, and a second sample may be obtained from a subject after administration of the ligand or modified cells. The amount or concentration of cells expressing the target antigen in the first sample may be compared with the amount or concentration of cells expressing the target antigen in the second sample, in order to determine whether the amount or concentration of cells expressing the target antigen has increased, decreased, or remained the same following administration of the ligand or modified cell.

The effective amount for any particular application can vary depending on such factors as the disease or condition being treated, the particular composition being administered, the size of the subject, and/or the severity of the disease or condition. One can empirically determine the effective amount of a particular composition presented herein without necessitating undue experimentation.

The effective amount for any particular application can vary depending on such factors as the disease or condition being treated, the particular composition being administered, the size of the subject, and/or the severity of the disease or condition. One can empirically determine the effective amount of a particular composition presented herein without necessitating undue experimentation.

The terms “contacted” and “exposed,” when applied to a cell, tissue or organism, are used herein to describe the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing.

The administration of the pharmaceutical composition may precede, be concurrent with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered within of from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein and one or more agents may be employed.

Optimized and Personalized Therapeutic Treatment

The dosage and administration schedule of the ligand inducer may be optimized by determining the level of the disease or condition to be treated. For example, the size of any remaining solid tumor, or the level of targeted cells such as, for example, tumor cells that may remain in the patient, may be determined.

For example, determining that a patient has clinically relevant levels of tumor cells, or a solid tumor, after initial therapy, provides an indication to a clinician that it may be necessary to activate the chimeric-antigen receptor-expressing T cells by activating the cells by administering the multimeric ligand. In another example, determining that a patient has a reduced level of tumor cells or reduced tumor size after treatment with the multimeric ligand may indicate to the clinician that no additional dose of the multimeric ligand is needed. Similarly, after treatment with the multimeric ligand, determining that the patient continues to exhibit disease or condition symptoms, or suffers a relapse of symptoms may indicate to the clinician that it may be necessary to administer at least one additional dose of multimeric ligand. The term “dosage” is meant to include both the amount of the dose and the frequency of administration, such as, for example, the timing of the next dose. The term “dosage level” refers to the amount of the multimeric ligand administered in relation to the body weight of the subject. Thus increasing the dosage level would mean increasing the amount of the ligand administered relative to the subject's weight. In addition, increasing the concentration of the dose administered, such as, for example, when the multimeric ligand is administered using a continuous infusion pump would mean that the concentration administered (and thus the amount administered) per minute, or second, is increased.

Thus, for example, in certain embodiments where cells that express an inducible chimeric signaling polypeptide or an inducible chimeric antigen receptor polypeptide are administered to a patient, the methods comprise determining the presence or absence of a tumor size increase and/or increase in the number of tumor cells in a subject relative to the tumor size and/or the number of tumor cells following administration of the multimeric ligand, and administering an additional dose of the multimeric ligand to the subject in the event the presence of a tumor size increase and/or increase in the number of tumor cells is determined. In these embodiments, for example, the patient is initially treated with the therapeutic cells and ligand according to the methods provided herein. Following the initial treatment, the size of the tumor, or the number of tumor cells, may decrease relative to the time prior to the initial treatment. At a certain time after this initial treatment, the patient is again tested, or the patient may be continually monitored for disease symptoms. If it is determined that the size of the tumor, or the number of tumor cells, for example, is increased relative to the time just after the initial treatment, then the ligand may be administered for an additional dose. This monitoring and treatment schedule may continue, because the therapeutic cells that express inducible chimeric signaling polypeptides remain in the patient, although in a relatively inactive state in the absence of additional ligand.

In other examples where cells that express an inducible chimeric signaling polypeptide or an inducible chimeric antigen receptor polypeptide are administered to a patient, and where the target cell is not a tumor cell, the methods comprise determining the presence or absence of the concentration or number of target cells in a subject relative to the concentration or number of target cells following administration of the multimeric ligand, and administering an additional dose of the multimeric ligand to the subject in the event the presence of an increase in the concentration or number of target cells is determined. For this analysis, the concentration or number of target cells refers to the concentration or number of target cells in a sample obtained from the patient. these embodiments, for example, the patient is initially treated with the therapeutic cells and ligand according to the methods provided herein. Following the initial treatment, the concentration or the number of target cells may decrease relative to the time prior to the initial treatment. At a certain time after this initial treatment, the patient is again tested, or the patient may be continually monitored for disease symptoms. If it is determined that the concentration or number of target cells, for example, is increased relative to the time just after the initial treatment, then the ligand may be administered for an additional dose. This monitoring and treatment schedule may continue, because the therapeutic cells that express inducible chimeric signaling polypeptides remain in the patient, although in a relatively inactive state in the absence of additional ligand.

An indication of adjusting or maintaining a subsequent drug dose, such as, for example, a subsequent dose of the multimeric ligand, and/or the subsequent drug dosage, can be provided in any convenient manner. An indication may be provided in tabular form (e.g., in a physical or electronic medium) in some embodiments. For example, the size of the tumor cell, or the number or level of tumor cells in a sample may be provided in a table, and a clinician may compare the symptoms with a list or table of stages of the disease. The clinician then can identify from the table an indication for subsequent drug dose. In certain embodiments, an indication can be presented (e.g., displayed) by a computer, after the symptoms are provided to the computer (e.g., entered into memory on the computer). For example, this information can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

Methods as presented herein include without limitation the delivery of an effective amount of an activated cell, a nucleic acid, an expression construct encoding the same, or the multimeric ligand. An “effective amount” of the pharmaceutical composition, cell, nucleic acid, expression construct, or multimeric ligand, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers to evaluate the effectiveness of treatment and to control toxicity.

Enhancement of an Immune Response

In certain embodiments, an immune cell activation strategy is contemplated, that incorporates the manipulation of signaling costimulatory polypeptides that activate biological pathways, for example, immunological pathways, such as, for example, NF-kappaB pathways, Akt pathways, and/or p38 pathways. This immune cell activation system can be used in conjunction with or without standard vaccines to enhance the immune response.

For antigen presenting cell activation, such as dendritic cell activation, the immune cell activation system discussed herein replaces the requirement for CD4⁺ T cell help during APC activation (Bennett, S. R., et al., Nature, 1998, Jun. 4. 393: p. 478-80; Ridge, J. P., D. R. F, and P. Nature, 1998, Jun. 4. 393: p. 474-8; Schoenberger, S. P., et al., Nature, 1998, Jun. 4. 393: p. 480-3). Thus, the DC activation system presented herein enhances immune responses by circumventing the need for the generation of MHC class II-specific peptides.

For example, release of IFNγ by IL-12-stimulated NK cells can lead to upregulation of MHC on target cells along with improvments in antigen presentation. Furthermore, the release of chemokines such as MCP1, XCL1, XCL2, CCLS and CXCL10 by activated NK cells in an anti-tumor response can recruit and induce the differentiation of dendridic cells and induce the expansion of an adaptive immune response by native T lymphocytes as well as recruiting macrophage as part of an overall inflammatory response (Bottcher et al. (2018) Cell 172: p 1022-1037.

In certain embodiments the cells are in an animal, such as human, non-human primate, cow, horse, pig, sheep, goat, dog, cat, or rodent. The subject may be, for example, an animal, such as a mammal, for example, a human, non-human primate, cow, horse, pig, sheep, goat, dog, cat, or rodent. The subject may be, for example, human, for example, a patient suffering from an infectious disease, and/or a subject that is immunocompromised, or is suffering from a hyperproliferative disease.

In further embodiments, the expression construct and/or expression vector can be utilized as a composition or substance that activates cells. Such a composition that “activates cells” or “enhances the activity cells” refers to the ability to stimulate one or more activities associated with cells. For example, a composition, such as the expression construct or vector of the present methods, can stimulate upregulation of costimulatory molecules on cells, induce nuclear translocation of NF-kappaB in cells, activate toll- like receptors in cells, or other activities involving cytokines or chemokines.

The expression construct, expression vector and/or transduced cells can enhance or contribute to the effectiveness of a vaccine by, for example, enhancing the immunogenicity of weaker antigens such as highly purified or recombinant antigens, reducing the amount of antigen required for an immune response, reducing the frequency of immunization required to provide protective immunity, improving the efficacy of vaccines in subjects with reduced or weakened immune responses, such as newborns, the aged, and immunocompromised individuals, and enhancing the immunity at a target tissue, such as mucosal immunity, or promote cell-mediated or humoral immunity by eliciting a particular cytokine profile.

In certain embodiments, the cell is also contacted with an antigen. Often, the cell is contacted with the antigen ex vivo. Sometimes, the cell is contacted with the antigen in vivo. In some embodiments, the cell is in a subject and an immune response is generated against the antigen. Sometimes, the immune response is a cytotoxic T-lymphocyte (CTL) immune response. Sometimes, the immune response is generated against a tumor antigen. In certain embodiments, the cell is activated without the addition of an adjuvant.

In some embodiments, the cell is transduced with the nucleic acid ex vivo and administered to the subject by intradermal administration. In some embodiments, the cell is transduced with the nucleic acid ex vivo and administered to the subject by subcutaneous administration. Sometimes, the cell is transduced with the nucleic acid ex vivo. Sometimes, the cell is transduced with the nucleic acid in vivo.

In certain embodiments, the cell can be transduced ex vivo or in vivo with a nucleic acid that encodes the chimeric protein. The cell may be sensitized to the antigen at the same time the cell is contacted with the multimeric ligand, or the cell can be pre-sensitized to the antigen before the cell is contacted with the multimerization ligand. In some embodiments, the cell is contacted with the antigen ex vivo. In certain embodiments the cell is transduced with the nucleic acid ex vivo and administered to the subject by intradermal administration, and sometimes the cell is transduced with the nucleic acid ex vivo and administered to the subject by subcutaneous administration. The antigen may be a tumor antigen, and the CTL immune response can be induced by migration of the cell to a draining lymph node. A tumor antigen is any antigen such as, for example, a peptide or polypeptide, that triggers an immune response in a host. The tumor antigen may be a tumor-associated antigen that is associated with a neoplastic tumor cell.

In some embodiments, an immunocompromised individual or subject is a subject that has a reduced or weakened immune response. Such individuals may also include a subject that has undergone chemotherapy or any other therapy resulting in a weakened immune system, a transplant recipient, a subject currently taking immunosuppressants, an aging individual, or any individual that has a reduced and/or impaired CD4 T helper cells. It is contemplated that the present methods can be utilized to enhance the amount and/or activity of CD4 T helper cells in an immunocompromised subject.

Challenge with Target Antigens

In specific embodiments, prior to administering the transduced cell, the cells are challenged with antigens (also referred herein as “target antigens”). After challenge, the transduced, loaded cells are administered to the subject parenterally, intradermally, intranodally, or intralymphatically. Additional parenteral routes include, but are not limited to subcutaneous, intramuscular, intraperitoneal, intravenous, intraarterial, intramyocardial, transendocardial, transepicardial, intrathecal, intraprotatic, intratumor, and infusion techniques.

The target antigen, as used herein, is an antigen or immunological epitope on the antigen, which is crucial in immune recognition and ultimate elimination or control of the disease-causing agent or disease state in a mammal. The immune recognition may be cellular and/or humoral. In the case of intracellular pathogens and cancer, immune recognition may, for example, be a T lymphocyte response.

The target antigen may be derived or isolated from, for example, a pathogenic microorganism such as viruses including HIV, (Korber et al, eds HIV Molecular Immunology Database, Los Alamos National Laboratory, Los Alamos, N. Mex. 1977) influenza, Herpes simplex, human papilloma virus (U.S. Pat. No. 5,719,054), Hepatitis B (U.S. Pat. No. 5,780,036), Hepatitis C (U.S. Pat. No. 5,709,995), EBV, Cytomegalovirus (CMV) and the like. Target antigen may be derived or isolated from pathogenic bacteria such as, for example, from Chlamydia (U.S. Pat. No. 5,869,608), Mycobacteria, Legionella, Meningiococcus, Group A Streptococcus, Salmonella, Listeria, Hemophilus influenzae (U.S. Pat. No. 5,955,596) and the like.

Target antigen may be derived or isolated from, for example, pathogenic yeast including Aspergillus, invasive Candida (U.S. Pat. No. 5,645,992), Nocardia, Histoplasmosis, Cryptosporidia and the like.

Target antigen may be derived or isolated from, for example, a pathogenic protozoan and pathogenic parasites including but not limited to Pneumocystis carinii, Trypanosoma, Leishmania (U.S. Pat. No. 5,965,242), Plasmodium (U.S. Pat. No. 5,589,343) and Toxoplasma gondii. Target antigen includes an antigen associated with a preneoplastic or hyperplastic state. Target antigen may also be associated with, or causative of cancer. Such target antigen may be, for example, tumor specific antigen, tumor associated antigen (TAA) or tissue specific antigen, epitope thereof, and epitope agonist thereof. Such target antigens include but are not limited to carcinoembryonic antigen (CEA) and epitopes thereof such as CAP-1, CAP-1-6D and the like (GenBank Accession No. M29540), MART-1 (Kawakarni et al, J. Exp. Med. 180:347-352, 1994), MAGE-1 (U.S. Pat. No. 5,750,395), MAGE-3, GAGE (U.S. Pat. No. 5,648,226), GP-100 (Kawakami et al Proc. Nat'l Acad. Sci. USA 91:6458-6462, 1992), MUC-1, MUC-2, point mutated ras oncogene, normal and point mutated p53 oncogenes (Hollstein et al Nucleic Acids Res. 22:3551-3555, 1994), PSMA (Israeli et al Cancer Res. 53:227-230, 1993), tyrosinase (Kwon et al PNAS 84:7473-7477, 1987) TRP-1 (gp75) (Cohen et al Nucleic Acid Res. 18:2807-2808, 1990; U.S. Pat. No. 5,840,839), NY-ESO-1 (Chen et al PNAS 94: 1914-1918, 1997), TRP-2 (Jackson et al EMBOJ, 11:527-535, 1992), TAG72, KSA, CA-125, PSA, HER-2/neu/c-erb/B2, (U.S. Pat. No. 5,550,214), BRC-I, BRC-II, bcr-abl, pax3-fkhr, ews-fli-1, modifications of TAAs and tissue specific antigen, splice variants of TAAs, epitope agonists, and the like. Other TAAs may be identified, isolated and cloned by methods known in the art such as those disclosed in U.S. Pat. No. 4,514,506. Target antigen may also include one or more growth factors and splice variants of each. An antigen may be expressed more frequently in cancer cells than in non-cancer cells. The antigen may result from contacting the modified dendritic cell with a prostate specific membrane antigen, for example, a prostate specific membrane antigen (PSMA) or fragment thereof.

Prostate antigen (PA001) is a recombinant protein consisting of the extracellular portion of PSMA antigen. PSMA is a ˜100 kDa (84 kDa before glycosylation, 180 kDa as dimer) type II membrane protein with neuropeptidase and folate hydrolase activities, but the true function of PSMA is currently unclear. Carter R E, et al., Proc Natl Acad Sci USA. 93: 749-53, 1996; Israeli R S, et al., Cancer Res. 53: 227-30, 1993; Pinto J T, et al., Clin Cancer Res. 2: 1445-51, 1996.

Expression is largely, but not exclusively, prostate-specific and is maintained in advanced and hormone refractory disease. Israeli R S, et al., Cancer Res. 54: 1807-11, 1994. Weak non-prostatic detection in normal tissues has also been seen in the salivary gland, brain, small intestines, duodenal mucosa, proximal renal tubules and neuroendocrine cells in colonic crypts. Silver D A, et al., Clin Cancer Res. 3: 81-5, 1997; Troyer J K, et al., Int J Cancer. 62: 552-8, 1995. Moreover, PSMA is up-regulated following androgen deprivation therapy (ADT). Wright G L, Jr., et al., Urology. 48: 326-34, 1996. While most PSMA is expressed as a cytoplasmic protein, the alternatively-spliced transmembrane form is the predominate form on the apical surface of neoplastic prostate cells. Su S L, et al., Cancer Res. 55: 1441-3, 1995; Israeli R S, et al., Cancer Res. 54: 6306-10, 1994.

Moreover, PSMA is internalized following cross-linking and has been used to internalize bound antibody or ligand complexed with radionucleotides or viruses and other complex macromolecules. Liu H, et al., Cancer Res. 58: 4055-60, 1998; Freeman L M, et al., Q J Nucl Med. 46: 131-7, 2002; Kraaij R, et al., Prostate. 62: 253-9, 2005. Bander and colleagues demonstrated that pretreatment of tumors with microtubule inhibitors increases aberrant basal surface targeting and antibody-mediated internalization of PSMA. Christiansen J J, et al., Mol Cancer Ther. 4: 704-14, 2005. Tumor targeting may be facilitated by the observation of ectopic expression of PSMA in tumor vascular endothelium of not only prostate, but also renal and other tumors. Liu H, et al., Cancer Res. 57: 3629-34, 1997; Chang S S, et al., Urology. 57: 801-5, 2001; Chang S S, et al., Clin Cancer Res. 5: 2674-81, 1999.

PSMA is not found in the vascular endothelial cells of corresponding benign tissue. de la Taille A, et al., Cancer Detect Prey. 24: 579-88, 2000. Although one early histological study of metastatic prostate disease suggested that only ˜50% (8 of 18) of bone metastases (with 7 of 8 lymph node metastases) expressed PSMA, the more sensitive reagent, 177Lu-radiolabeled MoAb J591, targeted to the ectodomain of PSMA, could target all known sites of bone and soft tissue metastasis in 30 of 30 patients, suggesting near universal expression in advanced prostate disease. Bander N H, et al., J Clin Oncol. 23: 4591-601, 2005.

A prostate specific antigen, or PSA, is meant to include any antigen that can induce an immune response, such as, for example, a cytotoxic T lymphocyte response, against a PSA, for example, a PSMA, and may be specifically recognized by any anti-PSA antibody. PSAs used in the present method are capable of being used to load the cell, as assayed using conventional methods. Thus, “prostate specific antigen” or “PSA” may, for example, refer to a protein having the wild type amino acid sequence of a PSA, or a polypeptide that includes a portion of the a PSA protein,

A prostate specific membrane antigen, or PSMA, is meant to include any antigen that can induce an immune response, such as, for example, a cytotoxic T lymphocyte response, against PSMA, and may be specifically recognized by an anti-PSMA antibody. PSMAs used in the present method are capable of being used to load the cell, as assayed using conventional methods. Thus, “prostate specific membrane antigen” or “PSMA” may, for example, refer to a protein having the wild type amino acid sequence of PSMA, or a polypeptide that includes a portion of the PSMA protein. Also included are variants of any of the foregoing, including, for example, those having substitutions and deletions. Proteins, polypeptides, and peptides having differential post-translational processing, such as differences in glycosylation, from the wild type PSMA, may also be used in the present methods. Further, various sugar molecules that are capable of inducing an immune response against PSMA, are also contemplated.

A PSA, for example, a PSMA, polypeptide may be used to load the modified cell. In certain embodiments, the modified cell is contacted with a PSMA polypeptide fragment. In some embodiments, the PSA, for example, PSMA polypeptide fragment does not include the signal peptide sequence. In other embodiments, the modified cell is contacted with a PSA, for example, PSMA polypeptide fragment comprising substitutions or deletions of amino acids in the polypeptide, and the fragment is sufficient to load cells.

A prostate specific protein antigen, or s PSPA, also referred to in this specification as a prostate specific antigen, or a PSA, is meant to include any antigen that can induce an immune response, such as, for example, a cytotoxic T lymphocyte response, against a prostate specific protein antigen. This includes, for example, a prostate specific protein antigen or Prostate Specific Antigen. PSPAs used in the present method are capable of being used to load the cell, as assayed using conventional methods. Prostate Specific Antigen, or PSA, may, for example, refer to a protein having the wild type amino acid sequence of a PSA, or a polypeptide that includes a portion of the PSA protein,

A prostate specific membrane antigen, or PSMA, is meant to include any antigen that can induce an immune response, such as, for example, a cytotoxic T lymphocyte response, against PSMA, and may be specifically recognized by an anti-PSMA antibody. PSMAs used in the present method are capable of being used to load the cell, as assayed using conventional methods. Thus, “prostate specific membrane antigen” or “PSMA” may, for example, refer to a protein having the wild type amino acid sequence of PSMA, or a polypeptide that includes a portion of the PSMA protein. Also included are variants of any of the foregoing, including, for example, those having substitutions and deletions. Proteins, polypeptides, and peptides having differential post-translational processing, such as differences in glycosylation, from the wild type PSMA, may also be used in the present methods. Further, various sugar molecules that are capable of inducing an immune response against PSMA, are also contemplated.

A PSPA, for example, a PSMA, polypeptide may be used to load the modified cell. In certain embodiments, the modified cell is contacted with a PSMA polypeptide fragment. In some embodiments, the PSA, for example, PSMA polypeptide fragment does not include the signal peptide sequence. In other embodiments, the modified cell is contacted with a PSPA, for example, PSMA polypeptide fragment comprising substitutions or deletions of amino acids in the polypeptide, and the fragment is sufficient to load cells.

A tumor antigen is any antigen such as, for example, a peptide or polypeptide, that triggers an immune response in a host against a tumor. The tumor antigen may be a tumor-associated antigen that is associated with a neoplastic tumor cell.

A prostate cancer antigen, or PCA, is any antigen such as, for example, a peptide or polypeptide, that triggers an immune response in a host against a prostate cancer tumor. A prostate cancer antigen may, or may not, be specific to prostate cancer tumors. A prostate cancer antigen may also trigger immune responses against other types of tumors or neoplastic cells. A prostate cancer antigen includes, for example, prostate specific protein antigens, prostate specific antigens, and prostate specific membrane antigens.

The cell may be contacted with tumor antigen, such as PSA, for example, PSMA polypeptide, by various methods, including, for example, pulsing immature DCs with unfractionated tumor lysates, MHC-eluted peptides, tumor-derived heat shock proteins (HSPs), tumor associated antigens (TAAs (peptides or proteins)), or transfecting DCs with bulk tumor mRNA, or mRNA coding for TAAs (reviewed in Gilboa, E. & Vieweg, J., Immunol Rev 199, 251-63 (2004); Gilboa, E, Nat Rev Cancer 4, 401-11 (2004)).

For organisms that contain a DNA genome, a gene encoding a target antigen or immunological epitope thereof of interest is isolated from the genomic DNA. For organisms with RNA genomes, the desired gene may be isolated from cDNA copies of the genome. If restriction maps of the genome are available, the DNA fragment that contains the gene of interest is cleaved by restriction endonuclease digestion by routine methods. In instances where the desired gene has been previously cloned, the genes may be readily obtained from the available clones. Alternatively, if the DNA sequence of the gene is known, the gene can be synthesized by any of the conventional techniques for synthesis of deoxyribonucleic acids.

Genes encoding an antigen of interest can be amplified, for example, by cloning the gene into a bacterial host. For this purpose, various prokaryotic cloning vectors can be used. Examples are plasmids pBR322, pUC and pEMBL.

The genes encoding at least one target antigen or immunological epitope thereof can be prepared for insertion into the plasmid vectors designed for recombination with a virus by standard techniques. In general, the cloned genes can be excised from the prokaryotic cloning vector by restriction enzyme digestion. In most cases, the excised fragment will contain the entire coding region of the gene. The DNA fragment carrying the cloned gene can be modified as needed, for example, to make the ends of the fragment compatible with the insertion sites of the DNA vectors used for recombination with a virus, then purified prior to insertion into the vectors at restriction endonuclease cleavage sites (cloning sites).

Antigen loading of cells, such as, for example, dendritic cells, with antigens may be achieved, for example, by contacting cells, such as, for example, dendritic cells or progenitor cells with an antigen, for example, by incubating the cells with the antigen. Loading may also be achieved, for example, by incubating DNA (naked or within a plasmid vector) or RNA that code for the antigen; or with antigen-expressing recombinant bacterium or viruses (e.g., vaccinia, fowlpox, adenovirus or lentivirus vectors). Prior to loading, the antigen may be covalently conjugated to an immunological partner that provides T cell help (e.g., a carrier molecule). Alternatively, a dendritic cell may be pulsed with a non-conjugated immunological partner, separately or in the presence of the polypeptide. Antigens from cells or MHC molecules may be obtained by acid-elution or other methods (see Zitvogel L, et al., J Exp Med 1996. 183:87-97). The cells may be transduced or transfected with the chimeric protein-encoding nucleotide sequence according to the present methods either before, after, or at the same time as the cells are loaded with antigen. In particular embodiments, antigen loading is subsequent to transduction or transfection.

In further embodiments, the transduced cell is transfected with tumor cell mRNA. The transduced transfected cell is administered to an animal to effect cytotoxic T lymphocytes and natural killer cell anti-tumor antigen immune response and regulated using dimeric FK506 and dimeric FK506 analogs. The tumor cell mRNA may be, for example, mRNA from a prostate tumor cell.

In some embodiments, the transduced cell may be loaded by pulsing with tumor cell lysates. The pulsed transduced cells are administered to an animal to effect cytotoxic T lymphocytes and natural killer cell anti-tumor antigen immune response and regulated using dimeric FK506 and dimeric FK506 analogs. The tumor cell lysate may be, for example, a prostate tumor cell lysate.

Immune Cells and Cytotoxic T Lymphocyte Response

T-lymphocytes may be activated by contact with the cell that comprises the expression vector discussed herein, where the cell has been challenged, transfected, pulsed, or electrofused with an antigen.

T cells express a unique antigen binding receptor on their membrane (T-cell receptor), which can only recognize antigen in association with major histocompatibility complex (MHC) molecules on the surface of other cells. There are several populations of T cells, such as T helper cells and T cytotoxic cells. T helper cells and T cytotoxic cells are primarily distinguished by their display of the membrane bound glycoproteins CD4 and CD8, respectively. T helper cells secret various lymphokines that are crucial for the activation of B cells, T cytotoxic cells, macrophages and other cells of the immune system. In contrast, a naïve CD8 T cell that recognizes an antigen-MHC complex proliferates and differentiates into an effector cell called a cytotoxic CD8 T lymphocyte (CTL). CTLs eliminate cells of the body displaying antigen, such as virus-infected cells and tumor cells, by producing substances that result in cell lysis.

CTL activity can be assessed by methods discussed herein, for example. For example, CTLs may be assessed in freshly isolated peripheral blood mononuclear cells (PBMC), in a phytohaemaglutinin-stimulated IL-2 expanded cell line established from PBMC (Bernard et al., AIDS, 12(16):2125-2139, 1998) or by T cells isolated from a previously immunized subject and restimulated for 6 days with DC infected with an adenovirus vector containing antigen using standard 4 hour 51Cr release microtoxicity assays. One type of assay uses cloned T-cells. Cloned T-cells have been tested for their ability to mediate both perforin and Fas ligand-dependent killing in redirected cytotoxicity assays (Simpson et al., Gastroenterology, 115(4):849-855, 1998). The cloned cytotoxic T lymphocytes displayed both Fas- and perforin-dependent killing. Recently, an in vitro dehydrogenase release assay has been developed that takes advantage of a new fluorescent amplification system (Page, B., et al., Anticancer Res. 1998 July-August; 18(4A):2313-6). This approach is sensitive, rapid, and reproducible and may be used advantageously for mixed lymphocyte reaction (MLR). It may easily be further automated for large-scale cytotoxicity testing using cell membrane integrity, and is thus considered. In another fluorometric assay developed for detecting cell-mediated cytotoxicity, the fluorophore used is the non-toxic molecule AlamarBlue (Nociari et al., J. Immunol. Methods, 213(2): 157-167, 1998). The AlamarBlue is fluorescently quenched (i.e., low quantum yield) until mitochondrial reduction occurs, which then results in a dramatic increase in the AlamarBlue fluorescence intensity (i.e., increase in the quantum yield). This assay is reported to be extremely sensitive, specific and requires a significantly lower number of effector cells than the standard 51Cr release assay.

Other immune cells that can be induced by the present methods include natural killer cells (NK). NKs are lymphoid cells that lack antigen-specific receptors and are part of the innate immune system. Typically, infected cells are usually destroyed by T cells alerted by foreign particles bound to the cell surface MHC. However, virus-infected cells signal infection by expressing viral proteins that are recognized by antibodies. These cells can be killed by NKs. In tumor cells, if the tumor cells lose expression of MHC I molecules, then it may be susceptible to NKs.

Formulations and Routes for Administration to Patients

Where clinical applications are contemplated, it will be necessary to prepare pharmaceutical compositions—expression constructs, expression vectors, fused proteins, transduced cells, activated T cells, transduced and loaded T cells—in a form appropriate for the intended application. Generally, this will entail preparing compositions that are essentially free of pyrogens, as well as other impurities that could be harmful to humans or animals.

The multimeric ligand, such as, for example, rimiducid, may be delivered, for example at doses of about 0.01 to 1 mg/kg subject weight, of about 0.05 to 0.5 mg/kg subject weight, 0.1 to 2 mg/kg subject weight, of about 0.05 to 1.0 mg/kg subject weight, of about 0.1 to 5 mg/kg subject weight, of about 0.2 to 4 mg/kg subject weight, of about 0.3 to 3 mg/kg subject weight, of about 0.3 to 2 mg/kg subject weight, or about 0.3 to 1 mg/kg subject weight, for example, about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 mg/kg subject weight. In some embodiments, the ligand is provided at 0.4 mg/kg per dose, for example at a concentration of 5 mg/mL. Vials or other containers may be provided containing the ligand at, for example, a volume per vial of about 0.25 ml to about 10 ml, for example, about 0.25, 0.5, 1, 1.5, 2, 2.5, 3, 3.5, 4, 4.5, 5, 5.5, 6, 6.5, 7, 7.5, 8, 8.5, 9, 9.5, or 10 ml, for example, about 2 ml.

Rimiducid for Injection

AP1903 API is manufactured by Alphora Research Inc. and AP1903 Drug Product for Injection is made by Formatech Inc. It is formulated as a 5 mg/mL solution of AP1903 in a 25% solution of the non-ionic solubilizer Solutol HS 15 (250 mg/mL, BASF). At room temperature, this formulation is a clear, slightly yellow solution. Upon refrigeration, this formulation undergoes a reversible phase transition, resulting in a milky solution. This phase transition is reversed upon re-warming to room temperature. The fill is 2.33 mL in a 3 mL glass vial (˜10 mg AP1903 for Injection total per vial).

Rimiducid is removed from the refrigerator the night before the patient is dosed and stored at a temperature of approximately 21° C. overnight, so that the solution is clear prior to dilution. The solution is prepared within 30 minutes of the start of the infusion in glass or polyethylene bottles or non-DEHP bags and stored at approximately 21° C. prior to dosing.

All study medication is maintained at a temperature between 2 degrees C. and 8 degrees C., protected from excessive light and heat, and stored in a locked area with restricted access.

Administration

In one example, patients are administered a single fixed dose of rimiducid for Injection (0.4 mg/kg) via IV infusion over 2 hours, using a non-DEHP, non-ethylene oxide sterilized infusion set. The dose of rimiducid is calculated individually for all patients, and is not be recalculated unless body weight fluctuates by ≥10c/o. The calculated dose is diluted in 100 mL in 0.9% normal saline before infusion.

Patients are observed for 15 minutes following the end of the infusion for untoward adverse effects.

One may generally desire to employ appropriate salts and buffers to render delivery vectors stable and allow for uptake by target cells. Buffers also may be employed when recombinant cells are introduced into a patient. Aqueous compositions comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. A pharmaceutically acceptable carrier includes any and all solvents, dispersion media, coatings, antibacterial and antifungal agents, isotonic and absorption delaying agents and the like. The use of such media and agents for pharmaceutically active substances is known. Except insofar as any conventional media or agent is incompatible with the vectors or cells, its use in therapeutic compositions is contemplated. Supplementary active ingredients also can be incorporated into the compositions.

The active compositions may include classic pharmaceutical preparations. Administration of these compositions will be via any common route so long as the target tissue is available via that route. This includes, for example, oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, discussed herein.

The pharmaceutical forms suitable for injectable use include sterile aqueous solutions or dispersions and sterile powders for the extemporaneous preparation of sterile injectable solutions or dispersions. In all cases the form is sterile and is fluid to the extent that easy syringability exists. It is stable under the conditions of manufacture and storage and is preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In certain examples, isotonic agents, for example, sugars or sodium chloride may be included. Prolonged absorption of the injectable compositions can be brought about by the use in the compositions of agents delaying absorption, for example, aluminum monostearate and gelatin.

For oral administration, the compositions may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including, for example: gels, pastes, powders and slurries. The active ingredient may be added in a therapeutically effective amount to a paste dentifrice that may include, for example, water, binders, abrasives, flavoring agents, foaming agents, and humectants.

The compositions may be formulated in a neutral or salt form. Pharmaceutically-acceptable salts include, for example, the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media can be employed. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations may meet sterility, pyrogenicity, and general safety and purity standards as required by FDA Office of Biologics standards.

The administration schedule may be determined as appropriate for the patient and may, for example, comprise a dosing schedule where the nucleic acid is administered at week 0, followed by induction by administration of the chemical inducer of dimerization, followed by administration of additional inducer when needed to obtain an effective therapeutic result or, for example, at 2, 4, 6, 8, 10, 12, 14, 16, 18, 20 intervals thereafter fora total of, for example, 2, 4, 6, 8, 10, 12, 14, 16, 18, 20, 22, 24, 26, 28, or 30, 40, 50, 60, 70, 80, 90, or 100 weeks.

The administration schedule may be determined as appropriate for the patient and may, for example, comprise a dosing schedule where the nucleic acid-transduced T cell or other cell is administered at week 0, followed by induction by administration of the chemical inducer of dimerization, followed by administration of additional inducer when needed to obtain an effective therapeutic result or, for example, at 2, 4, 6, 8, 10, 12, 14, 16, 18, 20 intervals thereafter for a total of, for example, 2, 4, 6, 8, 10, 12, 14, 16, 18, 20, 22, 24, 26, 28, or 30, 40, 50, 60, 70, 80, 90, or 100 weeks.

Although for administration of transduced T cells, one dose is likely to be sufficient, followed by multiple doses of ligand, T cells may be provided more than once, or other cells, such as the non-dendritic cells and non-B cells discussed herein may also be administered multiple times. In addition, nucleic acids targeted to the non-T cell aspects of the present technology may also be administered more than one time for optimum therapeutic efficacy. Therefore, for example, The administration schedule may be determined as appropriate for the patient and may, for example, comprise a dosing schedule where the nucleic acid or nucleic acid-transduced cell is administered at week 0, followed by induction by administration of the chemical inducer of dimerization, followed by administration of additional nucleic acid or nucleic acid-transduced cell and inducer at 2 week intervals thereafter fora total of, for example, 2, 4, 6, 8, 10, 12, 14, 16, 18, 20, 22, 24, 26, 28, or 30 weeks.

Other dosing schedules include, for example, a schedule where one dose of the cells and one dose of the inducer are administered. In another example, the schedule may comprise administering the cells and the inducer are administered at week 0, followed by the administration of additional cells and inducer at 4 week intervals, for a total of, for example, 4, 8, 12, 16, 20, 24, 28, or 32 weeks.

Administration of a dose of cells may occur in one session, or in more than one session, but the term dose may refer to the total amount of cells administered before administration of the ligand.

If needed, the method may further include additional leukaphereses to obtain more cells to be used in treatment.

Methods for Treating a Disease

The present methods also encompass methods of treatment or prevention of a disease caused by pathogenic microorganisms and/or a hyperproliferative disease.

Diseases that may be treated or prevented include diseases caused by viruses, bacteria, yeast, parasites, protozoa, cancer cells and the like. The pharmaceutical composition (transduced T cells, expression vector, expression construct, etc.) may be used as a generalized immune enhancer (T cell activating composition or system) and as such has utility in treating diseases. Exemplary diseases that can be treated and/or prevented include, but are not limited, to infections of viral etiology such as HIV, influenza, Herpes, viral hepatitis, Epstein Bar, polio, viral encephalitis, measles, chicken pox, Papilloma virus etc.; or infections of bacterial etiology such as pneumonia, tuberculosis, syphilis, etc.; or infections of parasitic etiology such as malaria, trypanosomiasis, leishmaniasis, trichomoniasis, amoebiasis, etc.

Preneoplastic or hyperplastic states which may be treated or prevented using the pharmaceutical composition (transduced T cells, expression vector, expression construct, etc.) include but are not limited to preneoplastic or hyperplastic states such as colon polyps, Crohn's disease, ulcerative colitis, breast lesions and the like.

Cancers, including solid tumors, which may be treated using the pharmaceutical composition include, but are not limited to primary or metastatic melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemias, uterine cancer, breast cancer, prostate cancer, ovarian cancer, pancreatic cancer, colon cancer, multiple myeloma, neuroblastoma, NPC, bladder cancer, cervical cancer and the like.

Other hyperproliferative diseases, including solid tumors, that may be treated using the T cell and other therapeutic cell activation system presented herein include, but are not limited to rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis.

Methods of treatment may include methods for prophylactic or therapeutic purposes. When provided prophylactically, the pharmaceutical composition, for example, the expression construct, expression vector, fused protein, transduced cells, activated immune cells, transduced or loaded immune cells, is provided in advance of any detected or reported symptom. The prophylactic administration of pharmaceutical composition serves to prevent or ameliorate any subsequent infection or disease. When provided therapeutically, the pharmaceutical composition is provided at or after the onset of a symptom of infection or disease. Thus the compositions presented herein may be provided either prior to the anticipated exposure to a disease-causing agent or disease state or after the initiation of the infection or disease. Thus provided herein are methods for prophylactic treatment of solid tumors such as those found in cancer, or for example, but not limited to, prostate cancer, using the nucleic acids and ligands discussed herein.

For example, methods are provided of prophylactically preventing or reducing the size of a tumor, or reducing the concentration or number of target cells, in a subject comprising administering a nucleic acid comprising a promoter operably linked to a polynucleotide that encodes a chimeric signaling polypeptide, and a nucleic acid that encodes a CAR or a recombinant TCR. The chimeric signaling polypeptide may, or may not comprise a membrane targeting region, and may optionally be inducible or constitutive. The chimeric signaling polypeptide may also be provided as part of the CAR polypeptide. The nucleic acid, and optionally the multimeric ligand are administered in an amount effect to prevent or reduce the size of a tumor, or the concentration or number of target cells in a subject. The term multimerization region or multimeric ligand binding region may be used in place of the term ligand binding region for purposes of this application.

Solid tumors from any tissue or organ may be treated using the present methods, including, for example, any tumor expressing PSA, for example, PSMA, in the vasculature, for example, solid tumors present in, for example, lungs, bone, liver, prostate, or brain, and also, for example, in breast, ovary, bowel, testes, colon, pancreas, kidney, bladder, neuroendocrine system, soft tissue, boney mass, and lymphatic system. Other solid tumors that may be treated include, for example, glioblastoma, and malignant myeloma.

The term “unit dose” as it pertains to the inoculum refers to physically discrete units suitable as unitary dosages for mammals, each unit containing a predetermined quantity of pharmaceutical composition calculated to produce the desired immunogenic effect in association with the required diluent. The specifications for the unit dose of an inoculum are dictated by and are dependent upon the unique characteristics of the pharmaceutical composition and the particular immunologic effect to be achieved.

An effective amount of the pharmaceutical composition would be the amount that achieves this selected result of enhancing the immune response, and such an amount could be determined. For example, an effective amount of for treating an immune system deficiency could be that amount necessary to cause activation of the immune system, resulting in the development of an antigen specific immune response upon exposure to antigen. The term is also synonymous with “sufficient amount.”

The effective amount for any particular application can vary depending on such factors as the disease or condition being treated, the particular composition being administered, the size of the subject, and/or the severity of the disease or condition. One can empirically determine the effective amount of a particular composition presented herein without necessitating undue experimentation. Thus, for example, in one embodiment, the transduced T cells or other cells are administered to a subject in an amount effective to, for example, induce an immune response, or, for example, to reduce the size of a tumor or reduce the amount of tumor vasculature.

In some embodiments, multiple doses of multimeric ligand are administered to the subject, with an escalation of dosage levels among the multiple doses. In some embodiments, the escalation of dosage levels increases the level of CAR-T cell activity, or recombinant TCR-T cell activity, and therefore increases the therapeutic effect, such as, for example, the reduction in the amount or concentration of target cells, such as, for example, tumor cells. In some embodiments, the dose is escalated from 0.01 to 1 mg/kg. In some embodiments, the doses are administered in increments of about 15 to 30 minutes. In some embodiments, the multimeric ligand is administered using a continuous infusion pump, and the concentration of multimeric ligand is increased during the infusion. In some embodiments, the multimeric ligand is administered in separate doses 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart, or 1, 2, 3, 4, 5, 6, 7, 8, 9, 10, or 11 months apart, or 1, 2, 3, 4, 5, 6, 7, 8, 9, or 10 years apart.

In some embodiments, personalized treatment is provided wherein the stage or level of the disease or condition is determined before administration of the multimeric ligand, before the administration of an additional dose of the multimeric ligand, or in determining method and dosage involved in the administration of the multimeric ligand. These methods may be used in any of the methods of the present application. Where these methods of assessing the patient before administering the ligand are discussed in the context of, for example, the treatment of a subject with a solid tumor, it is understood that these methods may be similarly applied to the treatment of other conditions and diseases. Thus, for example, in some embodiments of the present application, the method comprises administering the modified cells of the present application to a subject, and further comprises determining the appropriate dose of multimeric ligand to achieve the effective level of reduction of tumor size. In some examples, a smaller dose may be sufficient to activate the CAR-T cell, by inducing a sufficient level of costimulatory molecule activity to achieve the required therapeutic result. In some examples, a higher dose may be necessary to achieve a higher level of costimulation of CAR-T cell activity. The amount of ligand may be determined, for example, based on the subject's clinical condition, weight, and/or gender or other relevant physical characteristic. By controlling the amount of multimeric ligand administered to the subject, the likelihood of adverse events such as, for example, a cytokine storm may be reduced. The anti-tumor activity of modified cells that express the inducible chimeric signaling polypeptide along with a CAR may be modulated using appropriate dosages of the multimeric ligand. Thus provided in certain embodiments are methods where the modified cell is administered to a subject, and a dosage of multimeric ligand is administered; following this first administration, the method may comprise identifying a presence or absence of a condition in the patient that requires an increase or decrease in the level of CAR-T cell activity, which may be achieved by an additional dose of multimeric ligand in either greater or lower concentrations than the first dose. Thus the method comprises administering a multimeric ligand that binds to the multimeric ligand binding region of the inducible chimeric signaling polypeptide, maintaining a subsequent dosage of the multimeric ligand, or adjusting a subsequent dosage of the multimeric ligand to the patient based on the presence or absence of the condition identified in the patient.

The term “dosage” is meant to include both the amount of the dose and the frequency of administration, such as, for example, the timing of the next dose. The term “dosage level” refers to the amount of the multimeric ligand administered in relation to the body weight of the subject. Thus increasing the dosage level would mean increasing the amount of the ligand administered relative to the subject's weight. In addition, increasing the concentration of the dose administered, such as, for example, when the multimeric ligand is administered using a continuous infusion pump would mean that the concentration administered (and thus the amount administered) per minute, or second, is increased.

An indication of adjusting or maintaining a subsequent drug dose, such as, for example, a subsequence dose of the multimeric ligand, and/or the subsequent drug dosage, can be provided in any convenient manner. An indication may be provided in tabular form (e.g., in a physical or electronic medium) in some embodiments. For example, the disease or condition symptoms may be provided in a table, and a clinician may compare the symptoms with a list or table of stages of the disease. The clinician then can identify from the table an indication for subsequent drug dose. In certain embodiments, an indication can be presented (e.g., displayed) by a computer, after the symptoms or the stage is provided to the computer (e.g., entered into memory on the computer).

For example, this information can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

Methods as presented herein include without limitation the delivery of an effective amount of an activated cell, a nucleic acid, or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers to evaluate the effectiveness of treatment and to control toxicity.

An effective amount of the pharmaceutical composition when used to induce apoptosis in cells that express an inducible Caspase 9 polypeptide, such as the multimeric ligand presented herein, would be the amount that achieves this selected result of inducing apoptosis in the Caspase-9-expressing cells T cells, such that over 60%, 70%, 80%, 85%, 90%, 95%, or 97%, or that under 80%, 70%, 60%, 50%, 40%, 30%, 20%, or 10% of the therapeutic cells are killed. The term is also synonymous with “sufficient amount.” The effective amount where the pharmaceutical composition is the modified T cell may also be the amount that achieves the desired therapeutic response, such as, the reduction of tumor size, the decrease in the level of tumor cells, or the decrease in the concentration or number of target cells, compared to the time before the ligand inducer is administered.

The effective amount for any particular application can vary depending on such factors as the disease or condition being treated, the particular composition being administered, the size of the subject, and/or the severity of the disease or condition. One can empirically determine the effective amount of a particular composition presented herein without necessitating undue experimentation.

A. Genetic Based Therapies

In certain embodiments, a cell is provided with an expression construct capable of providing a chimeric signaling polypeptide or chimeric antigen receptor polypeptide, such as those discussed herein, and, for example, in a T cell. The lengthy discussion of expression vectors and the genetic elements employed therein is incorporated into this section by reference. In certain examples, the expression vectors may be viral vectors, such as adenovirus, adeno-associated virus, herpes virus, vaccinia virus and retrovirus. In another example, the vector may be a lysosomal-encapsulated expression vector.

Gene delivery may be performed in both in vivo and ex vivo situations. For viral vectors, one generally will prepare a viral vector stock. Examples of viral vector-mediated gene delivery ex vivo and in vivo are presented in the present application. For in vivo delivery, depending on the kind of virus and the titer attainable, one will deliver, for example, about 1, 2, 3, 4, 5, 6, 7, 8, or 9×104, 1, 2, 3, 4, 5, 6, 7, 8, or 9×105, 1, 2, 3, 4, 5, 6, 7, 8, or 9×106, 1, 2, 3, 4, 5, 6, 7, 8, or 9×107, 1, 2, 3, 4, 5, 6, 7, 8, or 9×108, 1, 2, 3, 4, 5, 6, 7, 8, or 9×109, 1, 2, 3, 4, 5, 6, 7, 8, or 9×1010, 1, 2, 3, 4, 5, 6, 7, 8, or 9×1011 or 1, 2, 3, 4, 5, 6, 7, 8, or 9×1012 infectious particles to the patient. Similar figures may be extrapolated for liposomal or other non-viral formulations by comparing relative uptake efficiencies. Formulation as a pharmaceutically acceptable composition is discussed below. The multimeric ligand, such as, for example, rimiducid, may be delivered, for example at doses of about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 mg/kg subject weight.

B. Cell Based Therapy

Another therapy that is contemplated is the administration of transduced or transfected immune cells, such as T cells. The immune cells may be transduced in vitro. Formulation as a pharmaceutically acceptable composition is discussed herein.

In cell based therapies, the transduced cells may be, for example, transfected with target antigen nucleic acids, such as mRNA or DNA or proteins; pulsed with cell lysates, proteins or nucleic acids; or electrofused with cells. The cells, proteins, cell lysates, or nucleic acid may derive from cells, such as tumor cells or other pathogenic microorganism, for example, viruses, bacteria, protozoa, etc.

C. Combination Therapies

In order to increase the effectiveness of the expression vectors presented herein, it may be desirable to combine these compositions and methods with an agent effective in the treatment of the disease.

In certain embodiments, anti-cancer agents may be used in combination with the present methods. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

In further embodiments antibiotics can be used in combination with the pharmaceutical composition to treat and/or prevent an infectious disease. Such antibiotics include, but are not limited to, amikacin, aminoglycosides (e.g., gentamycin), amoxicillin, amphotericin B, ampicillin, antimonials, atovaquone sodium stibogluconate, azithromycin, capreomycin, cefotaxime, cefoxitin, ceftriaxone, chloramphenicol, clarithromycin, clindamycin, clofazimine, cycloserine, dapsone, doxycycline, ethambutol, ethionamide, fluconazole, fluoroquinolones, isoniazid, itraconazole, kanamycin, ketoconazole, minocycline, ofloxacin), para-aminosalicylic acid, pentamidine, polymixin definsins, prothionamide, pyrazinamide, pyrimethamine sulfadiazine, quinolones (e.g., ciprofloxacin), rifabutin, rifampin, sparfloxacin, streptomycin, sulfonamides, tetracyclines, thiacetazone, trimethaprim-sulfamethoxazole, viomycin or combinations thereof. More generally, such an agent would be provided in a combined amount with the expression vector effective to kill or inhibit proliferation of a cancer cell and/or microorganism. This process may involve contacting the cell(s) with an agent(s) and the pharmaceutical composition at the same time or within a period of time wherein separate administration of the pharmaceutical composition and an agent to a cell, tissue or organism produces a desired therapeutic benefit. This may be achieved by contacting the cell, tissue or organism with a single composition or pharmacological formulation that includes both the pharmaceutical composition and one or more agents, or by contacting the cell with two or more distinct compositions or formulations, wherein one composition includes the pharmaceutical composition and the other includes one or more agents.

The terms “contacted” and “exposed,” when applied to a cell, tissue or organism, are used herein to describe the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing. The administration of the pharmaceutical composition may precede, be concurrent with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered within of from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein and one or more agents may be employed.

In some embodiments, the chemotherapeutic agent may be Taxotere (docetaxel), or another taxane, such as, for example, cabazitaxel. The chemotherapeutic may be administered either before, during, or after treatment with the cells and inducer. For example, the chemotherapeutic may be administered about 1 year, 11, 10, 9, 8, 7, 6, 5, or 4 months, or 18, 17, 16, 15, 14, 13, 12, 11, 10, 9, 8, 7, 6, 5, 4, 3, 2, weeks or 1 week prior to administering the first dose of activated nucleic acid. Or, for example, the chemotherapeutic may be administered about 1 week or 2, 3, 4, 5, 6, 7, 8, 9, 10, 11, 12, 13, 14, 15, 16, 17, or 18 weeks or 4, 5, 6, 7, 8, 9, 10, or 11 months or 1 year after administering the first dose of cells or inducer.

Administration of a chemotherapeutic agent may comprise the administration of more than one chemotherapeutic agent. For example, cisplatin may be administered in addition to Taxotere or other taxane, such as, for example, cabazitaxel.

Cytokine Measurement for Optimized and Personalized Treatment

Cytokines are a large and diverse family of polypeptide regulators produced widely throughout the body by cells of diverse origin. Cytokines are small secreted proteins, including peptides and glycoproteins, which mediate and regulate immunity, inflammation, and hematopoiesis. They are produced de novo in response to an immune stimulus. Cytokines generally (although not always) act over short distances and short time spans and at low concentration. They generally act by binding to specific membrane receptors, which then signal the cell via signaling proteins, often tyrosine kinases of the Janus family or coupled G proteins to alter cell behavior (e.g., gene expression). Responses to cytokines include, for example, increasing or decreasing expression of membrane proteins (including cytokine receptors), proliferation, blockage or promotion of apoptosis, differentiation and secretion of effector molecules.

The term “cytokine” is a general description of a large family of proteins and glycoproteins. Other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on cells that secrete them (autocrine action), on nearby cells (paracrine action), or in some instances on distant cells (endocrine action).

Examples of cytokines include, without limitation, interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18 and the like), interferons (e.g., IFN-beta, IFN-gamma and the like), tumor necrosis factors (e.g., TNF-alpha, TNF-beta and the like), lymphokines, monokines and chemokines; growth factors (e.g., transforming growth factors (e.g., TGF-alpha, TGF-beta and the like)); colony-stimulating factors (e.g. GM-CSF, granulocyte colony-stimulating factor (G-CSF) etc.); IP-10, MCP-1, and the like.

A cytokine often acts via a cell-surface receptor counterpart. Subsequent cascades of intracellular signaling then alter cell functions. This signaling may include upregulation and/or downregulation of several genes and their transcription factors, resulting in the production of other cytokines, an increase in the number of surface receptors for other molecules, or the suppression of their own effect by feedback inhibition.

Treatment using immune system activating cells discussed herein may be optimized by determining the concentration of certain cytokine biomarkers, such as, for example, cytokines discussed herein, including, for example, IL-2, IP-10, IL-5, and MCP-1, and, for example, IL-6, IL6-sR, or VCAM-1 during the course of treatment. IL-6 refers to interleukin 6. IL-6sR refers to the IL-6 soluble receptor, the levels of which often correlate closely with levels of IL-6. VCAM-1 refers to vascular cell adhesion molecule. Different patients having different stages or types of cancer, may react differently to various therapies. The response to treatment may be monitored by following the cytokine concentrations or levels in various body fluids or tissues. The determination of the concentration, level, or amount of a cytokine polypeptide may include detection of the full length polypeptide, or a fragment or variant thereof. The fragment or variant may be sufficient to be detected by, for example, immunological methods, mass spectrometry, nucleic acid hybridization, and the like. Optimizing treatment for individual patients may help to avoid side effects as a result of overdosing, may help to determine when the treatment is ineffective and to change the course of treatment, or may help to determine when doses may be increased. Technology discussed herein optimizes therapeutic methods for treating solid tumor cancers by allowing a clinician to track a biomarker, such as, for example, IL-6, IL-6sR, or VCAM-1, and determine whether a subsequent dose of a drug or vaccine for administration to a subject may be maintained, reduced or increased, and to determine the timing for the subsequent dose. Technology discussed herein optimizes therapeutic methods for treating solid tumor cancers by allowing a clinician to assay the level of or track a biomarker, such as, for example, IL-2, IP-10, IL-5, or MCP-1, and determine whether a subsequent dose of a drug or vaccine for administration to a subject may be maintained, reduced or increased, to determine the timing for the subsequent dose, or to determine which inducible or constitutive chimeric signaling polypeptide should be selected for the therapeutic cells.

Treatment for solid tumor cancers, including, for example, prostate cancer, may also be optimized by determining the concentration of urokinase-type plasminogen activator receptor (uPAR), hepatocyte growth factor (HGF), epidermal growth factor (EGF), or vascular endothelial growth factor (VEGF) during the course of treatment. Different patients having different stages or types of cancer, may react differently to various therapies. The levels of uPAR, HGF, EGF, and VEGF over the course of treatment for subject 1003 were measured. Subject 1003 shows systemic perturbation of hypoxic factors in serum, which may indicate a positive response to treatment.

Without limiting the interpretation of this observation, this may indicate the secretion of hypoxic factors by tumors in response to treatment. Thus, the response to treatment may be monitored, for example, by following the uPAR, HGF, EGF, or VEGF concentrations or levels in various body fluids or tissues. The determination of the concentration, level, or amount of a polypeptide, such as, uPAR, HGF, EGF, or VEGF may include detection of the full length polypeptide, or a fragment or variant thereof. The fragment or variant may be sufficient to be detected by, for example, immunological methods, mass spectrometry, nucleic acid hybridization, and the like. Optimizing treatment for individual patients may help to avoid side effects as a result of overdosing, may help to determine when the treatment is ineffective and to change the course of treatment, or may help to determine when doses may be increased. Technology discussed herein optimizes therapeutic methods for treating solid tumor cancers by allowing a clinician to track a biomarker, such as, for example, uPAR, HGF, EGF, or VEGF, and determine whether a subsequent dose of a drug or vaccine for administration to a subject may be maintained, reduced or increased, and to determine the timing for the subsequent dose.

For example, it has been determined that amount or concentration of certain biomarkers changes during the course of treatment of solid tumors. Predetermined target levels of such biomarkers, or biomarker thresholds may be identified in normal subject, are provided, which allow a clinician to determine whether a subsequent dose of a drug administered to a subject in need thereof, such as a subject with a solid tumor, such as, for example, a prostate tumor, may be increased, decreased or maintained. A clinician can make such a determination based on whether the presence, absence or amount of a biomarker is below, above or about the same as a biomarker threshold, respectively, in certain embodiments.

For example, determining that an over-represented biomarker level is significantly reduced and/or that an under-represented biomarker level is significantly increased after drug treatment or vaccination provides an indication to a clinician that an administered drug is exerting a therapeutic effect. By “level” is meant the concentration of the biomarker in a fluid or tissue, or the absolute amount in a tissue. Based on such a biomarker determination, a clinician could make a decision to maintain a subsequent dose of the drug or raise or lower the subsequent dose, including modifying the timing of administration. The term “drug” includes traditional pharmaceuticals, such as small molecules, as well as biologics, such as nucleic acids, antibodies, proteins, polypeptides, modified cells and the like. In another example, determining that an over-represented biomarker level is not significantly reduced and/or that an under-represented biomarker level is not significantly increased provides an indication to a clinician that an administered drug is not significantly exerting a therapeutic effect. Based on such a biomarker determination, a clinician could make a decision to increase a subsequent dose of the drug. Given that drugs can be toxic to a subject and exert side effects, methods provided herein optimize therapeutic approaches as they provide the clinician with the ability to “dial in” an efficacious dosage of a drug and minimize side effects. In specific examples, methods provided herein allow a clinician to “dial up” the dose of a drug to a therapeutically efficacious level, where the dialed up dosage is below a toxic threshold level. Accordingly, treatment methods discussed herein enhance efficacy and reduce the likelihood of toxic side effects.

Cytokines may be detected as full-length (e.g., whole) proteins, polypeptides, metabolites, messenger RNA (mRNA), complementary DNA (cDNA), and various intermediate products and fragments of the foregoing (e.g., cleavage products (e.g., peptides, mRNA fragments)). For example, IL-6 protein may be detected as the complete, full-length molecule or as any fragment large enough to provide varying levels of positive identification. Such a fragment may comprise amino acids numbering less than 10, from 10 to 20, from 20 to 50, from 50 to 100, from 100 to 150, from 150 to 200 and above. Likewise, VCAM-1 protein can be detected as the complete, full-length amino acid molecule or as any fragment large enough to provide varying levels of positive identification. Such a fragment may comprise amino acids numbering less than 10, from 10 to 20, from 20 to 50, from 50 to 100, from 100 to 150 and above.

In certain embodiments, cytokine mRNA may be detected by targeting a complete sequence or any sufficient fragment for specific detection. An mRNA fragment may include fewer than 10 nucleotides or any larger number. A fragment may comprise the 3′ end of the mRNA strand with any portion of the strand, the 5′ end with any portion of the strand, and any center portion of the strand.

Detection may be performed using any suitable method, including, without limitation, mass spectrometry (e.g., matrix-assisted laser desorption ionization mass spectrometry (MALDI-MS), electrospray mass spectrometry (ES-MS)), electrophoresis (e.g., capillary electrophoresis), high performance liquid chromatography (HPLC), nucleic acid affinity (e.g., hybridization), amplification and detection (e.g., real-time or reverse-transcriptase polymerase chain reaction (RT-PCR)), and antibody assays (e.g., antibody array, enzyme-linked immunosorbant assay (ELISA)). Examples of IL-6 and other cytokine assays include, for example, those provided by Millipore, Inc., (Milliplex Human Cytokine/Chemokine Panel). Examples of IL6-sR assays include, for example, those provided by Invitrogen, Inc. (Soluble IL-6R: (Invitrogen Luminex® Bead-based assay)). Examples of VCAM-1 assays include, for example, those provided by R & D Systems ((CD106) ELISA development Kit, DuoSet from R&D Systems (#DY809)).

Sources of Biomarkers

The presence, absence or amount of a biomarker can be determined within a subject (e.g., in situ) or outside a subject (e.g., ex vivo). In some embodiments, presence, absence or amount of a biomarker can be determined in cells (e.g., differentiated cells, stem cells), and in certain embodiments, presence, absence or amount of a biomarker can be determined in a substantially cell-free medium (e.g., in vitro). The term “identifying the presence, absence or amount of a biomarker in a subject” as used herein refers to any method known in the art for assessing the biomarker and inferring the presence, absence or amount in the subject (e.g., in situ, ex vivo or in vitro methods).

A fluid or tissue sample often is obtained from a subject for determining presence, absence or amount of biomarker ex vivo. Non-limiting parts of the body from which a tissue sample may be obtained include leg, arm, abdomen, upper back, lower back, chest, hand, finger, fingernail, foot, toe, toenail, neck, rectum, nose, throat, mouth, scalp, face, spine, throat, heart, lung, breast, kidney, liver, intestine, colon, pancreas, bladder, cervix, testes, muscle, skin, hair, tumor or area surrounding a tumor, and the like, in some embodiments. A tissue sample can be obtained by any suitable method known in the art, including, without limitation, biopsy (e.g., shave, punch, incisional, excisional, curettage, fine needle aspirate, scoop, scallop, core needle, vacuum assisted, open surgical biopsies) and the like, in certain embodiments. Examples of a fluid that can be obtained from a subject includes, without limitation, blood, cerebrospinal fluid, spinal fluid, lavage fluid (e.g., bronchoalveolar, gastric, peritoneal, ductal, ear, arthroscopic), urine, interstitial fluid, feces, sputum, saliva, nasal mucous, prostate fluid, lavage, semen, lymphatic fluid, bile, tears, sweat, breast milk, breast fluid, fluid from region of inflammation, fluid from region of muscle wasting and the like, in some embodiments.

A sample from a subject may be processed prior to determining presence, absence or amount of a biomarker. For example, a blood sample from a subject may be processed to yield a certain fraction, including without limitation, plasma, serum, buffy coat, red blood cell layer and the like, and biomarker presence, absence or amount can be determined in the fraction. In certain embodiments, a tissue sample (e.g., tumor biopsy sample) can be processed by slicing the tissue sample and observing the sample under a microscope before and/or after the sliced sample is contacted with an agent that visualizes a biomarker (e.g., antibody). In some embodiments, a tissue sample can be exposed to one or more of the following non-limiting conditions: washing, exposure to high salt or low salt solution (e.g., hypertonic, hypotonic, isotonic solution), exposure to shearing conditions (e.g., sonication, press (e.g., French press)), mincing, centrifugation, separation of cells, separation of tissue and the like. In certain embodiments, a biomarker can be separated from tissue and the presence, absence or amount determined in vitro. A sample also may be stored for a period of time prior to determining the presence, absence or amount of a biomarker (e.g., a sample may be frozen, cryopreserved, maintained in a preservation medium (e.g., formaldehyde)).

A sample can be obtained from a subject at any suitable time of collection after a drug is delivered to the subject. For example, a sample may be collected within about one hour after a drug is delivered to a subject (e.g., within about 5, 10, 15, 20, 25, 30, 35, 40, 45, 55 or 60 minutes of delivering a drug), within about one day after a drug is delivered to a subject (e.g., within about 2, 3, 4, 5, 6, 7, 8, 9, 10, 11, 12, 13, 14, 15, 16, 17, 18, 19, 20, 21, 22, 23 or 24 hours of delivering a drug) or within about two weeks after a drug is delivered to a subject (e.g., within about 2, 3, 4, 5, 6, 7, 8, 9, 10, 11, 12, 13 or 14 days of delivering the drug). A collection may be made on a specified schedule including hourly, daily, semi-weekly, weekly, bi-weekly, monthly, bi-monthly, quarterly, and yearly, and the like, for example. If a drug is administered continuously over a time period (e.g., infusion), the delay may be determined from the first moment of drug is introduced to the subject, from the time the drug administration ceases, or a point in-between (e.g., administration time frame midpoint or other point).

Biomarker Detection

The presence, absence or amount of one or more biomarkers may be determined by any suitable method known in the art, and non-limiting determination methods are discussed herein. Determining the presence, absence or amount of a biomarker sometimes comprises use of a biological assay. In a biological assay, one or more signals detected in the assay can be converted to the presence, absence or amount of a biomarker. Converting a signal detected in the assay can comprise, for example, use of a standard curve, one or more standards (e.g., internal, external), a chart, a computer program that converts a signal to a presence, absence or amount of biomarker, and the like, and combinations of the foregoing.

Biomarker detected in an assay can be full-length biomarker, a biomarker fragment, an altered or modified biomarker (e.g., biomarker derivative, biomarker metabolite), or sum of two or more of the foregoing, for example. Modified biomarkers often have substantial sequence identity to a biomarker discussed herein. For example, percent identity between a modified biomarker and a biomarker discussed herein may be in the range of 15-20%, 20-30%, 31-40%, 41-50%, 51-60%, 61-70%, 71-80%, 81-90% and 91-100%, (e.g. 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 percent identity). A modified biomarker often has a sequence (e.g., amino acid sequence or nucleotide sequence) that is 90% or more identical to a sequence of a biomarker discussed herein. Percent sequence identity can be determined using alignment methods known in the art.

Detection of biomarkers may be performed using any suitable method known in the art, including, without limitation, mass spectrometry, antibody assay (e.g., ELISA), nucleic acid affinity, microarray hybridization, Northern blot, reverse PCR and RT-PCR. For example, RNA purity and concentration may be determined spectrophotometrically (260/280>1.9) on a Nanodrop 1000. RNA quality may be assessed using methods known in the art (e.g., Agilent 2100 Bioanalyzer; RNA 6000 Nano LabChip® and the like).

Indication for Adjusting or Maintaining Subsequent Drug Dose

An indication for adjusting or maintaining a subsequent drug dose can be based on the presence or absence of a biomarker. For example, when (i) low sensitivity determinations of biomarker levels are available, (ii) biomarker levels shift sharply in response to a drug, (iii) low levels or high levels of biomarker are present, and/or (iv) a drug is not appreciably toxic at levels of administration, presence or absence of a biomarker can be sufficient for generating an indication of adjusting or maintaining a subsequent drug dose.

An indication for adjusting or maintaining a subsequent drug dose often is based on the amount or level of a biomarker. An amount of a biomarker can be a mean, median, nominal, range, interval, maximum, minimum, or relative amount, in some embodiments. An amount of a biomarker can be expressed with or without a measurement error window in certain embodiments. An amount of a biomarker in some embodiments can be expressed as a biomarker concentration, biomarker weight per unit weight, biomarker weight per unit volume, biomarker moles, biomarker moles per unit volume, biomarker moles per unit weight, biomarker weight per unit cells, biomarker volume per unit cells, biomarker moles per unit cells and the like. Weight can be expressed as femtograms, picograms, nanograms, micrograms, milligrams and grams, for example. Volume can be expressed as femtoliters, picoliters, nanoliters, microliters, milliliters and liters, for example. Moles can be expressed in picomoles, nanomoles, micromoles, millimoles and moles, for example. In some embodiments, unit weight can be weight of subject or weight of sample from subject, unit volume can be volume of sample from the subject (e.g., blood sample volume) and unit cells can be per one cell or per a certain number of cells (e.g., micrograms of biomarker per 1000 cells). In some embodiments, an amount of biomarker determined from one tissue or fluid can be correlated to an amount of biomarker in another fluid or tissue, as known in the art.

An indication for adjusting or maintaining a subsequent drug dose often is generated by comparing a determined level of biomarker in a subject to a predetermined level of biomarker. A predetermined level of biomarker sometimes is linked to a therapeutic or efficacious amount of drug in a subject, sometimes is linked to a toxic level of a drug, sometimes is linked to presence of a condition, sometimes is linked to a treatment midpoint and sometimes is linked to a treatment endpoint, in certain embodiments. A predetermined level of a biomarker sometimes includes time as an element, and in some embodiments, a threshold is a time-dependent signature. For example, an IL-6 or IL6-sR level of about 8-fold more than a normal level, or greater (e.g. about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75-fold more than a normal level) may indicate that the dosage of the drug or the frequency of administration may be increased in a subsequent administration.

The term “dosage” is meant to include both the amount of the dose and the frequency of administration, such as, for example, the timing of the next dose. An IL-6 or IL-6sR level less than about 8-fold more than a normal level (e.g. about 7, 6, 5, 4, 3, 2, or 1-fold more than a normal level, or less than or equal to a normal level) may indicate that the dosage may be maintained or decreased in a subsequent administration. A VCAM-1 level of about 8 fold more than a normal level, or greater (e.g. e.g. about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75-fold more than a normal level) may indicate that the dosage of the drug may be increased in a subsequent administration. A VCAM-1 level less than about 8-fold more than a normal level (e.g. about 7, 6, 5, 4, 3, 2, or 1-fold more than a normal level, or less than or equal to a normal level) may indicate that the dosage may be maintained or decreased in a subsequent administration. A normal level of IL-6, IL-6sR, or VCAM-1 may be assessed in a subject not diagnosed with a solid tumor or the type of solid tumor under treatment in a patient.

Other indications for adjusting or maintaining a drug dose include, for example, a perturbation in the concentration of an individual secreted factor, such as, for example, GM-CSF, MIP-1alpha, MIP-1beta, MCP-1, IFN-gamma, RANTES, EGF or HGF, or a perturbation in the mean concentration of a panel of secreted factors, such as two or more of the markers selected from the group consisting of GM-CSF, MIP-1alpha, MIP-1beta, MCP-1, IFN-gamma, RANTES, EGF and HGF. This perturbation may, for example, consist of an increase, or decrease, in the concentration of an individual secreted factor by at least 5%, 10%, 20%, 30%, 40%, 50%, 60%, 70%, 80%, 90%, or 100% or an increase or decrease in the mean relative change in serum concentration of a panel of secreted factors by at least 5%, 10%, 20%, 30%, 40%, 50%, 60%, 70%, 80%, 90%, or 100%. This increase may, or may not, be followed by a return to baseline serum concentrations before the next administration. The increase or decrease in the mean relative change in serum concentration may involve, for example, weighting the relative value of each of the factors in the panel. Also, the increase or decrease may involve, for example, weighting the relative value of each of the time points of collected data. The weighted value for each time point, or each factor may vary, depending on the state or the extent of the cancer, metastasis, or tumor burden. An indication for adjusting or maintaining the drug dose may include a perturbation in the concentration of an individual secreted factor or the mean concentration of a panel of secreted factors, after 1, 2, 3, 4, 5, 6, 7, 8, 9, or 10 or more administrations. For example, where it is observed that over the course of treatment, for example, 6 administrations of a drug or the vaccines or compositions discussed herein, that the concentration of an individual secreted factor or the mean concentration of a panel of secreted factors is perturbed after at least one administration, then this may be an indication to maintain, decrease, or increase the frequency of administration or the subsequent dosage, or it may be an indication to continue treatment by, for example, preparing additional drug, adenovirus vaccine, or adenovirus transfected or transduced cells.

Some treatment methods comprise (i) administering a drug to a subject in one or more administrations (e.g., 1, 2, 3, 4, 5, 6, 7, 8, 9 or 10 doses), (ii) determining the presence, absence or amount of a biomarker in or from the subject after (i), (iii) providing an indication of increasing, decreasing or maintaining a subsequent dose of the drug for administration to the subject, and (iv) optionally administering the subsequent dose to the subject, where the subsequent dose is increased, decreased or maintained relative to the earlier dose(s) in (i). In some embodiments, presence, absence or amount of a biomarker is determined after each dose of drug has been administered to the subject, and sometimes presence, absence or amount of a biomarker is not determined after each dose of the drug has been administered (e.g., a biomarker is assessed after one or more of the first, second, third, fourth, fifth, sixth, seventh, eighth, ninth or tenth dose, but not assessed every time after each dose is administered).

An indication for adjusting a subsequent drug dose can be considered a need to increase or a need to decrease a subsequent drug dose. An indication for adjusting or maintaining a subsequent drug dose can be considered by a clinician, and the clinician may act on the indication in certain embodiments. In some embodiments, a clinician may opt not to act on an indication. Thus, a clinician can opt to adjust or not adjust a subsequent drug dose based on the indication provided.

An indication of adjusting or maintaining a subsequent drug dose, and/or the subsequent drug dosage, can be provided in any convenient manner. An indication may be provided in tabular form (e.g., in a physical or electronic medium) in some embodiments. For example, a biomarker threshold may be provided in a table, and a clinician may compare the presence, absence or amount of the biomarker determined for a subject to the threshold. The clinician then can identify from the table an indication for subsequent drug dose. In certain embodiments, an indication can be presented (e.g., displayed) by a computer after the presence, absence or amount of a biomarker is provided to computer (e.g., entered into memory on the computer). For example, presence, absence or amount of a biomarker determined for a subject can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount. A subsequent dose can be determined based on certain factors other than biomarker presence, absence or amount, such as weight of the subject, one or more metabolite levels for the subject (e.g., metabolite levels pertaining to liver function) and the like, for example.

In other embodiments, following administration of the modified cells or nucleic acid, wherein the modified cells or nucleic acid express the inducible Caspase-9 polypeptide, in the event of a need to reduce the number of modified cells or in vivo modified cells, the multimeric ligand may be administered to the patient. In these embodiments, the methods comprise determining the presence or absence of a negative symptom or condition, such as Graft vs Host Disease, or off target toxicity, and administering a dose of the multimeric ligand. The methods may further comprise monitoring the symptom or condition and administering an additional dose of the multimeric ligand in the event the symptom or condition persists. This monitoring and treatment schedule may continue while the therapeutic cells that express the modified chimeric antigen receptor polypeptides or chimeric signaling polypeptides remain in the patient.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

A subject can be prescreened to determine whether or not the presence, absence or amount of a particular biomarker may be determined. Non-limiting examples of prescreens include identifying the presence or absence of a genetic marker (e.g., polymorphism, particular nucleotide sequence); identifying the presence, absence or amount of a particular metabolite. A prescreen result can be used by a clinician in combination with the presence, absence or amount of a biomarker to determine whether a subsequent drug dose may be adjusted or maintained.

Antibodies and Small Molecules

In some embodiments, an antibody or small molecule is provided for use as a control or standard in an assay, or a therapeutic, for example. In some embodiments, an antibody or other small molecule configured to bind to a cytokine or cytokine receptor, including without limitation IL-6, IL-6sR, and alter the action of the cytokine, or it may be configured to bind to VCAM-1. In certain embodiments an antibody or other small molecule may bind to an mRNA structure encoding for a cytokine or receptor.

The term small molecule as used herein means an organic molecule of approximately 800 or fewer Daltons. In certain embodiments small molecules may diffuse across cell membranes to reach intercellular sites of action. In some embodiments a small molecule binds with high affinity to a biopolymer such as protein, nucleic acid, or polysaccharide and may sometimes alter the activity or function of the biopolymer. In various embodiments small molecules may be natural (such as secondary metabolites) or artificial (such as antiviral drugs); they may have a beneficial effect against a disease (such as drugs) or may be detrimental (such as teratogens and carcinogens). By way of non-limiting example, small molecules may include ribo- or deoxyribonucleotides, amino acids, monosaccharides and small oligomers such as dinucleotides, peptides such as the antioxidant glutathione, and disaccharides such as sucrose.

The term antibody as used herein is to be understood as meaning a gamma globulin protein found in blood or other bodily fluids of vertebrates, and used by the immune system to identify and neutralize foreign objects, such as bacteria and viruses. Antibodies typically include basic structural units of two large heavy chains and two small light chains.

Specific binding to an antibody requires an antibody that is selected for its affinity for a particular protein. For example, polyclonal antibodies raised to a particular protein, polymorphic variants, alleles, orthologs, and conservatively modified variants, or splice variants, or portions thereof, can be selected to obtain only those polyclonal antibodies that are specifically immunoreactive with GM-CSF, TNF-alpha or NF-kappa-B modulating protein and not with other proteins. This selection may be achieved by subtracting out antibodies that cross-react with other molecules.

Methods as presented herein include without limitation the delivery of an effective amount of an activated cell, a nucleic acid. or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers to evaluate the effectiveness of treatment and to control toxicity.

### Examples

Examples herein that discuss the methods for transforming or transfecting cells in vitro, or ex vivo, provide examples of, but do not limit, the use of nucleic acids that express chimeric polypeptides. Examples of the delivery of the transduced or transfected cells, and ligand inducer, to laboratory animals or human subjects provide examples of, but do not limit, the the direct administration of nucleic acids expressing chimeric polypeptides, tumor antigens, and ligand inducer to subjects in need thereof.

In addition, the following sections, in particular, examples 21 et seq., provide examples of methods of expressing an inducible chimeric signaling polypeptide in therapeutic cells, for example, T cells, and methods of using the transformed cells. Methods of expressing inducible polypeptides, use of the transduced or transfected cells, and assays are discussed, for example, in Spencer, D. M., et al., Science 262: 1019-1024 (1993); U.S. Pat. No. 7,404,950, entitled “Induced Activation in Dendritic Cells,” issued Jul. 29, 2008; U.S. patent application Ser. No. 13/087,329, entitled “Methods for Treating Solid Tumors,” filed Apr. 14, 2011; and U.S. patent application Ser. No. 13/112,739, entitled “Methods for Inducing Selective Apoptosis, filed May 20, 2011, which are hereby incorporated by reference herein in their entirety.

The examples set forth below illustrate certain embodiments and do not limit the technology.

### Example 1: Fusion of FKBP12 with MyD88-X Signaling Domains Confers Drug-Inducible Signal Transduction

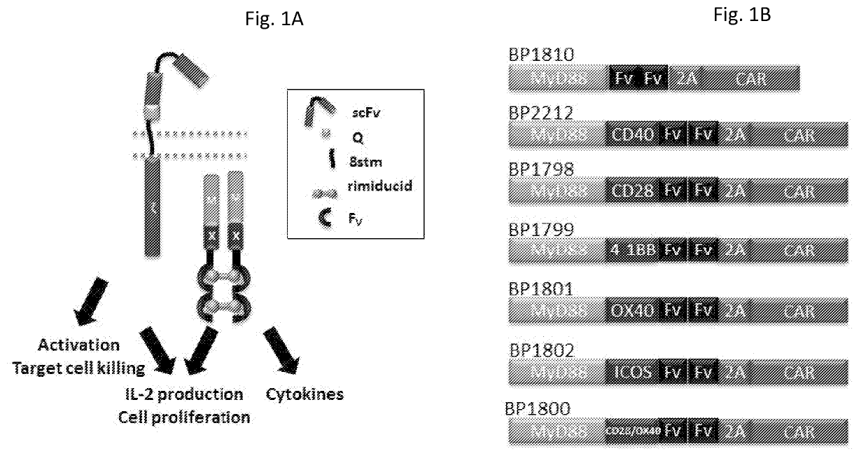

The present example discusses the design and assay of cells that express inducible chimeric signaling polypeptides that comprise MyD88. For each of the retroviral vectors shown in FIG. 1, the CAR cistron was coexpressed with the inducible MyD88 fusion cistron from the same mRNA, and was cleaved into distinct protein products through the action of a T2A peptide sequence derived from the Thoseaasigna virus. The inducible chimeric signaling polypeptides either included, or did not include, membrane targeting regions, leader polypeptides that promote N-terminal modifications to localize the chimeric polypeptide to the plasma membrane. Examples of membrane targeting regions are discussed herein, and in the present example, the leader polypeptide is myristoylation targeting sequence, a signal sequence directing the myristoylation of glycine at the N-terminus (Myr).

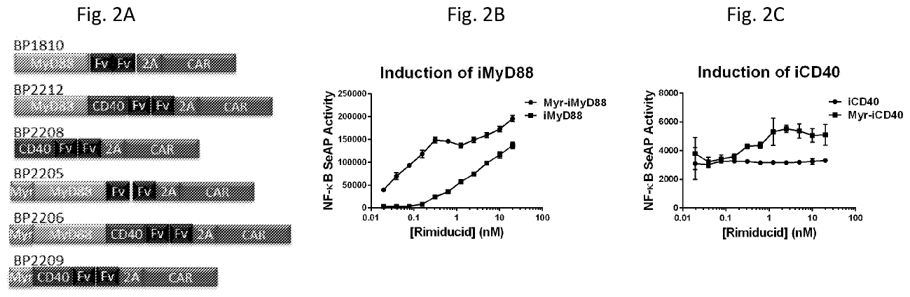

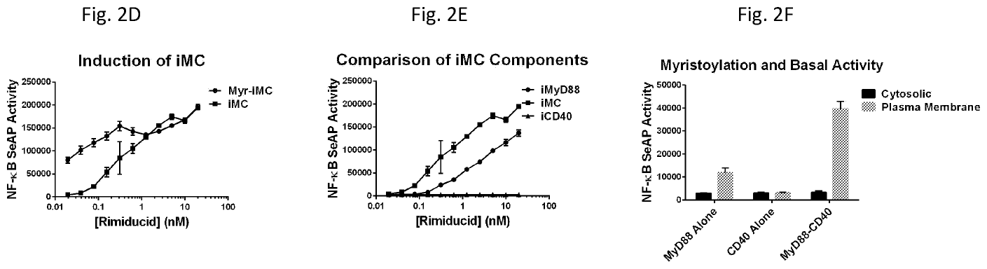

FIG. 2 provides data obtained from assays of signaling activity by inducible MyD88, inducible CD40, and inducible MyD88/CD40 chimeric polypeptides. Synergistic activation of the signaling pathway to the NF-κB transcription factor was observed in HEK-293 cells expressing the chimeric inducible MyD88/CD40 polypeptide (iMC). Costimulatory signals from iMC were more robust when the signaling domains were localized at or near the plasma membrane (myr-iMC) than those produced by cytosolic iMC in the absence of the multimeric ligand rimiducid. In the absence of rimiducid, significant basal NF-κB activity was observed in cells that expressed the myristoylated inducible MyD88 chimeric polypeptide (myr-iMyD88). The basal activity of the non-myristoylated inducible MyD88 (iMD88) in the absence of rimiducid was low, permitting a larger window of inducibility relative to that from Myr-iM. The low basal activity of the non-myristoylated CD40 (iCD40) was similar with or without the myristoylation region. The myr-iCD40 had detectible but weak signaling in the presence of increasing amounts of rimiducid but the non-myristoylated iCD40 was not inducible. When CD40 was fused with MyD88, such as in iMC, a robust synergistic signaling was observed. In the presence of increasing rimiducid concentration, Myr-iMC and cytosolic iMC homodimerized through their FKBP12 components and strong signaling was generated. Myr-iMC, however, had stronger basal activity than iMC.

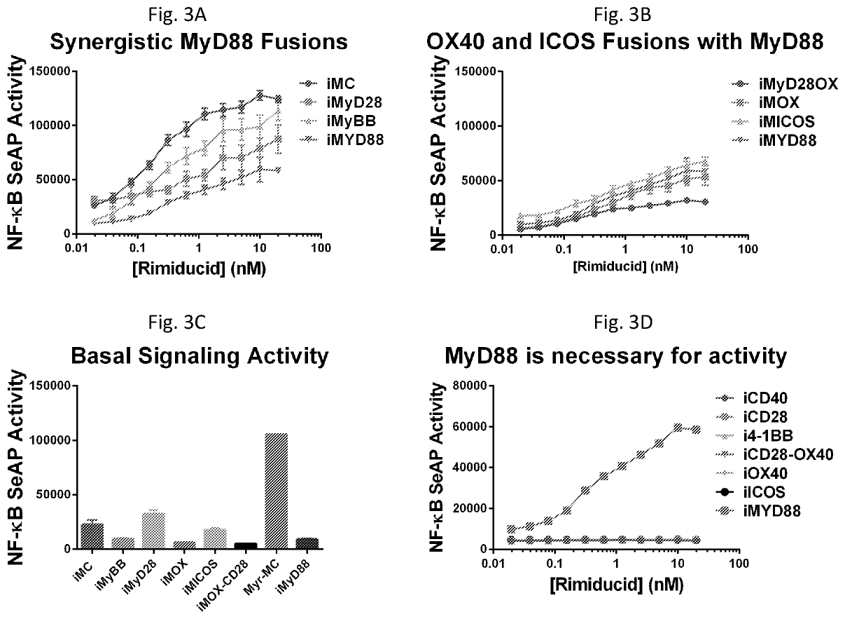

FIG. 3 provides data comparing the activity of various chimeric inducible MyD88 polypeptides, where the CD40 signaling moiety is replaced with that of alternative costimulatory receptors. The chimeric inducible MyD88 polypeptides in these assays did not include myristoylation regions. Substitution of CD40 with the alternative costimulatory receptor polypeptides altered both the degree of NF-κB induction with rimiducid, and the basal activity observed without induction. iMC provided the strongest maximal signaling activity and was readily induced with subnanomolar concentrations of rimiducid. MyD28 (MyD88-CD28) had a high basal activity without rimiducid but signaling was relatively poorly induced by drug and maximal activity was lower. Similarly, fusion with OX40 (iMOX) did not give a synergistic augmentation of MyD88 inducible activity and fusion with ICOS (to give iMICOS) had very poor inducibility. Interestingly, the fusion of MyD88 with 4-1BB to generate iMyBB gave robust inducible signaling activity comparable, but slightly lower than iMC, and basal activity was lower with iMyBB than iMC or iMyD28. Therefore, iMyBB provides a greater window of inducibility in culture than iMC. Together, these findings indicate that the strength of inducible signaling activity can be controlled both by the level of dimerizing ligand and the identity of the signaling component fused with MyD88. Little to no signaling activity was observed if the alternative costimulatory domains were fused directly with tandem FKBP12s but without MyD88.

Methods

HEK 293 cells (1.5×10⁵) were seeded on a 100-mm tissue culture dish in 10 mL DMEM4500, supplemented with glutamine, penicillin/streptomycin and 10% fetal calf serum. After 16-30 hours incubation, cells were transfected using Novagen's GeneJuice® protocol. Briefly, for each transfection, 0.5 mL OptiMEM was pipeted into a 1.5-mL microcentrifuge tube and 15 μL GeneJuice reagent added followed by 5 sec. vortexing. Samples were rested 5 minutes to settle the GeneJuice suspension. DNA (5 μg total) was added to each tube and mixed by pipetting up and down four times. Samples were allowed to rest for 5 minutes for GeneJuice-DNA complex formation and the suspension added dropwise to one dish of 293T cells. A typical transfection contains 1 μg NFkB-SEAP, 4 μg CAR-encoding plasmid (pBP1798, pBP2212 or similar) or 1 μg NFkB-SEAP, 1 μg CAR-encoding plasmid and 3 μg of an irrelevant carrier plasmid pBP1099.

24 hours following transfection, 293 cells were split to 96-well plates and incubated with dilutions of rimiducid. Briefly, 100 μL media was added to each well of a 96-well flat-bottom plate. Rimiducid was diluted in tubes to a concentration 4× the top concentration in the gradient to be place on the plate. 100 μL of dimerizing ligand was added to each of four wells on the far right of the plate (assays are thereby performed in quadruplicate). 100 μL from each drug-containing well was then transferred to the adjacent well and the cycle repeated 10 times to produce a serial two-fold step gradient. The last wells were untreated and serve as a control for basal reporter activity. Transfected 293 cells were then trypsinized, washed with complete media, suspended in media and 100 μL aliquoted to each well containing drug (or no drug). Cells were incubated 24 hours.

24 hours after drug stimulation, 96-well plates were wrapped to prevent evaporation and incubated at 65° C. for 2 hours to inactivate endogenous and serum phosphatases while the heat-stable SeAP reporter remained. 100 μL samples from each well were loaded into individual wells of a 96-well assay plate with black sides (Greiner). Samples were incubated with 0.5 mM 4-methylumbelliferyl phosphate (4-MUP) in 0.5 M diethanolamine at pH 10.0 for 4 to 16 hours. Phosphatase activity was measured by fluorescence with excitation at 355 nm and emission at 460 nm. Data was transferred to a Microsoft Excel spreadsheet for tabulation and graphed with GraphPad Prism.

### Example 2: Alteration of CAR-T Cell Signaling with Inducible MyD88-X Fusions

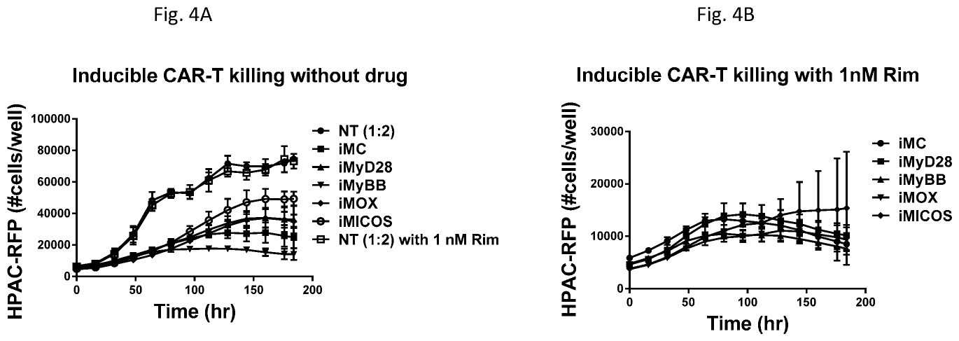

Recombinant retroviruses were produced from the constructs outlined in FIG. 1 and transduced into primary peripheral blood mononuclear cells (PBMCs) enriched in T cells. The cells also expressed chimeric antigen receptors, including a scFv specific for the tumor antigen, PSCA (Prostate Stem Cell Antigen). The ability of the transduced cells to target and kill the PSCA-expressing pancreatic tumor cell line, HPAC, was determined by microscopic observation and quantitation of the growth of the tumor line every three hours for seven days (FIG. 4). The capacity of CAR-T cells to kill a target is determined by its recognition of the tumor antigen and by the capacity of costimulatory signals to augment cytokine release and stimulate proliferation to an extent that overwhelms the growth of the tumor cells. At low effector (the CAR-T cells) to target ratios, the number of growing T cells becomes limiting and the relative capacity of costimulatory signals to drive T cell proliferation and survival can be determined indirectly by tumor cell killing. In the absence of rimiducid, the basal costimulatory activity of iM-X fusions, including iMyD88-4-1BB (iMyBB), iMyDD-CD28 (iMyD28), iMyD88-OX40 (iMOX), and iMyD88-ICOS (iMICOS), was higher than that of a first generation CAR alone but at an effector to target ratio of 1:30 several fusions were unable to control tumor cell growth. Inducible MyD88-ICOS (iMICOS) was least effective in this format, iMyBB (inducible MyD88-4-1BB) was most effective to control the tumor, and marginally more effective than iMC. In the presence of 1 nM rimiducid to drive dimerization and activation of the inducible chimeric signaling polypeptides, each fusion was capable of nearly complete control of the tumor cells, with only iMICOS permitting some escapees. These experiments indicate that the signals necessary to drive CAR-T proliferation and function can be modulated by alteration of the costimulatory domain fused with MyD88.

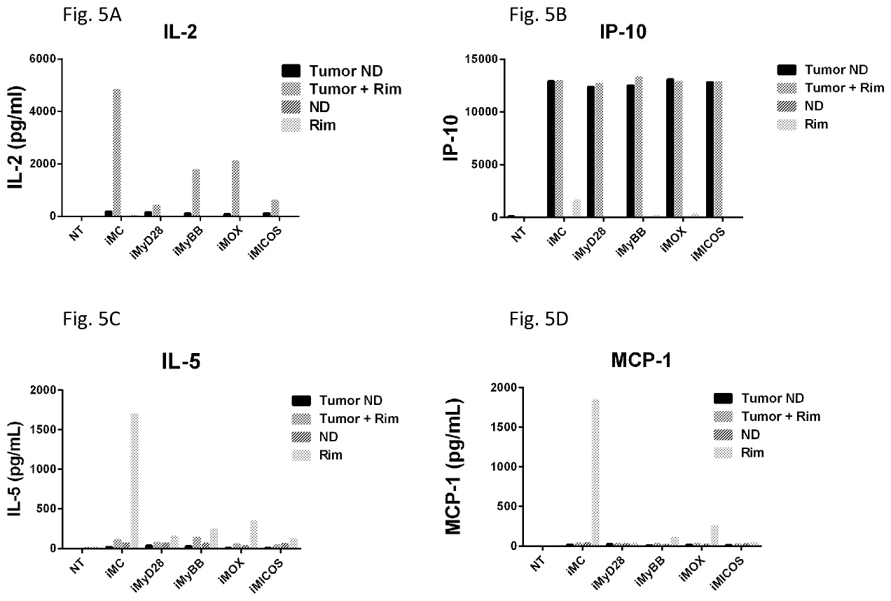

The presence of tumor cells activates CAR-expressing T cells, providing “Signal 1,” principally NF-AT activity, which collaborates with costimulatory signals derived from the inducible chimeric signaling polypeptides (“Signal 2’). FIG. 5 provides the results of cytokine secretion assays performed for each of the iM-X expressing CAR-T cell populations, in the presence and absence of target tumor cells. Additionally, cytokine secretion was determined with and without rimiducid to activate the inducible chimeric signaling polypeptide. In FIG. 5, “ND” indicates no drug, that is, no rimiducid, and “NT” indicates cells that are not transduced. Significant differences were observed in the production of cytokines using the different iM-X fusions (FIG. 5) indicating that these signaling pathways control distinct outcomes in the pattern of gene expression, while providing relatively small differences in NF-κB signaling.

When tumor was present to contribute signal 1, IP-10, an inducer of interferon gene expression, was strongly expressed in CAR-T cells that expressed the chimeric signaling polypeptide, in the presence or absence of rimiducid. In contrast, IP-10 was not detectably expressed in non-transduced cells. In the absence of the target tumor, only the CAR-T cells that expressed inducible MyD88-CD40 and that were stimulated with rimiducid produced detectable levels of IP-10.

IL-2 expression requires both signal 1 from the tumor and costimulation. iMC provided strong drug-dependent costimulation while iMICOS and iMyD28 only weakly supported IL-2 production. iMyBB and iMOX, each containing TNF-β related signaling domains fused with MyD88, supported substantial IL-2 production but less than iMC. A different pattern was observed for secretion of other cytokines such as the Th2 cytokine IL-5 and the homing factor MCP-1. Secretion of these factors was strongly induced by rimiducid only with iMC and not other costimulatory fusions with MyD88, even in the presence of target tumor cells.

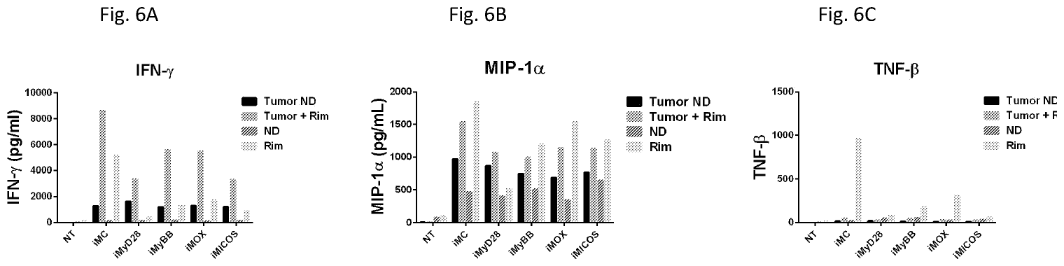

Secretion of some other cytokines was less dependent on the identity of the signaling domain fused with MyD88 (FIG. 6). The Th1 cytokine Interferon-Y⁻ (IFN-Y⁻) exhibited a pronounced drug dependent induction for secretion in cells that expressed the iMC, iMyBB, and iMOX polypeptides in the presence of target tumor cells. This rimiducid induction was not as strong with iMyD28 and iMICOS. In the absence of the target tumor, iMC strongly supported signaling to generate IFN-Y⁻ but the other fusions were relatively weak, even when induced with rimiducid. This pattern was also exhibited with the homing factor MIP1α. Tumor necrosis factor-β, another Th1 cytokine, did not display this signal one-dependent augmentation by induced MyD88 responses, but instead displayed a pattern of cytokine expression and secretion more akin to that produced by Th2 cells, such as IL-5, which are strongly induced only by iMC. This supported the notion that the differences in expression is more a consequence of intrinsic cell signaling by various MyD88 fusions rather than a bias toward differentiation between T helper subtypes.

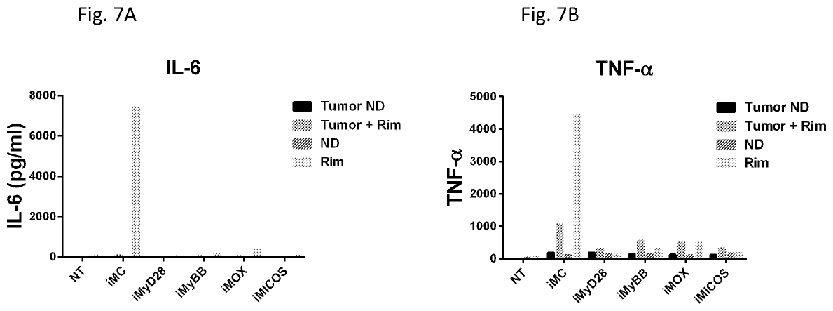

Therapies that utilize CAR-T cells to eradicate tumor burdens that have the discrete presence of tumor specific antigens are a promising development for the treatment of cancer. The efficacy of T-cell dependent tumor cell killing is frequently accompanied by the toxic effect of toxic inflammatory cytokine secretion by hyperactivated and highly proliferative anti-tumor CAR-T cells. iMC transduced CAR-T cells displayed robust secretion of the inflammatory cytokines IL-6 and TNF-α principally in the absence of tumor, but in the case of TNF-α, secretion was notable with tumor present. The significant secretion of IL-6 and TNF-α in the absence of tumor was greatly reduced if the CAR-T cells were transduced to express an inducible chimeric signaling polypeptide, rather than iMC, notably MyBB or MOX (FIG. 7). This finding raises the possibility that inducible chimeric signaling polypeptides may be useful antitumor agents that have severely reduced side systemic toxicity to the patient.

Methods

Production of Retroviruses and transduction of Peripheral Blood Mononuclear Cells (PBMCs): HEK 293T cells (1.5×10⁵) were seeded on a 100-mm tissue culture dish in 10 mL DMEM4500, supplemented with glutamine, penicillin/streptomycin and 10% fetal calf serum. After 16-30 hours incubation, cells were transfected using Novagen's GeneJuice® protocol. Briefly, for each transfection, 0.5 mL OptiMEM (LifeTechnologies) was pipeted into a 1.5-mL microcentrifuge tube and 30 μL GeneJuice reagent added followed by 5 sec. vortexing. Samples were rested 5 minutes to settle the GeneJuice suspension. DNA (15 μg total) was added to each tube and mixed by pipetting up and down four times. Samples were allowed to rest for 5 minutes for GeneJuice-DNA complex formation and the suspension added dropwise to one dish of 293T cells. A typical transfection included these plasmids to produce replication incompetent retrovirus: 3.75 μg plasmid containing gag-pol (pEQ-PAM3(-E)), 2.5 μg plasmid containing viral envelope (e.g., RD114), Retrovirus encoding inducible chimeric signaling polypeptides and CARs, or inducible chimeric caspase-9 polypeptides=3.75 μg. Virus production is permitted to continue for 48-72 hours prior to transduction.

PBMCs were stimulated with anti-CD3 and anti-CD28 antibodies precoated to wells of tissue culture plates. 24 hours after plating, 100 U/ml IL-2 was added to the culture. On day 2 or three supernatant containing retrovirus from transfected 293T cells was filtered at 0.45 μm and centrifuged on non-TC treated plates precoated with Retronectin (10 μl per well in 1 ml of PBS per 1 cm² of surface). Plates were centrifuged at 2000 g for 2 hours at room temperature. CD3/CD28 blasts were resuspended at 2.5×10⁵ cells/ml in complete media, supplemented with 100 U/ml IL-2 and centrifuged on the plate at 1000×g for 10 minutes at room temperature. After 3-4 days incubation cells were counted and transduction efficiency measured by flow cytometry using the appropriate marker antibodies (typically anti—CD34 recognizing an epitope attached to the CAR). Cells were maintained in complete media supplemented with 100 U/ml IL-2, refed cells every 2-3 days with fresh media and IL-2 and split as needed to expand the cells.

T Cell Anti-Tumor Assay:

The HPAC PSCA⁺ tumor cells were stably labeled with nuclear-localized RFP protein using the NucLight™ Red Lentivirus Reagent (Essen Bioscience, 4625). To set up the coculture, 4000 HPAC-RFP cells were seeded per well of 96-well plates in 100 μl of CTL medium without IL-2 for at least 4 hours to allow tumor cells to adhere. After transduction with the appropriate retrovirus and having been allowed to rest for at least 7 days in culture, T were seeded according to various E:T ratios in the HPAC-RFP-containing 96-well plates. Rimiducid was also added to the some cultures to reach 300 μl total volume per well. Each plate was set up in duplicates, one plate to monitor with the IncuCyte and one plate for supernatant collection for ELISA assay on day 2. The plates were centrifuged for 5 min at 400×g and placed inside the IncuCyte (Dual Color Model 4459) to monitor red fluorescence (and green fluorescence if T cells were labeled with GFP-Ffluc) every 2-3 hours for a total of 7 days at 10× objective. Image analysis was performed using the “HPAC-RFP-_TcellsGFP_10×_MLD” processing definition. On day 7, HPAC-RFP cells were analyzed using the “Red Object Count (1/well)” metric. Also on day 7, 0 or 10 nM of suicide drug were added to each well of the coculture and placed back in the IncuCyte to monitor T cell elimination. On day 8, Tcell-GFP cells were analyzed using the “Total Green Object Integrated Intensity” metric. Each condition was performed at least in duplicates and each well was imaged at 4 different locations.

Multiplex analysis of cytokine level: 3×10⁴ transduced chimeric antigen receptor-expressing T cells (BP1798, 1799, 1801, 1802, 1810, 2210) or non-transduced controls from the same donor were added to each well of a 24 well plate either alone or preseeded with HPAC tumor lines recognized by the PSCA CAR. Wells were untreated or stimulated with 1 nM rimiducid. Each condition was duplicated. At 24 and 48 hours 200 μL culture supernatant was removed from each well. Production of inflammatory cytokines and chemokines was measured using a multiplex, magnetic bead array assay (MilliPlex, EMD Millipore, Germany) analyzed on a Luminex 200 machine (EMD Millipore, Germany) following the manufacturer's protocol. Briefly, 25 μl of cell culture supernatant or appropriately diluted serum sample collected at different time points was incubated with equal amount of the magnetic bead-bound, fluorescently-labeled antibody cocktail at room temperature for two hours with constant shaking and protected from light. A five-point standard curve was created using a mixture of peptides with known concentration provided in the kit. After washing, 25 μl of detection antibody cocktail was added and incubated at room temperature for one hour, then 25 μl of fluorescently-labeled Streptavidin was added and incubated for 30 minutes. After washing, the bead mixture was re-suspended in 150 μl of sheath fluid and the fluorescent signal was read on a Luminex 200 machine. Results were transferred to Excel (Microsoft) and graphed with Prism (GraphPad).

### Example 3: Generation of MyD88 Fusions with Costimulatory Domains that have Constitutive Activity

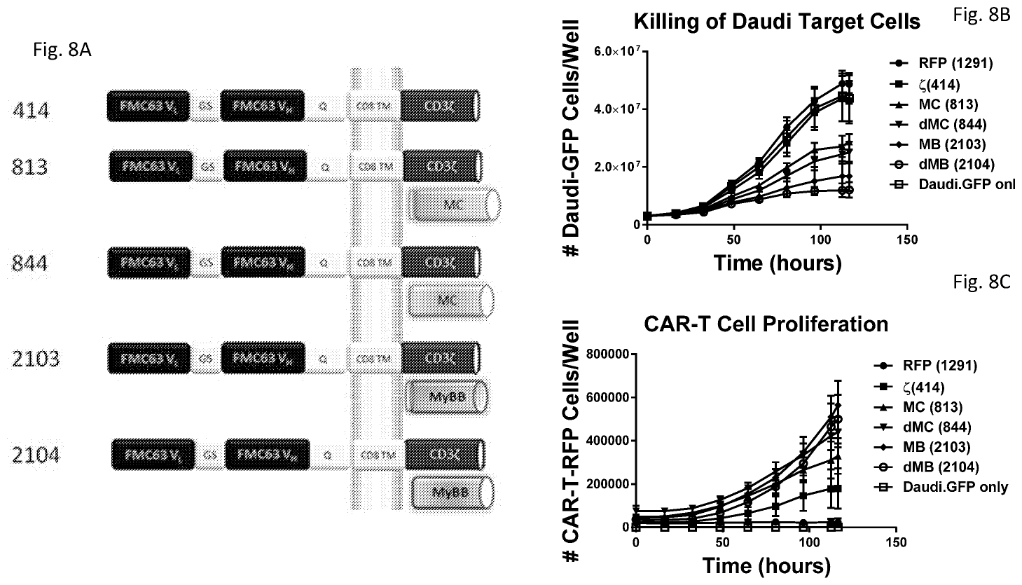

The observations described in FIGS. 4 and 7 indicate that iMyBB-expressing CAR-T cells produce a robust anti-tumor cell effect similar to iMC-expressing CAR-T cells, but with a much lower release of inflammatory cytokines. The effect of non-inducible chimeric signaling polypeptides comprising MyBB was tested as part of a nucleic acid that expressed both the chimeric signaling polypeptide, without the multimeric ligand binding region, along with an inducible chimeric Caspase-9 polypeptide, and a CAR, in a T cell. (FIG. 8). These CD19-targeted CAR-T cells were incubated with Burkitt Lymphoma Daudi cells at an effector cell to target cell (E:T) ratio of 1:25 and the capacity of the CAR-T cells to proliferate and control the proliferation of the tumor by lysis was determined microscopically over five days. Both MyBB and MC containing CAR-T cells proliferated well when exposed to tumor. At a lower E:T ratio MyBB encoding CAR-T cells expanded somewhat faster than MC CAR-Ts. Each controlled tumor completely at an E:T of 1:5 but with increasing tumor burden to control, MyBB expressing CAR-T cells were slightly more effective than MC CAR-T cells (FIG. 8).

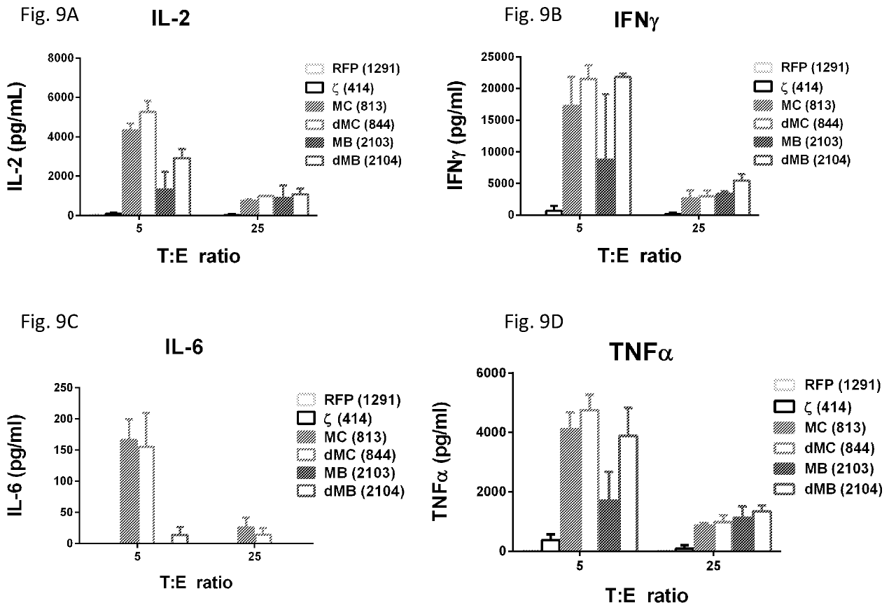

The pattern of cytokine production exhibited by non-inducible MC and MyBB containing CAR-T cells in this format was similar to that observed in the inducible MC and MyBB containing CAR-T cells displayed in FIGS. 5-7. Despite the robust proliferation observed in coculture with tumor targets, IL-2 secretion from the MyBB enabled CAR-T cells was about half that of MC CAR-Ts (FIG. 9). Interferon secretion was comparable but the inflammatory cytokine IL-6 was markedly reduced in the MyBB enabled CAR-T coculture. This finding, combined with the indication that inflammatory cytokines are reduced in iMyBB enabled CAR-Ts (FIG. 7), may indicate that that CAR-T therapy utilizing MyBB will have less adverse events resulting from toxic cytokine release with similar efficacy to MC enabled CAR-T cells. Furthermore, fusion of other signaling domains other than CD40, CD28, OX40, 4-1BB, or ICOS, may facilitate the user directed production of distinct phenotypic outcomes in CAR-T cells including, for example, their proliferation, capacity to differentiate into memory cell populations, interaction with the tumor microenvironment and their expression and secretion of cytokines.

Methods

To measure Raji (a CD19 positive Burkitt lymphoma line) cell anti-tumor activity populations of cells were determined by flow cytometry rather than Incucyte as the cells do not adhere to a plate. Raji cells (ATCC) labeled by stable expression of Green Fluorescent Protein (Raji-GFP) are a Burkitt's lymphoma cell line that express CD19 on the cell surface and are a target for an anti-CD19 CAR. 50000 Raji-GFP cells were seeded on a 24 well plate with 10000 CAR-T cells, a 1:5 E:T ratio. Media supernatant was taken at 48 hours for determination of cytokine release by activated CAR-T cells. The degree of tumor killing was determined at 7 days and 14 days by flow cytometry (Galeos, Beckman-Coulter) by the proportion of GFP labeled tumor cells and CD3 labeled T cells.

### Example 4: Phenotyping of CAR-Expressing T Cells that Express the MyD88 Fusion Inducible Chimeric Signaling Polypeptides

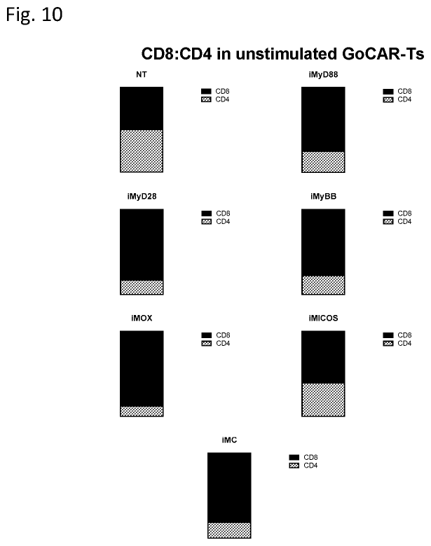

Following activation with plate-bound antibodies (αCD3/αCD28), naïve T cells derived from peripheral blood mononuclear cells (PBMCs) differentiate spontaneously into discrete lineages that can be determined by the expression of marker proteins on the cell surface. Furthermore, preexisting CD4⁺ helper T cells (Th) and CD8⁺ cytotoxic T lymphocytes (CTLs) may selectively expand when transduced to become CAR-T cells. This expansion and the differentiated phenotype is likely to be influenced by the signaling of the costimulatory domains introduced to the CAR-T cells. To determine the influence of MC and the effect of replacement of the CD40 domain on MyD88, PBMCs were transduced with constructs 1798 expressing inducible MyD88/CD28 (iMyD28), 1799 (iMyBB), 1801 (MyD88/OX40 (iMOX)), 1802 (MyD88/ICOS (iMICOS)), 1810 (MyD88 alone (iMyD88)), and 2210 (MyD88/CD40 (iMC)). These CAR-T cells were allowed to expand with IL-2 for three weeks in the absence of rimiducid and populations were stained with fluorescent antibodies recognizing specific cell surface markers indicative of their T cell subtype with the results determined by flow cytometry on a Gallios cytometer and Kaluza software (Beckman Coulter). The selective expansion of CD8⁺ CAR-T cells over CD4 CAR-T cells carrying MyD88 was evident in FIG. 10. Untransduced cells (NT) were maintained at an approximately 1:1 ratio over three weeks, but CAR-T cells containing MyD88 alone were biased toward CD8 cells at a 3:1 ratio. Addition of CD40 to MyD88 (iMC) expanded this bias for CD8 cells to 4:1. Fusion of CD28 or 41BB (MyBB) to MyD88 yielded a similar proportion of CD8 cells as MC while MOX CAR-T cells were 89% CD8, and MICOS CAR-T cells had only a 60:40 split of CD8:CD4. These results indicated that signaling from MyD88 and from combinations of MyD88 and distinct signaling nodes (in combination with a 1^{st} generation CAR) can bias the expansion of CD8 cells relative to CD4 cells.

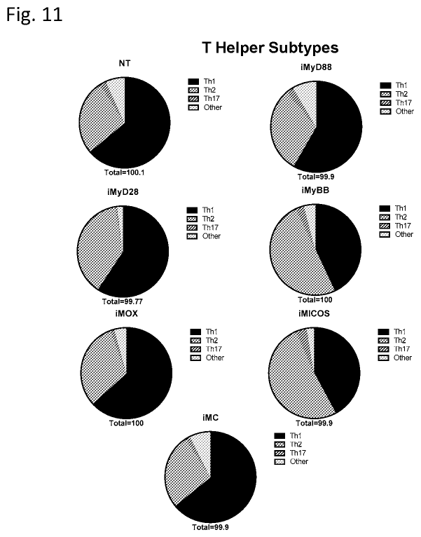

CD4 positive T helper cells are present in several lineages that differentially secrete cytokines influencing their microenvironment. These include Th1 cells that are useful to drive CD8 cell responses and Th2 cells that drive humoral and inflammatory responses. Smaller but important populations include T_{reg} cells that are broadly inhibitory, Th₁₇ cells that secrete IL-17 and Th₉ cells that secrete IL-9. These cells can be identified in a flow cytometer by labeling with antibodies directed to lineage-specific cell surface markers and by fixation and intracellular labeling of the cytokines they were attempting to secrete. To determine the effect of alteration of tonic, basal MyD88 directed costimulatory signals in combination with other signaling moieties, PBMCs were mock transduced (NT) or transduced with chimeric antigen-expressing constructs 1798 expressing inducible MyD88/CD28 (iMyD28), 1799 (iMyBB), 1801 (MyD88/OX40 (iMOX)), 1802 (MyD88/ICOS (iMICOS)), 1810 (MyD88 alone (iMyD88)), and 2210 (MyD88/CD40 (iMC)). CD4⁺ cells were gated and the expression CXCR3 used to separate Th1 from Th2 cells and the expression of CCR6high used to identify Th₁₇ cells. The proportions of these cells with respect to their transduced costimulatory moieties are represented on FIG. 11. Th1 cells composed about two-thirds of the T helper population in untransduced cells. Expression of iMyD88, or most of the iMyD88 fusions did not alter this selective outgrowth phenotype. However, expression of iMICOS or iMyBB led to about a 50:50 ratio of Th1:Th2. Presence of the small proportion of Tregs in these populations was further reduced when 41-BB, ICOS, or CD28 signaling domains were fused with MyD88. These results support the notion that lineage direction can be influenced by introduced signaling domains in naïve human T cell populations and in CAR-T cells.

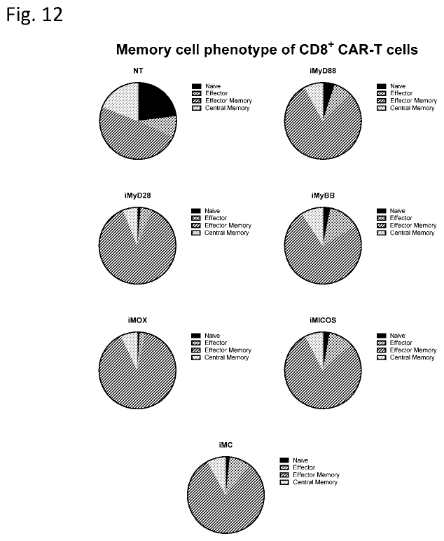

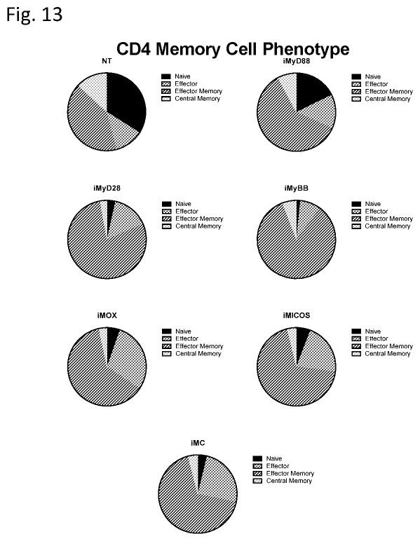

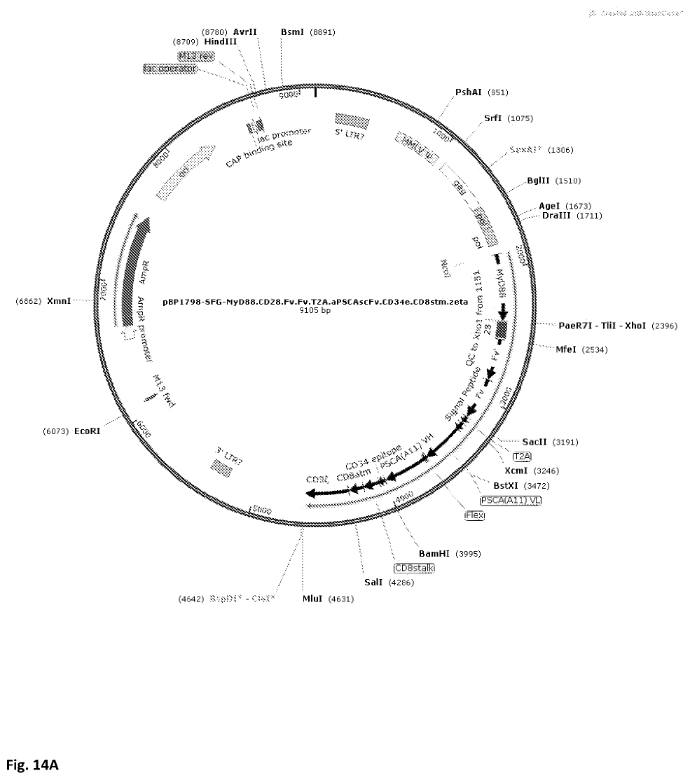

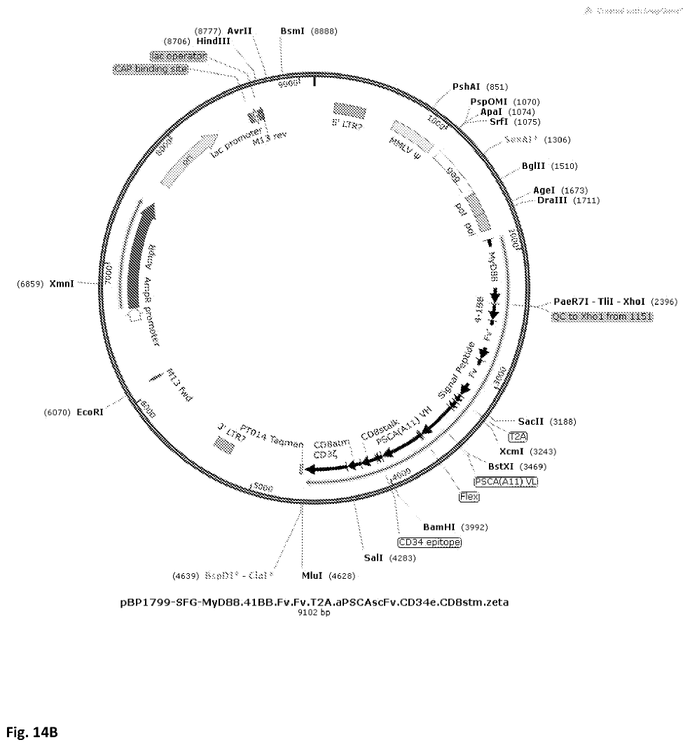

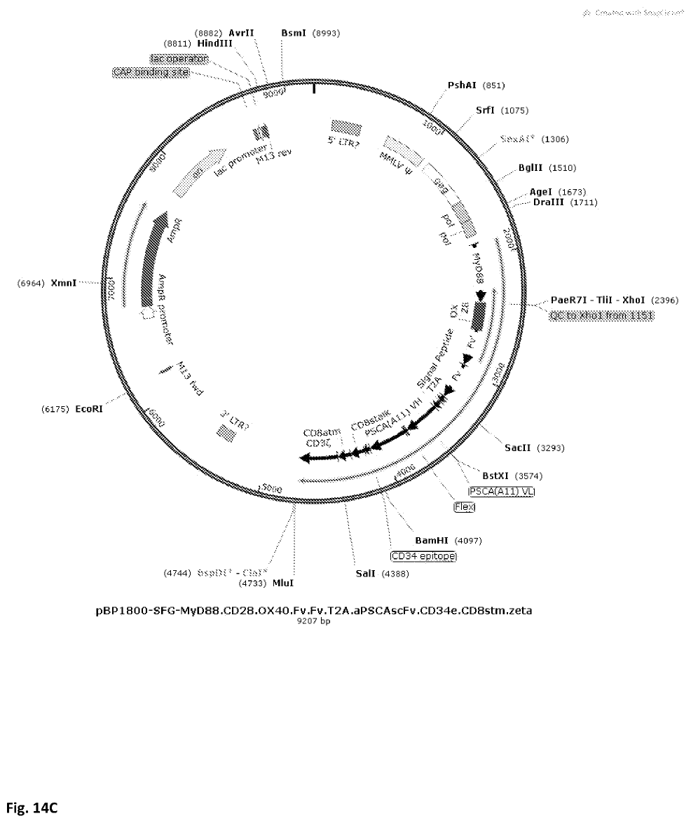

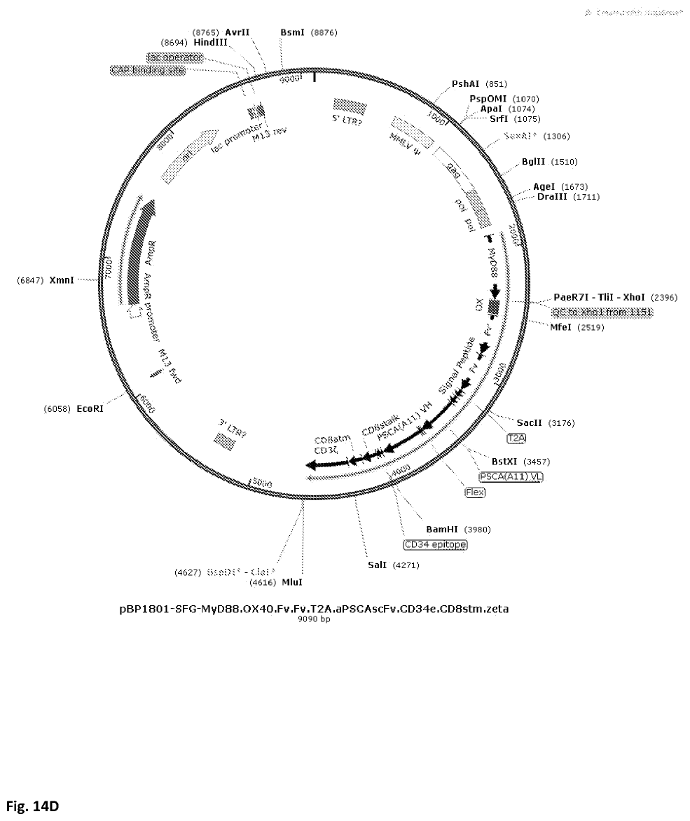

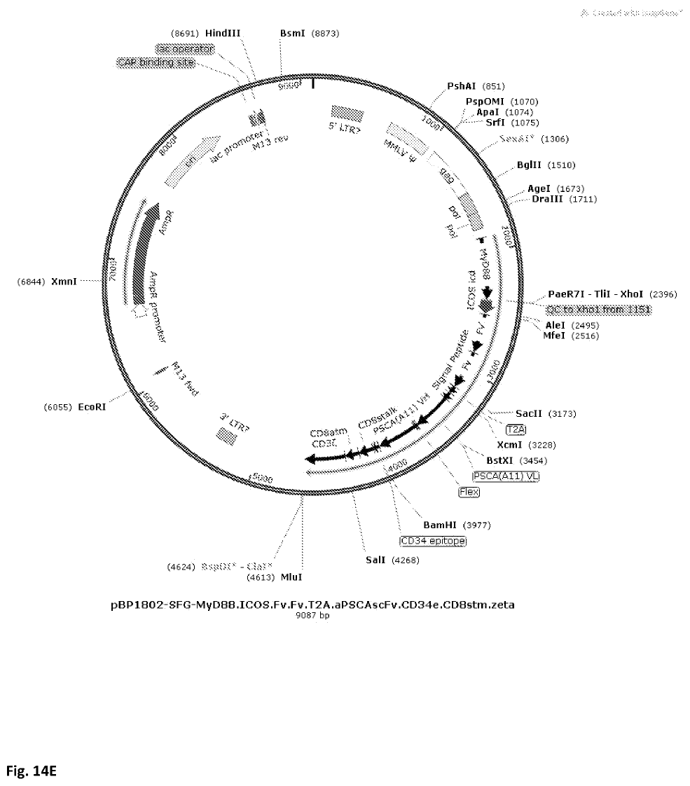

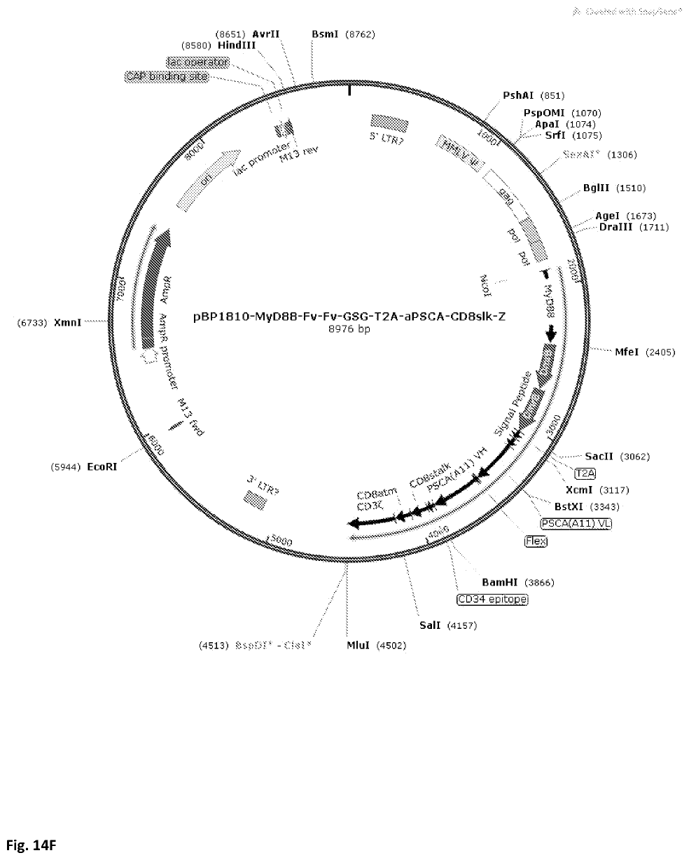

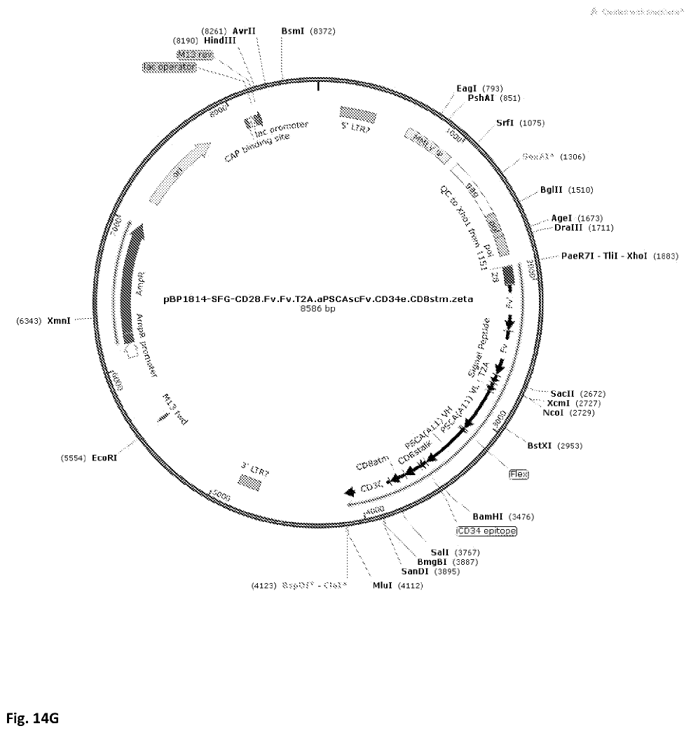

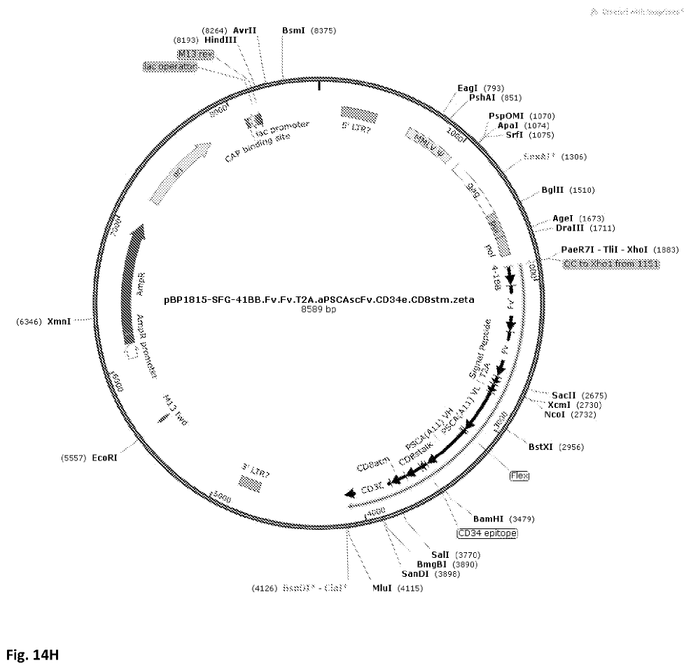

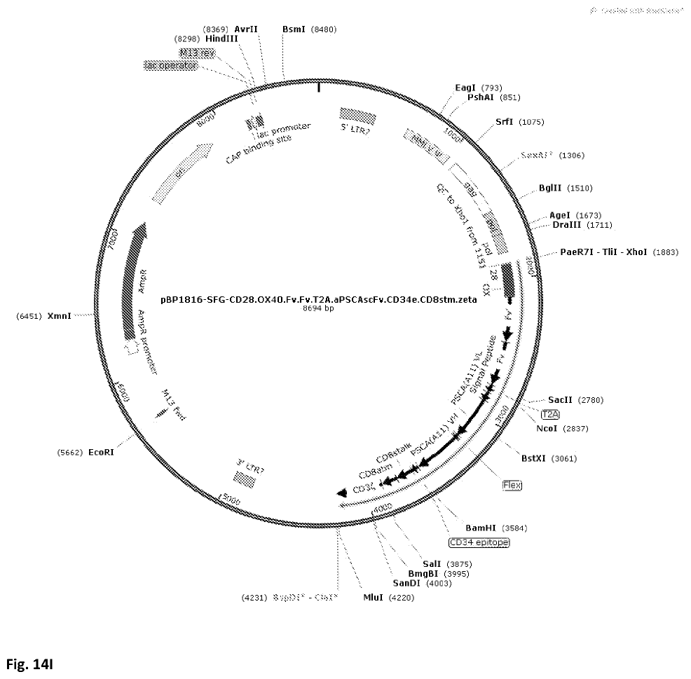

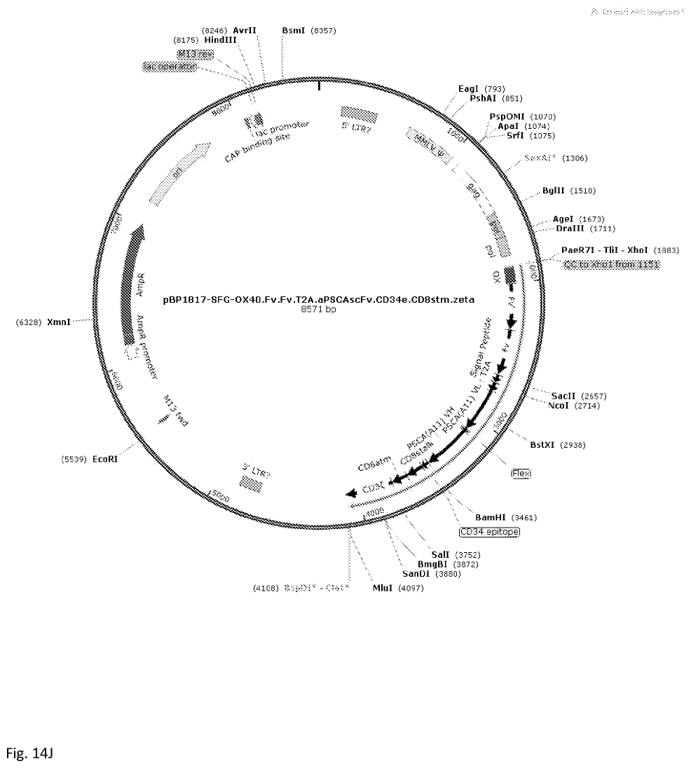

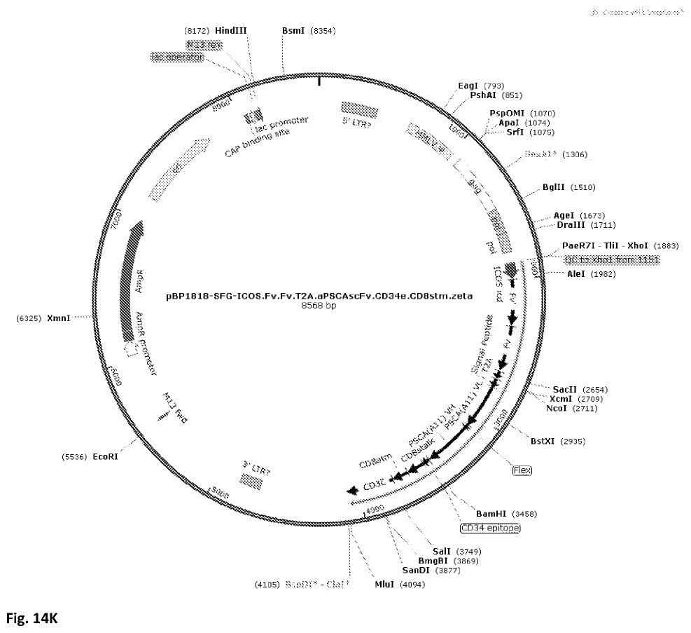

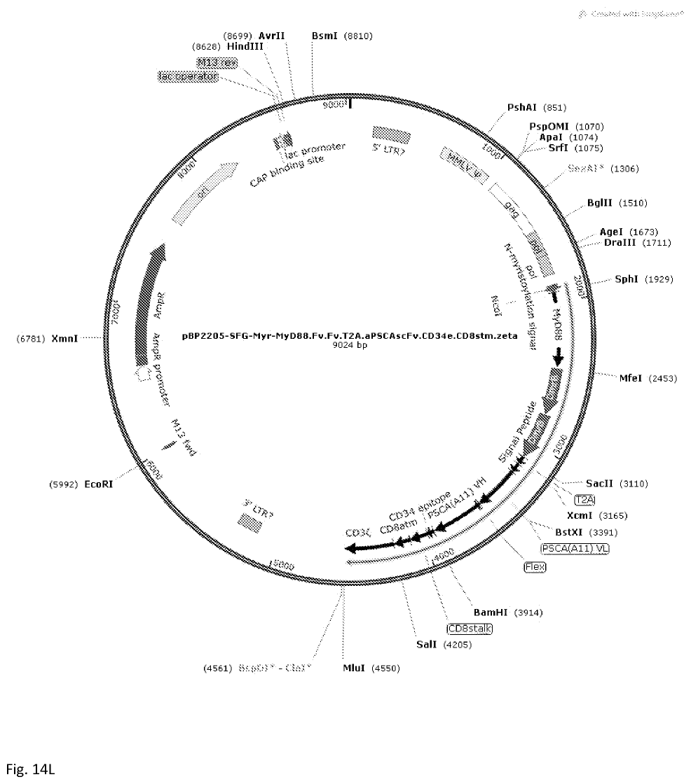

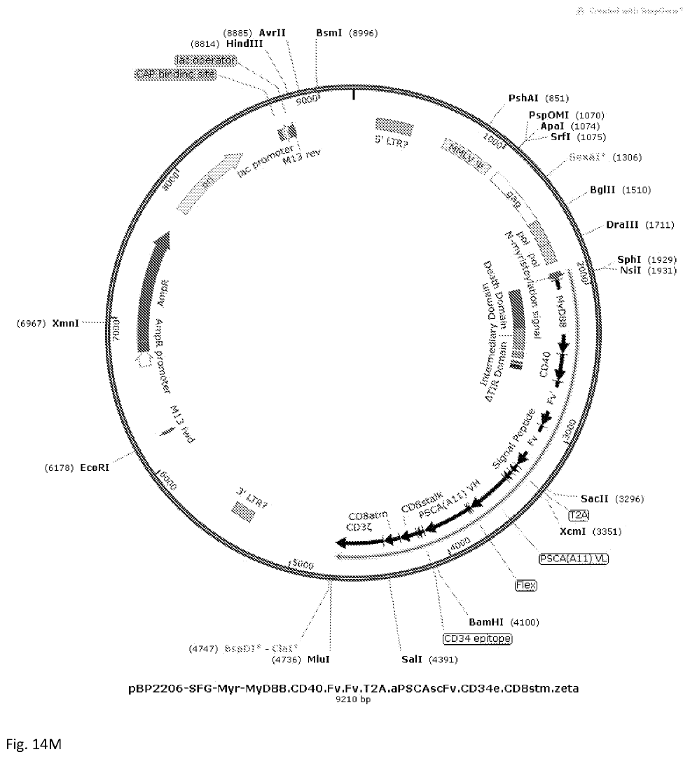

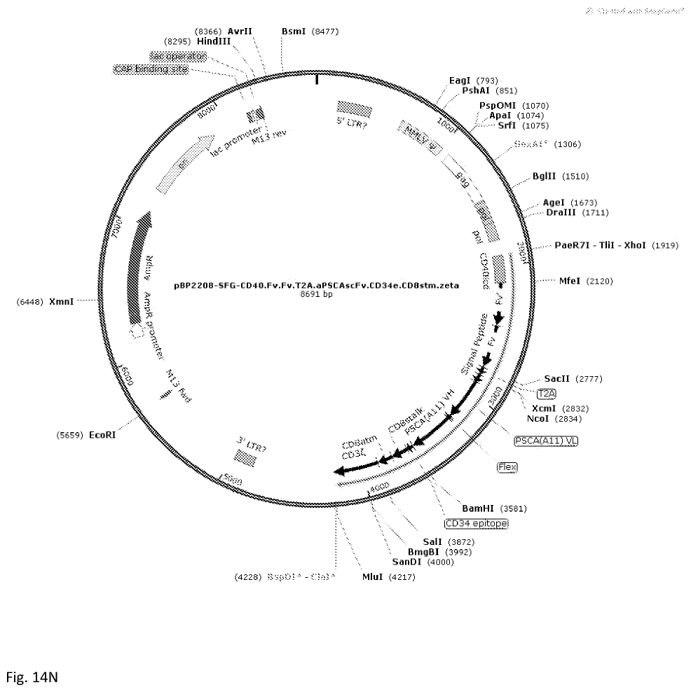

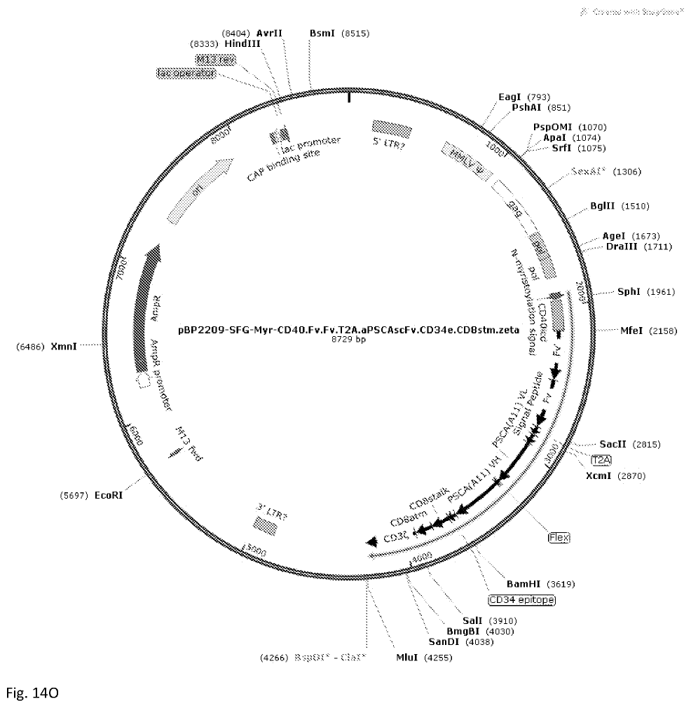

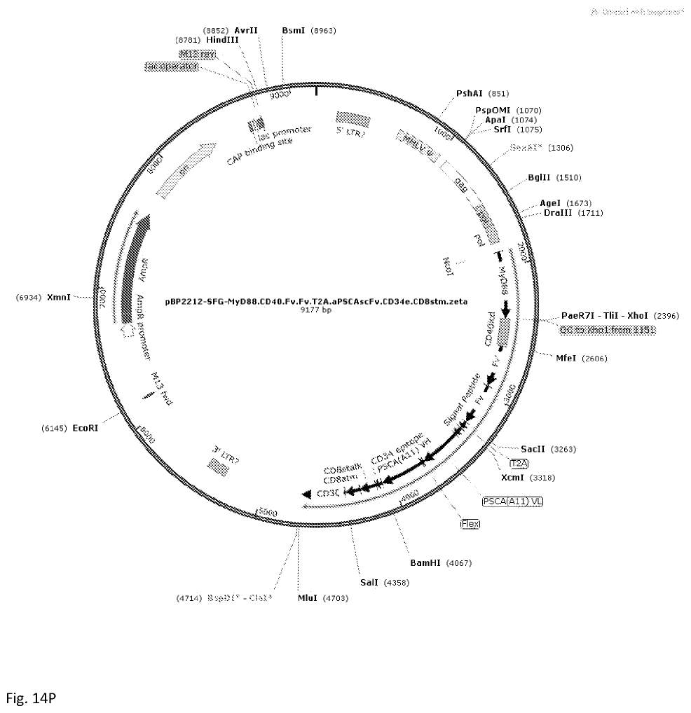

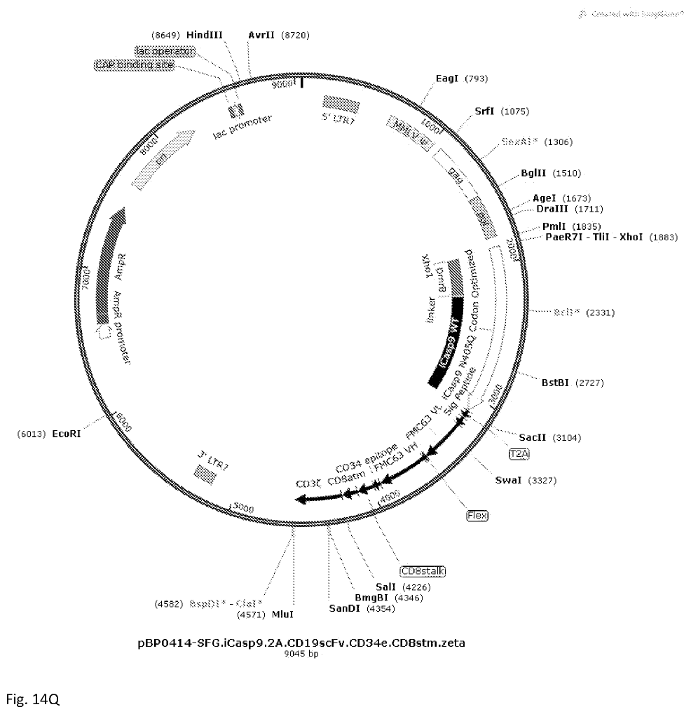

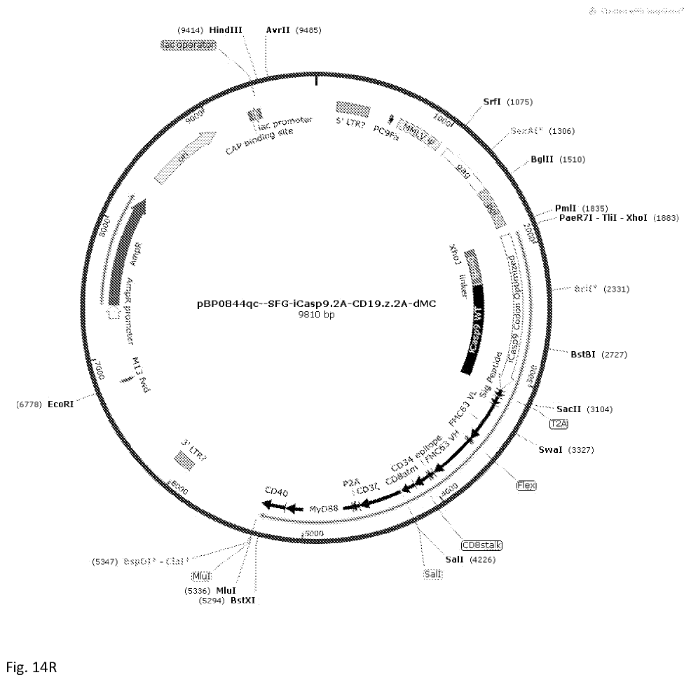

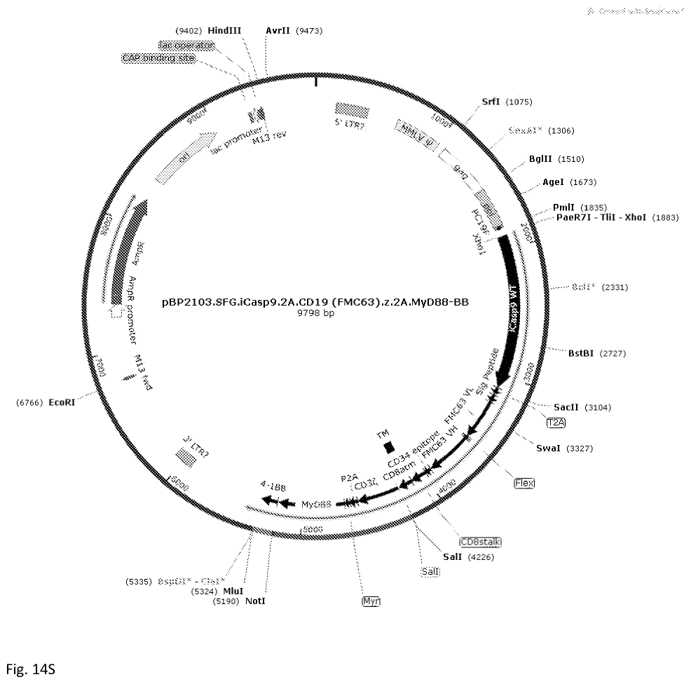

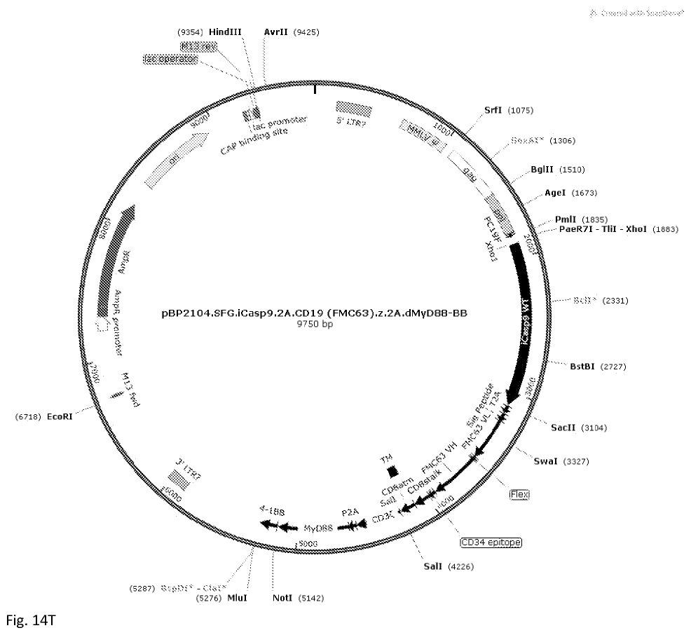

Following activation with antibodies to T cell receptor and CD28, PBMCs will proliferate over the course of 21 days or more in the presence of IL-2. Many T cells will also differentiate from a naïve state to ‘effector’ cells poised to respond to cells expressing their cognate antigen and to ‘memory’ cells that have responded to antigen but are poised to respond further to antigenic challenge. The identity of these cells can be determined by the presence of marker proteins on their cell surface visualized with fluorescently labeled antibodies on a flow cytometer. Memory cells can be further divided into ‘effector memory’ and ‘central memory’ and other populations. The differentiation of CAR-T cells along one or another T cell lineage may be controlled in part by costimulatory signals introduced to the cells. PBMCs were transduced with chimeric antigen receptor-encoding constructs 1798 expressing inducible MyD88/CD28 (iMyD28), 1799 (iMyBB), 1801 (MyD88/OX40 (iMOX)), 1802 (MyD88/ICOS (iMICOS)), 1810 (MyD88 alone (iMyD88)), and 2210 (MyD88/CD40 (iMC)). These CAR-T cells were allowed to expand with IL-2 for three weeks in the absence of rimiducid and populations were stained with fluorescent antibodies recognizing specific cell surface markers indicative of their memory cell phenotype and T cell subtype with the results determined by flow cytometry on a Gallios cytometer and Kaluza software (Beckman Coulter). The differentiation of the CD8⁺ cells that predominated the transduced populations is displayed in FIG. 12. The mock transduced (NT) cells differentiated to about 50% effector memory cells with about 20% naïve and central memory cells. Expression of MyD88 alone or with any other signaling domain tested increased the proportion of effector memory cells and decreased the number of naïve cells (probably indicating that these cells were highly active from tonic MyD88 signaling). The pattern of differentiation in CD4⁺ cells is presented in FIG. 13. This relatively small population of cells (FIG. 11) also predominantly differentiated to effector memory cells with a somewhat higher proportion of effector cells (compared with CD8⁺ cells) remaining in the culture at 3 weeks. These studies indicate that the pattern of differentiation of T cells in culture can be influence by transgenic expression of iMyD88 and furthermore that the pattern of differentiation can be altered subtly by fusion of iMyD88 with different signaling domains.

### Example 5: Fusion of FKBP12 with Membrane-Targeted MyD88-X Signaling Domains Confers Drug-Inducible Signal Transduction

The present example discusses the design and assay of cells that express inducible chimeric signaling polypeptides that comprise MyD88 and costimulatory polypeptides. Methods for preparing modified cells that express the chimeric signaling polypeptides, and assays, are essentially as discussed herein, and in Examples 1-4. Among the chimeric signaling polypeptides of the present example, are chimeric polypeptides that comprise a truncated MyD88 polypeptide, a costimulatory polypeptide cytoplasmic signaling region, and a myristoylation region. In some examples, the modified cells also express a chimeric antigen receptor, for example, CARs directed against PSCA.

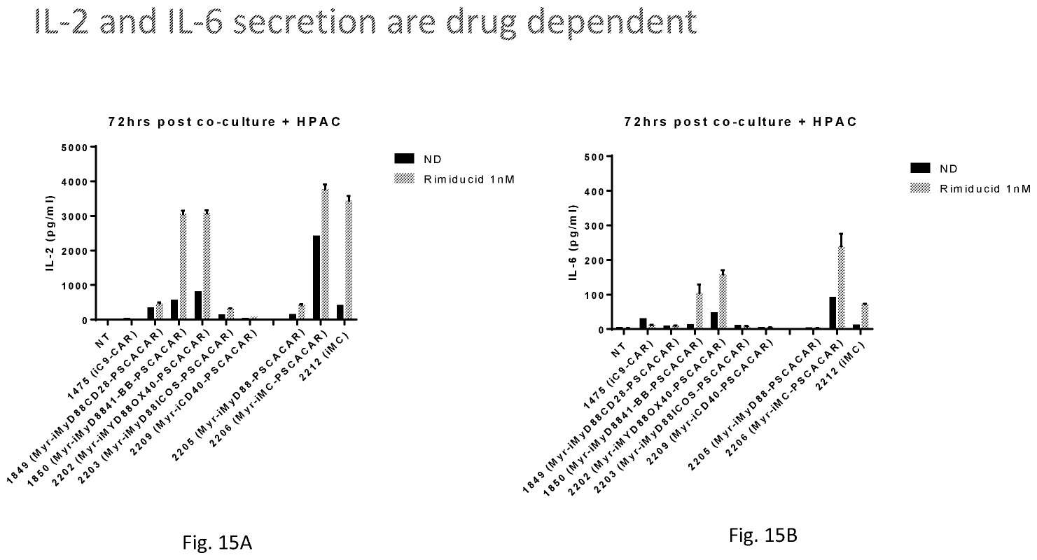

IL-2 and IL-6 secretion in modified T cells that comprised chimeric signaling polypeptides discussed herein, was assayed in the presence, or absence of rimiducid (FIG. 15A and FIG. 15B). The modified cells also comprised chimeric antigen receptors that comprise antigen recognition regions that recognize PSCA. Cells were assayed 72 hours post co-culture with HPAC cells, a target of PSCA. ND=no dimerizer (no rimiducid). NT=not transfected. IL-2 and IL-6 secretion appeared to be rimiducid dependent in most of the modified cells of the assay. The level of IL-2 and IL-6 secretion in modified cells that expressed a myristoylated inducible MyD88-CD28 chimeric signaling polypeptide (1849) or a myristoylated inducible CD40 chimeric polypeptide (2209), along with a PSCACAR, was not induced in the presence of 1 nM rimiducid.

The retroviral vectors used to transfect the cells comprised polynucleotides that encoded the following polypeptides:

1475 iC9

1849: Myr-iMyD88

1850: Myr-iMyD88-4-1BB+PSCACAR

2202: Myr-iMyD88-OX40+PSCACAR

2203: Myr-iMyD88-ICOS+PSCACAR

2209: Myr-iCD40+PSCACAR

2205: Myr-iMyD88+PSCACAR

2206: Myr-iMyD88-CD40+PSCACAR

2212: Myr-iMyD88-CD40

Where two polypeptides are indicated (e.g., iMyD88-PSCACAR), the two polypeptides are expressed on two polynucleotides of the expression vector, under the control of the same promoter, and separated by a cleavable 2A linker).

Myr=myristoylation region as provided herein.

iC9=FKBP12v36-Caspase9

iMyD88=Fv′Fvls-truncated MyD88

iCD40=FvFvls-CD40 cytoplasmic domain

PSCACAR=chimeric antigen receptor that recognizes PSCA

HPAC: human pancreatic cancer cell line

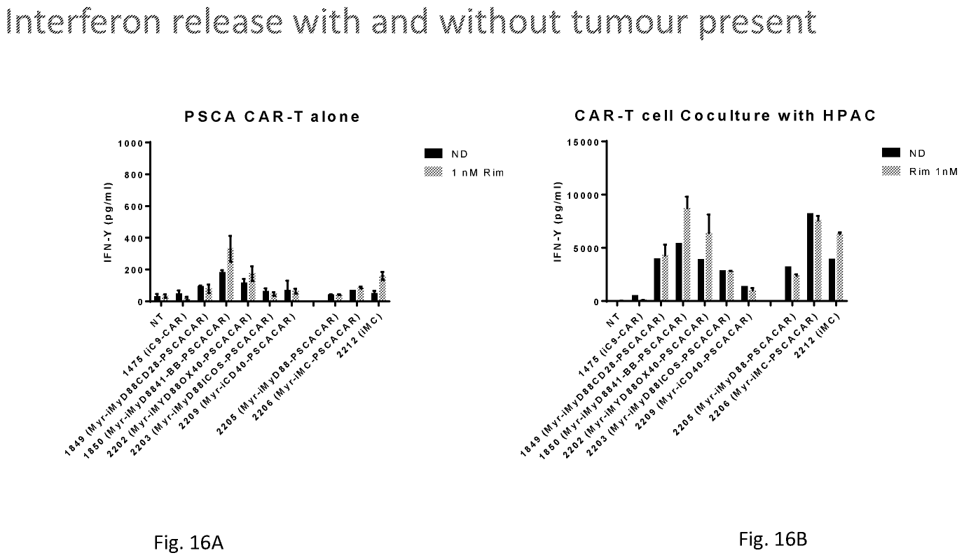

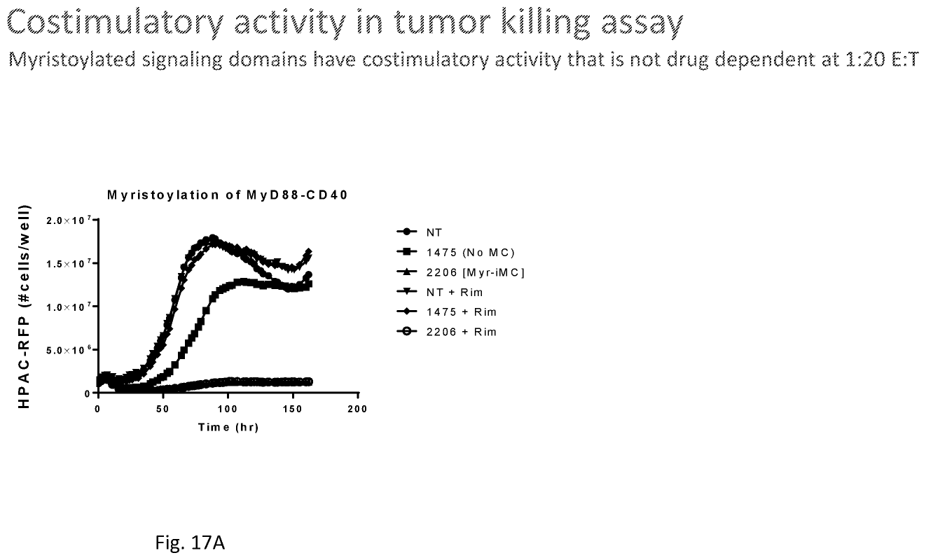

Interferon gamma secretion in modified T cells that comprised chimeric signaling polypeptides discussed herein, was assayed in the presence, or absence of rimiducid (FIG. 16A and FIG. 16B). The modified cells were assays either alone, or as part of a coculture with HPAC cells. The myristoylated inducible chimeric signaling polypeptides assayed produced interferon gamma levels similar to those produced to myristoylated inducible truncated MyD88/CD40 chimeric polypeptides (iMC). IL-2 and IL-6 secretion was more drug dependent than interferon gamma secretion in these assays for most of the inducible chimeric signaling polypeptide switches. Although myristoylated inducible CD40 had some activity in the tumor-killing assay, myristoylated inducible CD40, and myristoylated inducible ICOS had low signaling in this assay.

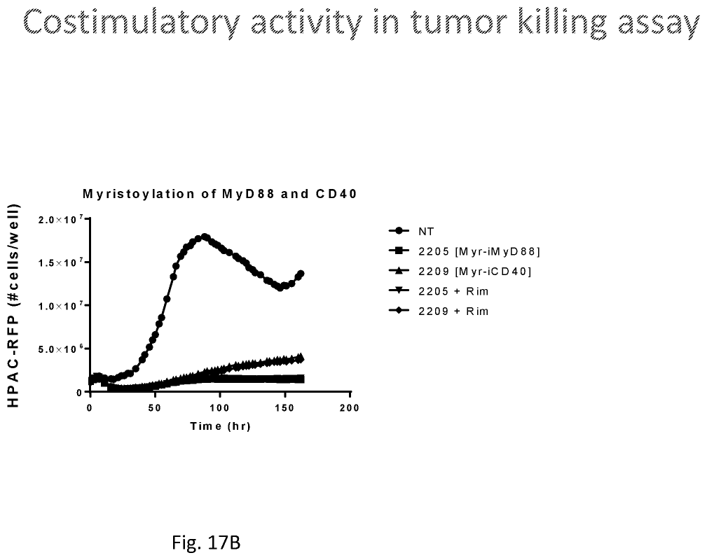

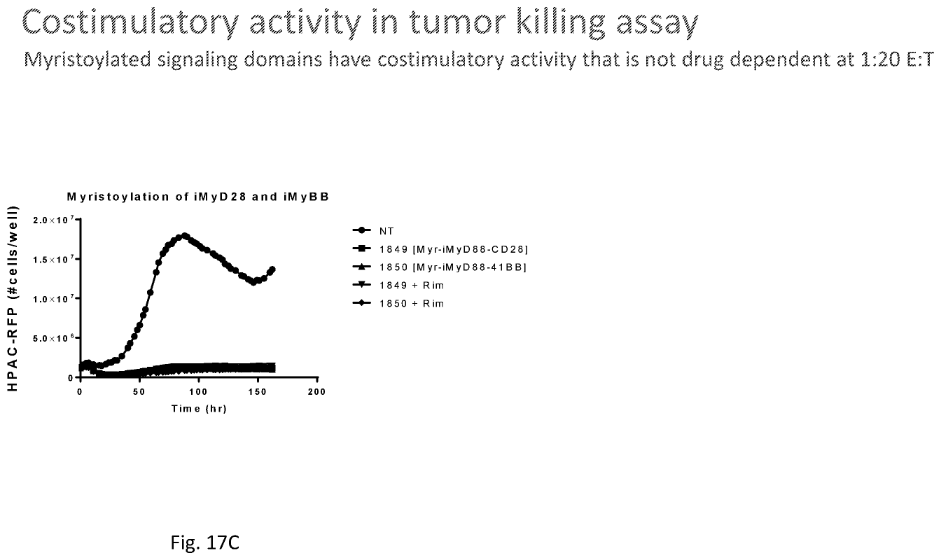

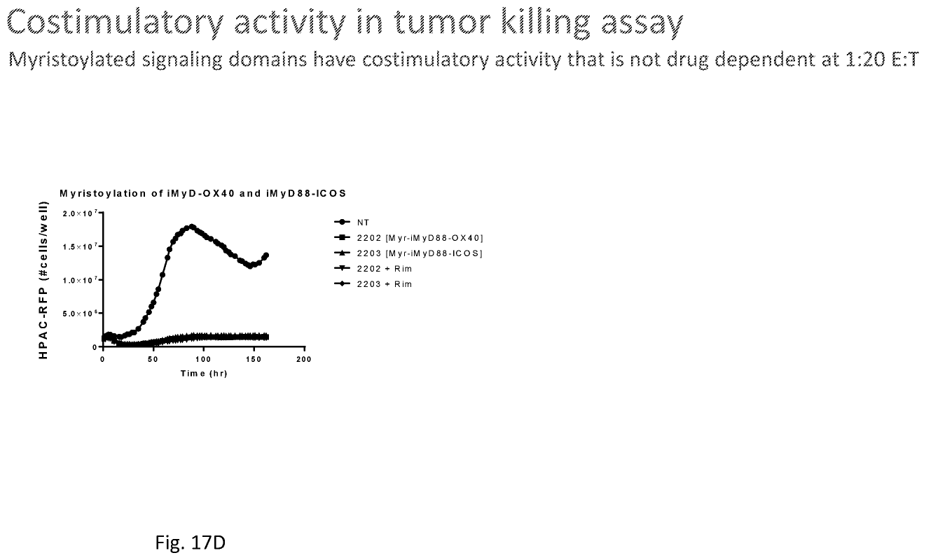

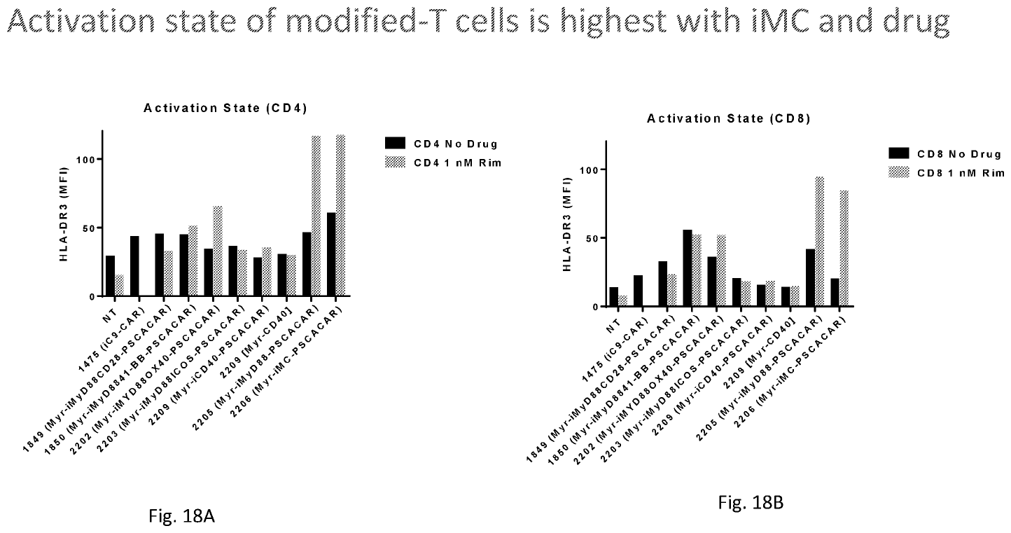

Costimulatory activity of the myristoylated inducible chimeric signaling polypeptides was assayed in a tumor killing assay. The signaling domains demonstrated costimulatory activity that was not drug dependent at 1:20 E:T HPAC cells, as measured by RFP fluorescence. Myristoylation of the inducible chimeric signaling polypeptides yielded an active fusion polypeptide that was capable of driving costimulation to support 7 day tumor control of HPAC pancreatic cancer cells. Control construct 1475 (no MyD88, CD40, or other costimulatory molecule) containing a CAR without costimulation provided poor control, though greater than non-transduced T cells. All of the inducible chimeric signaling polypeptides tested here, comprising MyD88, controlled HPAC proliferation with, or without, rimiducid directed dimerization. (FIG. 17A-D). The activation state of the modified cells, in the absence of the tumor target, for example, in the absence of the HPAC cells, was assayed (FIG. 18A (CD4⁺ T cells) and FIG. 18B (CD8⁺ T cells)).

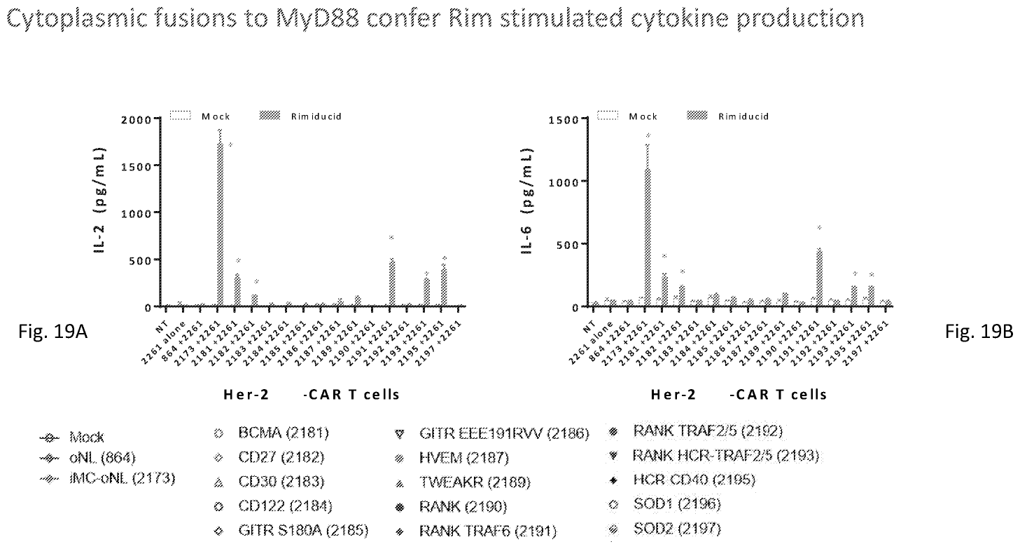

As further examples of the utility of fusing cell signaling domains with MyD88 and Fv to generate inducible go switches, several alternative switches were created. To assay their capacity to promode ligand-dependent T cell activation, IL-2 and IL-6 secretion in modified T cells that comprised chimeric signaling polypeptides discussed herein, was assayed in the presence, or absence of rimiducid. For this set of assays, modified cells co-expressed the following unmyristoylated and therefore cytoplasmic inducible chimeric signaling polypeptides, along with a CAR having an antigen recognition moiety that recognizes Her-2 (Fi. 19A and FIG. 19B). Go-Switches containing Fv-Fv-Fusions to MyD88 and the indicated signaling domains yields an active fusion product that is capable of driving costimulation to support an immune response against HER2 expressing cancer cells. In each case addition of rimiducid to dimerize the fusion protein stimulated the CAR-T cells to activate production of the cytokines IL-2 and IL-6.

Methods: Human primary T cells were transduced or mock transduced (NT) with retroviruses produced from the plasmid 2261 encoding a first generation CAR directed against HER2 and cotransduced with retrovirus encoding the fusion between MyD88 and the indicated signaling domain followed by Fv ligand binding domains. After a rest period of eight days HER2 expressing tumor were cocultured at and effector to target ratio of 1:5. Supernatants from the cocultures in the presence or absence of 1 nM rimiducid were assayed for cytokine levels by ELISA.

2261: HER2CAR (first generation; 4D5)

2181: iMyD88-BCMA

2182: iMyD88-CD27

2183: iMyD88-CD30

2184: iMyD88-CD122

2185: iMyD88-GITR S180A (removing a site of phosphorylation on GITR that recruits proapototic signaling molecules)

2186: iMyD88-GITR EEE191RVV (removing a site of phosphorylation on GITR that recruits proapototic signaling molecules)

2187: iMyD88-HVEM

2189: iMyD88-TWEAKR

2190: iMyD88-RANK

2191: iMyD88-RANK TRAF6 (comprising the TRAF6 binding site of RANK).

2192: iMyD88-RANK TRAF2/5

2193: iMyD88-RANK HCR-TRAF 2/5 (comprising the TRAF2/5 binding site of RANK)

2195: iMyD88-CD40-HCR-CD40 (comprising a portion of CD40, and the HCR region of RANK (see, e.g., Takaguchi, Y., et al., Genes to Cells, 2009, 14:1331-1345 (Molecular Biology Society of Japan/Blackwell Publishing Ltd.).

2196: iMyD88-SOD1

2197: iMyD88-SOD2

BTN3A1 is butyrophilin, subfamily 3, member A1. It plays a role in T-cell activation and in the adaptive immune response. Regulates the proliferation of activated T-cells. Regulates the release of cytokines and IFNG by activated T-cells. Mediates the response of T-cells toward infected and transformed cells that are characterized by high levels of phosphorylated metabolites, such as isopentenyl pyrophosphate. It is detected on T-cells, natural killer cells, dendritic cells and macrophages (at protein level). Ubiquitous. Highly expressed in heart, pancreas and lung, Moderately expressed in placenta, liver and muscle Dectin-1 is a member of the C-type lectin/C-type lectin-like domain (CTL/CTLD) superfamily. It functions as a pattern-recognition receptor for a variety of β-1,3-linked and β-1,6-linked glucans from fungi and plants, and in this way plays a role in innate immune response. It becomes a homodimer necessary for the TLR2-mediated inflammatory response and for TLR2-mediated activation of NF-kappa-B. It enhances cytokine production in macrophages and dendritic cells. Mediates production of reactive oxygen species in the cell and mediates phagocytosis of C. albicans conidia. It binds T-cells in a way that does not involve their surface glycans and plays a role in T-cell activation. Stimulates T-cell proliferation. Expression is found on myeloid dendritic cells, monocytes, macrophages and B cells

ITGA5 (a.k.a Integrin alpha 4/CD49d/ITGA4) belongs to the integrin alpha chain family that becomes heterodimer. Integrin alpha-5/beta-1 is a receptor for fibronectin and fibrinogen. ITGA5:ITGB1 acts as a receptor for fibrillin-1 (FBN1) and mediates R-G-D-dependent cell adhesion to FBN1

ITA4 (a.k.a. Integrin alpha 5/CD49e/FNRA) is a heterodimer of an alpha and a beta subunit of integrin. The alpha subunit can sometimes be cleaved into two non-covalently associated fragments. Alpha-4 associates with either beta-1 or beta-7. Alpha-4 interacts with PXN, LPXN, and TGFB1I1/HIC5. Interacts with CSPG4 through CSPG4 chondroitin sulfate glycosaminoglycan. Interacts with JAML; integrin alpha-4/beta-1 may regulate leukocyte to endothelial cells adhesion by controlling JAML homodimerization. ITGA4:ITGB1 is found in a ternary complex with CX3CR1 and CX3CL1. It is expressed on activated endothelial cells integrin VLA-4 triggers homotypic aggregation for most VLA-4-positive leukocyte cell lines. It may also participate in cytolytic T-cell interactions with target cells. ITGA4:ITGB1 binds to fractalkine (CX3CL1) and may act as its coreceptor in CX3CR1-dependent fractalkine signaling (PubMed:23125415). ITGA4:ITGB1 binds to PLA2G2A via a site (site 2) which is distinct from the classical ligand-binding site (site 1) and this induces integrin conformational changes and enhanced ligand binding to site.

RANK88 is a variant of RANK with the following modifications:

| | |
|---|---|
| 1. | KKVAKKPTNKAPHPKQEPQEINFPDDLPG before RANK |
| 2. | Contains RANK-HCR domain and 5′ portion |
| | of TRAF 2/5 |

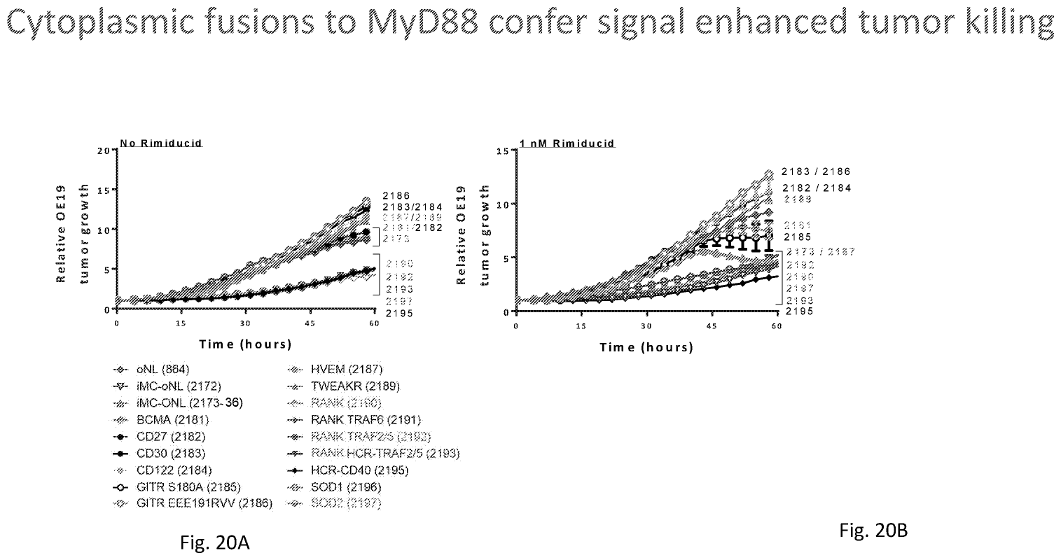

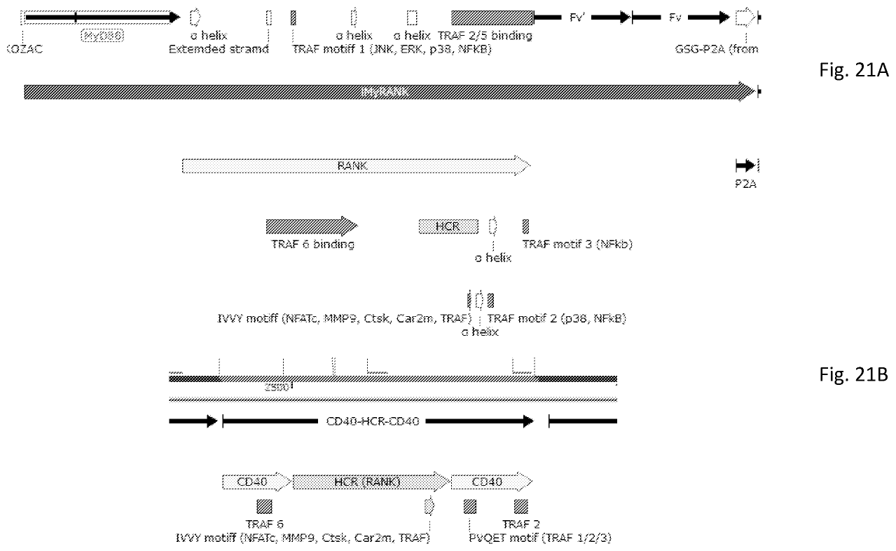

Modified T cells that expressed both the indicated myristoylated inducible signaling polypeptides and the HER2-CAR were assayed in an OE19 tumor-cell killing assay in the absence or presence of rimiducid (FIG. 20A and FIG. 20B). Go-Switches containing Fv-Fv-Fusions to MyD88 and the indicated signaling domains yields an active fusion product that is capable of driving costimulation to support 7 day tumor control of HER2 positive cancer cells.

Methods: Human primary T cells were transduced or mock transduced (NT) with retroviruses produced from the plasmid 2261 encoding a first generation CAR directed against HER2 and cotransduced with retrovirus encoding the fusion between MyD88 and the indicated signaling domain followed by Fv ligand binding domains. After a rest period of eight days HER2 expressing tumor were cocultured at and effector to target ratio of 1:15. Outgrowth of the labeled tumor cells was measured in real time in an Incucyte microscope chamber.

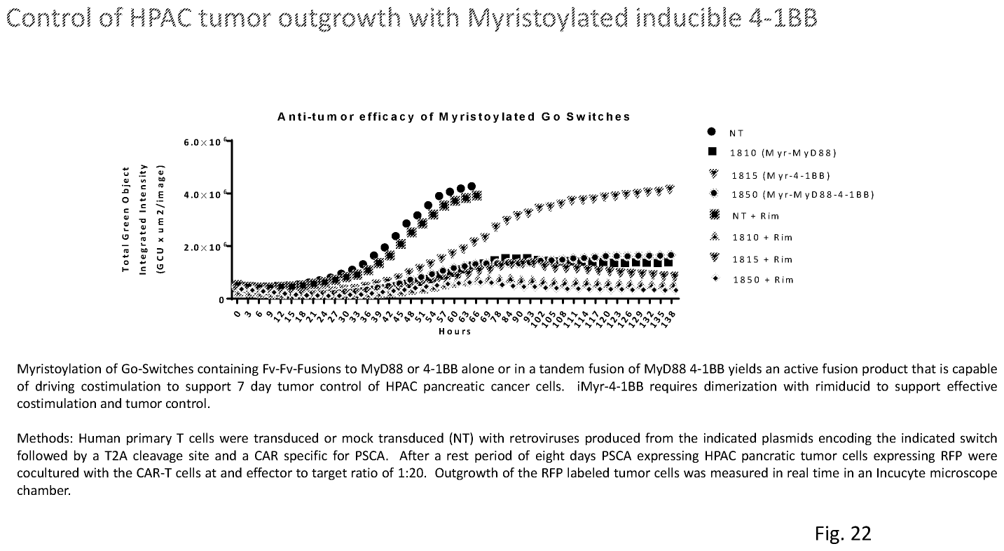

In some examples, generating a membrane-tethered switch makes possible cell stimulation without MyD88 fusion. In FIG. 22 Myristoylation of Go-Switches containing Fv-Fv-Fusions to MyD88 or 4-1BB alone or in a tandem fusion of MyD88 4-1BB yields an active fusion product that is capable of driving costimulation to support 7 day tumor control of HPAC pancreatic cancer cells. iMyr-4-1BB requires dimerization with rimiducid to support effective costimulation and tumor control. Methods: Human primary T cells were transduced or mock transduced (NT) with retroviruses produced from the indicated plasmids encoding the indicated switch followed by a T2A cleavage site and a CAR specific for PSCA. After a rest period of eight days PSCA expressing HPAC pancratic tumor cells expressing RFP were cocultured with the CAR-T cells at and effector to target ratio of 1:20. Outgrowth of the RFP labeled tumor cells was measured in real time in an Incucyte microscope chamber.

The following plasmid and polypeptide sequences provide examples of sequences of polypeptides of the present embodiments. It is understood that certain costimulatory polypeptides may be substituted with others (that is, for example, a 4-1BB polypeptide cytoplasmic signaling region may be substituted with an OX40 polypeptide cytoplasmic signaling region, or the like; costimulatory polypeptide cytoplasmic signaling region amino acid and nucleotide sequences may be inserted, for example, at the “Additional signaling domain X” portion of the chimeric signling polypeptide sequence provided herein. Although the antigen recognition region of the chimeric antigen receptor provided in the following example binds to PSCA, it is understood that other antigen recognition regions that bind to antigens other than PSCA may be provided herein.

SFG-Myr-MyD88.Domain.Fv.Fv.T2A.aPSCAscFv.CD34e.CD8stm.zeta

| Fragment | Nucleotide | Peptide |
|---|---|---|
| Myristoy- | SEQ ID NO: 3 | SEQ ID NO: 4 |
| lation | ATGGGGAGTAGCAAGAGCAAGCCTAAGGACC | MGSSKSKPKDPSQR |
| peptide | CCAGCCAGCGC | |
| Linker | AGAGCATGC | RAC |
| MyD88 | SEQ ID NO: 1 | SEQ ID NO: 2 |
| | ATGgctgcaggaggtcccggcgcggggtctg | MAAGGPGAGSAAPVS |
| | cggccccggtctcctccacatcctcccttcc | STSSLPLAALNMRVR |
| | cctggctgctctcaacatgcgagtgcggcgc | RRLSLFLNVRTQVAA |
| | cgcctgtctctgttcttgaacgtgcggacac | DVVTALAEEMDFEYL |
| | aggtggcggccgactggaccgcgctggcgga | EIRQLETQADPTGRL |
| | ggagatggactttgagtacttggagatccgg | LDAVVQGRPGASVGR |
| | caactggagacacaagcggaccccactggca | LLDLLTKLGRDDVLL |
| | ggctgctggacgcctggcagggacgccctgg | ELGPSIEEDCQKYIL |
| | cgcctctgtaggccgactgctcgatctgctt | KQQQEEAEKPLQVAA |
| | accaagctgggccgcgacgacgtgctgctgg | VDSSVPRTAELAGIT |
| | agctgggacccagcattgaggaggattgcca | TLDDPLGHMPERFDA |
| | aaagtatatcttgaagcagcagcaggaggag | FICYCPSDI |
| | gctgagaagcctttacaggtggccgctgtag | |
| | acagcagtgtcccacggacagcagagctggc | |
| | gggcatcaccacacttgatgaccccctgggg | |
| | catatgcctgagcgtttcgatgccttcatct | |
| | gctattgccccagcgacatc | |
| Linker | Ctcgag | LE |
| Additional | | |
| Signaling | | |
| domain X | | |
| Linker | ggatctggcCAATTG | GSGQL |
| FKBPᵥ′ | SEQ ID NO: 5 | SEQ ID NO: 6 |
| | ggcgtccaagtcgaaaccattagtcccggcg | GVQVETISPGDGRTF |
| | atggcagaacatttcctaaaaggggacaaac | PKRGQTCVVHYTGML |
| | atgtgtcgtccattatacaggcatgttggag | EDGKKVDSSRDRNKP |
| | gacggcaaaaaggtggacagtagtaGaGAtc | FKFMLGKQEVIRGVV |
| | GcAAtAAaCCtTTcAAaTTcATGtTgGGaAA | EEGVAQMSVGQRAKL |
| | aCAaGAaGTcATtaGgGGaTGGGAgGAgGGc | TISPDYAYGATGHPG |
| | GTgGCtCAaATGtccGTcGGcCAacGcGCtA | IIPPHATLVFDVELL |
| | AgCTcACcATcagcCCcGAcTAcGCaTAcGG | KLE |
| | cGCtACcGGaCAtCCcGGaATtATtCCcCCt | |
| | CAcGCtACctTgGTgTTtGAcGTcGAaCTgt | |
| | TgAAgCTcGAa | |
| Linker | gtcgag | VE |
| FKBPᵥ | SEQ ID NO: 7 | SEQ ID NO: 8 |
| | ggagtgcaggtggagactatctccccaggag | GVQVETISPGDGRTF |
| | acgggcgcaccttccccaagcgcggccagac | PKRGQTCVVHYTGML |
| | ctgcgtggtgcactacaccgggatgcttgaa | EDGKKVDSSRDRNKP |
| | gatggaaagaaagttgattcctcccgggaca | FKFMLGKQEVIRGVV |
| | gaaacaagccctttaagtttatgctaggcaa | EEGVAQMSVGQRAKL |
| | gcaggaggtgatccgaggctgggaagaaggg | TISPDYAYGATGHPG |
| | gttgcccagatgagtgtgggtcagagagcca | IIPPHATLVFDVELL |
| | aactgactatatctccagattatgcctatgg | KLE |
| | tgccactgggcacccaggcatcatcccacca | |
| | catgccactctcgtcttcgatgtggagcttc | |
| | taaaactggaa | |
| Linker | ccgcGG | PR |
| T2A | SEQ ID NO: 9 | SEQ ID NO: 10 |
| | GAGGGCAGAGGCAGCCTCCTGACATGTGGGG | EGRGSLLTCGDVEEN |
| | ACGTCGAGGAGAACCCTGGCCCA | PGP |
| Linker | CCTTGG | PVV |
| Signal | ATGGAGTTCGGATTGAGCTGGCTGTTCCTGG | MEFGLSVVLFLVAIL |
| Peptide | TGGCAATACTCAAGGGCGTTCAATGTTCACG | KGVQCSR |
| | G | |
| PSCA(A11) V_{L} | GACATCCAACTGACGCAAAGCCCATCTACAC | DIQLTQSPSTLSASM |
| | TCAGCGCTAGCATGGGGGACAGGGTCACAAT | GDRVTITCSASSSVR |
| | CACGTGCTCTGCCTCAAGTTCCGTTAGGTTT | FIHVVYQQKPGKAPK |
| | ATCCATTGGTATCAGCAGAAACCTGGAAAGG | RLIYDTSKLASGVPS |
| | CCCCAAAAAGACTGATCTATGATACCAGCAA | RFSGSGSGTDFTLTI |
| | GCTGGCTTCCGGAGTGCCCTCAAGGTTCTCA | SSLQPEDFATYYCQQ |
| | GGATCTGGCAGTGGGACCGATTTCACCCTGA | VVGSSPFTFGQGTKV |
| | CAATTAGCAGCCTTCAGCCAGAGGATTTCGC | EIK |
| | AACCTATTACTGTCAGCAATGGGGGTCCAGC | |
| | CCATTCACTTTCGGCCAAGGAACAAAGGTGG | |
| | AGATAAAA | |
| Flex | GGCGGAGGAAGCGGAGGTGGGGGC | GGGSGGGG |
| Linker | CAGGTG | qv |
| PSCA(A11) V_{H} | GAGGTGCAGCTCGTGGAGTATGGCGGGGGCC | EVQLVEYGGGLVQPG |
| | TGGTGCAGCCTGGGGGTAGTCTGAGGCTCTC | GSLRLSCAASGFNIK |
| | CTGCGCTGCCTCTGGCTTTAACATTAAAGAC | DYYIHVVVRQAPGKG |
| | TACTACATACATTGGGTGCGGCAGGCCCCAG | LEVVVAVVIDPENGD |
| | GCAAAGGGCTCGAATGGGTGGCCTGGATTGA | TEFVPKFQGRATMSA |
| | CCCTGAGAATGGTGACACTGAGTTTGTCCCC | DTSKNTAYLQMNSLR |
| | AAGTTTCAGGGCAGAGCCACCATGAGCGCTG | AEDTAVYYCKTGGFV |
| | ACACAAGCAAAAACACTGCTTATCTCCAAAT | VGQGTLVTVSS |
| | GAATAGCCTGCGAGCTGAAGATACAGCAGTC | |
| | TATTACTGCAAGACGGGAGGATTCTGGGGCC | |
| | AGGGAACTCTGGTGACAGTTAGTTCC | |
| Linker | GGATCC | gs |
| CD34 | GAACTTCCTACTCAGGGGACTTTCTCAAACG | ELPTQGTFSNVSTNV |
| epitope | TTAGCACAAACGTAAGT | S |
| CD8 stalk | GAACTTCCTACTCAGGGGACTTTCTCAAACG | PAPRPPTPAPTIASQ |
| | TTAGCACAAACGTAAGT | PLSLRPEACRPAAGG |
| | | AVHTRGLDFACD |
| CD8 | ATCTATATCTGGGCACCTCTCGCTGGCACCT | IYIVVAPLAGTCGVL |
| transmembrane | GTGGAGTCCTTCTGCTCAGCCTGGTTATTAC | LLSLVITLYCNHRNR |
| | TCTGTACTGTAATCACCGGAATCGCCGCCGC | RRVCKCPR |
| | GTTTGTAAGTGTCCCAGG | |
| Linker | GTCGAC | VD |
| CD3ζ | AGAGTGAAGTTCAGCAGGAGCGCAGACGCCC | RVKFSRSADAPAYQQ |
| | CCGCGTACCAGCAGGGCCAGAACCAGCTCTA | GQNQLYNELNLGRRE |
| | TAACGAGCTCAATCTAGGACGAAGAGAGGAG | EYDVLDKRRGRDPEM |
| | TACGATGTTTTGGACAAGAGACGTGGCCGGG | GGKPRRKNPQEGLYN |
| | ACCCTGAGATGGGGGGAAAGCCGAGAAGGAA | ELQKDKMAEAYSEIG |
| | GAACCCTCAGGAAGGCCTGTACAATGAACTG | MKGERRRGKGHDGLY |
| | CAGAAAGATAAGATGGCGGAGGCCTACAGTG | QGLSTATKDTYDALH |
| | AGATTGGGATGAAAGGCGAGCGCCGGAGGGG | MQALPPR |
| | CAAGGGGCACGATGGCCTTTACCAGGGTCTC | |
| | AGTACAGCCACCAAGGACACCTACGACGCCC | |
| | TTCACATGCAAGCTCTTCCACCTCGT | |
| STOP | TGA | stop |

Fusions of Myr-MyD88 with ‘X’

| pBP | X candidates | Amino acid sequence of “X” |
|---|---|---|
| 2206 | CD40 | SEQ ID NO: 12 |
| | | KKVAKKPTNKAPHPKQEPOEINFPDD |
| | | LPGSNTAAPVQETLHGCQPVTQEDGK |
| | | ESRISVQERQ |
| 1849 | CD28 | SEQ ID NO: 14 |
| | | RSKRSRLLHSDYMNMTPRRPGPTRKH |
| | | YQPYAPPRDFAAYRSGSGQL |
| 1850 | 4-1BB | SEQ ID NO: 16 |
| | | KRGRKKLLYIFKQPFMRPVQTTQEED |
| | | GCSCRFPEEEEGGCEL |
| 2202 | OX40 | SEQ ID NO: 18 |
| | | RDQRLPPDAHKPPGGGSFRTPIQEEQ |
| | | ADAHSTLAKIGSG |
| 2203 | ICOS | SEQ ID NO: 20 |
| | | TKKKYSSSVHDPNGEYMFMRAVNTAK |
| | | KSRLTDVTL |

SFG-MyD88.Signalingdomain‘X’.Fv.Fv.T2A.OrangeNanoLanternReporter

| Fragment | Nucleotide | Peptide |
|---|---|---|
| MyD88 | ATGgctgcaggaggtcccggcgcggggtctgcg | MAAGGPGAGSAAPVSS |
| | gccccggtctcctccacatcctcccttcccctg | TSSLPLAALNMRVRRR |
| | gctgctctcaacatgcgagtgcggcgccgcctg | LSLFLNVRTQVAADVV |
| | tctctgttcttgaacgtgcggacacaggtggcg | TALAEEMDFEYLEIRQ |
| | gccgactggaccgcgctggcggaggagatggac | LETQADPTGRLLDAVV |
| | tttgagtacttggagatccggcaactggagaca | QGRPGASVGRLLDLLT |
| | caagcggaccccactggcaggctgctggacgcc | KLGRDDVLLELGPSIE |
| | tggcagggacgccctggcgcctctgtaggccga | EDCQKYILKQQQEEAE |
| | ctgctcgatctgcttaccaagctgggccgcgac | KPLQVAAVDSSVPRTA |
| | gacgtgctgctggagctgggacccagcattgag | ELAGITTLDDPLGHMP |
| | gaggattgccaaaagtatatcttgaagcagcag | ERFDAFICYCPSDI |
| | caggaggaggctgagaagcctttacaggtggcc | |
| | gctgtagacagcagtgtcccacggacagcagag | |
| | ctggcgggcatcaccacacttgatgaccccctg | |
| | gggcatatgcctgagcgtttcgatgccttcatc | |
| | tgctattgccccagcgacatc | |
| Linker | Ctcgag | LE |
| Signaling | | |
| domain X | | |
| Linker | ggatctggcCAATTG | GSGQL |
| FKBPᵥ′ | ggcgtccaagtcgaaaccattagtcccggcgat | GVQVETISPGDGRTFP |
| | ggcagaacatttcctaaaaggggacaaacatgt | KRGQTCVVHYTGMLED |
| | gtcgtccattatacaggcatgttggaggacggc | GKKVDSSRDRNKPFKF |
| | aaaaaggtggacagtagtaGaGAtcGcAAtAAa | MLGKQEVIRGVVEEGV |
| | CCtTTcAAaTTcATGtTgGGaAAaCAaGAaGTc | AQMSVGQRAKLTISPD |
| | ATtaGgGGaTGGGAgGAgGGcGTgGCtCAaATG | YAYGATGHPGIIPPHA |
| | tccGTcGGcCAacGcGCtAAgCTcACcATcagc | TLVFDVELLKLE |
| | CCcGAcTAcGCaTAcGGcGCtACcGGaCAtCCc | |
| | GGaATtATtCCcCCtCAcGCtACctTgGTgTTt | |
| | GAcGTcGAaCTgtTgAAgCTcGAa | |
| Linker | gtcgag | VE |
| FKBPᵥ | ggagtgcaggtggagactatctccccaggagac | GVQVETISPGDGRTFP |
| | gggcgcaccttccccaagcgcggccagacctgc | KRGQTCVVHYTGMLED |
| | gtggtgcactacaccgggatgcttgaagatgga | GKKVDSSRDRNKPFKF |
| | aagaaagttgattcctcccgggacagaaacaag | MLGKQEVIRGVVEEGV |
| | ccctttaagtttatgctaggcaagcaggaggtg | AQMSVGQRAKLTISPD |
| | atccgaggctgggaagaaggggttgcccagatg | YAYGATGHPGIIPPHA |
| | agtgtgggtcagagagccaaactgactatatct | TLVFDVELLKLE |
| | ccagattatgcctatggtgccactgggcaccca | |
| | ggcatcatcccaccacatgccactctcgtcttc | |
| | gatgtggagcttctaaaactggaa | |
| Linker | gcggccgctgggcgcgcc | AAAGRA |
| P2A | ggatctggAGCAACGAATTTTTCCCTGCTGAAA | GSGATNFSLLKQAGDV |
| | CAGGCAGGGGACGTAGAGGAAAATCCTGGTCCT | EENPGP |
| Linker | GCATGC | AC |
| hmKuspira | ATGGTCAGTGTGATCAAGCCAGAAATGAAGATG | MVSVIKPEMKMRYYMD |
| Orange | CGGTATTATATGGATGGGTCTGTAAACGGCCAC | GSVNGHEFTIEGEGTG |
| | GAATTTACCATCGAGGGCGAGGGGACGGGACGC | RPYEGHQEMTLRVTMA |
| | CCCTATGAGGGACACCAGGAAATGACTCTCAGG | EGGPMPFAFDLVSHVF |
| | GTGACAATGGCTGAGGGCGGCCCAATGCCATTT | CYGHRVFTKYPEEIPD |
| | GCTTTTGACCTGGTTTCACACGTGTTTTGCTAC | YFKQAFPEGLSVVERS |
| | GGACACAGGGTGTTCACTAAGTACCCAGAGGAG | LEFEDGGSASVSAHIS |
| | ATCCCGGATTATTTTAAACAGGCTTTTCCCGAG | LRGNTFYHKSKFTGVN |
| | GGCCTGAGCTGGGAAAGATCTCTGGAGTTTGAA | FPADGPIMQNQSVDVV |
| | GACGGGGGTAGCGCCAGCGTCTCAGCCCACATC | EPSTEKITASDGVLKG |
| | AGTCTTAGGGGAAATACCTTCTATCACAAGAGC | DVTMYLKLEGGGNHKC |
| | AAATTCACAGGGGTGAATTTTCCAGCTGATGGT | QMKTTYKAAKEILEMP |
| | CCAATCATGCAGAACCAGAGCGTGGATTGGGAA | GDHYIGHRLVRKTEGN |
| | CCTAGCACGGAGAAAATCACAGCCAGTGATGGG | ITEQVEDAVAHS |
| | GTGCTCAAAGGAGATGTGACCATGTATTTGAAA | |
| | CTGGAGGGGGGCGGCAATCATAAGTGTCAAATG | |
| | AAAACTACTTATAAAGCTGCTAAGGAAATCCTC | |
| | GAAATGCCGGGAGATCACTACATAGGGCATCGC | |
| | CTCGTCAGGAAAACTGAGGGGAACATTACAGAG | |
| | CAAGTGGAGGACGCCGTGGCCCATAGC | |
| Linker | ggtacc | gt |
| Renilla | AAGGTTTATGACCCTGAACAGCGCAAGAGAATG | KVYDPEQRKRMITGPQ |
| Luciferase | ATAACCGGCCCACAGTGGTGGGCACGATGTAAG | VVVVARCKQMNVLDSF |
| 8.6-535 | CAAATGAATGTACTCGACAGCTTTATTAACTAC | INYYDSEKHAENAVIF |
| | TATGACAGCGAAAAACATGCTGAGAACGCCGTG | LHGNATSSYLVVRHVV |
| | ATCTTCCTGCACGGCAACGCAACCAGCAGTTAC | PHIEPVARCIIPDLIG |
| | CTGTGGAGACACGTTGTACCTCACATTGAGCCT | MGKSGKSGNGSYRLLD |
| | GTTGCACGCTGCATCATCCCAGACTTGATTGGC | HYKYLTAVVFELLNLP |
| | ATGGGAAAATCTGGAAAGTCTGGGAATGGATCT | KKIIFVGHDVVGSALA |
| | TACAGACTGCTCGACCACTATAAGTATCTCACT | FHYAYEHQDRIKAIVH |
| | GCTTGGTTCGAGCTGCTGAACCTCCCAAAAAAA | MESVVDVIESVVMGVV |
| | ATCATCTTTGTGGGTCACGACTGGGGAAGTGCC | PDIEEELALIKSEEGE |
| | CTGGCATTCCACTATGCCTATGAACATCAGGAT | KMVLENNFFVETLLPS |
| | CGCATTAAAGCCATCGTCCATATGGAAAGTGTC | KIMRKLEPEEFAAYLE |
| | GTGGACGTGATCGAGAGTTGGATGGGATGGCCT | PFKEKGEVRRPTLSVV |
| | GACATCGAGGAGGAGCTGGCTCTCATCAAAAGC | PREIPLVKGGKPDVVQ |
| | GAAGAGGGGGAAAAGATGGTGTTGGAGAACAAC | IVRNYNAYLRASDDLP |
| | TTCTTTGTCGAGACCCTGCTGCCGTCTAAGATT | KLFIESDPGFFSNAIV |
| | ATGAGGAAGCTGGAACCAGAGGAATTTGCAGCC | EGAKKFPNTEFVKVKG |
| | TATTTGGAGCCCTTCAAGGAAAAGGGGGAGGTG | LHFLQEDAPDEMGKYI |
| | AGAAGGCCCACCCTCTCCTGGCCCCGCGAGATA | KSFVERVLKNEQ |
| | CCCCTGGTGAAGGGCGGGAAGCCTGACGTCGTC | |
| | CAGATCGTGCGGAATTACAACGCATATCTCAGA | |
| | GCCTCCGACGACCTGCCCAAGCTTTTTATTGAA | |
| | AGCGACCCTGGCTTTTTTTCAAATGCTATTGTC | |
| | GAGGGTGCCAAGAAGTTTCCAAATACCGAGTTC | |
| | GTGAAGGTGAAGGGACTGCATTTCTTGCAGGAG | |
| | GATGCCCCCGACGAGATGGGAAAATACATCAAG | |
| | TCTTTCGTGGAGCGCGTACTGAAGAATGAGCAG | |
| Stop | Taa | Stop translation |

Fusions of MyD88 with ‘X’

| pBP | X candidates | Amino acid sequence of “X” |
|---|---|---|
| 2173 | CD40 | KKVAKKPTNKAPHPKQEPQEINFPDDLPGSNTAAPVQETLHGCQP |
| | | VTQEDGKESRISVQERQ |
| 2181 | BCMA | SEQ ID NO: 22 |
| | | RKINSEPLKDEFKNTGSGLLGMANIDLEKSRTGDEIILPRGLEYT |
| | | VEECTCEDCIKSKPKVDSDHCFPLPAMEEGATILVTTKTNDYCKS |
| | | LPAALSATEIEKSISAR |
| 2182 | CD27 | SEQ ID NO: 24 |
| | | QRRKYRSNKGESPVEPAEPCRYSCPREEEGSTIPIQEDYRKPEPA |
| | | CSP |
| 2183 | CD30 | SEQ ID NO: 26 |
| | | HRRACRKRIRQKLHLCYPVQTSQPKLELVDSRPRRSSTQLRSGAS |
| | | VTEPVAEERGLMSQPLMETCHSVGAAYLESLPLQDASPAGGPSSP |
| | | RDLPEPRVSTEHTNNKIEKIYIMKADTVIVGTVKAELPEGRGLAG |
| | | PAEPELEEELEADHTPHYPEQETEPPLGSCSDVMLSVEEEGKEDP |
| | | LPTAASGK |
| 2184 | CD122 | SEQ ID NO: 28 |
| | | NCRNTGPVVLKKVLKCNTPDPSKFFSQLSSEHGGDVQKVVLSSPF |
| | | PSSSFSPGGLAPEISPLEVLERDKVTQMPPSLQERVPRDVVDPQP |
| | | LGPPTPGVPDLVDFQPPPELVLREAGEEVPDAGPREGVSFPVVSR |
| | | PPGQGEFRALNARLPLNTDAYLSLQELQGQDPTHLV |
| 2185 | GITR S180A | SEQ ID NO: 30 |
| | | QLGLHIVVQLRSQCMVVPRETQLLLEVPPATEDARSCQFPEEERG |
| | | ERSAEEKGRLGDLVVV |
| 2186 | GITR | SEQ ID NO: 32 |
| | | QLGLHIVVQLRSQCMVVPRETQLLLEVPPSTEDARSCQFPRVVRG |
| | | ERSAEEKGRLGDLVVV |
| 2187 | HVEM | SEQ ID NO: 34 |
| | | CVKRRKPRGDVVKVIVSVQRKRQEAEGEATVIEALQAPPDVTTVA |
| | | VEETIPSFTGRSPNH |
| 2188 | TACI | SEQ ID NO: 36 |
| | | CVKRRKPRGDVVKVIVSVQRKRQEAEGEATVIEALQAPPDVTTVA |
| | | VEETIPSFTGRSPNH |
| 2189 | TWEAKR | SEQ ID NO: 38 |
| 2190 | RANK | SEQ ID NO: 40 |
| | | CYRKKGKALTANLVVHVVINEACGRLSGDKESSGDSCVSTHTANF |
| | | GQQGACEGVLLLTLEEKTFPEDMCYPDQGGVCQGTCVGGGPYAQG |
| | | EDARMLSLVSKTEIEEDSFRQMPTEDEYMDRPSQPTDQLLFLTEP |
| | | GSKSTPPFSEPLEVGENDSLSQCFTGTQSTVGSESCNCTEPLCRT |
| | | DVVTPMSSENYLQKEVDSGHCPHVVAASPSPNVVADVCTGCRNPP |
| | | GEDCEPLVGSPKRGPLPQCAYGMGLPPEEEASRTEARDQPEDGAD |
| | | GRLPSSARAGAGSGSSPGGQSPASGNVTGNSNSTFISSGQVMNFK |
| | | GDIIVVYVSQTSQEGAAAAAEPMGRPVQEETLARRDSFAGNGPRF |
| | | PDPCGGPEGLREPEKASRPVQEQGGAKA |
| 2191 | RANK TRAF6 | SEQ ID NO: 42 |
| | | MLSLVSKTEIEEDSFRQMPTEDEYMDRPSQPTDQLLFLTEPGSKS |
| | | TPPFSEPLEVGENDSLSQCFTGTQSTVGSESCNCTEPLCRTDVVT |
| | | PMSSENYLQKEV |
| 2192 | RANK | SEQ ID NO: 44 |
| | TRAF2/5 | SNSTFISSGQVMNFKGDIIVVYVSQTSQEGAAAAAEPMGRPVQEE |
| | | TLARRDSFAGNGPRFPDPCGGPEGLREPEKASRPVQEQGGAKA |
| 2193 | RANK HCR- | SEQ ID NO: 46 |
| | TRAF2/5 | PEDGADGRLPSSARAGAGSGSSPGGQSPASGNVTGNSNSTFISSG |
| | | QVMNFKGDIIVVYVSQTSQEGAAAAAEPMGRPVQEETLARRDSFA |
| | | GNGPRFPDPCGGPEGLREPEKASRPVQEQGGAKA |
| 2194 | RANK | SEQ ID NO: 48 |
| | TRAF2/5 | CYRKKGKALTANLVVHVVINEACGRLSGDKESSGDSCVSTHTANF |
| | | GQQGACEGVLLLTLEEKTFPEDMCYPDQGGVCQGTCVGGGPYAQG |
| | | EDARMLSLVSKTEIEEDSFRQMPTEDEYMDRPSQPTDQLLFLTEP |
| | | GSKSTPPFSEPLEVGENDSLSQCFTGTQSTVGSESCNCTEPLCRT |
| | | DVVTPMSSENYLQKEVDSGHCPHVVAASPSPNVVADVCTGCRNPP |
| | | GEDCEPLVGSPKRGPLPQCAYGMGLPPEEEASRTEARDQPEDGAD |
| | | GRLPSSARAGAGSGSSPGGQSPASGNVTGNSNSTFISSGQVMNFK |
| | | GDIIVVYVSQTSQEGAAAAAEPMGRPVQEETLARRDSFAGNGPRF |
| | | PDPCGGPEGLREPEKASRPVQEQGGAKA |
| 2195 | CD4O-HCR- | SEQ ID NO: 50 |
| | CD40 | KKVAKKPTNKAPHPKQEPQEINFPDDLPGPEDGADGRLPSSARAG |
| | | AGSGSSPGGQSPASGNVTGNSNSTFISSGQVMNFKGDIIVVYVSQ |
| | | TSQESNTAAPVQETLHGCQPVTQEDGKESRISVQERQV |
| 2196 | SOD 1 | SEQ ID NO: 52 |
| | | MATKAVCVLKGDGPVQGIINFEQKESNGPVKVVVGSIKGLTEGLH |
| | | GFHVHEFGDNTAGCTSAGPHFNPLSRKHGGPKDEERHVGDLGNVT |
| | | ADKDGVADVSIEDSVISLSGDHCIIGRTLVVHEKADDLGKGGNEE |
| | | STKTGNAGSRLACGVIGIAQ |
| 2197 | SOD 2 | SEQ ID NO: 54 |
| | | MLSRAVCGTSRQLAPALGYLGSRQKHSLPDLPYDYGALEPHINAQ |
| | | IMQLHHSKHHAAYVNNLNVTEEKYQEALAKGRFQAERREAVPGRG |
| | | DPREPGPIRTGLSVEENSLRICTGSEFSRHDSLSFKHMVYLIVEG |
| | | VPRVVV* |
| 2198 | SOD 3 | SEQ ID NO: 56 |
| | | VVTGEDSAEPNSDSAEVVIRDMYAKVTEIVVQEVMQRRDDDGALH |
| | | AACQVQPSATLDAAQPRVTGVVLFRQLAPRAKLDAFFALEGFPTE |
| | | PNSSSRAIHVHQFGDLSQGCESTGPHYNPLAVPHPQHPGDFGNFA |
| | | VRDGSLVVRYRAGLAASLAGPHSIVGRAVVVHAGEDDLGRGGNQA |
| | | SVENGNAGRRLACCVVGVCGPGLVVERQAREHSERKKRRRESECK |
| | | AA |
| 2606 | RANK88 | SEQ ID NO: 58 |
| | | PEDGADGRLPSSARAGAGSGSSPGGQSPASGNVTGNSNSTFISSG |
| | | QVMNFKGDIIVVYVSQTSQESNTAAPVQETLHGCQPVTQEDGKES |
| | | RISVQERQ |
| 2607 | 4-1BB | SEQ ID NO: 60 |
| | | KRGRKKLLYIFKQPFMRPVQTTQEEDGCSCRFPEEEEGGCEL |
| 2608 | OX40 | SEQ ID NO: 62 |
| | | ALYLLRRDQRLPPDAHKPPGGGSFRTPIQEEQADAHSTLAKI |
| 2609 | IL15 Ra | SEQ ID NO: 64 |
| | | KSRQTPPLASVEMEAMEALPVTVVGTSSRDEDLENCSHHL |
| 2610 | ICOS | SEQ ID NO: 66 |
| | | TGTTGGCTCACCAAAAAGAAGTATTCTTCATCAGTGCATGACCCT |
| | | AACGGCGAGTATATGTTTATGCGGGCGGTCAATACGGCCAAGAAG |
| | | TCCCGGCTTACCGACGTTACTTTG |
| 2611 | BTN3A1 | SEQ ID NO: 68 |
| | | QQQEEKKTQFRKKKREQELREMAVVSTMKQEQSTRVKLLEELRVV |
| | | RSIQYASRGERHSAYNEVVKKALFKPADVILDPKTANPILLVSED |
| | | QRSVQRAKEPQDLPDNPERFNVVHYCVLGCESFISGRHYVVEVEV |
| | | GDRKEVVHIGVCSKNVQRKGVVVKMTPENGFVVTMGLTDGNKYRT |
| | | LTEPRTNLKLPKPPKKVGVFLDYETGDISFYNAVDGSHIHTFLDV |
| | | SFSEALYPVFRILTLEPTALTICPA |
| 2612 | BTN3A1 | SEQ ID NO: 70 |
| | B30.2 | RGERHSAYNEVVKKALFKPADVILDPKTANPILLVSEDQRSVQRA |
| | domain | KEPQDLPDNPERFNVVHYCVLGCESFISGRHYVVEVEVGDRKEVV |
| | only | HIGVCSKNVQRKGVVVKMTPENGFVVTMGLTDGNKYRTLTEPRTN |
| | | LKLPKPPKKVGVFLDYETGDISFYNAVDGSHIHTFLDVSFSEALY |
| | | PVFRILTLEPTALTICPA |
| 2615 | CD86 | SEQ ID NO: 72 |
| | | KVVKKKKRPRNSYKCGTNTMEREESEQTKKREKIHIPERSDEAQR |
| | | VFKSSKTSSCDKSDTCF |
| 2617 | CD74Ra | SEQ ID NO: 74 |
| | | MHRRRSRSCREDQKPVMDDQRDLISNNEQLPMLGRRPGAPESKCS |
| 2619 | Dectin | SEQ ID NO: 76 |
| | | MEYHPDLENLDEDGYTQLHFDSQSNTRIAVVSEKGSCAASPPVVR |
| 2620 | CD40 DPP4 | SEQ ID NO: 78 |
| | | KKVAKKPTNKAPHPKQEPQEINFPDDLPGSNTAAPVQETLHGCQP |
| | | VTQEDGKESRISVQERQ |
| 2621 | ITA4 | SEQ ID NO: 80 |
| | | AGFFKRQYKSILQEENRRDSVVSYINSKSNDD |
| 2622 | ITGA5 | SEQ ID NO: 82 |
| | | KLGFFKRSLPYGTAMEKAQLKPPATSDA |
| 2623 | TCL1A | SEQ ID NO: 84 |
| | | RKVVCQYQKEIMERPPPFKPPPPPIKYTCIQEPNESDLPYHEMET |
| | | L |
| 2638 | CTLA4 | SEQ ID NO: 86 |
| | | AVSLSKMLKKRSPLTTGVYVKMPPTEPECEKQFQPYFIPIN |
| 2639 | TIM1 | SEQ ID NO: 88 |
| | | KKYFFKKEVQQLSVSFSSLQIKALQNAVEKEVQAEDNIYIENSLY |
| | | ATD |
| 2640 | TREMBL2 | SEQ ID NO: 90 |
| | | KKRHMASYSMCSDPSTRDPPGRPEPYVEVYLI |

pBP2261-SFG-aHER2scFv.CD34e.CD8stm.zeta

| Fragment | Nucleotide | Peptide |
|---|---|---|
| Signal | ATGGATTTTCAAGTGCAGATTTTCAGCTTTCT | MDFQVQIFSFLLISAS |
| Peptide | CTTGATCAGTGCcAGCGTGATCATGAGCAGGG | VIMSRG |
| | GC | |
| HER2(4D5) V_{L} | GACATCCAGATGACTCAATCACCCTCAAGTCT | DIQMTQSPSSLSASVG |
| | GAGTGCATCAGTGGGCGACCGAGTAACAATTA | DRVTITCRASQDVNTA |
| | CGTGCCGGGCCTCTCAGGATGTCAACACCGCC | VAVVYQQKPGKAPKLL |
| | GTGGCCTGGTAtCAACAAAAACCTGGAAAGGC | IYSASFLYSGVPSRFS |
| | CCCTAAGCTCCTCATATATAGCGCCTCATTCC | GSRSGTDFTLTISSLQ |
| | TTTATTCTGGTGTACCTAGTCGCTTTTCAGGG | PEDFATYYCQQHYTTP |
| | TCAAGATCAGGAACCGATTTTACACTTACAAT | PTFGQGTKVEIKRTGS |
| | CTCTAGTCTGCAACCAGAGGATTTTGCTACCT | TSGSGKPGSGEGS |
| | ACTATTGCCAGCAACACTATACAACGCCGCCT | |
| | ACATTCGGGCAGGGGACGAAGGTGGAGATTAA | |
| | ACGCACT | |
| Flex | GGCTCAACcAGTGGGTCAGGCAAGCCTGGgTC | GSTSGSGKPGSGEGS |
| | CGGCGAGGGCTCC | |
| HER2(4D5) V_{H} | GAGGTACAACTGGTTGAAAGCGGAGGAGGGCT | EVQLVESGGGLVQPGG |
| | GGTTCAGCCAGGCGGGTCACTCCGCCTCTCCT | SLRLSCAASGFNIKDT |
| | GTGCGGCGTCCGGGTTCAACATAAAGGACACC | YIHVVVRQAPGKGLEV |
| | TATATCCATTGGGTTCGCCAGGCCCCCGGAAA | VVARIYPTNGYTRYAD |
| | GGGATTGGAGTGGGTTGCTCGAATTTATCCAA | SVKGRFTISADTSKNT |
| | CTAATGGGTACACCCGCTATGCTGATTCCGTG | AYLQMNSLRAEDTAVY |
| | AAGGGTCGCTTTACGATTAGTGCCGACACTTC | YCSRVVGGDGFYAMDF |
| | gAAAAACACAGCTTACTTGCAAATGAACAGTC | VVGQGTLVTVSS |
| | TCCGCGCCGAGGACACCGCAGTATACTATTGC | |
| | AGTAGGTGGGGGGGAGACGGGTTTTATGCTAT | |
| | GGATtTCTGGGGACAAGGTACTCTGGTAACCG | |
| | TGAGCAGT | |
| Linker | GGATCC | gs |
| CD34 | GAACTTCCTACTCAGGGGACTTTCTCAAACGT | ELPTQGTFSNVSTNVS |
| epitope | TAGCACAAACGTAAGT | |
| CD8 stalk | GAACTTCCTACTCAGGGGACTTTCTCAAACGT | PAPRPPTPAPTIASQP |
| | TAGCACAAACGTAAGT | LSLRPEACRPAAGGAV |
| | | HTRGLDFACD |
| CD8 | ATCTATATCTGGGCACCTCTCGCTGGCACCTG | IYIVVAPLAGTCGVLL |
| transmembrane | TGGAGTCCTTCTGCTCAGCCTGGTTATTACTC | LSLVITLYCNHRNRRR |
| | TGTACTGTAATCACCGGAATCGCCGCCGCGTT | VCKCPR |
| | TGTAAGTGTCCCAGG | |
| Linker | GTCGAC | VD |
| CD3ζ | AGAGTGAAGTTCAGCAGGAGCGCAGACGCCCC | RVKFSRSADAPAYQQG |
| | CGCGTACCAGCAGGGCCAGAACCAGCTCTATA | QNQLYNELNLGRREEY |
| | ACGAGCTCAATCTAGGACGAAGAGAGGAGTAC | DVLDKRRGRDPEMGGK |
| | GATGTTTTGGACAAGAGACGTGGCCGGGACCC | PRRKNPQEGLYNELQK |
| | TGAGATGGGGGGAAAGCCGAGAAGGAAGAACC | DKMAEAYSEIGMKGER |
| | CTCAGGAAGGCCTGTACAATGAACTGCAGAAA | RRGKGHDGLYQGLSTA |
| | GATAAGATGGCGGAGGCCTACAGTGAGATTGG | TKDTYDALHMQALPPR |
| | GATGAAAGGCGAGCGCCGGAGGGGCAAGGGGC | |
| | ACGATGGCCTTTACCAGGGTCTCAGTACAGCC | |
| | ACCAAGGACACCTACGACGCCCTTCACATGCA | |
| | AGCTCTTCCACCTCGT | |
| STOP | TGA | stop |

### Example 6: Selective Apoptosis of the Modified Cells

The modified cells may be provided with a mechanism to remove some, or all of the cells if the patient experiences negative effects, and there is a need to reduce, or stop treatment. This mechanism cells may be used for the modified cells discussed herein. In some examples, the cells may be provided with this ability where the CAR is directed against antigens that have previously caused, or are at risk to cause, lethal on-target, off-organ toxicity, where there is a need for an option to rapidly terminate therapy. Methods and compositions for selective apoptosis are discussed in, for example, U.S. Pat. No. 9,089,520, titled Methods for Inducing Selective Apoptosis, to Brenner, M. K., issued Jul. 28, 2015; U.S. Pat. No. 9,434,935, titled Modified Caspase Polypeptides and Uses Thereof, to Spencer, D., et al., issued Sep. 6, 2016; U.S. patent application Ser. No. 14/296,404, filed Jun. 4, 2014, titled Methods for Inducing Partial Apoptosis Using Caspase Polypeptides, by Slawin, K., et al., published as US2016-0151465A1 on Jun. 2, 2016; International Patent Application PCT/US2014/040964, filed Jun. 4, 2014, published as WO2014/197638 on Feb. 5, 2015; U.S. patent application Ser. No. 14/640,553, filed Mar. 6, 2015, titled Caspase Polypeptides having Modified Activity and Uses Thereof, by Spencer, D., et al., published as US-2015-0328292A1 on Nov. 19, 2015; and International Patnet Application PCT/US15/19186 filed Mar. 6, 2015, published as WO2015/134877 on Sep. 11, 2015, each of which is incorporated by reference herein in its entirety.

A chimeric signaling polypeptide discussed herein may, for example, be encoded by a nucleic acid vector as part of a single polypeptide that also encodes an inducible Caspase-9 polypeptide. Examples of chimeric polypeptides that may be expressed in the modified cells are provided in FIG. 10. In this example, a single polypeptide is encoded by the nucleic acid vector. The inducible Caspase-9 polypeptide is separated from the CAR polypeptide during translation, due to skipping of a peptide bond. (Donnelly, M L 2001, J. Gen. Virol. 82:1013-25).

Vector Construction and Confirmation of Expression

A safety switch that can be stably and efficiently expressed in human T cells is presented herein. Expression vectors suitable for use as a therapeutic agent were constructed that included a modified human Caspase-9 activity fused to a human FK506 binding protein (FKBP), such as, for example, FKBP12v36. The Caspase-9/FKBP12 hybrid activity can be dimerized using a small molecule pharmaceutical. Full length, truncated, and modified versions of the Caspase-9 activity were fused to the ligand binding domain, multimerizing, dimerizing, dimerization, or multimerization region, and inserted into the retroviral vector MSCV.IRES.GRP, which also allows expression of the fluorescent marker, GFP.

The full-length inducible Caspase-9 molecule (F′-F-C-Casp9) includes 2, 3, or more FK506 binding proteins (FKBPs—for example, FKBP12v36 variants) linked with a Ser-Gly-Gly-Gly-Ser-Gly linker to the small and large subunit of the caspase molecule. Full-length inducible Caspase-9 (F′F-C-Casp9.I.GFP) has a full-length Caspase-9, also includes a caspase recruitment domain (CARD; GenBank NM001 229) linked to 2 12-kDa human FK506 binding proteins (FKBP12; GenBank AH002 818) that contain an F36V mutation. The amino acid sequence of one or more of the FKBPs (F′) was codon-wobbled (e.g., the 3rd nucleotide of each amino acid codon was altered by a silent mutation that maintained the originally encoded amino acid) to prevent homologous recombination when expressed in a retrovirus. F′F-C-Casp9C3S includes a cysteine to serine mutation at position 287 that disrupts its activation site. In constructs F′F-Casp9, F-C-Casp9, and F′-Casp9, either the caspase activation domain (CARD), one FKBP, or both, were deleted, respectively. All constructs were cloned into MSCV.IRES.GFP as EcoRI-XhoI fragments. Coexpression of the inducible Caspase-9 constructs of the expected size with the marker gene GFP in transfected 293T cells was demonstrated by Western blot using a Caspase-9 antibody specific for amino acid residues 299-318, present both in the full-length and truncated caspase molecules as well as a GFP-specific antibody.

An initial screen indicated that the full length iCasp9 could not be maintained stably at high levels in T cells, possibly due to transduced cells being eliminated by the basal activity of the transgene. The CARD domain is involved in physiologic dimerization of Caspase-9 molecules, by a cytochrome C and adenosine triphosphate (ATP)—driven interaction with apoptotic protease-activating factor 1 (Apaf-1). Because of the use of a CID to induce dimerization and activation of the suicide switch, the function of the CARD domain is superfluous in this context and removal of the CARD domain was investigated as a method of reducing basal activity.

Using the iCasp9 Suicide Gene to Improve the Safety of Allodepleted T Cells after Haploidentical Stem Cell Transplantation

Presented in this example are expression constructs and methods of using the expression constructs to improve the safety of allodepleted T cells after haploidentical stem cell transplantation. Similar methods may be used to express the Caspase-9 expression constructs in non allodepleted cells. A retroviral vector encoding iCasp9 and a selectable marker (truncated CD19) was generated as a safety switch for donor T cells. Even after allodepletion (using anti-CD25 immunotoxin), donor T cells could be efficiently transduced, expanded, and subsequently enriched by CD19 immunomagnetic selection to >90% purity. The engineered cells retained anti-viral specificity and functionality, and contained a subset with regulatory phenotype and function. Activating iCasp9 with a small-molecule dimerizer rapidly produced >90% apoptosis. Although transgene expression was downregulated in quiescent T cells, iCasp9 remained an efficient suicide gene, as expression was rapidly upregulated in activated (alloreactive) T cells.

Materials and Methods

Generation of Allodepleted T Cells

Allodepleted cells were generated from healthy volunteers as previously presented. Briefly, peripheral blood mononuclear cells (PBMCs) from healthy donors were co-cultured with 30 Gγ-irradiated recipient Epstein Barr virus (EBV)-transformed lymphoblastoid cell lines (LCL) at responder-to-stimulator ratio of 40:1 in serum-free medium (AIM V; Invitrogen, Carlsbad, Calif.). After 72 hours, activated T cells that expressed CD25 were depleted from the co-culture by overnight incubation in RFT5-SMPT-dgA immunotoxin. Allodepletion was considered adequate if the residual CD3⁺CD25⁺ population was <1% and residual proliferation by 3H-thymidine incorporation was <10%.

Plasmid and Retrovirus

SFG.iCasp9.2A.CD19 consists of inducible Caspase-9 (iCasp9) linked, via a cleavable 2A-like sequence, to truncated human CD19. iCasp9 consists of a human FK506-binding protein (FKBP12; GenBank AH002 818) with an F36V mutation, connected via a Ser-Gly-Gly-Gly-Ser-Gly linker to human Caspase-9 (CASP9; GenBank NM 001229). The F36V mutation increases the binding affinity of FKBP12 to the synthetic homodimerizer, AP20187 or rimiducid. The caspase recruitment domain (CARD) has been deleted from the human Caspase-9 sequence because its physiological function has been replaced by FKBP12, and its removal increases transgene expression and function. The 2A-like sequence encodes an 20 amino acid peptide from Thosea asigna insect virus, which mediates >99% cleavage between a glycine and terminal proline residue, resulting in 19 extra amino acids in the C terminus of iCasp9, and one extra proline residue in the N terminus of CD19. CD19 consists of full-length CD19 (GenBank NM 001770) truncated at amino acid 333 (TDPTRRF), which shortens the intracytoplasmic domain from 242 to 19 amino acids, and removes all conserved tyrosine residues that are potential sites for phosphorylation.

A stable PG13 clone producing Gibbon ape leukemia virus (Gal-V) pseudotyped retrovirus was made by transiently transfecting Phoenix Eco cell line (ATCC product #SD3444; ATCC, Manassas, Va.) with SFG.iCasp9.2A.CD19. This produced Eco-pseudotyped retrovirus. The PG13 packaging cell line (ATCC) was transduced three times with Eco-pseudotyped or retrovirus to generate a producer line that contained multiple SFG.iCasp9.2A.CD19 proviral integrants per cell. Single cell cloning was performed, and the PG13 clone that produced the highest titer was expanded and used for vector production.

Retroviral Transduction

Culture medium for T cell activation and expansion consisted of 45% RPMI 1640 (Hyclone, Logan, Utah), 45% Clicks (Irvine Scientific, Santa Ana, Calif.) and 10% fetal bovine serum (FBS; Hyclone). Allodepleted cells were activated by immobilized anti-CD3 (OKT3; Ortho Biotech, Bridgewater, N.J.) for 48 hours before transduction with retroviral vector Selective allodepletion was performed by co-culturing donor PBMC with recipient EBV-LCL to activate alloreactive cells: activated cells expressed CD25 and were subsequently eliminated by anti-CD25 immunotoxin. The allodepleted cells were activated by OKT3 and transduced with the retroviral vector 48 hours later.

Immunomagnetic selection was performed on day 4 of transduction; the positive fraction was expanded for a further 4 days and cryopreserved.

In small-scale experiments, non-tissue culture-treated 24-well plates (Becton Dickinson, San Jose, Calif.) were coated with OKT3 1 g/ml for 2 to 4 hours at 37° C. Allodepleted cells were added at 1×10⁶ cells per well. At 24 hours, 100 U/ml of recombinant human interleukin-2 (IL-2) (Proleukin; Chiron, Emeryville, Calif.) was added. Retroviral transduction was performed 48 hours after activation. Non-tissue culture-treated 24-well plates were coated with 3.5 μg/cm2 recombinant fibronectin fragment (CH-296; Retronectin; Takara Mirus Bio, Madison, Wis.) and the wells loaded twice with retroviral vector-containing supernatant at 0.5 ml per well for 30 minutes at 37° C., following which OKT3-activated cells were plated at 5×10⁵ cells per well in fresh retroviral vector-containing supernatant and T cell culture medium at a ratio of 3:1, supplemented with 100 U/ml IL-2. Cells were harvested after 2 to 3 days and expanded in the presence of 50 U/ml IL-2.

Scaling-Up Production of Gene-Modified Allodepleted Cells

Scale-up of the transduction process for clinical application used non-tissue culture-treated T75 flasks (Nunc, Rochester, N.Y.), which were coated with 10 ml of OKT3 1 μg/ml or 10 ml of fibronectin 7 μg/ml at 4° C. overnight. Fluorinated ethylene propylene bags corona-treated for increased cell adherence (2PF-0072AC, American Fluoroseal Corporation, Gaithersburg, Md.) were also used. Allodepleted cells were seeded in OKT3-coated flasks at 1×10⁶ cells/ml. 100 U/ml IL-2 was added the next day. For retroviral transduction, retronectin-coated flasks or bags were loaded once with 10 ml of retrovirus-containing supernatant for 2 to 3 hours. OKT3-activated T cells were seeded at 1×10⁶ cells/ml in fresh retroviral vector-containing medium and T cell culture medium at a ratio of 3:1, supplemented with 100 U/ml IL-2. Cells were harvested the following morning and expanded in tissue-culture treated T75 or T175 flasks in culture medium supplemented with between about 50 to 100 U/ml IL-2 at a seeding density of between about 5×10⁵ cells/ml to 8×10⁵ cells/ml.

CD19 Immunomagnetic Selection

Immunomagnetic selection for CD19 was performed 4 days after transduction. Cells were labeled with paramagnetic microbeads conjugated to monoclonal mouse anti-human CD19 antibodies (Miltenyi Biotech, Auburn, Calif.) and selected on MS or LS columns in small scale experiments and on a CliniMacs Plus automated selection device in large scale experiments. CD19-selected cells were expanded for a further 4 days and cryopreserved on day 8 post transduction. These cells were referred to as “gene-modified allodepleted cells”.

Immunophenotyping and Pentamer Analysis

Flow cytometric analysis (FACSCalibur and CellQuest software; Becton Dickinson) was performed using the following antibodies: CD3, CD4, CD8, CD19, CD25, CD27, CD28, CD45RA, CD45RO, CD56 and CD62L. CD19-PE (Clone 4G7; Becton Dickinson) was found to give optimum staining and was used in all subsequent analysis. A Non-transduced control was used to set the negative gate for CD19. An HLA-pentamer, HLA-B8-RAKFKQLL (Proimmune, Springfield, Va.) was used to detect T cells recognizing an epitope from EBV lytic antigen (BZLF1). HLA-A2-NLVPMVATV pentamer was used to detect T cells recognizing an epitope from CMV-pp65 antigen.

Induction of Apoptosis with Chemical Inducer of Dimerization, AP20187

Suicide gene functionality was assessed by adding a small molecule synthetic homodimerizer, AP20187 (Ariad Pharmaceuticals; Cambridge, Mass.), at 10 nM final concentration the day following CD19 immunomagnetic selection. Rimiducid may also be used. Cells were stained with annexin V and 7-amino¬actinomycin (7-AAD) (BD Pharmingen) at 24 hours and analyzed by flow cytometry. Cells negative for both annexin V and 7-AAD were considered viable, cells that were annexin V positive were apoptotic, and cells that were both annexin V and 7-AAD positive were necrotic. The percentage killing induced by dimerization was corrected for baseline viability as follows:

Percentage killing=100%−(% Viability in AP20187-treated cells÷% Viability in non-treated cells).

Assessment of Transgene Expression Following Extended Culture and Reactivation

Cells were maintained in T cell medium containing 50 U/ml IL-2 until 22 days after transduction. A portion of cells was reactivated on 24-well plates coated with 1 g/ml OKT3 and 1 μg/ml anti-CD28 (Clone CD28.2, BD Pharmingen, San Jose, Calif.) for 48 to 72 hours. CD19 expression and suicide gene function in both reactivated and non-reactivated cells were measured on day 24 or 25 post transduction.

In some experiments, cells also were cultured for 3 weeks post transduction and stimulated with 30G-irradiated allogeneic PBMC at a responder: stimulator ratio of 1:1. After 4 days of co-culture, a portion of cells was treated with 10 nM AP20187. Killing was measured by annexin V/7-AAD staining at 24 hours, and the effect of dimerizer on bystander virus-specific T cells was assessed by pentamer analysis on AP20187-treated and untreated cells.

Optimal culture conditions for maintaining the immunological competence of suicide gene-modified T cells must be determined and defined for each combination of safety switch, selectable marker and cell type, since phenotype, repertoire and functionality can all be affected by the stimulation used for polyclonal T cell activation, the method for selection of transduced cells, and duration of culture.

Phase I Clinical Trial of Allodepleted T Cells Transduced with Inducible Caspase-9 Suicide Gene after Haploidentical Stem Cell Transplantation

This example presents results of a phase 1 clinical trial using an alternative suicide gene strategy. Briefly, donor peripheral blood mononuclear cells were co-cultured with recipient irradiated EBV-transformed lymphoblastoid cells (40:1) for 72 hrs., allodepleted with a CD25 immunotoxin and then transduced with a retroviral supernatant carrying the iCasp9 suicide gene and a selection marker (ΔCD19); ΔCD19 allowed enrichment to >90% purity via immunomagnetic selection.

An Example of a Protocol for Generation of a Cell Therapy Product is Provided Herein.

Source Material

Up to 240 ml (in 2 collections) of peripheral blood was obtained from the transplant donor according to established protocols. In some cases, dependent on the size of donor and recipient, a leukopheresis was performed to isolate sufficient T cells. 10-30cc of blood also was drawn from the recipient and was used to generate the Epstein Barr virus (EBV)-transformed lymphoblastoid cell line used as stimulator cells. In some cases, dependent on the medical history and/or indication of a low B cell count, the LCLs were generated using appropriate 1st degree relative (e.g., parent, sibling, or offspring) peripheral blood mononuclear cells.

Generation of Allodepleted Cells

Allodepleted cells were generated from the transplant donors as presented herein. Peripheral blood mononuclear cells (PBMCs) from healthy donors were co-cultured with irradiated recipient Epstein Barr virus (EBV)-transformed lymphoblastoid cell lines (LCL) at responder-to-stimulator ratio of 40:1 in serum-free medium (AIM V; Invitrogen, Carlsbad, Calif.). After 72 hours, activated T cells that express CD25 were depleted from the co-culture by overnight incubation in RFT5-SMPT-dgA immunotoxin. Allodepletion is considered adequate if the residual CD3⁺CD25⁺ population was <1% and residual proliferation by 3H-thymidine incorporation was <10%.

Retroviral Production

A retroviral producer line clone was generated for the iCasp9-ΔCD19 construct. A master cell-bank of the producer also was generated. Testing of the master-cell bank was performed to exclude generation of replication competent retrovirus and infection by Mycoplasma, HIV, HBV, HCV and the like. The producer line was grown to confluency, supernatant harvested, filtered, aliquoted and rapidly frozen and stored at −80° C. Additional testing was performed on all batches of retroviral supernatant to exclude Replication Competent Retrovirus (RCR) and issued with a certificate of analysis, as per protocol.

Transduction of Allodepleted Cells

Allodepleted T-lymphocytes were transduced using Fibronectin. Plates or bags were coated with recombinant Fibronectin fragment CH-296 (Retronectin™, Takara Shuzo, Otsu, Japan). Virus was attached to retronectin by incubating producer supernatant in coated plates or bags. Cells were then transferred to virus coated plates or bags. After transduction allodepleted T cells were expanded, feeding them with IL-2 twice a week to reach the sufficient number of cells as per protocol.

CD19 Immunomagnetic Selection

Immunomagnetic selection for CD19 was performed 4 days after transduction. Cells are labeled with paramagnetic microbeads conjugated to monoclonal mouse anti-human CD19 antibodies (Miltenyi Biotech, Auburn, Calif.) and selected on a CliniMacs Plus automated selection device.

Depending upon the number of cells required for clinical infusion cells were either cryopreserved after the CliniMacs selection or further expanded with IL-2 and cryopreserved on day 6 or day 8 post transduction.

Freezing

Aliquots of cells were removed for testing of transduction efficiency, identity, phenotype and microbiological culture as required for final release testing by the FDA. The cells were cryopreserved prior to administration according to protocol.

Study Drugs

RFT5-SMPT-dgA

RFT5-SMPT-dgA is a murine IgG1 anti-CD25 (IL-2 receptor a chain) conjugated via a hetero-bifunctional crosslinker [N-succinimidyloxycarbonyl-α-methyl-d- (2-pyridylthio) toluene] (SMPT) to chemically deglycosylated ricin A chain (dgA). RFT5-SMPT-dgA is formulated as a sterile solution at 0.5 mg/ml.

Synthetic Homodimerizer, Rimiducid

Mechanism of Action: rimiducid-inducible cell death is achieved by expressing a chimeric protein comprising the pro-domain-deleted portion of human (Caspase-9) protein receptor, which signals apoptotic cell death, fused to a drug-binding domain derived from human FK506-binding protein (FKBP). This chimeric protein remains quiescent inside cells until administration of rimiducid, which cross-links the FKBP domains, initiating caspase signaling and apoptosis.

Toxicology: rimiducid has been evaluated as an Investigational New Drug (IND) by the FDA and has successfully completed a phase I clinical safety study. No significant adverse effects were noted when rimiducid was administered over a 0.01 mg/kg to 1.0 mg/kg dose range.

Pharmacology/Pharmacokinetics: Patients received 0.4 mg/kg of rimiducid as a 2 h infusion-based on published PK data which show plasma concentrations of 10 ng/mL-I275 ng/mL over the 0.01 mg/kg to 1.0 mg/kg dose range with plasma levels falling to 18% and 7% of maximum at 0.5 and 2 hrs post dose.

Side Effect Profile in Humans: No serious adverse events occurred during the Phase 1 study in volunteers. The incidence of adverse events was very low following each treatment, with all adverse events being mild in severity. Only one adverse event was considered possibly related to AP1903. This was an episode of vasodilatation, presented as “facial flushing” for 1 volunteer at the 1.0 mg/kg AP1903 dosage. This event occurred at 3 minutes after the start of infusion and resolved after 32 minutes duration. All other adverse events reported during the study were considered by the investigator to be unrelated or to have improbable relationship to the study drug. These events included chest pain, flu syndrome, halitosis, headache, injection site pain, vasodilatation, increased cough, rhinitis, rash, gum hemorrhage, and ecchymosis.

Patients developing grade 1 GvHD were treated with 0.4 mg/kg AP1903 as a 2-hour infusion. Protocols for administration of rimiducid to patients grade 1 GvHD were established as follows. Patients developing GvHD after infusion of allodepleted T cells are biopsied to confirm the diagnosis and receive 0.4 mg/kg of rimiducid as a 2 h infusion. Patients with Grade I GvHD received no other therapy initially, however if they showed progression of GvHD conventional GvHD therapy was administered as per institutional guidelines. Patients developing grades 2-4 GvHD were administered standard systemic immunosuppressive therapy per institutional guidelines, in addition to the rimiducid dimerizer drug.

Instructions for preparation and infusion: rimiducid for injection is obtained as a concentrated solution of 2.33 ml in a 3 ml vial, at a concentration of 5 mg/mi, (i.e., 10.66 mg per vial). Prior to administration, the calculated dose was diluted to 100 mL in 0.9% normal saline for infusion. Rimiducid for injection (0.4 mg/kg) in a volume of 100 ml was administered via IV infusion over 2 hours, using a non-DEHP, non-ethylene oxide sterilized infusion set and infusion pump.

The iCasp9 suicide gene expression construct (e.g., SFG.iCasp9.2A.ΔCD19) consists of inducible Caspase-9 (iCasp9) linked, via a cleavable 2A-like sequence, to truncated human CD19 (ΔCD19). iCasp9 includes a human FK506-binding protein (FKBP12; GenBank AH002 818) with an F36V mutation, connected via a Ser-Gly-Gly-Gly linker to human Caspase-9 (CASP9; GenBank NM 001229). The F36V mutation may increase the binding affinity of FKBP12 to the synthetic homodimerizer, AP20187 or AP1903. The caspase recruitment domain (CARD) has been deleted from the human Caspase-9 sequence and its physiological function has been replaced by FKBP12. The replacement of CARD with FKBP12 increases transgene expression and function. The 2A-like sequence encodes an 18 amino acid peptide from Thosea Asigna insect virus, which mediates >99% cleavage between a glycine and terminal proline residue, resulting in 17 extra amino acids in the C terminus of iCasp9, and one extra proline residue in the N terminus of CD19. ΔCD19 consists of full length CD19 (GenBank NM 001770) truncated at amino acid 333 (TDPTRRF), which shortens the intracytoplasmic domain from 242 to 19 amino acids, and removes all conserved tyrosine residues that are potential sites for phosphorylation.

In Vivo Studies

Three patients received iCasp9⁺ T cells after haplo-CD34⁺ stem cell transplantation (SCT), at dose levels between about 1×10⁶ to about 3×10⁶ cells/kg.

Infused T cells were detected in vivo by flow cytometry (CD3⁺ΔCD19⁺) or qPCR as early as day 7 after infusion, with a maximum fold expansion of 170±5 (day 29±9 after infusion. Two patients developed grade I/II GvHD and rimiducid administration caused >90% ablation of CD3⁺ ΔCD19⁺ cells, within 30 minutes of infusion, with a further log reduction within 24 hours, and resolution of skin and liver GvHD within 24 hrs, showing that iCasp9 transgene was functional in vivo.

Ex vivo experiments confirmed this data. Furthermore, the residual allodepleted T cells were able to expand and were reactive to viruses (CMV) and fungi (Aspergillus fumigatus) (IFN-γ production). These in vivo studies found that a single dose of dimerizer drug can reduce or eliminate the subpopulation of T cells causing GvHD, but can spare virus specific CTLs, which can then re-expand.

Immune Reconstitution

Depending on availability of patient cells and reagents, immune reconstitution studies (Immunophenotyping, T and B cell function) may be obtained at serial intervals after transplant. Several parameters measuring immune reconstitution resulting from icaspase transduced allodepleted T cells will be analyzed. The analysis includes repeated measurements of total lymphocyte counts, T and CD19 B cell numbers, and FACS analysis of T cell subsets (CD3, CD4, CD8, CD16, CD19, CD27, CD28, CD44, CD62L, CCR7, CD56, CD45RA, CD45RO, alpha/beta and gamma/delta T cell receptors). Depending on the availability of a patients T cells T regulatory cell markers such as CD41CD251FoxP3 also are analyzed. Approximately 10-60 ml of patient blood is taken, when possible, 4 hours after infusion, weekly for 1 month, monthly×9 months, and then at 1 and 2 years. The amount of blood taken is dependent on the size of the recipient and does not exceed 1-2 cc/kg in total (allowing for blood taken for clinical care and study evaluation) at any one blood draw.

Modified Caspase-9 Polypeptides with Lower Basal Activity and Minimal Loss of Ligand IC50

Basal signaling, signaling in the absence of agonist or activating agent, is prevalent in a multitude of biomolecules. For example, it has been observed in more than 60 wild-type G protein coupled receptors (GPCRs) from multiple subfamilies [1], kinases, such as ERK and abl [2], surface immunoglobulins [3], and proteases. Basal signaling has been hypothesized to contribute to a vast variety of biological events, from maintenance of embryonic stem cell pluripotency, B cell development and differentiation [4-6], T cell differentiation [2, 7], thymocyte development [8], endocytosis and drug tolerance [9], autoimmunity [10], to plant growth and development [11]. While its biological significance is not always fully understood or apparent, defective basal signaling can lead to serious consequences. Defective basal Gₛ protein signaling has led to diseases, such as retinitis pigmentosa, color blindness, nephrogenic diabetes insipidus, familial ACTH resistance, and familial hypocalciuric hypercalcemia [12, 13].

Even though homo-dimerization of wild-type initiator Caspase-9 is energetically unfavorable, making them mostly monomers in solution [14-16], the low-level inherent basal activity of unprocessed Caspase-9 [15, 17] is enhanced in the presence of the Apaf-1-based “apoptosome”, its natural allosteric regulator [6]. Moreover, supra-physiological expression levels and/or co-localization could lead to proximity-driven dimerization, further enhancing basal activation. The modified cells of the present application may comprise nucleic acids coding for a chimeric Caspase-9 polypeptide having lower basal signaling activity. Examples of Caspase-9 mutants with lower basal signaling are provided in the table below. Polynucleotides comprising Caspase-9 mutants with lower basal signaling may be expressed in the modified cells used for cell therapy herein. In these examples, the modified cells may include a safety switch, comprising a polynucleotide encoding a lower basal signaling chimeric Caspase-9 polypeptide. In the event of an adverse event following administration of the modified cells comprising the chimeric signaling polypeptides or chimeric antigen receptors herein, Caspase-9 activity may be induced by administering the dimerizer to the patient, thus inducing apoptosis and clearance of some, or all of the modified cells. In some examples, the amount of dimerizer administered may be determined as an amount designed to remove the highest amount, at least 80% or 90% of the modified cells. In other examples, the amount of dimerizer administered may be determined as an amount designed to remove only a portion of the modified cells, in order to alleviate negative symptoms or conditions, while leaving a sufficient amount of therapeutic modified cells in the patient, in order to continue therapy. Methods for using chimeric Caspase-9 polypeptides to induce apoptosis are discussed in PCT Application Number PCT/US2011/037381 by Malcolm K. Brenner et al., titled Methods for Inducing Selective Apoptosis, filed May 20, 2011, and in U.S. patent application Ser. No. 13/112,739 by Malcolm K. Brenner et al., titled Methods for Inducing Selective Apoptosis, filed May 20, 2011, issued Jul. 28, 2015 as U.S. Pat. No. 9,089,520. Chimeric caspase polypeptides having modified basal activity are discussed in PCT Application Serial Number PCT/US2014/022004 by David Spencer et al., titled Modified Caspase Polypeptides and Uses Thereof, filed Mar. 7, 2014, published Oct. 9, 2014 as WO2014/164348, and in U.S. patent application Ser. No. 13/792,135 by David Spencer et al., titled Modified Caspase Polypeptides and Uses Thereof, filed Mar. 7, 2014; and in U.S. patent application Ser. No. 14/640,553 by Spencer et al., filed Mar. 6, 2015. Methods for inducing partial apoptosis of the therapeutic modified cells are discussed in PCT Application Number PCT/US14/040964 by Kevin Slawin et al., titled Methods for Inducing Partial Apoptosis Using Caspase Polypeptides, filed Jun. 4, 2014, published Dec. 11, 2014 as WO2014/197638, and in U.S. patent application Ser. No. 14/296,404 by Kevin Slawin et al., titled Methods for Inducing Partial Apoptosis Using Caspase Polypeptides, filed Jun. 4, 2014. These patent applications and publications are all incorporated by reference herein in their entireties.

TABLE 5
Caspase Mutant Classes and Basal Activity

| | | Cleavage | | | |
|---|---|---|---|---|---|
| | | sites & | | Double | |
| Basal | Homodimerization | XIAP | | mutants, | |
| Activity | domain | Interaction | Phosphorylation | Misc. | Total mutants |
| Decreased | | | S144A | | 80 |
| basal and | | | S144D | | *, predicted |
| similar IC₅₀ | | T317S | S196D | | |
| Decreased | N405Q | D330A | S183A | D330A- | Bold, Tested |
| basal but | | | | N405Q | in T cells |
| higher IC₅₀ | ⁴⁰²GCFNF⁴⁰⁶ISAQT | D330E | S195A | D330A- | |
| | (Casp-10) | | | S144A | |
| | F404Y | D330G | S196A | D330A- | |
| | | | | S144D | |
| | F406A | D330N | | D330A- | |
| | | | | S183A | |
| | F406W | D330S | | D330A- | |
| | | | | S196A | |
| | F406Y | D330V | | N405Q- | |
| | | | | S144A | |
| | N405Qco | L329E | | N405Q- | |
| | | | | S144D | |
| | | T317A | | N405Q- | |
| | | | | S196D | |
| | | | | N405Q- | |
| | | | | T317S | |
| | | | | *N405Q- | |
| | | | | S144Aco | |
| | | | | *N405Q- | |
| | | | | T317Sco | |
| Decreased | F404T | D315A | Y153A | | |
| basal but | F404W | A316G | Y153F | | |
| much higher | N405F | F319W | S307A | | |
| IC₅₀ | F406T | | | | |
| Similar | C403A | ³¹⁶ATPF³¹⁹A | | | |
| basal and | | VPI | | | |
| IC₅₀ | | (SMAC/ | | | |
| | | Diablo) | | | |
| | C403S | T317C | | | |
| | C403T | P318A | | | |
| | N405A | F319A | | | |
| Increased | N405T | T317E | | D330A- | |
| basal | | | | N405T | |
| | | F326K | | | |
| | | D327G | | | |
| | | D327K | | | |
| | | D327R | | | |
| | | Q328K | | | |
| | | Q328R | | | |
| | | L329G | | | |
| | | L329K | | | |
| | | A331K | | | |
| Catalytically | ⁴⁰²GCFNF⁴⁰⁶AAAAA | | | C285A | |
| dead | ⁴⁰²GCFNF⁴⁰⁶YCSTL | | | D315A- | |
| | (Casp-2) | | | D330A | |
| | ⁴⁰²GCFNF⁴⁰⁶CIVSM | | | D330A- | |
| | (Casp-3) | | | Y153A | |
| | ⁴⁰²GCFNF⁴⁰⁶QPTFT | | | D330A- | |
| | (Casp-8) | | | Y153F | |
| | G402A | | | D330A- | |
| | | | | T317E | |
| | G402I | | | | |
| | G402Q | | | | |
| | G402Y | | | | |
| | C403P | | | | |
| | F404A | | | | |
| | F404S | | | | |
| | F406L | | | | |

Literature References Cited or Providing Additional Support to the Present Example
1. Seifert, R. and K. Wenzel-Seifert, Constitutive activity of G-protein-coupled receptors: cause of disease and common property of wild-type receptors. Naunyn Schmiedebergs Arch Pharmacol, 2002. 366(5): p. 381-416.
2. Roose, J. P., et al., T cell receptor-independent basal signaling via Erk and Abl kinases suppresses RAG gene expression. PLoS Biol, 2003. 1(2): p. E53.
3. Tze, L. E., et al., Basal immunoglobulin signaling actively maintains developmental stage in immature B cells. PLoS Biol, 2005. 3(3): p. e82.
4. Schram, B. R., et al., B cell receptor basal signaling regulates antigen-induced Ig light chain rearrangements. J Immunol, 2008. 180(7): p. 4728-41.
5. Randall, K. L., et al., Dock8 mutations cripple B cell immunological synapses, germinal centers and long-lived antibody production. Nat Immunol, 2009. 10(12): p. 1283-91.
6. Kouskoff, V., et al., B cell receptor expression level determines the fate of developing B lymphocytes: receptor editing versus selection. Proc Natl Acad Sci USA, 2000. 97(13): p. 7435-9.
7. Hong, T., et al., A simple theoretical framework for understanding heterogeneous differentiation of CD4⁺ T cells. BMC Syst Biol, 2012. 6: p. 66.
8. Rudd, M. L., A. Tua-Smith, and D. B. Straus, Lck SH3 domain function is required for T-cell receptor signals regulating thymocyte development. Mol Cell Biol, 2006. 26(21): p. 7892-900.
9. Sorkin, A. and M. von Zastrow, Endocytosis and signalling: intertwining molecular networks. Nat Rev Mol Cell Biol, 2009. 10(9): p. 609-22.
10. Luning Prak, E. T., M. Monestier, and R. A. Eisenberg, B cell receptor editing in tolerance and autoimmunity. Ann N Y Acad Sci, 2011. 1217: p. 96-121.
11. Boss, W. F., et al., Basal signaling regulates plant growth and development. Plant Physiol, 2010. 154(2): p. 439-43.
12. Tao, Y. X., Constitutive activation of G protein-coupled receptors and diseases: insights into mechanisms of activation and therapeutics. Pharmacol Ther, 2008. 120(2): p. 129-48.
13. Spiegel, A. M., Defects in G protein-coupled signal transduction in human disease. Annu Rev Physiol, 1996. 58: p. 143-70.
14. Shiozaki, E. N., et al., Mechanism of XIAP-mediated inhibition of Caspase-9. Mol Cell, 2003. 11(2): p. 519-27.
15. Renatus, M., et al., Dimer formation drives the activation of the cell death protease Caspase-9. Proc Natl Acad Sci USA, 2001. 98(25): p. 14250-5.
16. Shi, Y., Mechanisms of Caspase activation and inhibition during apoptosis. Mol Cell, 2002. 9(3): p. 459-70.
17. Shiozaki, E. N., J. Chai, and Y. Shi, Oligomerization and activation of Caspase-9, induced by Apaf-1 CARD. Proc Natl Acad Sci USA, 2002. 99(7): p. 4197-202.
18. Straathof, K. C., et al., An inducible Caspase-9 safety switch for T-cell therapy. Blood, 2005. 105(11): p. 4247-54.
19. MacCorkle, R. A., K. W. Freeman, and D. M. Spencer, Synthetic activation of Caspases: artificial death switches. Proc Natl Acad Sci USA, 1998. 95(7): p. 3655-60.
20. Di Stasi, A., et al., Inducible apoptosis as a safety switch for adoptive cell therapy. N Engl J Med, 2011. 365(18): p. 1673-83.
21. Chang, W. C., et al., Modifying ligand-induced and constitutive signaling of the human 5-HT4 receptor. PLoS One, 2007. 2(12): p. e1317.
22. Bloom, J. D. and F. H. Arnold, In the light of directed evolution: pathways of adaptive protein evolution. Proc Natl Acad Sci USA, 2009. 106 Suppl 1: p. 9995-10000.
23. Boatright, K. M. and G. S. Salvesen, Mechanisms of Caspase activation. Curr Opin Cell Biol, 2003. 15(6): p. 725-31.
24. Boatright, K. M., et al., A unified model for apical Caspase activation. Mol Cell, 2003. 11(2): p. 529-41.
25. Chao, Y., et al., Engineering a dimeric Caspase-9: a re-evaluation of the induced proximity model for Caspase activation. PLoS Biol, 2005. 3(6): p. e183.
26. Stennicke, H. R., et al., Caspase-9 can be activated without proteolytic processing. J Biol Chem, 1999. 274(13): p. 8359-62.
27. Brady, S. C., L. A. Allan, and P. R. Clarke, Regulation of Caspase-9 through phosphorylation by protein kinase C zeta in response to hyperosmotic stress. Mol Cell Biol, 2005. 25(23): p. 10543-55.
28. Martin, M. C., et al., Protein kinase A regulates Caspase-9 activation by Apaf-1 downstream of cytochrome c. J Biol Chem, 2005. 280(15): p. 15449-55.
29. Cardone, M. H., et al., Regulation of cell death protease Caspase-9 by phosphorylation. Science, 1998. 282(5392): p. 1318-21.
30. Raina, D., et al., c-Abl tyrosine kinase regulates Caspase-9 autocleavage in the apoptotic response to DNA damage. J Biol Chem, 2005. 280(12): p. 11147-51.
31. Papworth, C., Bauer, J. C., Braman, J. and Wright, D. A., Site-directed mutagenesis in one day with >80% efficiency. Strategies, 1996. 9(3): p. 3-4.
32. Spencer, D. M., et al., Functional analysis of Fas signaling in vivo using synthetic inducers of dimerization. Curr Biol, 1996. 6(7): p. 839-47.
33. Hsiao, E. C., et al., Constitutive Gs activation using a single-construct tetracycline-inducible expression system in embryonic stem cells and mice. Stem Cell Res Ther, 2011. 2(2): p. 11.
34. Waldner, C., et al., Double conditional human embryonic kidney cell line based on FLP and PhiC31 mediated transgene integration. BMC Res Notes, 2011. 4: p. 420.

The following patents, applications, and patent publications may contain material and methods that may be used in the examples herein, such as, for example, U.S. Pat. No. 7,404,950, issued Jul. 29, 2008, to Spencer, D. et al.; U.S. Pat. No. 8,691,210, issued Apr. 8 2004 to Spencer, et al.; U.S. patent application Ser. No. 12/532,196 by Spencer et al., filed Sep. 21, 2009; PCT application PCT/US2009/057738 to Spencer et al., published on Apr. 24, 2008 as WO2010/033949; U.S. patent application Ser. No. 13/087,329 by Slawin et al., filed Apr. 14, 2011; PCT application PCT/US2011/032572, published on Oct. 20, 2011 as WO2011/130566; U.S. patent application Ser. No. 14/210,324 by Spencer et al., filed Mar. 13, 2014; PCT application number PCT/US2014/026734 by Spencer et al., published as WO2014/251960 on Feb. 5, 2015; U.S. application Ser. No. 14/622,018, by Foster et al., filed Feb. 13, 2015; PCT application number PCT/US2015/015829 by Foster et al., published as WO2015/123527 on Aug. 20, 2015 are all hereby incorporated by reference herein in their entirety.

### Example 7: Using the Inducible Chimeric Signaling Polypeptide in Human Cells for Therapy

Presented in this example are expression constructs and methods of using the expression constructs in human cells. Although this example refers to the inducible chimeric stimulating molecule, vectors coding for the inducible chimeric signaling polypeptides, chimeric signaling polypeptides and modified chimeric antigen polypeptides of the present application may also be used, including any appropriate modifications. In some examples, the method may be modified to provide alternative multimeric ligand binding regions. In some examples, the method may be modified to provide inducible chimeric signaling polypeptides that lack a membrane targeting region.

These methods may be adapted for other cells, such as, for example NK and NKT cells, as well as tumor-infiltrating lymphocytes, and may also be adapted for chimeric signaling polypeptides that comprise other costimulatory polypeptide cytoplasmic regions as discussed herein.

Materials and Methods

Large-Scale Generation of Gene-Modified T Cells

T cells are generated from healthy volunteers, using standard methods. Briefly, peripheral blood mononuclear cells (PBMCs) from healthy donors or cancer patients are activated for expansion and transduction using soluble αCD3 and αCD28 (Miltenyi Biotec, Auburn, Calif.). PBMCs are resuspended in Cellgenix DC media supplemented with 100 U/ml IL-2 (Cellgenix) at 1×10⁶ cells/ml and stimulated with 0.2 μg/ml αCD3 and 0.5 μg/ml αCD28 soluble antibody. Cells are then cultured at 37° C., 5% CO₂ for 4 days. On day four, 1 ml of fresh media containing IL-2 is added. On day 7, cells are harvested and resuspended in Cellgenix DC media for transduction.

Plasmid and Retrovirus

The SFG plasmid consists of inducible CSM linked, via a cleavable 2A-like sequence, to truncated human CD19. The inducible CSM consists of a human FK506-binding protein (FKBP12; GenBank AH002 818) with an F36V mutation, connected via a Ser-Gly-Gly-Gly-Ser linker to a human CSM. The F36V mutation increases the binding affinity of FKBP12 to the synthetic homodimerizer, AP20187 or rimiducid. The 2A-like sequence encodes a 20 amino acid peptide from Thosea asigna insect virus, which mediates >95% cleavage between a glycine and terminal proline residue, resulting in 19 extra amino acids in the C terminus of iCSM, and one extra proline residue in the N terminus of CD19. CD19 consists of full-length CD19 (GenBank NM 001770) truncated at amino acid 333 (TDPTRRF), which shortens the intracytoplasmic domain from 242 to 19 amino acids, and removes all conserved tyrosine residues that are potential sites for phosphorylation.

A stable PG13-based clone producing Gibbon ape leukemia virus (Gal-V) pseudotyped retrovirus is made by transiently transfecting Phoenix Eco cell line (ATCC product #SD3444; ATCC, Manassas, Va.) with the SFG plasmid. This produces Eco-pseudotyped retrovirus. The PG13 packaging cell line (ATCC) is transduced three times with Eco-pseudotyped retrovirus to generate a producer line that contained multiple SFG plasmid proviral integrants per cell. Single cell cloning is performed, and the PG13 clone that produced the highest titer is expanded and used for vector production.

Retroviral Transduction

Culture medium for T cell activation and expansion is serum-free Cellgenix DC medium (Cellgenix) supplemented by 100 U/ml IL-2 (Cellgenix). T cells are activated by soluble anti-CD3 and anti-CD28 (Miltenyi Biotec) for 7 days before transduction with retroviral vector. Immunomagnetic selection of ΔCD19, if necessary, is performed on day 4 after transduction; the positive fraction was expanded for a further 2 days and cryopreserved.

Scaling-Up Production of Gene-Modified Allodepleted Cells

Scale-up of the transduction process for clinical application use non-tissue culture-treated T75 flasks (Nunc, Rochester, N.Y.), which are coated with 10 ml of anti-CD3 0.5 μg/ml and anti-CD28 0.2 μg/ml or 10 ml of fibronectin 7 μg/ml at 4° C. overnight. Fluorinated ethylene propylene bags corona-treated for increased cell adherence (2PF-0072AC, American Fluoroseal Corporation, Gaithersburg, Md.) are also used. PBMCs are seeded in anti-CD3, anti-CD28-coated flasks at 1×10⁶ cells/ml in media supplemented with 100 U/ml IL-2. For retroviral transduction, retronectin-coated flasks or bags are loaded once with 10 ml of retrovirus-containing supernatant for 2 to 3 hours. Activated T cells are seeded at 1×10⁶ cells/ml in fresh retroviral vector-containing medium and T cell culture medium at a ratio of 3:1, supplemented with 100 U/ml IL-2. Cells are harvested the following morning and expanded in tissue-culture treated T75 or T175 flasks in culture medium supplemented with 100 U/ml IL-2 at a seeding density of between about 5×10⁵ cells/ml to 8×10⁵ cells/ml.

CD19 Immunomagnetic Selection

Immunomagnetic Selection for CD19 May, for Example, be Performed 4 Days after Transduction. Cells are labeled with paramagnetic microbeads conjugated to monoclonal mouse anti-human CD19 antibodies (Miltenyi Biotech, Auburn, Calif.) and selected on MS or LS columns in small scale experiments and on a CliniMacs Plus automated selection device in large scale experiments. CD19-selected cells are expanded for a further 4 days and cryopreserved on day 8 post transduction. These cells are referred to as “gene-modified cells”.

Immunophenotyping and Pentamer Analysis

Flow cytometric analysis (FACSCalibur and CellQuest software; Becton Dickinson) is performed using the following antibodies: CD3, CD4, CD8, CD19, CD25, CD27, CD28, CD45RA, CD45RO, CD56 and CD62L. CD19-PE (Clone 4G7; Becton Dickinson) is found to give optimum staining and was used in all subsequent analysis. A non-transduced control is used to set the negative gate for CD19. CAR expression is assessed using anti-F(ab′)2 (Jackson ImmunoResearch Laboratories, West Grove, Pa.).

Statistical Analysis

Paired, 2-tailed Student's t test is used to determine the statistical significance of differences between samples. All data are represented as mean±1 standard deviation.

### Example 8: Treatment of a Leukemia Patient

The present example of the treatment of a leukemia patient having advanced treatment refractory leukemia, using the methods of the present application, may also be applied to other conditions or diseases, such as, for example, other hyperproliferative diseases or solid tumors. The methods may be used essentially as discussed, with the understanding that the single chain variable fragment may vary according to the target antigen. Although this example refers to the inducible chimeric stimulating molecule, vectors coding for the inducible chimeric signaling polypeptides, chimeric signaling polypeptides and modified chimeric antigen polypeptides of the present application may also be used, including any appropriate modifications. In some examples, the method may be modified to provide alternative multimeric ligand binding regions. In some examples, the method may be modified to provide inducible chimeric signaling polypeptides that lack a membrane targeting region. Further, it is understood that the methods may be modified as appropriate to treat patients for whom recombinant TCR therapy, using a recombinant TCR as the heterologous polypeptide expressed in the cells, rather than a CAR.

T cells are transduced with a nucleic acid comprising a polynucleotide coding for a chimeric signaling polypeptide or inducible chimeric signaling polypeptide of the present application. The T cells are also transduced with a nucleic acid comprising a polynucleotide coding for a chimeric antigen receptor. In other examples, the nucleic acid used to transduce the T cells may include, for example, a polynucleotide coding for the chimeric antigen receptor. The chimeric antigen receptor comprises a single chain variable fragment that recognizes CD19.

The patient undergoes lymphodepletive conditioning, followed by administration of the transduced CD19-targeted T cells. The T cells may be autologous, allogeneic, or non-allogeneic. The dose may be provided, for example, daily, twice a week, or weekly. Because of the concern that an unregulated, too rapid rate of T cell expansion, activation, and tumor cell killing may lead to a more severe cytokine storm that unnecessarily harms the patient, the dosing schedule is designed to achieve a complete recovery at a rate that limits toxicity and does not cause extensive harm to the patient, for example, keeping the patient out of the intensive care unit at a hospital.

### Example 9: Materials and Methods

Methods herein discuss the use of an inducible MyD88/CD40 construct, but may also be used for the non-inducible chimeric signaling polypeptides, the chimeric signaling polypeptides, and the chimeric antigen receptor polypeptides of the present application with appropriate modifications. In some examples, the method may be modified to provide alternative multimeric ligand binding regions. In some examples, the method may be modified to provide inducible chimeric signaling polypeptides that lack a membrane targeting region.

Cell lines, media and reagents. 293T (HEK 293T/17) and Capan-1, Raji, and Daudi cell lines were obtained from the American Type Culture Collection. Cell lines were maintained in DMEM (Invitrogen, Grand Island, N.Y.) supplemented with 10% fetal calf serum (FCS) and 2 mM glutamax (Invitrogen) at 37° C. and 5% CO₂. T cells generated from peripheral blood mononuclear cells (PBMC) were cultured in 45% RPMI 1640, 45% Click's media (Invitrogen) supplemented with 10% fetal bovine serum (FBS), 2 mM glutamax (T cell media; TCM) and 100 U/ml IL-2 (Miltenyi Biotec), unless otherwise noted. Clinical grade rimiducid was diluted in ethanol to a 100 mM working solution for in vitro assays, or 0.9% saline for animal studies.

Retroviral and plasmid constructs. Inducible MyD88/CD40 (iMC) comprising the myristoylation targeting sequence (M) (20), the TLR adaptor molecule MyD88, the CD40 cytoplasmic region, and 2 tandem ligand-binding FKBP12v36 domains (Fv′Fv) were cloned in-frame with 2A-ΔCD19 in the SFG retroviral backbone using Gibson assembly (New England Biolabs, Ipswich, Mass.) to generate SFG-M.MyD88/CD40.Fv′Fv-2A-ΔCD19. Similarly, a control vector was generated that contained only the myristoylation sequence and tandem FKBP12v36 binding domains (SFG-M.Fv′Fv-2A-ΔCD19). Additional retroviral vectors were constructed using a synthetic DNA approach (Integrated DNA Technologies, San Diego, Calif.) to generate MyD88 or CD40 only constructs, termed SFG-M.MyD88.Fv′.Fv-2A-ΔCD19 or SFG-M.CD40.Fv′.Fv-2A-ΔCD19, respectively. Two first generation CARs recognizing CD19 or PSCA were synthesized. The CD19 CAR was designed using the anti-CD19 single chin fragment variable (scFv), FMC63, while PSCA recognition was achieved using the murine scFv bm2B3. Both CARs included the IgG1 CH2CH3 spacer region, the CD28 transmembrane domain and CD3ζ cytoplasmic domain (PSCA.ζ) as discussed in Anurathapan et al., 2013 (13). A second generation CAR was constructed by PCR amplification that contained the CD28 transmembrane and cytoplasmic domain (PSCA.28.ζ). To generate a PSCA CAR that contained MC, MyD88/CD40 was synthesized and inserted 5′ to CD3ζ. For co-culture assays, Capan-1 tumor cells were modified by piggyBac transposase with a plasmid to express GFP (pIRII-GFP-2A-puromycin) and stably selected with 1 μg/ml puromycin.

Retroviral supernatant. Retroviral supernatants were produced by transient co-transfection of 293T cells with the SFG vector plasmid, Peg-Pam-e plasmid containing the sequence for MoMLV gag-pol and the RD114 plasmid encoding the RD114 envelope using GeneJuice (EMD Biosciences, Gibbstown, N.J.) transfection reagent as recommended by the manufacturer as previously discussed (14) Supernatant containing the retrovirus was collected 48 and 72 hours after transfection.

Generation of activated T cells. Using peripheral blood mononuclear cells (PBMC) obtained from the Gulf Coast Blood Bank (Houston, Tex.) anti-CD3/anti-CD28-activated T cells were generated essentially as previously provided (22). Briefly, 5×10⁶ PBMC resuspended in TCM and stimulated on non-tissue culture-treated 24-well plates coated with 0.5 μg/ml each of anti-CD3 and anti-CD28 antibodies (Miltenyi Biotec) in the presence of 100 U/ml IL-2. On day 3, activated T cells were harvested and transduced with retrovirus vectors or expanded in media supplemented with IL-2 as discussed below.

Transduction of T cells. Non-tissue culture treated 24 well plates were coated with 7 μg/ml Retronectin (Takara Bio, Otsu, Shiga, Japan) overnight at 4° C. The wells were washed with phosphate-buffered saline then coated with retroviral supernatant. Subsequently, activated T cells were plated at 3×10⁶ cells per well in viral supernatant supplemented with 100 U/ml IL-2. After three days in culture, cells were harvested and expanded in tissue culture treated plates containing TCM plus 100 U/ml IL-2. For two or three-gene transductions, the protocol is identical to above except the wells were coated with equal amounts of each retroviral supernatant and activated T cells were then plated into each well containing equal amounts of viral supernatant supplemented with 100 U/ml IL-2.

Immunophenotyping. Gene-modified T cells were analyzed for iMC transgene expression 10-14 days post-transduction by using CD3-PerCP.Cy5 and CD19-PE (BioLegend). To detect CAR-transduced cells, T cells were also stained with an Fc-specific monoclonal antibody conjugated to APC (Jackson ImmunoResearch Laboratories, West Grove, Pa.), which recognizes the IgG1 CH₂CH₃ component of the receptor. All flow cytometry was performed using an LSRII flow cytometer (Becton Dickenson, East Rutherford, N.J.), and the data analyzed using FlowJo (Tree star, Ashland, Oreg.).

Cytokine and chemokine production. Production of IFN-γ, IL-2 and IL-6 by T cells modified with iMC, control vectors or CAR-modified T cells were analyzed by ELISA per the manufacturer's protocol (eBioscience, San Diego, Calif.). In this assay, non-transduced T cells and iMC- or control vector-modified T cells were activated with 10 nM rimiducid or vehicle (EtOH), and supernatants collected at 48 hours. For analysis of CAR-modified T cells, T cells were cocultured with Capan-1 tumor cells at 1:1 T cell to tumor cell ratios and supernatants were harvested at 24 and 48 hours.

Cytotoxicity assay. The specific cytotoxicity of CAR T cells against Capan-1 tumor cells was measured in 6 hour and 24 hour lactate dehydrogenase (LDH) assays per the manufacturer's recommendations (Clontech Laboratories, Mountain View, Calif.) using effector to target (E:T) ratios ranging from 10:1 to 0.5:1 and using Capan-1 as target cells.

Coculture experiment. To test the cytotoxicity, activation, proliferation and cytokine production following PSCA.ζ CAR activation, co-culture assays were performed co-culture assays with Capan-1-GFP tumor cells at E:T ratios of 10:1, 5:1, 1:1, 1:5 and 1:10 in TCM in the absence of exogenous IL-2. After 7 days, all residual cells were collected by trypsinization, counted and stained with CD3 and Fc-specific antibodies and analyzed by flow cytometry. In addition, similar co-culture experiments were performed using CD19 CAR with and without MC costimulation. CAR-modified T cells were cultured for 7 days with CD19⁺ Raji or Daudi cell lines and subsequently analyzed by flow cytometry for CD3⁺ and CD19⁺ cells.

Statistics. Data are represented as mean±SEM. Data were analyzed using unpaired Student's t test to calculate 2-tailed or 1-tailed P values to determine statistical significance in differences when comparing 2 treatment groups in all assays. Data were analyzed using GraphPad Prism version 5.0 software (GraphPad).

### Example 10: Design and Activity of MyD88/CD40 Chimeric Antigen Receptors

Methods herein relate to the use of a modified CAR comprising a MyD88/CD40 construct, but may also be used for the modified CAR polypeptides of the present application with appropriate modifications. In some examples, a costimulatory polypeptide cytoplasmic region, for example, an OX40, CD28, 4-1BB, or ICOS cytoplasmic region is substituted for the CD40 cytoplasmic region of the CAR polypeptide.

Design of MC-CAR Constructs

MC (without rimiducid-binding FKBPv36 regions) was subcloned into the PSCA.ζ to emulate the position of the CD28 endodomain. Retrovirus was generated for each of the three constructs, transduced human T cells and subsequently measured transduction efficiency demonstrating that PSCA.MC.ζ could be expressed. To confirm that T cells bearing each of these CAR constructs retained their ability to recognize PSCA⁺ tumor cells, 6-hour cytotoxicity assays were performed, which showed lysis of Capan-1 target cells. Therefore, the addition of MC into the cytoplasmic region of a CAR molecule does not affect CAR expression or the recognition of antigen on target cells.

MC costimulation enhances T cell killing, proliferation and survival in CAR-modified T cells As demonstrated in short-term cytotoxicity assays, each of the three CAR designs showed the capacity to recognize and lyse Capan-1 tumor cells. Cytolytic effector function in effector T cells is mediated by the release of pre-formed granzymes and perforin following tumor recognition, and activation through CD3ζ is sufficient to induce this process without the need for costimulation. First generation CAR T cells (e.g., CARs constructed with only the CD3 cytoplasmic region) can lyse tumor cells; however, survival and proliferation is impaired due to lack of costimulation. Hence, the addition of CD28 or 4-1BB costimulating domains constructs has significantly improved the survival and proliferative capacity of CAR T cells.

To examine whether MC can similarly provide costimulating signals affecting survival and proliferation, coculture assays were performed with PSCA⁺ Capan-1 tumor cells under high tumor:T cell ratios (1:1, 1:5, 1:10 T cell to tumor cell). When T cell and tumor cell numbers were equal (1:1), there was efficient killing of Capan-1-GFP cells from all three constructs compared to non-transduced control T cells. However, when the CAR T cells were challenged with high numbers of tumor cells (1:10), there was a significant reduction of Capan-1-GFP tumor cells only when the CAR molecule contained either MC or CD28.

To further examine the mechanism of costimulation by these two CARs cell viability and proliferation was assayed. PSCA CARs containing MC or CD28 showed improved survival compared to non-transduced T cells and the CD3ζ only CAR, and T cell proliferation by PSCA.MC.ζ and PSCA.28.ζ was significantly enhanced. As other groups have shown that CARs that contain costimulating signaling regions produce IL-2, a key survival and growth molecule for T cells (4), ELISAs were performed on supernatants from CAR T cells challenged with Capan-1 tumor cells. Although PSCA.28.ζ produced high levels of IL-2, PSCA.MC.ζ signaling also produced significant levels of IL-2, which likely contributes to the observed T cell survival and expansion in these assays. Additionally, IL-6 production by CAR-modified T cells was examined, as IL-6 has been implicated as a key cytokine in the potency and efficacy of CAR-modified T cells (15). In contrast to IL-2, PSCA.MC.ζ produced higher levels of IL-6 compared to PSCA.28.ζ, consistent with the observations that iMC activation in primary T cells induces IL-6. Together, these data suggest that costimulation through MC produces similar effects to that of CD28, whereby following tumor cell recognition, CAR-modified T cells produce IL-2 and IL-6, which enhance T cell survival

Immunotherapy using CAR-modified T cells holds great promise for the treatment of a variety of malignancies. While CARs were first designed with a single signaling domain (e.g., CD3ζ),(16-19) clinical trials evaluating the feasibility of CAR immunotherapy showed limited clinical benefit.(1,2,20,21) This has been primarily attributed to the incomplete activation of T cells following tumor recognition, which leads to limited persistence and expansion in vivo.(22) To address this deficiency, CARs have been engineered to include another stimulating domain, often derived from the cytoplasmic portion of T cell costimulating molecules including CD28, 4-1BB, OX40, ICOS and DAP10, (4,23-30) which allow CAR T cells to receive appropriate costimulation upon engagement of the target antigen. Indeed, clinical trials conducted with anti-CD19 CARs bearing CD28 or 4-1BB signaling domains for the treatment of refractory acute lymphoblastic leukemia (ALL) have demonstrated impressive T cell persistence, expansion and serial tumor killing following adoptive transfer. (6-8)

CD28 costimulation provides a clear clinical advantage for the treatment of CD19⁺ lymphomas. Savoldo and colleagues conducted a CAR-T cell clinical trial comparing first (CD19.ζ) and second generation CARs (CD19.28.) and found that CD28 enhanced T cell persistence and expansion following adoptive transfer.31 One of the principal functions of second generation CARs is the ability to produce IL-2 that supports T cell survival and growth through activation of the NFAT transcription factor by CD3ζ (signal 1), and NF-κB (signal 2) by CD28 or 4-1BB.32 This suggested other molecules that similarly activated NF-κB might be paired with the CD3ζ chain within a CAR molecule. Our approach has employed a T cell costimulating molecule that was originally developed as an adjuvant for a dendritic cell (DC) vaccine.(12,33) For full activation or licensing of DCs, TLR signaling is usually involved in the upregulation of the TNF family member, CD40, which interacts with CD40L on antigen-primed CD4⁺ T cells. Because iMC was a potent activator of NF-κB in DCs, transduction of T cells with CARs that incorporated MyD88 and CD40 might provide the required costimulation (signal 2) to T cells, and enhance their survival and proliferation.

A set of experiments was performed to examine whether MyD88, CD40 or both components were required for optimum T cell stimulation using the iMC molecule. Remarkably, it was found that neither MyD88 nor CD40 could sufficiently induce T cell activation, as measured by cytokine production (IL-2 and IL-6), but when combined as a single fusion protein, could induce potent T cell activation. A PSCA CAR incorporating MC was constructed and subsequently compared its function against a first (PSCA.ζ) and second generation (PSCA.28.ζ) CAR. Here it was found that MC enhanced survival and proliferation of CAR T cells to a comparable level as the CD28 endodomain, suggesting that costimulation was sufficient. While PSCA.MC.ζ CAR-transduced T cells produced lower levels of IL-2 than PSCA.28.ζ, the secreted levels were significantly higher than non-transduced T cells and T cells transduced with the PSCA.ζ CAR. On the other hand, PSCA.MC.ζ CAR-transduced T cells secreted significantly higher levels of IL-6, an important cytokine associated with T cell activation, than PSCA.28.ζ transduced T cells, indicating that MC conferred unique properties to CAR function that may translate to improved tumor cell killing in vivo. While molecular analyses of the relevant signaling pathways still needs to be performed, these experiments indicate that MC can activate NF-κB (signal 2) following antigen recognition by the extracellular CAR domain.

Apart from survival and growth advantages, MC-induced costimulation may also provide additional functions to CAR-modified T cells. Medzhitov and colleagues recently demonstrated that MyD88 signaling was critical for both Th1 and Th17 responses and that it acted via IL-1 to render CD4⁺ T cells refractory to regulatory T cell (Treg)-driven inhibition.(34) Experiments with iMC show that IL-1α and β are secreted following rimiducid activation (data not shown). In addition, Martin et al demonstrated that CD40 signaling in CD8⁺ T cells via Ras, PI3K and protein kinase C, result in NF-κB-dependent induction of cytotoxic mediators granzyme and perforin that lyse CD4⁺CD25⁺ Treg cells (35). Thus, MyD88 and CD40 co-activation may render CAR-T cells resistant to the immunosuppressive effects of Treg cells, a function that could be critically important in the treatment of solid tumors and other types of cancers.

In summary, MC can be incorporated into a CAR molecule and primary T cells transduced with retrovirus can express PSCA.MC.ζ without overt toxicity or CAR stability issues. Further, MC appears to provide similar costimulation to that of CD28, where transduced T cells show improved survival, proliferation and tumor killing compared to T cells transduced with a first generation CAR. Additional experiments to determine whether MC adds additional benefits to CARs, such as resistance to the inhibitory effects of Treg cells may be considered.

### Example 11: Chimeric Signaling Polypeptides Co-Expressed in T Cells with CARS

The following example provides methods used for a MyD88/CD40 chimeric stimulating molecule. This method may be modified as appropriate to assay the inducible chimeric signaling polypeptides discussed herein. In some examples, the method may be modified to provide alternative multimeric ligand binding regions. In some examples, the method may be modified to provide inducible chimeric signaling polypeptides that lack a membrane targeting region. The method may also be modified to include the expression of a different heterologous protein or polypeptide such as, for example, a recombinant TCR or another heterologous polypeptide discussed herein, instead of, or in additions to, the CAR.

A MyD88/CD40 chimeric stimulating molecule may also be expressed in a cell along with a CAR, which may, for example, comprise the scFv polypeptide, and the CD3-ζ chain. In this method, the CSM molecule is used in combination with a CAR, thereby segregating CAR signaling into two separate functions. This second function, provided by the CAR, provides antigen-specific cytotoxicity to the engineered T cells. For example, a CAR with specificity against PSMA may be expressed in T cells along with a MyD88/CD40 chimeric stimulating molecule. Also, the MyD88/CD40 CSM and the CAR portions may be transfected or transduced into the cells either on the same vector, in cis, or on separate vectors, in trans. Thus, the two polypeptides may be expressed using two nucleic acids, such as, for example, two plasmids or two viruses, and the T cells may be, for example, transfected twice, or in particular embodiments, the two nucleic acids may be co-transfected. In other embodiments, the two polypeptides may be expressed in one nucleic acid, such as, for example, in the same plasmid or virus. The nucleic acid may express the two polypeptides using two separate promoters, one for the CAR and one for the CSM. Or, in other embodiments, the two polypeptides may be expressed using the same promoter. In this embodiment, the two polypeptides may be separated by a cleavable polypeptide, such as, for example, a 2A sequence. The engineered T may, for example, be administered to a subject to generate a specific immune response, for example one directed against a prostate cancer tumor.

In some embodiments, the chimeric signaling polypeptide or inducible chimeric signaling polypeptide does not comprise CD40 or a CD40 cytoplasmic region. It is understood that the methods, constructs, polypeptide, and cells provided for the MyD88/CD40 chimeric stimulating molecules may be modified as necessary for expression and use of the MyD88 chimeric signaling and inducible signaling polypeptides discussed herein.

MyD88/CD40 basal activity was found to be sufficient to stimulate a T cell which expresses a CD19-binding chimeric antigen receptor. The MyD88/CD40 vector used in the assay expressed a chimeric inducible MyD88/CD40 polypeptide, and was designed to be inducible by rimiducid. In the absence of rimiducid, there was sufficient basal activity to provide costimulation to the CD19-CAR following encounter with tumor cells.

### REFERENCES

The following references are cited in, or provide additional information that may be relevant.
1. Till B G, Jensen M C, Wang J, et al: CD20-specific adoptive immunotherapy for lymphoma using a chimeric antigen receptor with both CD28 and 4-1BB domains: pilot clinical trial results. Blood 119:3940-50, 2012.
2. Pule M A, Savoldo B, Myers G D, et al: Virus-specific T cells engineered to coexpress tumor-specific receptors: persistence and antitumor activity in individuals with neuroblastoma. Nat Med 14:1264-70, 2008.
3. Kershaw M H, Westwood J A, Parker L L, et al: A phase I study on adoptive immunotherapy using gene-modified T cells for ovarian cancer. Clin Cancer Res 12:6106-15, 2006.
4. Carpenito C, Milone M C, Hassan R, et al: Control of large, established tumor xenografts with genetically retargeted human T cells containing CD28 and CD137 domains. Proc Natl Acad Sci USA 106:3360-5, 2009.
5. Song D G, Ye Q, Poussin M, et al: CD27 costimulation augments the survival and antitumor activity of redirected human T cells in vivo. Blood 119:696-706, 2012.
6. Kalos M, Levine B L, Porter D L, et al: T cells with chimeric antigen receptors have potent antitumor effects and can establish memory in patients with advanced leukemia. Sci Transl Med 3:95ra73, 2011.
7. Porter D L, Levine B L, Kalos M, et al: Chimeric antigen receptor-modified T cells in chronic lymphoid leukemia. N Engl J Med 365:725-33, 2011.
8. Brentjens R J, Davila M L, Riviere I, et al: CD19-targeted T cells rapidly induce molecular remissions in adults with chemotherapy-refractory acute lymphoblastic leukemia. Sci Transl Med 5:177ra38, 2013.
9. Pule M A, Straathof K C, Dotti G, et al: A chimeric T cell antigen receptor that augments cytokine release and supports clonal expansion of primary human T cells. Mol Ther 12:933-41, 2005.
10. Finney H M, Akbar A N, Lawson A D: Activation of resting human primary T cells with chimeric receptors: costimulation from CD28, inducible costimulator, CD134, and CD137 in series with signals from the TCR zeta chain. J Immunol 172:104-13, 2004.
11. Guedan S, Chen X, Madar A, et al: ICOS-based chimeric antigen receptors program bipolar TH17/TH1 cells. Blood, 2014.
12. Narayanan P, Lapteva N, Seethammagari M, et al: A composite MyD88/CD40 switch synergistically activates mouse and human dendritic cells for enhanced antitumor efficacy. J

Clin Invest 121:1524-34, 2011.
13. Anurathapan U, Chan R C, Hindi H F, et al: Kinetics of tumor destruction by chimeric antigen receptor-modified T cells. Mol Ther 22:623-33, 2014.
14. Craddock J A, Lu A, Bear A, et al: Enhanced tumor trafficking of GD2 chimeric antigen receptor T cells by expression of the chemokine receptor CCR2b. J Immunother 33:780-8, 2010.
15. Lee D W, Gardner R, Porter D L, et al: Current concepts in the diagnosis and management of cytokine release syndrome. Blood 124:188-95, 2014.
16. Becker M L, Near R, Mudgett-Hunter M, et al: Expression of a hybrid immunoglobulin-T cell receptor protein in transgenic mice. Cell 58:911-21, 1989.
17. Goverman J, Gomez S M, Segesman K D, et al: Chimeric immunoglobulin-T cell receptor proteins form functional receptors: implications for T cell receptor complex formation and activation. Cell 60:929-39, 1990.
18. Gross G, Waks T, Eshhar Z: Expression of immunoglobulin-T-cell receptor chimeric molecules as functional receptors with antibody-type specificity. Proc Natl Acad Sci USA 86:10024-8, 1989.
19. Kuwana Y, Asakura Y, Utsunomiya N, et al: Expression of chimeric receptor composed of immunoglobulin-derived V regions and T-cell receptor-derived C regions. Biochem Biophys Res Commun 149:960-8, 1987.
20. Jensen M C, Popplewell L, Cooper L J, et al: Antitransgene rejection responses contribute to attenuated persistence of adoptively transferred CD20/CD19-specific chimeric antigen receptor redirected T cells in humans. Biol Blood Marrow Transplant 16:1245-56, 2010.
21. Park J R, Digiusto D L, Slovak M, et al: Adoptive transfer of chimeric antigen receptor re-directed cytolytic T lymphocyte clones in patients with neuroblastoma. Mol Ther 15:825-33, 2007.
22. Ramos C A, Dotti G: Chimeric antigen receptor (CAR)-engineered lymphocytes for cancer therapy. Expert Opin Biol Ther 11:855-73, 2011.
23. Finney H M, Lawson A D, Bebbington C R, et al: Chimeric receptors providing both primary and costimulatory signaling in T cells from a single gene product. J Immunol 161:2791-7, 1998.
24. Hombach A, Weczarkowiecz A, Marquardt T, et al: Tumor-specific T cell activation by recombinant immunoreceptors: CD3 zeta signaling and CD28 costimulation are simultaneously required for efficient IL-2 secretion and can be integrated into one combined CD28/CD3 zeta signaling receptor molecule. J Immunol 167:6123-31, 2001.
25. Maher J, Brentjens R J, Gunset G, et al: Human T-lymphocyte cytotoxicity and proliferation directed by a single chimeric TCRzeta/CD28 receptor. Nat Biotechnol 20:70-5, 2002.
26. Imai C, Mihara K, Andreansky M, et al: Chimeric receptors with 4-1BB signaling capacity provoke potent cytotoxicity against acute lymphoblastic leukemia. Leukemia 18:676-84, 2004.
27. Wang J, Jensen M, Lin Y, et al: Optimizing adoptive polyclonal T cell immunotherapy of lymphomas, using a chimeric T cell receptor possessing CD28 and CD137 costimulatory domains. Hum Gene Ther 18:712-25, 2007.
28. Zhao Y, Wang Q J, Yang S, et al: A herceptin-based chimeric antigen receptor with modified signaling domains leads to enhanced survival of transduced T lymphocytes and antitumor activity. J Immunol 183:5563-74, 2009.
29. Milone M C, Fish J D, Carpenito C, et al: Chimeric receptors containing CD137 signal transduction domains mediate enhanced survival of T cells and increased antileukemic efficacy in vivo. Mol Ther 17:1453-64, 2009.
30. Yvon E, Del Vecchio M, Savoldo B, et al: Immunotherapy of metastatic melanoma using genetically engineered GD2-specific T cells. Clin Cancer Res 15:5852-60, 2009.
31. Savoldo B, Ramos C A, Liu E, et al: CD28 costimulation improves expansion and persistence of chimeric antigen receptor-modified T cells in lymphoma patients. J Clin Invest 121:1822-6, 2011.
32. Kalinski P, Hilkens C M, Werenga E A, et al: T-cell priming by type-1 and type-2 polarized dendritic cells: the concept of a third signal. Immunol Today 20:561-7, 1999.
33. Kemnade J O, Seethammagari M, Narayanan P, et al: Off-the-shelf Adenoviral-mediated Immunotherapy via Bicistronic Expression of Tumor Antigen and iMyD88/CD40 Adjuvant. Mol Ther, 2012.
34. Schenten D, Nish S A, Yu S, et al: Signaling through the adaptor molecule MyD88 in CD4⁺ T cells is required to overcome suppression by regulatory T cells. Immunity 40:78-90, 2014.
35. Martin S, Pahari S, Sudan R, et al: CD40 signaling in CD8⁺CD40⁺ T cells turns on contra-T regulatory cell functions. J Immunol 184:5510-8, 2010.

### Example 13: Expression of MyD88/CD40 Costimulating Molecules in T Cell-Receptor-Expressing Cells and Tumor Infiltrating Lymphocytes

The following example provides methods used for a MyD88/CD40 polypeptide; this method may be modified as appropriate to assay the inducible chimeric signaling polypeptides discussed herein. In some examples, the method may be modified to provide alternative multimeric ligand binding regions. In some examples, the method may be modified to provide inducible chimeric signaling polypeptides that lack a membrane targeting region.

The modified cells that express the MyD88/CD40 costimulating molecules provided herein may also express a T cell receptor. In these examples, the T cell receptor may be endogenous to the cell, or may be provided to the cell through transfection or transformation with a nucleic acid comprising a polynucleotide encoding a T cell receptor. In certain examples, the T cell receptor may be expressed on the same nucleic acid vector as the MyD88/CD40 costimulating molecule. In further examples, the T cell receptor may be expressed on the same nucleic acid vector as a chimeric inducible Caspase-9 molecule. Methods provided herein for constructing vectors which co-express, or separately express a chimeric antigen receptor, MyD88/CD40 costimulating molecule, or inducible Caspase-9 molecule, may be modified as appropriate for co-expression or separate expression of the T cell receptor, MyD88/CD40 costimulating molecule, or inducible Caspase-9 molecule. In some examples, the modified cells are tumor infiltrating lymphocytes.

### Example 14. Examples of Particular Nucleic Acid and Amino Acid Sequences

The following tables include examples of polypeptide and nucleotide sequences coding for the polypeptides of the chimeric signaling polypeptides of the present embodiments. It is understood that sequences of individual polypeptides provided in these examples, such as, for example, the truncated MyD88 polypeptides, costimulatory polypeptide cytoplasmic signaling regions, FKBP12 variant regions, and caspase polypeptides, may be used to construct other expression vectors that encode chimeric signaling polypeptides of the present embodiments.

Plasmid A pBP1798-SFG-MyD88.CD28.Fv.Fv.T2A.aPSCAscFv.CD34e.CD8stm.zeta (FIG. 10A)

| | SEQ | | SEQ | |
|---|---|---|---|---|
| Fragment | ID # | Nucleotide | ID # | Peptide |
| MyD88 | 1 | ATGGCTGCAGGAGGTCCCGGCGCGGGGTCT | 2 | MAAGGPGAGSAAPVSST |
| | | GCGGCCCCGGTCTCCTCCACATCCTCCCTT | | SSLPLAALNMRVRRRLS |
| | | CCCCTGGCTGCTCTCAACATGCGAGTGCGG | | LFLNVRTQVAADVVTAL |
| | | CGCCGCCTGTCTCTGTTCTTGAACGTGCGG | | AEEMDFEYLEIRQLETQ |
| | | ACACAGGTGGCGGCCGACTGGACCGCGCTG | | ADPTGRLLDAVVQGRPG |
| | | GCGGAGGAGATGGACTTTGAGTACTTGGAG | | ASVGRLLDLLTKLGRDD |
| | | ATCCGGCAACTGGAGACACAAGCGGACCCC | | VLLELGPSIEEDCQKYI |
| | | ACTGGCAGGCTGCTGGACGCCTGGCAGGGA | | LKQQQEEAEKPLQVAAV |
| | | CGCCCTGGCGCCTCTGTAGGCCGACTGCTC | | DSSVPRTAELAGITTLD |
| | | GATCTGCTTACCAAGCTGGGCCGCGACGAC | | DPLGHMPERFDAFICYC |
| | | GTGCTGCTGGAGCTGGGACCCAGCATTGAG | | PSDI |
| | | GAGGATTGCCAAAAGTATATCTTGAAGCAG | | |
| | | CAGCAGGAGGAGGCTGAGAAGCCTTTACAG | | |
| | | GTGGCCGCTGTAGACAGCAGTGTCCCACGG | | |
| | | ACAGCAGAGCTGGCGGGCATCACCACACTT | | |
| | | GATGACCCCCTGGGGCATATGCCTGAGCGT | | |
| | | TTCGATGCCTTCATCTGCTATTGCCCCAGC | | |
| | | GACATC | | |
| Linker | | CTCGAG | | LE |
| | | AGGAGTAAGAGGAGCAGGCTCCTGCACAGT | | RSKRSRLLHSDYMNMTP |
| CD28 | | GACTACATGAACATGACTCCCCGCCGCCCC | | RRPGPTRKHYQPYAPPR |
| signaling | | GGGCCCACCCGCAAGCATTACCAGCCCTAT | | DFAAYRSGSGQL |
| domain | | GCCCCACCACGCGACTTCGCAGCCTATCGC | | |
| | | TCC | | |
| Linker | | GGATCTGGCCAATTG | | GSGQL |
| FKBPᵥ′ | | GGCGTCCAAGTCGAAACCATTAGTCCCGGC | | GVQVETISPGDGRTFPK |
| | | GATGGCAGAACATTTCCTAAAAGGGGACAA | | RGQTCVVHYTGMLEDGK |
| | | ACATGTGTCGTCCATTATACAGGCATGTTG | | KVDSSRDRNKPFKFMLG |
| | | GAGGACGGCAAAAAGGTGGACAGTAGTAGA | | KQEVIRGVVEEGVAQMS |
| | | GATCGCAATAAACCTTTCAAATTCATGTTG | | VGQRAKLTISPDYAYGA |
| | | GGAAAACAAGAAGTCATTAGGGGATGGGAG | | TGHPGIIPPHATLVFDV |
| | | GAGGGCGTGGCTCAAATGTCCGTCGGCCAA | | ELLKLE |
| | | CGCGCTAAGCTCACCATCAGCCCCGACTAC | | |
| | | GCATACGGCGCTACCGGACATCCCGGAATT | | |
| | | ATTCCCCCTCACGCTACCTTGGTGTTTGAC | | |
| | | GTCGAACTGTTGAAGCTCGAA | | |
| Linker | | GTCGAG | | VE |
| FKBPᵥ | | GGAGTGCAGGTGGAGACTATCTCCCCAGGA | | GVQVETISPGDGRTFPK |
| | | GACGGGCGCACCTTCCCCAAGCGCGGCCAG | | RGQTCVVHYTGMLEDGK |
| | | ACCTGCGTGGTGCACTACACCGGGATGCTT | | KVDSSRDRNKPFKFMLG |
| | | GAAGATGGAAAGAAAGTTGATTCCTCCCGG | | KQEVIRGVVEEGVAQMS |
| | | GACAGAAACAAGCCCTTTAAGTTTATGCTA | | VGQRAKLTISPDYAYGA |
| | | GGCAAGCAGGAGGTGATCCGAGGCTGGGAA | | TGHPGIIPPHATLVFDV |
| | | GAAGGGGTTGCCCAGATGAGTGTGGGTCAG | | ELLKLE |
| | | AGAGCCAAACTGACTATATCTCCAGATTAT | | |
| | | GCCTATGGTGCCACTGGGCACCCAGGCATC | | |
| | | ATCCCACCACATGCCACTCTCGTCTTCGAT | | |
| | | GTGGAGCTTCTAAAACTGGAA | | |
| Linker | | CCGCGG | | PR |
| T2A | | GAGGGCAGAGGCAGCCTCCTGACATGTGGG | | EGRGSLLTCGDVEENPG |
| | | GACGTCGAGGAGAACCCTGGCCCA | | P |
| Linker | | CCTTGG | | PVV |
| Signal | | ATGGAGTTCGGATTGAGCTGGCTGTTCCTG | | MEFGLSVVLFLVAILKG |
| Peptide | | GTGGCAATACTCAAGGGCGTTCAATGTTCA | | VQCSR |
| | | CGG | | |
| PSCA(A11) | | GACATCCAACTGACGCAAAGCCCATCTACA | | DIQLTQSPSTLSASMGD |
| V_{L} | | CTCAGCGCTAGCATGGGGGACAGGGTCACA | | RVTITCSASSSVRFIHV |
| | | ATCACGTGCTCTGCCTCAAGTTCCGTTAGG | | VYQQKPGKAPKRLIYDT |
| | | TTTATCCATTGGTATCAGCAGAAACCTGGA | | SKLASGVPSRFSGSGSG |
| | | AAGGCCCCAAAAAGACTGATCTATGATACC | | TDFTLTISSLQPEDFAT |
| | | AGCAAGCTGGCTTCCGGAGTGCCCTCAAGG | | YYCQQVVGSSPFTFGQG |
| | | TTCTCAGGATCTGGCAGTGGGACCGATTTC | | TKVEIK |
| | | ACCCTGACAATTAGCAGCCTTCAGCCAGAG | | |
| | | GATTTCGCAACCTATTACTGTCAGCAATGG | | |
| | | GGGTCCAGCCCATTCACTTTCGGCCAAGGA | | |
| | | ACAAAGGTGGAGATAAAA | | |
| Flex | | GGCGGAGGAAGCGGAGGTGGGGGC | | GGGSGGGG |
| Linker | | CAGGTG | | QV |
| PSCA(A11) | | GAGGTGCAGCTCGTGGAGTATGGCGGGGGC | | EVQLVEYGGGLVQPGGS |
| V_{H} | | CTGGTGCAGCCTGGGGGTAGTCTGAGGCTC | | LRLSCAASGFNIKDYYI |
| | | TCCTGCGCTGCCTCTGGCTTTAACATTAAA | | HVVVRQAPGKGLEVVVA |
| | | GACTACTACATACATTGGGTGCGGCAGGCC | | VVIDPENGDTEFVPKFQ |
| | | CCAGGCAAAGGGCTCGAATGGGTGGCCTGG | | GRATMSADTSKNTAYLQ |
| | | ATTGACCCTGAGAATGGTGACACTGAGTTT | | MNSLRAEDTAVYYCKTG |
| | | GTCCCCAAGTTTCAGGGCAGAGCCACCATG | | GFVVGQGTLVTVSS |
| | | AGCGCTGACACAAGCAAAAACACTGCTTAT | | |
| | | CTCCAAATGAATAGCCTGCGAGCTGAAGAT | | |
| | | ACAGCAGTCTATTACTGCAAGACGGGAGGA | | |
| | | TTCTGGGGCCAGGGAACTCTGGTGACAGTT | | |
| | | AGTTCC | | |
| Linker | | GGATCC | | GS |
| CD34 | | GAACTTCCTACTCAGGGGACTTTCTCAAAC | | ELPTQGTFSNVSTNVS |
| epitope | | GTTAGCACAAACGTAAGT | | |
| CD8 stalk | | GAACTTCCTACTCAGGGGACTTTCTCAAAC | | PAPRPPTPAPTIASQPL |
| | | GTTAGCACAAACGTAAGT | | SLRPEACRPAAGGAVHT |
| | | | | RGLDFACD |
| CD8 | | ATCTATATCTGGGCACCTCTCGCTGGCACC | | IYIVVAPLAGTCGVLLL |
| trans- | | TGTGGAGTCCTTCTGCTCAGCCTGGTTATT | | SLVITLYCNHRNRRRVC |
| membrane | | ACTCTGTACTGTAATCACCGGAATCGCCGC | | KCPR |
| | | CGCGTTTGTAAGTGTCCCAGG | | |
| Linker | | GTCGAC | | VD |
| CD3ζ | | AGAGTGAAGTTCAGCAGGAGCGCAGACGCC | | RVKFSRSADAPAYQQGQ |
| | | CCCGCGTACCAGCAGGGCCAGAACCAGCTC | | NQLYNELNLGRREEYDV |
| | | TATAACGAGCTCAATCTAGGACGAAGAGAG | | LDKRRGRDPEMGGKPRR |
| | | GAGTACGATGTTTTGGACAAGAGACGTGGC | | KNPQEGLYNELQKDKMA |
| | | CGGGACCCTGAGATGGGGGGAAAGCCGAGA | | EAYSEIGMKGERRRGKG |
| | | AGGAAGAACCCTCAGGAAGGCCTGTACAAT | | HDGLYQGLSTATKDTYD |
| | | GAACTGCAGAAAGATAAGATGGCGGAGGCC | | ALHMQALPPR |
| | | TACAGTGAGATTGGGATGAAAGGCGAGCGC | | |
| | | CGGAGGGGCAAGGGGCACGATGGCCTTTAC | | |
| | | CAGGGTCTCAGTACAGCCACCAAGGACACC | | |
| | | TACGACGCCCTTCACATGCAAGCTCTTCCA | | |
| | | CCTCGT | | |
| STOP | | TGA | | STOP |

The following sequences are examples of sequences of scFv regions of chimeric antigen receptors. These nucleotide and polypeptide sequences may be used to modify the specificity of a chimeric antigen receptor of the present embodiments. For example, an scFv portion providing specify to an antigen such as, for example, PSCA, or HER2 in the plasmids or sequences provided herein, may be substituted with an scFv portion providing specificity to different antigen, such as, for example, CD123, by substituting the CD123 scFv nucleotide sequence for the PSCA or HER2 scFv nucleotide sequence. Provided herein are nucleic acids and cells of the present embodiments that comprise polynucleotides that encode CARs comprising scFvs having amino acid sequences 90%, 92%, 94%, 95%, 97%, 99% or more identical to the amino acid sequences of the scFv sequences, provided herein, or having the amino acid sequences of the scFv sequences provided herein.

HER2 (4D5)

| Fragment | Nucleotide | Peptide |
|---|---|---|
| Signal Peptide | ATGGATTTTCAAGTGCAGATTTTCAGCTTTCTCT | MDFQVQIFSFLLISASVI |
| | TGATCAGTGCCAGCGTGATCATGAGCAGGGGC | MSRG |
| HER2(4D5) V_{L} | GACATCCAGATGACTCAATCACCCTCAAGTCTG | DIQMTQSPSSLSASVG |
| | AGTGCATCAGTGGGCGACCGAGTAACAATTAC | DRVTITCRASQDVNTAV |
| | GTGCCGGGCCTCTCAGGATGTCAACACCGCCG | AWYQQKPGKAPKLLIY |
| | TGGCCTGGTATCAACAAAAACCTGGAAAGGCCC | SASFLYSGVPSRFSGS |
| | CTAAGCTCCTCATATATAGCGCCTCATTCCTTTA | RSGTDFTLTISSLQPED |
| | TTCTGGTGTACCTAGTCGCTTTTCAGGGTCAAG | FATYYCQQHYTTPPTF |
| | ATCAGGAACCGATTTTACACTTACAATCTCTAGT | GQGTKVEIKRTGSTSG |
| | CTGCAACCAGAGGATTTTGCTACCTACTATTGC | SGKPGSGEGS |
| | CAGCAACACTATACAACGCCGCCTACATTCGGG | |
| | CAGGGGACGAAGGTGGAGATTAAACGCACT | |
| Flex | GGCTCAACCAGTGGGTCAGGCAAGCCTGGGTC | GSTSGSGKPGSGEGS |
| | CGGCGAGGGCTCC | |
| HER2(4D5) V_{H} | GAGGTACAACTGGTTGAAAGCGGAGGAGGGCT | EVQLVESGGGLVQPGG |
| | GGTTCAGCCAGGCGGGTCACTCCGCCTCTCCT | SLRLSCAASGFNIKDTYI |
| | GTGCGGCGTCCGGGTTCAACATAAAGGACACC | HWVRQAPGKGLEWVA |
| | TATATCCATTGGGTTCGCCAGGCCCCCGGAAA | RIYPTNGYTRYADSVKG |
| | GGGATTGGAGTGGGTTGCTCGAATTTATCCAAC | RFTISADTSKNTAYLQM |
| | TAATGGGTACACCCGCTATGCTGATTCCGTGAA | NSLRAEDTAVYYCSRW |
| | GGGTCGCTTTACGATTAGTGCCGACACTTCGAA | GGDGFYAMDFWGQGT |
| | AAACACAGCTTACTTGCAAATGAACAGTCTCCG | LVTVSS |
| | CGCCGAGGACACCGCAGTATACTATTGCAGTA | |
| | GGTGGGGGGGAGACGGGTTTTATGCTATGGAT | |
| | TTCTGGGGACAAGGTACTCTGGTAACCGTGAG | |
| | CAGT | |

GD2 (14g2a)

| Fragment | Nucleotide | Peptide |
|---|---|---|
| Signal Peptide | ATGCTGCTGCTCGTGACATCTCTGCTGCTGTGC | MLLLVTSLLLCELPHPA |
| | GAGCTGCCCCACCCCGCCTTTCTGCTGATCCC | FLLIP |
| | C | |
| GD2 (14d5a) | GATATCCTGCTGACCCAGACCCCTCTGAGCCTG | DILLTQTPLSLPVSLGD |
| V_{L} | CCTGTGTCTCTGGGCGATCAGGCCAGCATCAG | QASISCRSSQSLVHRN |
| | CTGCAGATCCAGCCAGAGCCTGGTGCACCGGA | GNTYLHWYLQKPGQS |
| | ACGGCAACACCTACCTGCACTGGTATCTGCAGA | PKLLIHKVSNRFSGVP |
| | AGCCCGGCCAGAGCCCCAAGCTGCTGATTCAC | DRFSGSGSGTDFTLKI |
| | AAGGTGTCCAACCGGTTCAGCGGCGTGCCCGA | SRVEAEDLGVYFCSQS |
| | CAGATTTTCTGGCAGCGGCTCCGGCACCGACT | THVPPLTFGAGTKLEL |
| | TCACCCTGAAGATCAGCCGGGTGGAAGCCGAG | K |
| | GACCTGGGCGTGTACTTCTGCAGCCAGTCCAC | |
| | CCACGTGCCCCCCCTGACATTTGGCGCCGGAA | |
| | CAAAGCTGGAACTGAAG | |
| Flex | GGCAGCACAAGCGGCAGCGGCAAGCCTGGATC | GSTSGSGKPGSGEGS |
| | TGGCGAGGGAAGCACCAAGGGC | TKG |
| GD2 (14d5a) | GAAGTGAAGCTGCAGCAGAGCGGCCCCTCTCT | EVKLQQSGPSLVEPGA |
| V_{H} | GGTGGAACCTGGCGCCTCTGTGATGATCTCCT | SVMISCKASGSSFTGY |
| | GCAAGGCCAGCGGCAGCTCCTTCACCGGCTAC | NMNWVRQNIGKSLEW |
| | AACATGAACTGGGTGCGCCAGAACATCGGCAA | IGAIDPYYGGTSYNQK |
| | GAGCCTGGAATGGATCGGCGCCATCGACCCCT | FKGRATLTVDKSSSTA |
| | ACTACGGCGGCACCAGCTACAACCAGAAGTTC | YMHLKSLTSEDSAVYY |
| | AAGGGCAGAGCCACCCTGACCGTGGACAAGAG | CVSGMEYWGQGTSVT |
| | CAGCTCCACCGCCTACATGCACCTGAAGTCCCT | VSS |
| | GACCAGCGAGGACAGCGCCGTGTACTACTGCG | |
| | TGTCCGGCATGGAATACTGGGGCCAGGGCACA | |
| | AGCGTGACCGTGTCCTCT | |

CD123 (32716)

| Fragment | Nucleotide | Peptide |
|---|---|---|
| Signal Peptide | ATGGAGTTCGGATTGAGCTGGCTGTTCCTGGTG | MEFGLSWLFLVAILKG |
| | GCAATACTCAAGGGCGTTCAATGTTCACGG | VQCSR |
| CD123 | CAGATCCAACTGGTGCAGTCAGGCCCGGAACT | QIQLVQSGPELKKPGE |
| (32716) V_{L} | GAAGAAGCCAGGGGAGACAGTCAAAATAAGTT | TVKISCKASGYIFTNYG |
| | GTAAAGCCAGCGGCTACATATTTACTAATTACG | MNWVKQAPGKSFKW |
| | GGATGAATTGGGTGAAGCAAGCGCCGGGCAAA | MGWINTYTGESTYSAD |
| | TCCTTTAAATGGATGGGGTGGATAAACACATAC | FKGRFAFSLETSASTA |
| | ACAGGAGAGTCAACGTACAGCGCGGACTTCAA | YLHINDLKNEDTATYFC |
| | AGGTCGATTCGCGTTCAGTCTCGAGACCAGCG | ARSGGYDPMDYWGQ |
| | CGAGTACAGCTTACCTCCACATCAACGATCTTA | GTSVTV |
| | AAAACGAAGACACGGCAACCTATTTTTGCGCCC | |
| | GGTCAGGCGGTTACGACCCTATGGACTATTGG | |
| | GGCCAAGGGACCTCCGTTACGGTA | |
| Flex | TCTTCAGGCGGTGGCGGGAGTGGTGGAGGAG | SSGGGGSGGGGSGG |
| | GCTCAGGCGGCGGGGGATCA | GGS |
| CD123 | GACATCGTACTGACCCAATCTCCCGCTAGCCTT | DIVLTQSPASLAVSLG |
| (32716) V_{H} | GCAGTATCCTTGGGTCAACGCGCTACAATAAGT | QRATISCRASESVDNY |
| | TGCCGGGCTAGTGAGTCCGTAGACAACTATGG | GNTFMHWYQQKPGQ |
| | CAACACCTTCATGCATTGGTACCAACAAAAACC | PPKLLIYRASNLESGIP |
| | AGGTCAGCCACCCAAACTTCTCATTTACAGAGC | ARFSGSGSRTDFTLTI |
| | GTCTAATCTCGAAAGCGGCATCCCTGCTCGATT | NPVEADDVATYYCQQ |
| | CTCTGGAAGCGGAAGTAGAACCGACTTTACACT | SNEDPPTFGAGTKLEL |
| | GACTATAAACCCCGTCGAAGCCGATGATGTTGC | KESKYGPPCP |
| | CACTTATTACTGTCAACAGAGCAATGAGGACCC | |
| | ACCGACATTCGGTGCTGGTACCAAGCTGGAGTT | |
| | GAAGGAGTCAAAATACGGGCCTCCCTGTCCC | |

CD33 (My9-6)

| Fragment | Nucleotide | Peptide |
|---|---|---|
| Signal Peptide | ATGGAGTTCGGATTGAGCTGGCTGTTCCTGGTG | MEFGLSWLFLVAILKG |
| | GCAATACTCAAGGGCGTTCAATGTTCACGG | VQCSR |
| CD33 (My9-6) | GAAATTGTGCTGACTCAGAGCCCGGGTAGCCT | EIVLTQSPGSLAVSPG |
| V_{L} | GGCCGTGTCCCCCGGAGAGCGAGTGACCATGA | ERVTMSCKSSQSVFFS |
| | GCTGTAAATCCAGCCAATCAGTTTTTTTTTCATC | SSQKNYLAWYQQIPG |
| | ATCTCAAAAAAACTATCTGGCATGGTACCAACA | QSPRLLIYWASTRESG |
| | GATACCCGGGCAGTCCCCACGGCTGCTGATTT | VPDRFTGSGSGTDFTL |
| | ACTGGGCATCAACACGCGAGAGCGGTGTGCCC | TISSVQPEDLAIYYCHQ |
| | GACAGATTCACCGGAAGCGGGAGCGGCACGGA | YLSSRTFGQGTKLEIK |
| | CTTCACACTTACCATCTCAAGCGTACAACCGGA | R |
| | GGACTTGGCTATCTATTACTGCCACCAATATCTT | |
| | TCCTCCAGAACATTCGGACAGGGAACGAAACTG | |
| | GAGATCAAAAGA | |
| Flex | GGCGGCGGGAGTGGGGGAGGAGGTCAGGTG | GGGSGGGGQV |
| CD33 (My9-6) | CAGGTGCAGCTGCAGCAGCCTGGAGCCGAGGT | QVQLQQPGAEVVKPG |
| V_{H} | GGTGAAGCCCGGCGCATCTGTGAAAATGTCTT | ASVKMSCKASGYTFTS |
| | GCAAGGCAAGCGGATATACATTTACTAGCTACT | YYIHWIKQTPGQGLEW |
| | ACATCCATTGGATCAAGCAAACCCCCGGACAG | VGVIYPGNDDISYNQK |
| | GGCCTCGAATGGGTGGGAGTTATTTACCCGGG | FQGKATLTADKSSTTA |
| | GAACGATGATATCTCTTATAATCAGAAATTCCAA | YMQLSSLTSEDSAVYY |
| | GGGAAAGCCACCCTGACTGCAGACAAATCAAG | CAREVRLRYFDVWGQ |
| | TACCACAGCCTATATGCAGCTCAGCTCCCTGAC | GTTVTVSS |
| | AAGCGAGGATTCCGCTGTGTACTACTGTGCCAG | |
| | GGAGGTTAGACTTCGATATTTTGATGTTTGGGG | |
| | GCAGGGAACTACCGTGACCGTGAGCAGC | |

### Example 15: Representative Embodiments

Provided hereafter are examples of certain embodiments of the technology.

A1. A nucleic acid comprising a promoter operably linked to a polynucleotide encoding an inducible chimeric signaling polypeptide, wherein the polypeptide comprises
a) one or more multimeric ligand binding regions that bind to a multimeric ligand;
b) a MyD88 polypeptide or a truncated MyD88 polypeptide lacking the TIR domain; and
c) a costimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions.

A1.1. The nucleic acid of embodiment A1, wherein the costimulatory polypeptide cytoplasmic signaling region is selected from the group consisting of CD27, CD28, ICOS, 4-1BB, RANK/TRANCE-R, and OX40.

A1.2. The nucleic acid of embodiment A1, wherein the costimulatory polypeptide cytoplasmic signaling region is selected from the group consisting of CD27, CD30, TweakR, TAC1, BCMA and HVEM.

A1.3. The nucleic acid of embodiment A1, wherein the costimulatory polypeptide cytoplasmic signaling region is selected from the group consisting of CD27, CD30, TweakR, BCMA and HVEM.

A1.4. The nucleic acid of embodiment A1, wherein the costimulatory polypeptide cytoplasmic signaling region is selected from the group consisting of CD28, 4-1BB, OX40, and ICOS.

A2. The nucleic acid of any one of embodiments A1-A1.4, wherein the polypeptide comprises a truncated MyD88 polypeptide lacking a TIR domain.

A3. The nucleic acid of any one of embodiments A1-A2, wherein the costimulatory polypeptide cytoplasmic signaling region is a 4-1BB cytoplasmic signaling region.

A4. The nucleic acid of any one of embodiments A1-A2, wherein the costimulatory polypeptide cytoplasmic signaling region is an OX40 cytoplasmic signaling region.

A5. The nucleic acid of any one of embodiments A1-A2, wherein the costimulatory polypeptide cytoplasmic signaling region is a CD28 cytoplasmic signaling region.

A6. The nucleic acid of any one of embodiments A1-A2, wherein the costimulatory polypeptide cytoplasmic signaling region is an ICOS cytoplasmic signaling region.

A7. The nucleic acid of any one of embodiments A1-A6, wherein the inducible chimeric signaling polypeptide comprises two multimeric ligand binding regions.

A7.1. The nucleic acid of any one of embodiments A1-A6, wherein the inducible chimeric signaling polypeptide comprises three multimeric ligand binding regions.

A7.2. The nucleic acid of any one of embodiments A1-A6, wherein the inducible chimeric signaling polypeptide comprises four or more multimeric ligand binding regions.

A7.3. The nucleic acid of any one of embodiments A1-A7.2, wherein one or more of the multimeric ligand binding regions comprises an FKBP12 variant polypeptide.

A7.3.1. The nucleic acid of any one of embodiments A1-A7, wherein the inducible chimeric signaling polypeptide comprises two or more multimeric ligand binding regions, wherein the multimeric ligand binding regions comprise FKBP12 variant polypeptides.

A7.3.2. The nucleic acid of embodiment A7.3.1., wherein the FKBP12 variant polypeptide binds with higher affinity to the multimeric ligand than the wild type FKBP12 polypeptide.

A7.4. The nucleic acid of any one of embodiments A7.3.1-A7.3.2, wherein one or more of the multimeric ligand binding regions comprises an FKBP12 variant polypeptide comprising an amino acid substitution at position 36 that binds with higher affinity to the multimeric ligand than the wild type FKBP12 polypeptide.

A7.5. The nucleic acid of any one of embodiments A1-A7.3, wherein each of the multimeric ligand binding regions comprises an FKBP12 variant polypeptide comprising an amino acid substitution at position 36 that binds with higher affinity to the multimeric ligand than the wild type FKBP12 polypeptide.

A7.6. The nucleic acid of any one of embodiments A7.4 or A7.5, wherein the amino acid substitutions at position 36 are selected from the group consisting of valine, isoleucine, leucine, and alanine.

A7.7. The nucleic acid of any one of embodiments A7.3 to A7.6, wherein one or more of the FKBP12 variant polypeptides is an FKBP12v36 polypeptide.

A7.8. The nucleic acid of any one of embodiments A7.3.1 to A7.6, wherein each of the FKBP12 variant polypeptides are FKBP12v36 polypeptides.

A8. The nucleic acid of any one of embodiments A1-A7.8, wherein the multimeric ligand binding region comprises two FKBP12 variant polypeptides, wherein each of the FKBP12 variant polypeptides comprises an amino acid substitution at position 36 that binds with higher affinity to the multimeric ligand than the wild type FKBP12 polypeptide.

A8.1. The nucleic acid of embodiment A8, wherein the amino acid substitutions at position 36 are selected from the group consisting of valine, isoleucine, leucine, and alanine.

A8.2. The nucleic acid of embodiment A8, wherein the inducible multimeric ligand binding region comprises two FKBP12v36 polypeptides.

A8.3. The nucleic acid of any one of embodiments A8-A8.2 wherein the multimeric ligand binding region comprises an Fv′Fvls polypeptide.

A8.4. The nucleic acid of any one of embodiments A7.7 to A8.2, wherein the multimeric ligand binding region comprises two FKBP12v36 polypeptides, wherein the first FKBP12v36 polypeptide is encoded by the nucleotide sequence of SEQ ID NO: 5, and the second FKBP12v36 polypeptide is encoded by the nucleotide sequence of SEQ ID NO: 7.

A9. The nucleic acid of any one of embodiments A1-A8.4, wherein the ligand is dimeric FK506, or a dimeric FK506-like analog.

A10. The nucleic acid of any one of embodiments A1-A9, wherein the ligand is rimiducid. A11. The nucleic acid of any one of embodiments A1-A9, wherein the ligand is AP20187.

A12-A14. Reserved

A15. The nucleic acid of any one of embodiments A1-A11, wherein the truncated MyD88 polypeptide comprises the amino acid sequence of the full length MyD88 polypeptide of SEQ ID NO: 907 lacking the TIR domain, or a functional fragment thereof.

A16. The nucleic acid of any one of embodiments A1-A15, wherein the truncated MyD88 polypeptide does not comprise contiguous amino acid residues 156 to the C-terminus of the full length MyD88 polypeptide.

A16.1. The nucleic acid of any one of embodiments A1-A15, wherein the truncated MyD88 polypeptide does not comprise contiguous amino acid residues 152 to the C-terminus of the full length MyD88 polypeptide.

A16.2. The nucleic acid of any one of embodiments A1-A15, wherein the truncated MyD88 polypeptide does not comprise contiguous amino acid residues 173 to the C-terminus of the full length MyD88 polypeptide.

A16.3. The nucleic acid of any one of embodiments A16-A16.2, wherein the full length MyD88 polypeptide comprises the amino acid sequence of SEQ ID NO: 907.

A17. The nucleic acid of any one of embodiments A1-A16.2, wherein the truncated MyD88 polypeptide consists of the amino acid sequence of SEQ ID NO: 2, or a functional fragment thereof.

A18. The nucleic acid of any one of embodiments A1-A17, wherein the cytoplasmic signaling region comprises an amino acid sequence selected from the group consisting of SEQ ID NOs: 6, 48, 90, and 174, or a functional fragment thereof.

A19. The nucleic acid of any one of embodiments A1-A17, wherein the cytoplasmic signaling region consists of an amino acid sequence selected from the group consisting of SEQ ID NOs: 6, 48, 90, and 174, or a functional fragment thereof.

A20. A nucleic acid comprising a promoter operably linked to a polynucleotide encoding an inducible chimeric signaling polypeptide, wherein the polypeptide comprises

a) a multimeric ligand binding region comprising two FKBP12v36 polypeptides;

b) a truncated MyD88 polypeptide lacking a TIR domain; and

c) a CD28 cytoplasmic signaling region.

A21. A nucleic acid comprising a promoter operably linked to a polynucleotide encoding an inducible chimeric signaling polypeptide, wherein the polypeptide comprises

a) a multimeric ligand binding region comprising two FKBP12v36 polypeptides;

b) a truncated MyD88 polypeptide lacking a TIR domain; and

c) a 4-1BB cytoplasmic signaling region.

A21.1. The nucleic acid of any one of embodiments A20 to A21, wherein the multimeric ligand binding region comprises two FKBP12v36 polypeptides, wherein the first FKBP12v36 polypeptide is encoded by the nucleotide sequence of SEQ ID NO: 5, and the second FKBP12v36 polypeptide is encoded by the nucleotide sequence of SEQ ID NO: 7.

A22. The nucleic acid of any one of embodiments A1-A21.1, wherein the inducible chimeric signaling polypeptide further comprises a membrane targeting region.

A23. The nucleic acid of embodiment A22, wherein the membrane targeting region is selected from the group consisting of a myristoylation region, a palmitoylation region, a prenylation region, and transmembrane sequences of receptors.

A24. The nucleic acid of embodiment A22, wherein the membrane-targeting region is a myristoylation region.

A25. The nucleic acid of any one of embodiments A1-A24, further comprising a polynucleotide coding for a heterologous protein.

A26. The nucleic acid of any one of embodiments A1-A24, further comprising a polynucleotide coding for a chimeric antigen receptor.

A27. The nucleic acid of embodiment A26, wherein the chimeric antigen receptor comprises (i) a transmembrane region, (ii) a T cell activation molecule, and (iii) an antigen recognition moiety.

A28. The nucleic acid of any one of embodiments A1-A24, further comprising a polynucleotide coding for a recombinant T cell receptor.

A29. The nucleic acid of any one of embodiments A25-A28, further comprising a polynucleotide encoding a linker polypeptide between the two polynucleotides, wherein the linker polypeptide separates the translation products of the two polynucleotides during or after translation.

A30. The nucleic acid of embodiment A29, wherein the linker polypeptide that separates the translation products of two polynucleotides is a 2A polypeptide.

A31. The nucleic acid of any one of embodiments A25-A28, further comprising a second promoter operably linked to the polynucleotide encoding the heterologous polypeptide, the chimeric antigen receptor or the recombinant T cell receptor.

A31.1. The nucleic acid of any one of embodiments A1-A6, A15-A19, or A22-A31, wherein the multimeric ligand binding region comprises a FKBP-rapamycin binding domain of mTOR (FRB) or FRB variant region.

A32. The nucleic acid of any one of embodiments A1-A6, A15-A19, or A22-A31, wherein the multimeric ligand binding region comprises a FKBP-rapamycin binding domain of mTOR (FRB) or FRB variant region; and a FKBP12 polypeptide region.

A33. The nucleic acid of any one of embodiments A31.1 or A32, wherein the FRB variant region is selected from the group consisting of KLW (T2098L), KTF (W2101F), and KLF (T2098L, W2101F).

A34. The nucleic acid of any one of embodiments A31.1-A33, wherein the FRB variant region is FRB_{L}

A35. The nucleic acid or cell of any one of embodiments A31.1-A33, wherein the FRB variant region binds to a rapalog selected from the group consisting of S-o,p-dimethoxyphenyl (DMOP)-rapamycin, R-Isopropoxyrapamycin, S-Butanesulfonamidorap, R and S C7-ethyloxyrapamycin, R and S C7-isopropyloxyrapamycin, R and S C7-isobutylrapamycin, R and S ethylcarbamaterapamycin, R and S C7-phenylcarbamaterapamycin, R and S C7-(3-methyl)indole rapamycin, temsirolimus, everolimus, zotarolimus, and R and S C7-(7-methyl)indole rapamycin.

A36. The nucleic acid of any one of embodiments A31.1-A35, further comprising a polynucleotide encoding a chimeric Caspase-9 polypeptide comprising a modified FKBP12 polypeptide that binds to rimiducid, and a Caspase-9 polypeptide.

A37. The nucleic acid of embodiment A36, wherein the Caspase-9 polypeptide lacks the CARD domain.

A38. The nucleic acid of embodiment A37, wherein the Caspase-9 polypeptide comprises the amino acid sequence of SEQ ID NO: 670

A39. The nucleic acid of embodiment A37, wherein the Caspase-9 polypeptide comprises the amino acid sequence of SEQ ID NO. 670 and further comprises an amino acid substitution selected from the group consisting of the caspase variants in Table 5.

A40. The nucleic acid of embodiment A39, wherein the amino acid substitution is selected from the group consisting of N405Q, D330A, and D330E

A41. The nucleic acid of any one of embodiments A36-A40, further comprising a polynucleotide encoding a linker polypeptide between two polynucleotides of the nucleic acid, wherein the linker polypeptide separates the translation products of the polynucleotide encoding the chimeric Caspase-9 polypeptide from the translation product of another polynucleotide of the nucleic acid.

A42. The nucleic acid of embodiment A41, wherein the linker polypeptide that separates the translation products of two polynucleotides is a 2A polypeptide.

A43. The nucleic acid of any one of embodiments A36-A42, wherein modified FKBP12 polypeptide comprises an amino acid substitution at position 36 that binds with higher affinity to the multimeric ligand than the wild type FKBP12 polypeptide.

A44. The nucleic acid of embodiment A43, wherein the amino acid substitution at position 36 is selected from the group consisting of valine, isoleucine, leucine, and alanine.

A45. The nucleic acid of any one of embodiments A36-A44, wherein the modified FKBP12 polypeptide is FKBP12v36.

A46. The nucleic acid of any one of embodiments A36-A45 wherein dimeric FK506, or a dimeric FK506-like analog binds to the modified FKBP12 polypeptide.

A47. The nucleic acid of any one of embodiments A36-A45, wherein AP20187 binds to the modified FKBP12 polypeptide.

A47.01 The nucleic acid of any one of embodiments A1-A47, wherein the inducible chimeric signaling polypeptided does not include a CD40 cytoplasmic region polypeptide.

A47.1. The nucleic acid of any one of embodiments A1-A47.01, wherein the nucleic acid is an isolated nucleic acid.

A47.2. The nucleic acid composition of any one of embodiments A1-A47.1, wherein the nucleic acid comprises a promoter sequence operably linked to the polynucleotide.

A47.3. The nucleic acid composition of any one of embodiments A1-A47.1, wherein the nucleic acid is contained within a viral vector.

A47.4. The nucleic acid composition of any one of embodiments A1-A47.3, wherein the viral vector is a retroviral vector.

A47.8. The nucleic acid composition of any one of embodiments A1-A47.2, wherein the nucleic acid is contained within a plasmid.

A49. An inducible chimeric signaling polypeptide encoded by a nucleic acid of any one of embodiments A1-A48.

B1. A modified cell, wherein the cell is transduced or transfected with a nucleic acid of any one of embodiments A1-A48.

B2. A modified cell, wherein the cell is transduced or transfected with
a) a nucleic acid of any one of embodiments A1-A48; and
b) a nucleic acid comprising a polynucleotide coding for a heterologous protein.

B3. A modified cell, wherein the cell is transduced or transfected with
a) a nucleic acid of any one of embodiments A1-A48; and
b) a nucleic acid comprising a polynucleotide coding for a chimeric antigen receptor.

B4. The modified cell of embodiment B3, wherein the chimeric antigen receptor comprises (i) a transmembrane region, (ii) a T cell activation molecule, and (iii) an antigen recognition moiety.

B5. A modified cell, wherein the cell is transduced or transfected with
a) a nucleic acid of any one of embodiments A1-A48; and
b) a nucleic acid comprising a polynucleotide coding for a recombinant T cell receptor.

B6. The modified cell of any one of embodiments B1-B5, wherein the cell is selected from the group consisting of T cell, tumor infiltrating lymphocyte, NK-T cell, invariant NK-T cell, gamma delta T cell, and NK cell.

B7. The modified cell of any one of embodiments B1-B5, wherein the cell is a T cell.

B7.1. The modified cell of any one of embodiments B1-B5, wherein the cell is an NK-T cell.

B7.1.1. The modified cell of any one of embodiments B1-B5, wherein the cell is an invariant NK-T cell.

B7.1.2. The modified cell of any one of embodiments B1-B5, wherein the cell is a gamma delta T cell.

B7.2. The modified cell of any one of embodiments B1-B5, wherein the cell is a tumor infiltrating lymphocyte, wherein the tumor infiltrating lymphocyte is not an antigen-presenting cell.

B8. The modified cell of any one of embodiments B1-B5, wherein the cell is an NK cell.

B9. The modified cell of any one of embodiments B1-B8, wherein the cell is obtained or prepared from bone marrow.

B10. The modified cell of any one of embodiments B1-B8, wherein the cell is obtained or prepared from umbilical cord blood.

B11. The modified cell of any one of embodiments B1-B8, wherein the cell is obtained or prepared from peripheral blood.

B12. The modified cell of any one of embodiments B1-B8, wherein the cell is obtained or prepared from peripheral blood mononuclear cells.

B13. The modified cell of any one of embodiments B1-B8, wherein the cell is a human cell.

B14. The modified cell of any one of embodiments B1-B8, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

C1. A method for expressing an inducible chimeric signaling polypeptide in a cell, comprising contacting a nucleic acid of any one of embodiments A1 to A48 with a cell under conditions in which the nucleic acid is incorporated into the cell, whereby the cell expresses the inducible chimeric signaling polypeptide from the incorporated nucleic acid.

C2. The method of embodiment C1, wherein the cell is contacted with a nucleic acid of any one of embodiments A25 or A29-A31, wherein the cell further expresses a heterologous protein.

C3. The method of embodiment C1, wherein the cell is contacted with a nucleic acid of any one of embodiments A26-A27, or A29-A31, wherein the cell further expresses a chimeric antigen receptor.

C4. The method of embodiment C1, wherein the cell is contacted with a nucleic acid of any one of embodiments A28-A31, wherein the cell further expresses a recombinant T cell receptor.

C5. The method of embodiment C1, wherein the cell is contacted with a nucleic acid of any one of embodiments A36-A48, wherein the cell expresses the chimeric Caspase-9 polypeptide.

C6. The method of any one of embodiments C1-C5, wherein the nucleic acid is contacted with the cell ex vivo.

C7. The method of any one of embodiments C1-C5, wherein the nucleic acid is contacted with the cell in vivo.

C8. The method of any one of embodiments C1-C7, wherein the cell is selected from the group consisting of T cell, tumor infiltrating lymphocyte, NK-T cell, invariant NK-T cell, gamma delta T cell, and NK cell.

C9. The method of any one of embodiments C1-C7, wherein the cell is a T cell.

C9.1. The method of any one of embodiments C1-C7, wherein the cell is an invariant NK-T cell.

C9.2. The method of any one of embodiments C1-C7, wherein the cell is a gamma-delta T cell.

C10. The method of any one of embodiments C1-C7, wherein the cell is selected from the group consisting of T cells, NK-T cells, and NK cells.

D, E. Reserved

F1. A nucleic acid comprising a promoter operably linked to a polynucleotide encoding a chimeric signaling polypeptide, wherein the polypeptide comprises
a) a MyD88 polypeptide or a truncated MyD88 polypeptide lacking a TIR domain; and
b) a costimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions.

F1.1. The nucleic acid of embodiment A1, wherein the costimulatory polypeptide cytoplasmic signaling region is selected from the group consisting of CD27, CD28, ICOS, 4-1BB, RANK/TRANCE-R, and OX40.

F1.2. The nucleic acid of embodiment A1, wherein the costimulatory polypeptide cytoplasmic signaling region is selected from the group consisting of CD27, CD30, TweakR, TAC1, BCMA and HVEM.

F1.3. The nucleic acid of embodiment A1, wherein the costimulatory polypeptide cytoplasmic signaling region is selected from the group consisting of CD27, CD30, TweakR, BCMA and HVEM.

F1.4. The nucleic acid of embodiment A1, wherein the costimulatory polypeptide cytoplasmic signaling region is selected from the group consisting of CD28, 4-1BB, OX40, and ICOS.

F2. The nucleic acid of any one of embodiments F1-F1.4, wherein the polypeptide comprises a truncated MyD88 polypeptide lacking a TIR domain.

F3. The nucleic acid of any one of embodiments F1-F2, wherein the costimulatory polypeptide cytoplasmic signaling region is a 4-1BB cytoplasmic signaling region.

F4. The nucleic acid of any one of embodiments F1-F2, wherein the costimulatory polypeptide cytoplasmic signaling region is an OX40 cytoplasmic signaling region.

F5. The nucleic acid of any one of embodiments F1-F2, wherein the costimulatory polypeptide cytoplasmic signaling region is a CD28 cytoplasmic signaling region.

F6. The nucleic acid of any one of embodiments F1-F2, wherein the costimulatory polypeptide cytoplasmic signaling region is an ICOS cytoplasmic signaling region.

F7-F14. Reserved.

F15. The nucleic acid of any one of embodiments F1-F14, wherein the truncated MyD88 polypeptide comprises the amino acid sequence of SEQ ID NO: 2 lacking the TIR domain, or a functional fragment thereof.

F16. The nucleic acid of any one of embodiments F1-F15, wherein the truncated MyD88 polypeptide does not comprise contiguous amino acid residues 156 to the C-terminus of the full length MyD88 polypeptide.

F16.1. The nucleic acid of any one of embodiments F1-F15, wherein the truncated MyD88 polypeptide does not comprise contiguous amino acid residues 152 to the C-terminus of the full length MyD88 polypeptide.

F16.2. The nucleic acid of any one of embodiments F1-F15, wherein the truncated MyD88 polypeptide does not comprise contiguous amino acid residues 173 to the C-terminus of the full length MyD88 polypeptide.

F16.3. The nucleic acid of any one of embodiments F16-F16.2, wherein the full length MyD88 polypeptide comprises the amino acid sequence of SEQ ID NO: 907.

F17. The nucleic acid of any one of embodiments F1-F16.3, wherein the truncated MyD88 polypeptide consists of the amino acid sequence of SEQ ID NO: 2, or a functional fragment thereof.

F18. The nucleic acid of any one of embodiments F1-F17, wherein the cytoplasmic signaling region comprises an amino acid sequence selected from the group consisting of SEQ ID NOs: 6, 48, 90, and 174, or a functional fragment thereof.

F19. The nucleic acid of any one of embodiments F1-F17, wherein the cytoplasmic signaling region consists of an amino acid sequence selected from the group consisting of SEQ ID NOs: 6, 48, 90, and 174, or a functional fragment thereof.

F20. A nucleic acid comprising a promoter operably linked to a polynucleotide encoding a chimeric signaling polypeptide, wherein the polypeptide comprises

a) a truncated MyD88 polypeptide lacking a TIR domain; and

b) a CD28 cytoplasmic signaling region.

F21. A nucleic acid comprising a promoter operably linked to a polynucleotide encoding a chimeric signaling polypeptide, wherein the polypeptide comprises

a) a truncated MyD88 polypeptide lacking a TIR domain; and

b) a 4-1BB cytoplasmic signaling region.

F22. The nucleic acid of any one of embodiments F1-F21, wherein the chimeric signaling polypeptide further comprises a membrane targeting region.

F23. The nucleic acid of embodiment F22, wherein the membrane targeting region is selected from the group consisting of a myristoylation region, a palmitoylation region, a prenylation region, and transmembrane sequences of receptors.

F24. The nucleic acid of embodiment F22, wherein the membrane-targeting region is a myristoylation region.

F25. The nucleic acid of any one of embodiments F1-F24, further comprising a polynucleotide coding for a heterologous protein.

F26. The nucleic acid of any one of embodiments F1-F24, further comprising a polynucleotide coding for a chimeric antigen receptor.

F27. The nucleic acid of embodiment F26, wherein the chimeric antigen receptor comprises (i) a transmembrane region, (ii) a T cell activation molecule, and (iii) an antigen recognition moiety.

F28. The nucleic acid of any one of embodiments F1-F24, further comprising a polynucleotide coding for a recombinant T cell receptor.

F29. The nucleic acid of any one of embodiments F25-F28, further comprising a polynucleotide encoding a linker polypeptide between the two polynucleotides, wherein the linker polypeptide separates the translation products of the two polynucleotides during or after translation.

F30. The nucleic acid of embodiment F29, wherein the linker polypeptide that separates the translation products of two polynucleotides is a 2A polypeptide.

F31. The nucleic acid of any one of embodiments F25-F28, further comprising a second promoter operably linked to the polynucleotide encoding the heterologous polypeptide, the chimeric antigen receptor or the recombinant T cell receptor.

F32. The nucleic acid of any one of embodiments F1-F24, further comprising a polynucleotide encoding a chimeric Caspase-9 polypeptide comprising a multimeric ligand binding region and a Caspase-9 polypeptide.

F33. The nucleic acid of any one of embodiments F25-F31, further comprising a polynucleotide encoding a chimeric Caspase-9 polypeptide comprising a multimeric ligand binding region and a Caspase-9 polypeptide.

F34. The nucleic acid of any one of embodiments F32-F33, wherein the Caspase-9 polypeptide lacks the CARD domain.

F35. The nucleic acid of embodiment F34, wherein the Caspase-9 polypeptide comprises the amino acid sequence of SEQ ID NO: 670

F36. The nucleic acid of embodiment F35, wherein the Caspase-9 polypeptide comprises the amino acid sequence of SEQ ID NO. 670 and further comprises an amino acid substitution selected from the group consisting of the caspase variants in Table 5.

F37. The nucleic acid of embodiment F36, wherein the amino acid substitution is selected from the group consisting of N405Q, D330A, and D330E

F38. The nucleic acid of any one of embodiments F32-F37, further comprising a polynucleotide encoding a linker polypeptide between two polynucleotides of the nucleic acid, wherein the linker polypeptide separates the translation products of the polynucleotide encoding the chimeric Caspase-9 polypeptide from the translation product of another polynucleotide of the nucleic acid.

F39. The nucleic acid of embodiment F39, wherein the linker polypeptide that separates the translation products of two polynucleotides is a 2A polypeptide.

F40. The nucleic acid of any one of embodiments F32-F39, wherein the multimeric ligand binding region comprises a FKBP12 polypeptide or FKBP12 variant polypeptide.

F41. The nucleic acid of embodiment F40, wherein multimeric ligand binding region comprises a FKBP12 variant polypeptide comprising an amino acid substitution at position 36 that binds with higher affinity to the multimeric ligand than the wild type FKBP12 polypeptide.

F42. The nucleic acid of embodiment F41, wherein the amino acid substitution at position 36 is selected from the group consisting of valine, isoleucine, leucine, and alanine.

F43. The nucleic acid of any one of embodiments F32-F42, wherein the multimeric ligand binding region comprises a FKBP12v36 region.

F44. The nucleic acid of any one of embodiments F32-F43, wherein the ligand is dimeric FK506, or a dimeric FK506-like analog.

F45. The nucleic acid of any one of embodiments F32-F44, wherein the ligand is rimiducid or AP20187.

F46. The nucleic acid of any one of embodiments F32-F39, wherein the multimeric ligand binding region comprises a FKBP-rapamycin binding domain of mTOR (FRB) or FRB variant region.

F47. The nucleic acid of embodiment F46, wherein the FRB variant region is selected from the group consisting of KLW (T2098L), KTF (W2101F), and KLF (T2098L, W2101F).

F48. The nucleic acid of any one of embodiments F46-F47, wherein the FRB variant region is FRB_{L}

F49. The nucleic acid or cell of any one of embodiments F46-FF48, wherein the FRB variant region binds to a rapalog selected from the group consisting of S-o,p-dimethoxyphenyl (DMOP)-rapamycin, R-Isopropoxyrapamycin, S-Butanesulfonamidorap, R and S C7-ethyloxyrapamycin, R and S C7-isopropyloxyrapamycin, R and S C7-isobutylrapamycin, R and S ethylcarbamaterapamycin, R and S C7-phenylcarbamaterapamycin, R and S C7-(3-methyl)indole rapamycin, temsirolimus, everolimus, zotarolimus, and R and S C7-(7-methyl)indole rapamycin.

F49.1. The nucleic acid of any one of embodiments F1-F49, wherein the chimeric signaling polypeptide does not include a CD40 cytoplasmic region polypeptide.

F50. The nucleic acid of any one of embodiments F1-F49.1, wherein the nucleic acid is an isolated nucleic acid.

F51. The nucleic acid composition of any one of embodiments F1-F50, wherein the nucleic acid comprises a promoter sequence operably linked to the polynucleotide.

F52. The nucleic acid composition of any one of embodiments F1-F51, wherein the nucleic acid is contained within a viral vector.

F53. The nucleic acid composition of embodiment F52, wherein the viral vector is a retroviral vector.

F54. The nucleic acid composition of any one of embodiments F1-F53, wherein the nucleic acid is contained within a plasmid.

F55. A chimeric signaling polypeptide encoded by a nucleic acid of any one of embodiments F1-F24.

G1. A modified cell, wherein the cell is transduced or transfected with a nucleic acid of any one of embodiments F1-F54.

G2. A modified cell, wherein the cell is transduced or transfected with
a) a nucleic acid of any one of embodiments F1-F54; and
b) a nucleic acid comprising a polynucleotide coding for a heterologous protein.

G3. A modified cell, wherein the cell is transduced or transfected with
a) a nucleic acid of any one of embodiments F1-F54; and
b) a nucleic acid comprising a polynucleotide coding for a chimeric antigen receptor.

G4. The modified cell of embodiment G3, wherein the chimeric antigen receptor comprises (i) a transmembrane region, (ii) a T cell activation molecule, and (iii) an antigen recognition moiety.

G5. A modified cell, wherein the cell s transduced or transfected with
a) a nucleic acid of any one of embodiments F1-F24; and
b) a nucleic acid comprising a polynucleotide coding for a recombinant T cell receptor.

G6. The modified cell of any one of embodiments G1-G5, wherein the cell is selected from the group consisting of T cell, tumor infiltrating lymphocyte, NK-T cell, invariant NK-T cell, gamma delta T cell, and NK cell.

G7. The modified cell of any one of embodiments G1-G5, wherein the cell is a T cell.

G8. The modified cell of any one of embodiments G1-G5, wherein the cell is a NK cell.

G8.1. The modified cell of any one of embodiments G1-G5, wherein the cell is an invariant NK-T cell.

G8.2. The modified cell of any one of embodiments G1-G5, wherein the cell is a gamma delta T cell.

G9. The modified cell of any one of embodiments G1-G8.2, wherein the cell is obtained or prepared from bone marrow.

G10. The modified cell of any one of embodiments G1-G8.2, wherein the cell is obtained or prepared from umbilical cord blood.

G11. The modified cell of any one of embodiments G1-G8.2, wherein the cell is obtained or prepared from peripheral blood.

G12. The modified cell of any one of embodiments G1-G8.2, wherein the cell is obtained or prepared from peripheral blood mononuclear cells.

G13. The modified cell of any one of embodiments G1-G8.2, wherein the cell is a human cell.

G14. The modified cell of any one of embodiments G1-G8.2, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

G15. The modified cell of any one of embodiments G1-G14, wherein the cell further comprises a nucleic acid comprising a polynucleotide encoding a chimeric Caspase-9 polypeptide comprising a multimeric ligand binding region and a Caspase-9 polypeptide.

G16. The modified cell of embodiment G15, wherein the Caspase-9 polypeptide lacks the CARD domain.

G17. The modified cell of embodiment G16, wherein the Caspase-9 polypeptide comprises the amino acid sequence of SEQ ID NO: 670.

G18. The modified cell of embodiment G17, wherein the Caspase-9 polypeptide comprises the amino acid sequence of SEQ ID NO. 670 and further comprises an amino acid substitution selected from the group consisting of the caspase variants in Table 5.

G19. The modified cell of embodiment G18, wherein the amino acid substitution is selected from the group consisting of N405Q, D330A, and D330E.

G20. The modified cell of any one of embodiments G15-G19, wherein the multimeric ligand binding region comprises a FKBP12 polypeptide or modified FKBP12 polypeptide.

G21. The modified cell of embodiment G20, wherein multimeric ligand binding region comprises a modified FKBP12 polypeptide comprising an amino acid substitution at position 36 that binds with higher affinity to the multimeric ligand than the wild type FKBP12 polypeptide.

G22. The modified cell of embodiment G21, wherein the amino acid substitution at position 36 is selected from the group consisting of valine, isoleucine, leucine, and alanine.

G23. The modified cell of any one of embodiments G15-G22, wherein the multimeric ligand binding region comprises a FKBP12v36 region.

G24. The modified cell of any one of embodiments G15-G23, wherein the ligand is dimeric FK506, or a dimeric FK506-like analog.

G25. The modified cell of any one of embodiments G15-G24, wherein the ligand is rimiducid or AP20187.

H1. A method for expressing a chimeric signaling polypeptide in a cell, comprising contacting a nucleic acid of any one of embodiments F1 to F54 with a cell under conditions in which the nucleic acid is incorporated into the cell, whereby the cell expresses the inducible chimeric signaling polypeptide from the incorporated nucleic acid.

H2. The method of embodiment H1, wherein the cell is contacted with a nucleic acid of any one of embodiments F25, F29-F31, or F33-F54, wherein the cell further expresses a heterologous protein.

H3. The method of embodiment H1, wherein the cell is contacted with a nucleic acid of any one of embodiments F26-F27, F29-F31, or F33-F54, wherein the cell further expresses a chimeric antigen receptor.

H4. The method of embodiment H1, wherein the cell is contacted with a nucleic acid of any one of embodiments F29-F31, or F33-F36, wherein the cell further expresses a recombinant T cell receptor.

H5. The method of embodiment H1, wherein the cell is contacted with a nucleic acid of any one of embodiments F33-F54, wherein the cell further expresses a chimeric Caspase-9 polypeptide comprising a multimeric ligand binding region and a Caspase-9 polypeptide.

H6. The method of any one of embodiments H1-H5, wherein the nucleic acid is contacted with the cell ex vivo.

H7. The method of any one of embodiments H1-H5, wherein the nucleic acid is contacted with the cell in vivo.

H8. The method of any one of embodiments H1-H7, wherein the cell is selected from the group consisting of T cell, tumor infiltrating lymphocyte, NK-T cell, invariant NK-T cell, gamma delta T cell, and NK cell.

H8.1. The method of any one of embodiments H1-H7, wherein the cell is a T cell.

H8.2. The method of any one of embodiments H1-H7, wherein the cell is a gamma delta T cell. H8.3. The method of any one of embodiments H1-H7, wherein the cell is a NK cell.

H9. The method of any one of embodiments H1-H7, wherein the cell is a invariant NK cell.

H10. The method of any one of embodiments H1-H7, wherein the cell is selected from the group consisting of T cells, NK cells, and NK-T cells.

I-J. Reserved.

K1. A nucleic acid comprising a promoter operably linked to a polynucleotide encoding an inducible chimeric antigen receptor polypeptide, wherein the polypeptide comprises
a) one or more multimeric ligand binding regions that bind to a multimeric ligand;
b) a MyD88 polypeptide or a truncated MyD88 polypeptide lacking a TIR domain;
c) a costimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
d) a transmembrane region;
e) a T cell activation molecule, and
f) an antigen recognition moiety.

K1.1. The nucleic acid of embodiment K1, wherein the costimulatory polypeptide cytoplasmic signaling region is selected from the group consisting of CD27, CD28, ICOS, 4-1BB, RANK/TRANCE-R, and OX40.

K1.2. The nucleic acid of embodiment K1, wherein the costimulatory polypeptide cytoplasmic signaling region is selected from the group consisting of CD27, CD30, TweakR, TAC1, BCMA and HVEM.

K1.3. The nucleic acid of embodiment K1, wherein the costimulatory polypeptide cytoplasmic signaling region is selected from the group consisting of CD27, CD30, TweakR, BCMA and HVEM.

K1.4. The nucleic acid of embodiment K1, wherein the costimulatory polypeptide cytoplasmic signaling region is selected from the group consisting of CD28, 4-1BB, OX40, and ICOS.

K1.5. The nucleic acid of any one of embodiments K1-K1.4, wherein the costimulatory polypeptide cytoplasmic signaling region is a 4-1BB cytoplasmic signaling region.

K1.6. The nucleic acid of any one of embodiments K1-K1.4, wherein the costimulatory polypeptide cytoplasmic signaling region is an OX40 cytoplasmic signaling region.

K1.7. The nucleic acid of any one of embodiments K1-K1.4, wherein the costimulatory polypeptide cytoplasmic signaling region is a CD28 cytoplasmic signaling region.

K1.8. The nucleic acid of any one of embodiments K1-K1.4, wherein the costimulatory polypeptide cytoplasmic signaling region is an ICOS cytoplasmic signaling region.

K1.9. A nucleic acid comprising a promoter operably linked to a polynucleotide encoding an inducible chimeric antigen receptor polypeptide, wherein the polypeptide comprises
a) a multimeric ligand binding region that binds to a multimeric ligand;
b) a MyD88 polypeptide or a truncated MyD88 polypeptide lacking a TIR domain;
c) a CD28 costimulatory polypeptide cytoplasmic signaling region;
d) a transmembrane region;
e) a T cell activation molecule, and
f) an antigen recognition moiety.

K1.10. A nucleic acid comprising a promoter operably linked to a polynucleotide encoding an inducible chimeric antigen receptor polypeptide, wherein the polypeptide comprises
a) a multimeric ligand binding region that binds to a multimeric ligand;
b) a MyD88 polypeptide or a truncated MyD88 polypeptide lacking a TIR domain;
c) a 4-1BB costimulatory polypeptide cytoplasmic signaling region;
d) a transmembrane region;
e) a T cell activation molecule, and
f) an antigen recognition moiety.

K1.11. The nucleic acid of any one of embodiments K1-K1.10, wherein the inducible chimeric antigen receptor polypeptide further comprises a stalk polypeptide.

K1.12. The nucleic acid of embodiment K1.11, wherein the stalk polypeptide is a CD8 stalk polypeptide.

K1.13. The nucleic acid of any one of embodiments K1-K1.12, wherein the T cell activation molecule is selected from the group consisting of an ITAM-containing, Signal 1 conferring molecule, a CD3ζ polypeptide, and an Fc epsilon receptor gamma (FcεR1γ) subunit polypeptide.

K1.14. The nucleic acid of any one of embodiments K1-K1.13, wherein the antigen recognition moiety binds to an antigen on a tumor cell.

K1.15. The nucleic acid of any one of embodiments K1-K1.13, wherein the antigen recognition moiety binds to an antigen on a cell involved in a hyperproliferative disease or to a viral or bacterial antigen.

K1.16. The nucleic acid of any one of embodiments K1-K1.13, wherein the antigen recognition moiety binds to an antigen selected from the group consisting of PSMA, PSCA, MUC1, CD19, ROR1, Mesothelin, GD2, CD123, MUC16, Her2/NE, CD20, CD30, BCMA, PRAME, NY-ESO-1, and EGFRvIII.

K1.17. The nucleic acid of any one of embodiments K1-K1.16, wherein the antigen recognition moiety is a single chain variable fragment.

K1.18. The nucleic acid of any one of embodiments K1-K1.17, wherein the transmembrane region is a CD28 transmembrane region or a CD8 transmembrane region.

K2. The nucleic acid of any one of embodiments K1-K1.18, wherein the polypeptide comprises a truncated MyD88 polypeptide lacking a TIR domain.

K3-K6. Reserved

K7. The nucleic acid of any one of embodiments K1-K6, wherein the inducible chimeric antigen receptor polypeptide comprises two multimeric ligand binding regions.

K7.1. The nucleic acid of any one of embodiments K1-K6, wherein the inducible chimeric antigen receptor polypeptide comprises three multimeric ligand binding regions.

K7.2. The nucleic acid of any one of embodiments K1-K6, wherein the inducible chimeric antigen receptor polypeptide comprises four or more multimeric ligand binding regions.

K7.3. The nucleic acid of any one of embodiments K1-K7.2, wherein one or more of the multimeric ligand binding regions comprises an FKBP12 polypeptide.

K7.4. The nucleic acid of any one of embodiments K1-K7.3, wherein one or more of the multimeric ligand binding regions comprises an FKBP12 variant polypeptide comprising an amino acid substitution at position 36 that binds with higher affinity to the multimeric ligand than the wild type FKBP12 polypeptide.

K7.5. The nucleic acid of any one of embodiments K1-K7.3, wherein each of the multimeric ligand binding regions comprises an FKBP12 variant polypeptide comprising an amino acid substitution at position 36 that binds with higher affinity to the multimeric ligand than the wild type FKBP12 polypeptide.

K7.6. The nucleic acid of any one of embodiments K7.4 or K7.5, wherein the amino acid substitutions at position 36 are selected from the group consisting of valine, isoleucine, leucine, and alanine.

K7.7. The nucleic acid of any one of embodiments K7.4 to K7.6, wherein one or more of the FKBP12 variant polypeptides is an FKBP12v36 polypeptide.

K7.8. The nucleic acid of any one of embodiments K7.4 to K7.6, wherein each of the FKBP12 variant polypeptides are FKBP12v36 polypeptides.

K8. The nucleic acid of any one of embodiments K1-K7.8, wherein the inducible chimeric antigen receptor polypeptide comprises two FKBP12 variant polypeptides, wherein each of the FKBP12 variant polypeptides comprises an amino acid substitution at position 36 that binds with higher affinity to the multimeric ligand than the wild type FKBP12 polypeptide.

K8.1. The nucleic acid of embodiment K8, wherein the amino acid substitutions at position 36 are selected from the group consisting of valine, isoleucine, leucine, and alanine.

K8.2. The nucleic acid of embodiment K8, wherein the inducible chimeric antigen receptor polypeptide comprises two FKBP12v36 polypeptides.

K8.3. The nucleic acid of any one of embodiments K8-K8.2 wherein the inducible chimeric antigen receptor polypeptide comprises an Fv′Fvls polypeptide.

K8.4. The nucleic acid of any one of embodiments K7.7 to K8.3, wherein the multimeric ligand binding region comprises two FKBP12v36 polypeptides, wherein the first FKBP12v36 polypeptide is encoded by the nucleotide sequence of SEQ ID NO: 5, and the second FKBP12v36 polypeptide is encoded by the nucleotide sequence of SEQ ID NO: 7.

K9. The nucleic acid of any one of embodiments K1-K8.4, wherein the ligand is dimeric FK506, or a dimeric FK506-like analog.

K10. The nucleic acid of any one of embodiments K1-K9, wherein the ligand is rimiducid.

K11. The nucleic acid of any one of embodiments K1-K9, wherein the ligand is AP20187.

K12-K14. Reserved

K15. The nucleic acid of any one of embodiments K1-K11, wherein the truncated MyD88 polypeptide comprises the amino acid sequence of the full length MyD88 polypeptide sequence of SEQ ID NO: 907 lacking the TIR domain, or a functional fragment thereof.

K16. The nucleic acid of any one of embodiments K1-K15, wherein the truncated MyD88 polypeptide does not comprise contiguous amino acid residues 156 to the C-terminus of the full length MyD88 polypeptide.

K16.1. The nucleic acid of any one of embodiments K1-K15, wherein the truncated MyD88 polypeptide does not comprise contiguous amino acid residues 152 to the C-terminus of the full length MyD88 polypeptide.

K16.2. The nucleic acid of any one of embodiments A1-A15, wherein the truncated MyD88 polypeptide does not comprise contiguous amino acid residues 173 to the C-terminus of the full length MyD88 polypeptide.

K16.3. The nucleic acid of any one of embodiments K16-K16.2, wherein the full length MyD88 polypeptide comprises the amino acid sequence of SEQ ID NO: 907.

K17. The nucleic acid of any one of embodiments K1-K16.3, wherein the truncated MyD88 polypeptide consists of the amino acid sequence of SEQ ID NO: 2, or a functional fragment thereof.

K18. The nucleic acid of any one of embodiments K1-K17, wherein the cytoplasmic signaling region comprises an amino acid sequence selected from the group consisting of SEQ ID NOs: 6, 48, 90, and 174, or a functional fragment thereof.

K19. The nucleic acid of any one of embodiments K1-K17, wherein the cytoplasmic signaling region consists of an amino acid sequence selected from the group consisting of SEQ ID NOs: 6, 48, 90, and 174, or a functional fragment thereof.

K19.01. The nucleic acid of any one of embodiments K1-K6, or K15-K19, wherein the multimeric ligand binding region comprises a FKBP-rapamycin binding domain of mTOR (FRB) or FRB variant region.

K19.1. The nucleic acid of any one of embodiments K1-K6, or K15-K19, wherein the multimeric ligand binding region comprises a FKBP-rapamycin binding domain of mTOR (FRB) or FRB variant region; and a FKBP12 polypeptide region.

K19.2. The nucleic acid of any one of embodiments K19.01 or K19.1, wherein the FRB variant region is selected from the group consisting of KLW (T2098L), KTF (W2101F), and KLF (T2098L, W2101F).

K19.3. The nucleic acid of any one of embodiments K19.01-K19.2, wherein the FRB variant region is FRB_{L}

K19.4. The nucleic acid or cell of any one of embodiments K19.01-K19.3, wherein the FRB variant region binds to a rapalog selected from the group consisting of S-o,p-dimethoxyphenyl (DMOP)-rapamycin, R-Isopropoxyrapamycin, S-Butanesulfonamidorap, R and S C7-ethyloxyrapamycin, R and S C7-isopropyloxyrapamycin, R and S C7-isobutylrapamycin, R and S ethylcarbamaterapamycin, R and S C7-phenylcarbamaterapamycin, R and S C7-(3-methyl)indole rapamycin, temsirolimus, everolimus, zotarolimus, and R and S C7-(7-methyl)indole rapamycin.

K19.5. The nucleic acid of any one of embodiments K19.01-K19.4, further comprising a polynucleotide encoding a chimeric Caspase-9 polypeptide comprising a modified FKBP12 polypeptide that binds to rimiducid, and a Caspase-9 polypeptide.

K19.6. The nucleic acid of embodiment K19.5, wherein the Caspase-9 polypeptide lacks the CARD domain.

K19.7. The nucleic acid of embodiment K19.6, wherein the Caspase-9 polypeptide comprises the amino acid sequence of SEQ ID NO: 670.

K19.8. The nucleic acid of embodiment K19.7, wherein the Caspase-9 polypeptide comprises the amino acid sequence of SEQ ID NO.670 and further comprises an amino acid substitution selected from the group consisting of the caspase variants in Table 5.

K19.9. The nucleic acid of embodiment K19.8, wherein the amino acid substitution is selected from the group consisting of N405Q, D330A, and D330E

K19.10. The nucleic acid of any one of embodiments K19.5-K19.10, further comprising a polynucleotide encoding a linker polypeptide between two polynucleotides of the nucleic acid, wherein the linker polypeptide separates the translation products of the polynucleotide encoding the chimeric Caspase-9 polypeptide from the translation product of another polynucleotide of the nucleic acid.

K19.11. The nucleic acid of embodiment K19.10, wherein the linker polypeptide that separates the translation products of two polynucleotides is a 2A polypeptide.

K19.12. The nucleic acid of any one of embodiments K19.5-K19.11, wherein modified FKBP12 polypeptide comprises an amino acid substitution at position 36 that binds with higher affinity to the multimeric ligand than the wild type FKBP12 polypeptide.

K19.13. The nucleic acid of embodiment K19.12, wherein the amino acid substitution at position 36 is selected from the group consisting of valine, isoleucine, leucine, and alanine.

K19.14. The nucleic acid of any one of embodiments K19.5-K19.13, wherein the modified FKBP12 polypeptide is FKBP12v36.

K19.15. The nucleic acid of any one of embodiments K19.5-K19.14 wherein dimeric FK506, or a dimeric FK506-like analog binds to the modified FKBP12 polypeptide.

K20. The nucleic acid of any one of embodiments K19.5-K19.15, wherein AP20187 binds to the modified FKBP12 polypeptide.

K20.1. The nucleic acid of any one of embodiments K1-K20, wherein the inducible chimeric antigen receptor polypeptide does not include a CD40 cytoplasmic region polypeptide.

K21. The nucleic acid of any one of embodiments K1-K20.1, wherein the nucleic acid is an isolated nucleic acid.

K22. The nucleic acid composition of any one of embodiments K1-K21, wherein the nucleic acid comprises a promoter sequence operably linked to the polynucleotide.

K23. The nucleic acid composition of any one of embodiments K1-K22, wherein the nucleic acid is contained within a viral vector.

K24. The nucleic acid composition of any one of embodiments K1-K23, wherein the viral vector is a retroviral vector.

K25. The nucleic acid composition of any one of embodiments K1-K24, wherein the nucleic acid is contained within a plasmid.

K26-K31. Reserved.

K32. An inducible chimeric antigen receptor polypeptide encoded by a nucleic acid of any one of embodiments K1-K21.

L1. A modified cell, wherein the cell is transduced or transfected with a nucleic acid of any one of embodiments K1-K31.

L2-L5. Reserved.

L6. The modified cell of any one of embodiments L1-L5, wherein the cell is selected from the group consisting of T cell, tumor infiltrating lymphocyte, NK-T cell, invariant NK-T cell, gamma delta T cell, and NK cell.

L6.1. The method of any one of embodiments L1-L5, wherein the cell is a gamma delta T cell. L6.2. The method of any one of embodiments L1-L5, wherein the cell is a NK cell.

L6.3. The method of any one of embodiments L1-L5, wherein the cell is a invariant NK-T cell.

L7. The modified cell of any one of embodiments L1-L5, wherein the cell is a T cell.

L8. The modified cell of any one of embodiments L1-L5, wherein the cell is selected from the group consisting of NK cells, NK-T cells, and T cells.

L9. The modified cell of any one of embodiments L1-L8, wherein the cell is obtained or prepared from bone marrow.

L10. The modified cell of any one of embodiments L1-L8, wherein the cell is obtained or prepared from umbilical cord blood.

L11. The modified cell of any one of embodiments L1-L8, wherein the cell is obtained or prepared from peripheral blood.

L12. The modified cell of any one of embodiments L1-L8, wherein the cell is obtained or prepared from peripheral blood mononuclear cells.

L13. The modified cell of any one of embodiments L1-L8, wherein the cell is a human cell.

L14. The modified cell of any one of embodiments L1-L8, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

M1. A method for expressing an inducible chimeric antigen receptor polypeptide in a cell, comprising contacting a nucleic acid of any one of embodiments K1 to K31 with a cell under conditions in which the nucleic acid is incorporated into the cell, whereby the cell expresses the inducible chimeric antigen receptor polypeptide from the incorporated nucleic acid.

M2. The method of embodiment M1, wherein the nucleic acid is contacted with the cell ex vivo.

M3. The method of embodiment M1, wherein the nucleic acid is contacted with the cell in vivo.

M4. The method of any one of embodiments M1-M3, wherein the cell is selected from the group consisting of T cell, tumor infiltrating lymphocyte, NK-T cell, invariant NK-T cell, gamma delta T cell, and NK cell.

M4.1. The method of any one of embodiments M1-L3, wherein the cell is a gamma delta T cell.

M4.2. The method of any one of embodiments M1-L3, wherein the cell is a NK cell.

M4.3. The method of any one of embodiments M1-L3, wherein the cell is a invariant NK-T cell.

M5. The method of any one of embodiments M1-M3, wherein the cell is a T cell.

M6. The method of any one of embodiments M1-M3, wherein the cell is selected from the group consisting of NK cells, NK-T cells, and T cells.

N-O. Reserved

P1. A nucleic acid comprising a promoter operably linked to a polynucleotide encoding a chimeric antigen receptor polypeptide, wherein the polypeptide comprises
a) a MyD88 polypeptide or a truncated MyD88 polypeptide lacking a TIR domain;
b) a costimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
c) a transmembrane region;
d) a T cell activation molecule, and
e) an antigen recognition moiety.

P1.1. The nucleic acid of embodiment P1, wherein the costimulatory polypeptide cytoplasmic signaling region is selected from the group consisting of CD27, CD28, ICOS, 4-1BB, RANK/TRANCE-R, and OX40.

P1.2. The nucleic acid of embodiment P1, wherein the costimulatory polypeptide cytoplasmic signaling region is selected from the group consisting of CD27, CD30, TweakR, TAC1, BCMA and HVEM.

P1.3. The nucleic acid of embodiment P1, wherein the costimulatory polypeptide cytoplasmic signaling region is selected from the group consisting of CD27, CD30, TweakR, BCMA and HVEM.

P1.4. The nucleic acid of embodiment P1, wherein the costimulatory polypeptide cytoplasmic signaling region is selected from the group consisting of CD28, 4-1BB, OX40, and ICOS.

P1.5. The nucleic acid of any one of embodiments P1-P1.4, wherein the chimeric antigen receptor polypeptide further comprises a stalk polypeptide.

P1.6. The nucleic acid of embodiment P1.5, wherein the stalk polypeptide is a CD8 stalk polypeptide.

P1.7. The nucleic acid of any one of embodiments P1-P1.6, wherein the T cell activation molecule is selected from the group consisting of an ITAM-containing, Signal 1 conferring molecule, a CD3ζ polypeptide, and an Fc epsilon receptor gamma (FcεR1γ) subunit polypeptide.

P1.8. The nucleic acid of any one of embodiments P1-P1.7, wherein the antigen recognition moiety binds to an antigen on a tumor cell.

P1.9. The nucleic acid of any one of embodiments P1-P1.7, wherein the antigen recognition moiety binds to an antigen on a cell involved in a hyperproliferative disease or to a viral or bacterial antigen.

P1.10. The nucleic acid of any one of embodiments P1-P1.7, wherein the antigen recognition moiety binds to an antigen selected from the group consisting of PSMA, PSCA, MUC1, CD19, ROR1, Mesothelin, GD2, CD123, MUC16, Her2/NE, CD20, CD30, BCMA, PRAME, NY-ESO-1, and EGFRvIII.

P1.11. The nucleic acid of any one of embodiments P1-P1.7, wherein the antigen recognition moiety is a single chain variable fragment.

P1.12. The nucleic acid of any one of embodiments P1-P1.11, wherein the transmembrane region is a CD28 transmembrane region or a CD8 transmembrane region.

P2. The nucleic acid of any one of embodiments P1-P1.12, wherein the polypeptide comprises a truncated MyD88 polypeptide lacking a TIR domain.

P3. The nucleic acid of any one of embodiments P1-P2, wherein the costimulatory polypeptide cytoplasmic signaling region is a 4-1BB cytoplasmic signaling region.

P4. The nucleic acid of any one of embodiments P1-P2, wherein the costimulatory polypeptide cytoplasmic signaling region is an OX40 cytoplasmic signaling region.

P5. The nucleic acid of any one of embodiments P1-P2, wherein the costimulatory polypeptide cytoplasmic signaling region is a CD28 cytoplasmic signaling region.

P6. The nucleic acid of any one of embodiments P1-P2, wherein the costimulatory polypeptide cytoplasmic signaling region is an ICOS cytoplasmic signaling region.

P7-P14. Reserved.

P15. The nucleic acid of any one of embodiments P1-P14, wherein the truncated MyD88 polypeptide comprises the amino acid sequence of SEQ ID NO: 2 lacking the TIR domain, or a functional fragment thereof.

P16. The nucleic acid of any one of embodiments P1-P15, wherein the truncated MyD88 polypeptide does not comprise contiguous amino acid residues 156 to the C-terminus of the full length MyD88 polypeptide.

P16.1. The nucleic acid of any one of embodiments P1-P15, wherein the truncated MyD88 polypeptide does not comprise contiguous amino acid residues 152 to the C-terminus of the full length MyD88 polypeptide.

P16.2. The nucleic acid of any one of embodiments P1-P15, wherein the truncated MyD88 polypeptide does not comprise contiguous amino acid residues 173 to the C-terminus of the full length MyD88 polypeptide.

P16.3. The nucleic acid of any one of embodiments P16-P16.2, wherein the full length MyD88 polypeptide comprises the amino acid sequence of SEQ ID NO: 907.

P17. The nucleic acid of any one of embodiments P1-P16.3, wherein the truncated MyD88 polypeptide consists of the amino acid sequence of SEQ ID NO:2, or a functional fragment thereof.

P18. The nucleic acid of any one of embodiments P1-P17, wherein the cytoplasmic signaling region comprises an amino acid sequence selected from the group consisting of SEQ ID NOs: 6, 48, 90, and 174, or a functional fragment thereof.

P19. The nucleic acid of any one of embodiments P1-P17, wherein the cytoplasmic signaling region consists of an amino acid sequence selected from the group consisting of SEQ ID NOs: 6, 48, 90, and 174, or a functional fragment thereof.

P22. The nucleic acid of any one of embodiments P1-P21, further comprising a polynucleotide encoding a chimeric Caspase-9 polypeptide comprising a multimeric ligand binding region and a Caspase-9 polypeptide.

P23. The nucleic acid of embodiment P22, wherein the Caspase-9 polypeptide lacks the CARD domain.

P24. The nucleic acid of embodiment P23, wherein the Caspase-9 polypeptide comprises the amino acid sequence of SEQ ID NO: 670.

P25. The nucleic acid of embodiment P24, wherein the Caspase-9 polypeptide comprises the amino acid sequence of SEQ ID NO. 670 and further comprises an amino acid substitution selected from the group consisting of the caspase variants in Table 5.

P26. The nucleic acid of embodiment P25, wherein the amino acid substitution is selected from the group consisting of N405Q, D330A, and D330E

P27. The nucleic acid of any one of embodiments P22-P26, further comprising a polynucleotide encoding a linker polypeptide between two polynucleotides of the nucleic acid, wherein the linker polypeptide separates the translation products of the polynucleotide encoding the chimeric Caspase-9 polypeptide from the translation product of another polynucleotide of the nucleic acid.

P28. The nucleic acid of embodiment P27, wherein the linker polypeptide that separates the translation products of two polynucleotides is a 2A polypeptide.

P29. The nucleic acid of any one of embodiments P22-P28, wherein the multimeric ligand binding region comprises a FKBP12 polypeptide or modified FKBP12 polypeptide.

P30. The nucleic acid of embodiment P29, wherein multimeric ligand binding region comprises a modified FKBP12 polypeptide comprising an amino acid substitution at position 36 that binds with higher affinity to the multimeric ligand than the wild type FKBP12 polypeptide.

P31. The nucleic acid of embodiment P30, wherein the amino acid substitution at position 36 is selected from the group consisting of valine, isoleucine, leucine, and alanine.

P32. The nucleic acid of any one of embodiments P22-P31, wherein the multimeric ligand binding region comprises a FKBP12v36 region.

P33. The nucleic acid of any one of embodiments P22-P32, wherein the ligand is dimeric FK506, or a dimeric FK506-like analog.

P34. The nucleic acid of any one of embodiments P22-P33, wherein the ligand is rimiducid or AP20187.

P35. The nucleic acid of any one of embodiments P22-P28, wherein the multimeric ligand binding region comprises a FKBP-rapamycin binding domain of mTOR (FRB) or FRB variant region.

P36. The nucleic acid of embodiment P35, wherein the FRB variant region is selected from the group consisting of KLW (T2098L), KTF (W2101F), and KLF (T2098L, W2101F).

P37. The nucleic acid of any one of embodiments P35-P36, wherein the FRB variant region is FRB_{L}

P38. The nucleic acid or cell of any one of embodiments P35-P37, wherein the FRB variant region binds to a rapalog selected from the group consisting of S-o,p-dimethoxyphenyl (DMOP)-rapamycin, R-Isopropoxyrapamycin, S-Butanesulfonamidorap, R and S C7-ethyloxyrapamycin, R and S C7-isopropyloxyrapamycin, R and S C7-isobutylrapamycin, R and S ethylcarbamaterapamycin, R and S C7-phenylcarbamaterapamycin, R and S C7-(3-methyl)indole rapamycin, temsirolimus, everolimus, zotarolimus, and R and S C7-(7-methyl)indole rapamycin.

P38.1. The nucleic acid or cell of any one of embodiments P1-P38, wherein the chimeric antigen receptor polypeptide does not include a CD40 cytoplasmic region polypeptide.

P39. The nucleic acid of any one of embodiments P1-P38.1, wherein the nucleic acid is an isolated nucleic acid.

P40. The nucleic acid of any one of embodiments P1-P39, wherein the nucleic acid is an isolated nucleic acid.

P41. The nucleic acid composition of any one of embodiments P1-P40, wherein the nucleic acid comprises a promoter sequence operably linked to the polynucleotide.

P42. The nucleic acid composition of any one of embodiments P1-P41, wherein the nucleic acid is contained within a viral vector.

P43. The nucleic acid composition of embodiment P42, wherein the viral vector is a retroviral vector.

P44. The nucleic acid composition of any one of embodiments P1-P43, wherein the nucleic acid is contained within a plasmid.

P45. A chimeric antigen receptor polypeptide encoded by a nucleic acid of any one of embodiments P1-P24.

Q1. A modified cell, wherein the cell is transduced or transfected with a nucleic acid of any one of embodiments P1-P43.

Q2-Q5. Reserved.

Q6. The modified cell of any one of embodiments Q1-Q5, wherein the cell is selected from the group consisting of T cell, tumor infiltrating lymphocyte, NK-T cell, invariant NK-T cell, gamma delta T cell, and NK cell.

Q6.1. The method of any one of embodiments Q1-Q5, wherein the cell is a gamma delta T cell.

Q6.2. The method of any one of embodiments Q1-Q5, wherein the cell is a NK cell.

Q6.3. The method of any one of embodiments Q1-Q5, wherein the cell is a invariant NK-T cell.

Q7. The modified cell of any one of embodiments Q1-Q5, wherein the cell is a T cell.

Q8. The modified cell of any one of embodiments Q1-Q5, wherein the cell is selected from the group consisting of K cells, NK-T cells, and T cells.

Q9. The modified cell of any one of embodiments Q1-Q8, wherein the cell is obtained or prepared from bone marrow.

Q10. The modified cell of any one of embodiments Q1-Q8, wherein the cell is obtained or prepared from umbilical cord blood.

Q11. The modified cell of any one of embodiments Q1-Q8, wherein the cell is obtained or prepared from peripheral blood.

Q12. The modified cell of any one of embodiments Q1-Q8, wherein the cell is obtained or prepared from peripheral blood mononuclear cells.

Q13. The modified cell of any one of embodiments Q1-Q8, wherein the cell is a human cell.

Q14. The modified cell of any one of embodiments Q1-Q8, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

Q15. The modified cell of any one of embodiments Q1-Q14, wherein the cell further comprises a nucleic acid comprising a polynucleotide encoding a chimeric Caspase-9 polypeptide comprising a multimeric ligand binding region and a Caspase-9 polypeptide.

Q16. The modified cell of embodiment Q15, wherein the Caspase-9 polypeptide lacks the CARD domain.

Q17. The modified cell of embodiment Q16, wherein the Caspase-9 polypeptide comprises the amino acid sequence of SEQ ID NO: 670.

Q18. The modified cell of embodiment Q17, wherein the Caspase-9 polypeptide comprises the amino acid sequence of SEQ ID NO. 670 and further comprises an amino acid substitution selected from the group consisting of the caspase variants in Table 5.

Q19. The modified cell of embodiment Q18, wherein the amino acid substitution is selected from the group consisting of N405Q, D330A, and D330E.

Q20. The modified cell of any one of embodiments Q15-Q19, wherein the multimeric ligand binding region comprises a FKBP12 polypeptide or modified FKBP12 polypeptide.

Q21. The modified cell of embodiment Q20, wherein multimeric ligand binding region comprises a modified FKBP12 polypeptide comprising an amino acid substitution at position 36 that binds with higher affinity to the multimeric ligand than the wild type FKBP12 polypeptide.

Q22. The modified cell of embodiment Q21, wherein the amino acid substitution at position 36 is selected from the group consisting of valine, isoleucine, leucine, and alanine.

Q23. The modified cell of any one of embodiments Q15-Q22, wherein the multimeric ligand binding region comprises a FKBP12v36 region.

Q24. The modified cell of any one of embodiments Q15-Q23, wherein the ligand is dimeric FK506, or a dimeric FK506-like analog.

Q25. The modified cell of any one of embodiments Q15-Q24, wherein the ligand is rimiducid or AP20187.

R1. A method for expressing an inducible chimeric antigen receptor polypeptide in a cell, comprising contacting a nucleic acid of any one of embodiments P1 to P34 with a cell under conditions in which the nucleic acid is incorporated into the cell, whereby the cell expresses the inducible chimeric signaling polypeptide from the incorporated nucleic acid.

R2. The method of embodiment R1, wherein the is contacted with a nucleic acid of any one of embodiments P22-P34, and the cell further expresses a chimeric Caspase-9 polypeptide comprising a multimeric ligand binding region and a Caspase-9 polypeptide.

R3. The method of any one of embodiments R1-R2, wherein the nucleic acid is contacted with the cell ex vivo.

R4. The method of any one of embodiment R1-R2, wherein the nucleic acid is contacted with the cell in vivo.

R5. The method of any one of embodiments R1-R4, wherein the cell is selected from the group consisting of T cell, tumor infiltrating lymphocyte, NK-T cell, invariant NK-T cell, gamma delta T cell, and NK cell.

R6. The method of any one of embodiments R1-R4, wherein the cell is a T cell.

R6.1. The method of any one of embodiments R1-R4, wherein the cell is a gamma delta T cell.

R6.2. The method of any one of embodiments R1-R4, wherein the cell is a NK cell.

R6.3. The method of any one of embodiments R1-R4, wherein the cell is a invariant NK-T cell.

R7. The method of any one of embodiments R1-R4, wherein the cell is selected from the group consisting of NK cells, NK-T cells, and T cells.

S1. A method for stimulating a cell-mediated immune response in a subject, comprising administering

a) a modified cell of any one of embodiments B1-B14 or L1-L14 to the subject; and

b) an effective amount of a multimeric ligand that binds to the multimeric ligand binding region to stimulate a cell-mediated immune response in the subject.

S2. The method of embodiment S1, wherein the cell expresses a chimeric antigen receptor, an inducible chimeric antigen receptor polypeptide of embodiment F36, or a recombinant T cell receptor, that binds to a target cell.

S3. The method of embodiment S2, wherein the target cell is a tumor cell.

S4. The method of any one of embodiments S2-S3, wherein the number or concentration of target cells in the subject is reduced following administration of the modified cell and the multimeric ligand.

S5. The method of any one of embodiments S2-S3, further comprising measuring the number or concentration of target cells in a first sample obtained from the subject before administering the modified cell or ligand, measuring the number or concentration of target cells in a second sample obtained from the subject after administration of the modified cell and ligand, and determining an increase or decrease of the number or concentration of target cells in the second sample compared to the number or concentration of target cells in the first sample.

S6. The method of embodiment S5, wherein the concentration of target cells in the second sample is decreased compared to the concentration of target cells in the first sample.

S7. The method of embodiment S5, wherein the concentration of target cells in the second sample is increased compared to the concentration of target cells in the first sample.

S8. The method of any one of embodiments S1-S7, wherein an additional dose of ligand is administered to the subject.

S9. The method of any one of embodiments S2-S8, wherein an effective amount of multimeric ligand is an amount effective to reduce the number or concentration of target cells and to reduce the symptoms of cytotoxicity.

S9.1. The method of any one of embodiments S1-S9, wherein the cell-mediated response is a T cell-mediated response.

S9.2. The method of any one of embodiments S1-S9, wherein the cell-mediated response is a NK cell or NK-T cell mediated response.

S9.3. The method of any one of embodiments S1-S9.2, wherein the proliferation and/or the survival of CD8⁺ T cells relative to CD4⁺ T cells leads to an alteration in the ratio of CD8⁺ T cells to CD4⁺ T cells in a sample of the subject following administration of the multimeric ligand, compared to the ratio of CD8⁺ T cells to CD4⁺ T cells in a sample of the subject before administration of the multimeric ligand.

S9.4. The method of embodiment S9.3, wherein the ratio of CD8⁺ T cells to CD4⁺ T cells is increased following administration of the multimeric ligand.

S9.5. The method of embodiment S9.3, wherein the ratio of CD8⁺ T cells to CD4⁺ T cells is decreased following administration of the multimeric ligand.

S9.6. The method of embodiment S9.3, wherein the ratio of CD8⁺ T cells to CD4⁺ T cells in the sample is 2:1 or greater.

S10. A method for treating a subject having a disease or condition associated with expression of a target antigen, comprising administering a multimeric ligand that binds to a multimeric ligand binding region, wherein
a) modified T cells circulating in the subject express (i) an inducible chimeric signaling polypeptide of embodiment A32 and a chimeric antigen receptor that binds to the target antigen; or (ii) an inducible chimeric antigen receptor polypeptide of embodiment K32 that binds to the target antigen;
b) the target antigen is present on target cells circulating in the subject; and
c) the number or concentration of target cells in the subject is reduced following administration of the multimeric ligand.

S11. The method of embodiment S10, wherein the target antigen is expressed by a tumor cell, and the chimeric antigen receptor or the inducible chimeric antigen receptor polypeptide binds to the tumor cell.

S12. The method of embodiment S2-S11, wherein following administration of the multimeric ligand, the number or concentration of target cells in the subject is determined, and (i) the administration of the multimeric ligand is discontinued or (ii) an additional dose of multimeric ligand is administered that is lower than the previous dose of multimeric ligand administered.

S13. The method of embodiment S2-S11, wherein following administration of the multimeric ligand, the number or concentration of target cells in the subject is determined, and an additional dose of multimeric ligand is administered that is higher than the previous dose of multimeric ligand administered.

S14. The method of embodiment S13 wherein the additional dose of multimeric ligand is from 120% to 200% greater than the previous dose.

S15. The method of embodiment S13, wherein the additional dose of multimeric ligand is about 150% greater than the previous dose.

S16. A method for providing anti-tumor immunity to a subject, comprising administering to the subject an effective amount of a modified cell of any one of embodiments B1-B14 or L1-L14 and administering a ligand that binds to the multimeric ligand binding region to provide anti-tumor immunity to the subject.

S17. A method for treating a subject having a disease or condition associated with expression of a target antigen, comprising administering to the subject an effective amount of a modified cell of any one of embodiments B1-B14 or L1-L14, and an effective amount of a ligand that binds to the multimeric ligand binding region.

S18. The method of embodiment S17, wherein the target antigen is a tumor antigen.

S19. The method of any one of embodiments S1-S18, wherein the modified cells are autologous T cells.

S20. The method of any one of embodiments S1-S18, wherein the modified cells are allogeneic T cells.

S21. A method for reducing the size of a tumor in a subject, comprising administering a modified cell of any one of embodiments B1-B14 or L1-L14 to the subject, wherein the cell expresses a chimeric antigen receptor or an inducible chimeric antigen receptor polypeptide comprising an antigen recognition moiety binds to an antigen on the tumor.

S22. The method of any one of embodiments S1-S21, wherein the modified cell is a tumor infiltrating lymphocyte or a T cell.

S23. The method of any one of embodiments S1-S22, wherein the modified cell is delivered to a tumor bed.

S24. The method of any one of embodiments S1-S23, further comprising determining whether an additional dose of the ligand should be administered to the subject.

S25. The method of any one of embodiments S10-S24 further comprising administering an additional dose of the ligand to the subject, wherein the disease or condition symptoms remain or are detected following a reduction in symptoms.

S26. The method of any one of embodiments S2-S25, wherein an effective amount of multimeric ligand is an amount effective to reduce the number or concentration of target antigen-expressing cells or the degree of tissue infiltration of the target antigen-expressing cells and to reduce the symptoms of cytotoxicity.

S27. The method of embodiment S1-S26, wherein following administration of the multimeric ligand, the level of cytoxicity symptoms is determined in the subject, and (i) the administration of the multimeric ligand is discontinued or (ii) an additional dose of multimeric ligand is administered that is lower than the previous dose of multimeric ligand administered.

S28. The method of embodiment S1-S26, wherein following administration of the multimeric ligand, the level of cytoxicity symptoms is determined in the subject, and an additional dose of multimeric ligand is administered that is higher than the previous dose of multimeric ligand administered.

S29. The method of embodiment S2-S28, wherein following administration of the multimeric ligand, the number or concentration of target antigen-expressing cells or the degree of tissue infiltration of the target antigen-expressing cells in the subject is determined, and (i) the administration of the multimeric ligand is discontinued or (ii) an additional dose of multimeric ligand is administered that is lower than the previous dose of multimeric ligand administered.

S30. The method of embodiment S2-S28, wherein following administration of the multimeric ligand, the number or concentration of target antigen-expressing cells or the degree of tissue infiltration of the target antigen-expressing cells in the subject is determined, and an additional dose of multimeric ligand is administered that is higher than the previous dose of multimeric ligand administered.

S31. The method of any one of embodiments S2-S30, further comprising identifying the presence, absence or stage of a condition or disease in a subject; and transmitting an indication to administer the ligand, maintain a subsequent dosage of the ligand, or adjust a subsequent dosage of the ligand administered to the subject based on the presence, absence or stage of the condition or disease identified in the subject.

S32. The method of any one of embodiments S1-S31, wherein the subject has been diagnosed as having a tumor.

S33. The method of any one of embodiments S1-S31, wherein the subject has cancer.

S34. The method of any one of embodiments S1-S31, wherein the subject has a solid tumor.

S35. The method of embodiment S33, wherein the cancer is present in the blood or bone marrow of the subject.

S36. The method of any one of embodiments S1-S31, wherein the subject has a blood or bone marrow disease.

S37. The method of any one of embodiments S1-S31, wherein the subject has been diagnosed with any condition or condition that can be alleviated by stem cell transplantation.

S38. The method of any one of embodiments S1-S31, wherein the subject has been diagnosed with sickle cell anemia or metachromatic leukodystrophy.

S39. The method of any one of embodiments S1-S31, wherein the subject has been diagnosed with a condition selected from the group consisting of a primary immune deficiency condition, hemophagocytosis lymphohistiocytosis (HAH) or other hemophagocytic condition, an inherited marrow failure condition, a hemoglobinopathy, a metabolic condition, and an osteoclast condition.

S40. The method of any one of embodiments S1-S31, wherein the subject has been diagnosed with a disease or condition selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/IL-10 Receptor Deficiency, GATA 2 deficiency, X-linked lymphoproliferative disease (XAP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.

S41. The method of any one of embodiments S1-S31, wherein the subject has been diagnosed with leukemia.

S42. The method of any one of embodiments S1-S31, wherein the subject has been diagnosed with an infection of viral etiology selected from the group consisting HIV, influenza, Herpes, viral hepatitis, Epstein Bar, polio, viral encephalitis, measles, chicken pox, Cytomegalovirus (CMV), adenovirus (ADV), HHV-6 (human herpesvirus 6, I), and Papilloma virus, or has been diagnosed with an infection of bacterial etiology selected from the group consisting of pneumonia, tuberculosis, and syphilis, or has been diagnosed with an infection of parasitic etiology selected from the group consisting of malaria, trypanosomiasis, leishmaniasis, trichomoniasis, and amoebiasis.

S43. The method of any one of embodiments S1-S42 wherein the ligand is rimiducid or AP21087

T1. A method for stimulating a cell-mediated immune response in a subject, comprising administering an effective amount of modified cells of any one of embodiments G15-G25 or Q15-Q25 to the subject.

T2. The method of embodiment T1, wherein the cell-mediated immune response is directed against a target cell.

T3. The method of any one of embodiments T1 or T2, wherein the modified cell comprises a chimeric antigen receptor, a chimeric antigen receptor polypeptide of embodiment P43, or a recombinant T cell receptor, that binds to an antigen on a target cell.

T4. The method of any one of embodiments T2 or T3, wherein the target cell is a tumor cell.

T5. The method of any one of embodiments T2-T4, wherein the number or concentration of target cells in the subject is reduced following administration of the modified cells.

T6. The method of any one of embodiments T2-T5, comprising measuring the number or concentration of target cells in a first sample obtained from the subject before administering the modified cell, measuring the number concentration of target cells in a second sample obtained from the subject after administration of the modified cell, and determining an increase or decrease of the number or concentration of target cells in the second sample compared to the number or concentration of target cells in the first sample.

T7. The method of embodiment T6, wherein the concentration of target cells in the second sample is decreased compared to the concentration of target cells in the first sample.

T8. The method of embodiment T6, wherein the concentration of target cells in the second sample is increased compared to the concentration or target cells in the first sample.

T9. The method of any one of embodiments T1-T8, wherein an additional dose of modified cells is administered to the subject.

T9.1. The method of any one of embodiments T1-T9, wherein the cell-mediated response is a T cell-mediated response.

T9.2. The method of any one of embodiments T1-T9, wherein the cell-mediated response is a NK cell or NK-T cell-mediated response.

T10. A method for providing anti-tumor immunity to a subject, comprising administering to the subject an effective amount of a modified cell of any one of embodiments G1-G25 or Q1-Q25.

T11. A method for treating a subject having a disease or condition associated with an elevated expression of a target antigen, comprising administering to the subject an effective amount of a modified cell of any one of embodiments G1-G25 or Q1-Q25.

T12. The method of embodiment T11, wherein the target antigen is a tumor antigen.

T13 A method for reducing the size of a tumor in a subject, comprising administering a modified cell of any one of embodiments to the subject, wherein the modified cell comprises a chimeric antigen receptor, a chimeric antigen receptor polypeptide of embodiment P43, or a recombinant T cell receptor, comprising an antigen recognition moiety that binds to an antigen on the tumor.

T14. The method of any one of embodiments T1-T13, wherein the subject has been diagnosed as having a tumor.

T15. The method of any one of embodiments T1-T13, wherein the subject has cancer.

T16. The method of embodiment T15, wherein the subject has a solid tumor or leukemia.

T17. The method of any one of embodiments T1-T16, wherein the modified cell is a tumor infiltrating lymphocyte or a T cell.

T18. The method of any one of embodiments T1-T17, wherein the modified cell is delivered to a tumor bed.

T19. The method of embodiment T15, wherein the cancer is present in the blood or bone marrow of the subject.

T20. The method of any one of embodiments T1-T17, wherein the subject has a blood or bone marrow disease.

T21. The method of any one of embodiments T1-T17, wherein the subject has been diagnosed with any condition or condition that can be alleviated by stem cell transplantation.

T22. The method of any one of embodiments T1-T17, wherein the subject has been diagnosed with sickle cell anemia or metachromatic leukodystrophy.

T23. The method of any one of embodiments T1-T17, wherein the subject has been diagnosed with a condition selected from the group consisting of a primary immune deficiency condition, hemophagocytosis lymphohistiocytosis (HLH) or other hemophagocytic condition, an inherited marrow failure condition, a hemoglobinopathy, a metabolic condition, and an osteoclast condition.

T24. The method of any one of embodiments T1-T17, wherein the subject has been diagnosed with a disease or condition selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/IL-10 Receptor Deficiency, GATA 2 deficiency, X-linked lymphoproliferative disease (XLP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.

T25. The method of any one of embodiments T1-T24, further comprising determining whether an additional dose of the modified cell should be administered to the subject.

T26. The method of any one of embodiments T1-T25, further comprising administering an additional dose of the modified cell to the subject, wherein the disease or condition symptoms remain or are detected following a reduction in symptoms.

T27. The method of any one of embodiments T1-26 further comprising identifying the presence, absence or stage of a condition or disease in a subject; and transmitting an indication to administer modified cell of any one of embodiments 68-93, maintain a subsequent dosage of the modified cell, or adjust a subsequent dosage of the modified cell administered to the patient based on the presence, absence or stage of the condition or disease identified in the subject.

T28. The method of any one of embodiments T1-T27, wherein the subject has been diagnosed with an infection of viral etiology selected from the group consisting HIV, influenza, Herpes, viral hepatitis, Epstein Bar, polio, viral encephalitis, measles, chicken pox, Cytomegalovirus (CMV), adenovirus (ADV), HHV-6 (human herpesvirus 6, I), and Papilloma virus, or has been diagnosed with an infection of bacterial etiology selected from the group consisting of pneumonia, tuberculosis, and syphilis, or has been diagnosed with an infection of parasitic etiology selected from the group consisting of malaria, trypanosomiasis, leishmaniasis, trichomoniasis, and amoebiasis.

T29. The method of any one of embodiments T1-T28, wherein the modified cell comprises a chimeric Caspase-9 polypeptide comprising a multimeric ligand binding region and a Caspase-9 polypeptide.

T30. The method of embodiment T29, further comprising administering a multimeric ligand that binds to the multimeric ligand binding region to the subject following administration of the modified cells to the subject.

T31. The method of embodiment T30, wherein after administration of the multimeric ligand, the number of modified cells comprising the chimeric Caspase-9 polypeptide is reduced.

T32. The method of embodiment T31, wherein the number of modified cells comprising the chimeric Caspase-9 polypeptide is reduced by 50%.

T33. The method of embodiment T31, wherein the number of modified cells comprising the chimeric Caspase-9 polypeptide is reduced by 75%.

T34. The method of embodiment T31, wherein the number of modified cells comprising the chimeric Caspase-9 polypeptide is reduced by 90%.

T35. The method of any one of embodiments T1-T28, comprising determining that the subject is experiencing a negative symptom following administration of the modified cells to the subject, and administering the ligand to reduce or alleviate the negative symptom.

T36. The method of any one of embodiments T29-T35, wherein the ligand is rimiducid or AP20187.

T37. The method of any one of embodiments T1-T36, wherein the modified cells are autologous T cells.

T38. The method of any one of embodiments T1-T36, wherein the modified cells are allogeneic T cells.

T39. The method of any one of embodiments T1-T38, wherein the modified cells are transfected or transduced in vivo.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s)

1. A modified cell comprising a nucleic acid that comprises a promoter operably linked to a polynucleotide encoding an inducible chimeric signaling polypeptide, wherein the polypeptide comprises
a) first ligand binding region;
b) a second ligand binding region;
c) a MyD88 polypeptide or a truncated MyD88 polypeptide lacking the TIR domain; and
d) a costimulatory polypeptide cytoplasmic signaling region selected from the group consisting of CD27, CD28, ICOS, 4-1BB, RANK, OX40, CD30, TAC1, CD122, GITR, GITR EEE191RVV, TweakR, BCMA, SOD1, SOD2, SOD3, CD86, C74Ra, Dectin, IL15Ra, ITA4, ITGA5, TCL1A, CTLA4, TIM1, TREMBL2, HVEM, BTN3A1 B30.2 domain, CD40-HCR-CD40, CD40DPP4, RANK88, RANK TRAF6, RANK TRAF2/5, RANK HCR-TRAF2/5, cytoplasmic signaling regions.

2. The modified cell of claim 1, wherein the polynucleotide encoding the inducible chimeric signaling polypeptide is a first polynucleotide, and the nucleic acid comprises a second polynucleotide encoding a chimeric antigen receptor.

3.-7. (canceled)

8. The modified cell claim 1, wherein the cell is a T cell.

9. The modified cell claim 1, wherein the cell is an NK cell.

10. The modified cell claim 1, wherein the cell is selected from the group consisting of invariant NK-T cells, gamma delta T cells and tumor infiltrating lymphocytes.

11.-21. (canceled)

22. The modified cell of claim 1, wherein the costimulatory polypeptide cytoplasmic signaling region is selected from the group consisting of CD28, 4-1BB, OX40, ICOS, BCMA, CD27, CD30, CD122, GITR S180A, GITR EEE191RVV, HVEM, TWEAKR, RANK, RANK TRAF6, RANK TRAF 2/5, RANK HCR-TRAF2/5, CD40-HCR-CD40, SOD1, SOD2, TAC1, SOD3, RANK88, IL15Ra, BTN3A1 (B30.2 domain), CD86, CD74Ra, Dectin, ITA4, ITGA5, TCL1A, CTLA4, TIM1, TREMBL2, CD40-HCR- CD40, and DPP4 polypeptide cytoplasmic signaling regions.

23. The modified cell of claim 1, wherein the costimulatory polypeptide cytoplasmic signaling region is selected from the group consisting of CD28, 4-1BB, OX40, ICOS, BCMA, CD27, CD30, CD122, GITR S180A, GITR EEE191RVV, HVEM, TWEAKR, RANK, RANK TRAF6, RANK TRAF 2/5, RANK HCR-TRAF2/5, SOD1, SOD2, TAC1, SOD3, RANK88, IL15Ra, BTN3A1 (B30.2 domain), CD86, CD74Ra, Dectin, ITA4, ITGA5, TCL1A, CTLA4, TIM1, TREMBL2, and DPP4 polypeptide cytoplasmic signaling regions.

24. The modified cell of claim 1, wherein the costimulatory polypeptide cytoplasmic signaling region is selected from the group consisting of CD28, 4-1BB, OX40, ICOS, BCMA, CD27, CD30, CD122, GITR S180A, GITR EEE191RVV, HVEM, TWEAKR, SOD1, SOD2, TAC1, SOD3, IL15Ra, BTN3A1 (B30.2 domain), CD86, CD74Ra, Dectin, ITA4, ITGA5, TCL1A, CTLA4, TIM1, TREMBL2, and DPP4 polypeptide cytoplasmic signaling regions.

25. The modified cell of claim 1, wherein the costimulatory polypeptide cytoplasmic signaling region is selected from the group consisting of RANK, RANK TRAF6, RANK TRAF 2/5, RANK HCR-TRAF2/5, CD40-HCR-CD40, and RANK88 polypeptide cytoplasmic signaling regions.

26. The modified cell of claim 1, wherein the costimulatory polypeptide cytoplasmic signaling region is selected from the group consisting of RANK, RANK TRAF6, RANK TRAF 2/5, RANK HCR-TRAF2/5, and RANK88 polypeptide cytoplasmic signaling regions.

27. The modified cell of claim 1, wherein the costimulatory polypeptide cytoplasmic signaling region is selected from the group consisting of CD27, CD28, ICOS, 4-1BB, RANK/TRANCE-R, OX40, CD30, TweakR, TAC1, BCMA and HVEM polypeptide cytoplasmic signaling regions.

28. The modified cell of claim 1, wherein the costimulatory polypeptide cytoplasmic signaling region is selected from the group consisting of CD27, CD28, ICOS, OX40, CD30, TweakR, TAC1, BCMA and HVEM polypeptide cytoplasmic signaling regions.

29. The modified cell of claim 1, wherein the costimulatory polypeptide cytoplasmic signaling region is selected from the group consisting of 4-1BB, RANK, OX40, and HVEM polypeptide cytoplasmic signaling regions.

30. (canceled)

31. The modified cell of claim 1, wherein the costimulatory polypeptide cytoplasmic signaling region is a 4-1BB polypeptide cytoplasmic signaling region.

32.-32.2. (canceled)

33. A pharmaceutical composition comprising the modified cell of claim 1.

34. The modified cell of claim 1, wherein the chimeric signaling polypeptide comprises a membrane targeting region.

35. The modified cell of claim 34, wherein the membrane targeting region is selected from the group consisting of a myristoylation region, a palmitoylation region, a prenylation region, and transmembrane sequences of receptors.

36. The modified cell of claim 34, wherein the membrane targeting region is a myristoylation region.

37. The modified cell of claim 36, wherein the membrane targeting region is a myristoylation region has an amino acid sequence of SEQ ID NO: 4, or an amino acid sequence 95% or more identical to SEQ ID NO:4.

37.1. (canceled)

38. The modified cell of claim 1, wherein the inducible chimeric signaling polypeptide comprises two FKBP12 variant polypeptide regions, a truncated MyD88 polypeptide, and a 4-1BB polypeptide cytoplasmic signaling region.

39. The modified cell of claim 38, wherein the inducible chimeric signaling polypeptide comprises a myristoylation region.

40. The modified cell of claim 38, wherein the inducible chimeric signaling polypeptide comprises no myristoylation region.

41.-54. (canceled)

55. The modified cell of claim 1, wherein the first ligand binding region comprises a FKBP12 variant polypeptide region.

56. The modified cell of claim 1, wherein the second ligand binding region comprises a FKBP12 variant polypeptide region.

57. The modified cell of claim 1, wherein the first ligand binding region and the second ligand binding region each comprises a FKBP12 variant polypeptide region.

58. The modified cell of claim 1, wherein the first ligand binding region or the second ligand binding region comprises a FKBP12 wild type polypeptide region.

59. The modified cell of claim 58, wherein the first ligand binding region and the second ligand binding region each comprises a FKBP12 wild type polypeptide region.

60. The modified cell of claim 1, wherein the first and second ligand binding regions together are Fv′Fvls.

61. The modified cell of claim 60, wherein Fv′Fvls comprises two linked polypeptides comprising the amino acid sequence of SEQ ID NO: 6 and the amino acid sequence of SEQ ID NO: 8.

62.-66. (canceled)

67. The modified cell of claim 1, wherein the first ligand binding region is an FK506 binding protein 12 (FKBP12) region and the second ligand binding region is an FKBP12-Rapamycin-binding domain of mTOR (FRB) region, or an FRB variant polypeptide region.

68.-163. (canceled)

164. The modified cell of claim 1, wherein the inducible chimeric signaling polypeptide comprises no membrane targeting region.


SEQUENCE LISTING (1031 sequences)

SEQ ID NO: 1 - DNA, 516 bp, Homo sapiens
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatc

SEQ ID NO: 2 - protein, 172 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 3 - DNA, 42 bp, Artificial Sequence
  source: /note=“Description of Artificial Sequence: Synthetic oligonucleotide”
atggggagtagcaagagcaagcctaaggaccccagccagcgc

SEQ ID NO: 4 - protein, 14 aa, Artificial Sequence
  source: /note=“Description of Artificial Sequence: Synthetic peptide”
MGSSKSKPKDPSQR

SEQ ID NO: 5 - DNA, 321 bp, Artificial Sequence
  source: /note=“Description of Artificial Sequence: Synthetic polynucleotide”
ggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaa

SEQ ID NO: 6 - protein, 107 aa, Artificial Sequence
  source: /note=“Description of Artificial Sequence: Synthetic polypeptide”
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 7 - DNA, 321 bp, Artificial Sequence
  source: /note=“Description of Artificial Sequence: Synthetic polynucleotide”
ggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 8 - protein, 107 aa, Artificial Sequence; identical to SEQ ID NO: 6
  source: /note=“Description of Artificial Sequence: Synthetic polypeptide”

SEQ ID NO: 9 - DNA, 54 bp, Thosea asigna virus
gagggcagaggcagcctcctgacatgtggggacgtcgaggagaaccctggccca

SEQ ID NO: 10 - protein, 18 aa, Thosea asigna virus
EGRGSLLTCGDVEENPGP

SEQ ID NO: 11 - skipped

SEQ ID NO: 12 - protein, 62 aa, Homo sapiens
KKVAKKPTNKAPHPKQEPQEINFPDDLPGSNTAAPVQETLHGCQPVTQEDGKESRISVQERQ

SEQ ID NO: 13 - skipped

SEQ ID NO: 14 - protein, 46 aa, Artificial Sequence
  source: /note=“Description of Artificial Sequence: Synthetic polypeptide”
RSKRSRLLHSDYMNMTPRRPGPTRKHYQPYAPPRDFAAYRSGSGQL

SEQ ID NO: 15 - skipped

SEQ ID NO: 16 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 17 - skipped

SEQ ID NO: 18 - protein, 39 aa, Artificial Sequence
  source: /note=“Description of Artificial Sequence: Synthetic polypeptide”
RDQRLPPDAHKPPGGGSFRTPIQEEQADAHSTLAKIGSG

SEQ ID NO: 19 - skipped

SEQ ID NO: 20 - protein, 35 aa, Artificial Sequence
  source: /note=“Description of Artificial Sequence: Synthetic polypeptide”
TKKKYSSSVHDPNGEYMFMRAVNTAKKSRLTDVTL

SEQ ID NO: 21 - skipped

SEQ ID NO: 22 - protein, 107 aa, Artificial Sequence
  source: /note=“Description of Artificial Sequence: Synthetic polypeptide”
RKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 23 - skipped

SEQ ID NO: 24 - protein, 48 aa, Artificial Sequence
  source: /note=“Description of Artificial Sequence: Synthetic polypeptide”
QRRKYRSNKGESPVEPAEPCRYSCPREEEGSTIPIQEDYRKPEPACSP

SEQ ID NO: 25 - skipped

SEQ ID NO: 26 - protein, 188 aa, Artificial Sequence
  source: /note=“Description of Artificial Sequence: Synthetic polypeptide”
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 27 - skipped

SEQ ID NO: 28 - protein, 167 aa, Artificial Sequence
  source: /note=“Description of Artificial Sequence: Synthetic polypeptide”
NCRNTGPWLKKVLKCNTPDPSKFFSQLSSEHGGDVQKWLSSPFPSSSFSPGGLAPEISPLEVLERDKVTQMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 29 - skipped

SEQ ID NO: 30 - protein, 58 aa, Artificial Sequence
  source: /note=“Description of Artificial Sequence: Synthetic polypeptide”
QLGLHIWQLRSQCMWPRETQLLLEVPPATEDARSCQFPEEERGERSAEEKGRLGDLWV

SEQ ID NO: 31 - skipped

SEQ ID NO: 32 - protein, 58 aa, Artificial Sequence
  source: /note=“Description of Artificial Sequence: Synthetic polypeptide”
QLGLHIWQLRSQCMWPRETQLLLEVPPSTEDARSCQFPRVVRGERSAEEKGRLGDLWV

SEQ ID NO: 33 - skipped

SEQ ID NO: 34 - protein, 60 aa, Artificial Sequence
  source: /note=“Description of Artificial Sequence: Synthetic polypeptide”
CVKRRKPRGDVVKVIVSVQRKRQEAEGEATVIEALQAPPDVTTVAVEETIPSFTGRSPNH

SEQ ID NO: 35 - skipped

SEQ ID NO: 36 - protein, 60 aa, Artificial Sequence; identical to SEQ ID NO: 34
  source: /note=“Description of Artificial Sequence: Synthetic polypeptide”

SEQ ID NO: 37-39 - skipped (3 entries)

SEQ ID NO: 40 - protein, 383 aa, Artificial Sequence
  source: /note=“Description of Artificial Sequence: Synthetic polypeptide”
CYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 41 - skipped

SEQ ID NO: 42 - protein, 101 aa, Artificial Sequence
  source: /note=“Description of Artificial Sequence: Synthetic polypeptide”
MLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEV

SEQ ID NO: 43 - skipped

SEQ ID NO: 44 - protein, 88 aa, Artificial Sequence
  source: /note=“Description of Artificial Sequence: Synthetic polypeptide”
SNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 45 - skipped

SEQ ID NO: 46 - protein, 124 aa, Artificial Sequence
  source: /note=“Description of Artificial Sequence: Synthetic polypeptide”
PEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 47 - skipped

SEQ ID NO: 48 - protein, 383 aa, Artificial Sequence; identical to SEQ ID NO: 40
  source: /note=“Description of Artificial Sequence: Synthetic polypeptide”

SEQ ID NO: 49 - skipped

SEQ ID NO: 50 - protein, 128 aa, Artificial Sequence
  source: /note=“Description of Artificial Sequence: Synthetic polypeptide”
KKVAKKPTNKAPHPKQEPQEINFPDDLPGPEDGADGRLPSSARAGAGSGSSPGGQSPASGNVTGNSNSTFISSGQVMNFKGDIIVVYVSQTSQESNTAAPVQETLHGCQPVTQEDGKESRISVQERQV

SEQ ID NO: 51 - skipped

SEQ ID NO: 52 - protein, 154 aa, Artificial Sequence [sequence omitted]

SEQ ID NO: 53 - skipped

SEQ ID NO: 54 - protein, 140 aa, Artificial Sequence [sequence omitted]

SEQ ID NO: 55 - skipped

SEQ ID NO: 56 - protein, 222 aa, Artificial Sequence
  source: /note=“Description of Artificial Sequence: Synthetic polypeptide”
WTGEDSAEPNSDSAEWIRDMYAKVTEIWQEVMQRRDDDGALHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVGVCGPGLWERQAREHSERKKRRRESECKAA

SEQ ID NO: 57 - skipped

SEQ ID NO: 58 - protein, 98 aa, Artificial Sequence
  source: /note=“Description of Artificial Sequence: Synthetic polypeptide”
PEDGADGRLPSSARAGAGSGSSPGGQSPASGNVTGNSNSTFISSGQVMNFKGDIIVVYVSQTSQESNTAAPVQETLHGCQPVTQEDGKESRISVQERQ

SEQ ID NO: 59 - skipped

SEQ ID NO: 60 - protein, 42 aa, Artificial Sequence; identical to SEQ ID NO: 16
  source: /note=“Description of Artificial Sequence: Synthetic polypeptide”

SEQ ID NO: 61 - skipped

SEQ ID NO: 62 - protein, 42 aa, Artificial Sequence
  source: /note=“Description of Artificial Sequence: Synthetic polypeptide”
ALYLLRRDQRLPPDAHKPPGGGSFRTPIQEEQADAHSTLAKI

SEQ ID NO: 63 - skipped

SEQ ID NO: 64 - protein, 39 aa, Artificial Sequence
  source: /note=“Description of Artificial Sequence: Synthetic polypeptide”
KSRQTPPLASVEMEAMEALPVTWGTSSRDEDLENCSHHL

SEQ ID NO: 65 - skipped

SEQ ID NO: 66 - DNA, 114 bp, Artificial Sequence
  source: /note=“Description of Artificial Sequence: Synthetic polynucleotide”
tgttggctcaccaaaaagaagtattcttcatcagtgcatgaccctaacggcgagtatatgtttatgcgggcggtcaatacggccaagaagtcccggcttaccgacgttactttg

SEQ ID NO: 67 - skipped

SEQ ID NO: 68 - protein, 242 aa, Artificial Sequence
  source: /note=“Description of Artificial Sequence: Synthetic polypeptide”
QQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 69 - skipped

SEQ ID NO: 70 - protein, 192 aa, Artificial Sequence
  source: /note=“Description of Artificial Sequence: Synthetic polypeptide”
RGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 71 - skipped

SEQ ID NO: 72 - protein, 61 aa, Artificial Sequence
  source: /note=“Description of Artificial Sequence: Synthetic polypeptide”
KWKKKKRPRNSYKCGTNTMEREESEQTKKREKIHIPERSDEAQRVFKSSKTSSCDKSDTCF

SEQ ID NO: 73 - skipped

SEQ ID NO: 74 - protein, 45 aa, Artificial Sequence
  source: /note=“Description of Artificial Sequence: Synthetic polypeptide”
MHRRRSRSCREDQKPVMDDQRDLISNNEQLPMLGRRPGAPESKCS

SEQ ID NO: 75 - skipped

SEQ ID NO: 76 - protein, 44 aa, Artificial Sequence
  source: /note=“Description of Artificial Sequence: Synthetic polypeptide”
MEYHPDLENLDEDGYTQLHFDSQSNTRIAVVSEKGSCAASPPWR

SEQ ID NO: 77 - skipped

SEQ ID NO: 78 - protein, 62 aa, Artificial Sequence; identical to SEQ ID NO: 12
  source: /note=“Description of Artificial Sequence: Synthetic polypeptide”

SEQ ID NO: 79 - skipped

SEQ ID NO: 80 - protein, 31 aa, Artificial Sequence
  source: /note=“Description of Artificial Sequence: Synthetic polypeptide”
AGFFKRQYKSILQEENRRDSWSYINSKSNDD

SEQ ID NO: 81 - skipped

SEQ ID NO: 82 - protein, 28 aa, Artificial Sequence
  source: /note=“Description of Artificial Sequence: Synthetic peptide”
KLGFFKRSLPYGTAMEKAQLKPPATSDA

SEQ ID NO: 83 - skipped

SEQ ID NO: 84 - protein, 45 aa, Artificial Sequence
  source: /note=“Description of Artificial Sequence: Synthetic polypeptide”
RKWCQYQKEIMERPPPFKPPPPPIKYTCIQEPNESDLPYHEMETL

SEQ ID NO: 85 - skipped

SEQ ID NO: 86 - protein, 41 aa, Artificial Sequence
  source: /note=“Description of Artificial Sequence: Synthetic polypeptide”
AVSLSKMLKKRSPLTTGVYVKMPPTEPECEKQFQPYFIPIN

SEQ ID NO: 87 - skipped

SEQ ID NO: 88 - protein, 48 aa, Artificial Sequence
  source: /note=“Description of Artificial Sequence: Synthetic polypeptide”
KKYFFKKEVQQLSVSFSSLQIKALQNAVEKEVQAEDNIYIENSLYATD

SEQ ID NO: 89 - skipped

SEQ ID NO: 90 - protein, 32 aa, Artificial Sequence
  source: /note=“Description of Artificial Sequence: Synthetic polypeptide”
KKRHMASYSMCSDPSTRDPPGRPEPYVEVYLI

SEQ ID NO: 91 - DNA, 321 bp, Homo sapiens
ggcgtgcaagtggaaactataagcccgggagacggccgcacatttcccaagagaggccagacctgcgtggtgcactatacaggaatgctggaggacgggaagaaattcgatagctcccgggatcgaaataagcctttcaaattcatgctgggcaagcaagaagtcatcagaggctgggaagaaggcgtcgcccagatgtccgtgggtcagcgcgccaagctgacaattagtccagattacgcctatggcgcaacaggccatcccggcatcatccccccacatgccacactcgtctttgatgtcgagctcctgaaactggag

SEQ ID NO: 92 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 93-128 - skipped (36 entries)

SEQ ID NO: 129 - DNA, 6 bp, Artificial Sequence
  source: /note=“Description of Artificial Sequence: Synthetic oligonucleotide”
ctcgag

SEQ ID NO: 130 - protein, 2 aa, Artificial Sequence
  source: /note=“Description of Artificial Sequence: Synthetic peptide”
LE

SEQ ID NO: 131 - DNA, 108 bp, Artificial Sequence
  source: /note=“Description of Artificial Sequence: Synthetic polynucleotide”
agggaccagaggctgccccccgatgcccacaagccccctgggggaggcagtttccggacccccatccaagaggagcaggccgacgcccactccaccctggccaagatc

SEQ ID NO: 132 - protein, 39 aa, Artificial Sequence; identical to SEQ ID NO: 18
  source: /note=“Description of Artificial Sequence: Synthetic polypeptide”

SEQ ID NO: 133-172 - skipped (40 entries)

SEQ ID NO: 173 - DNA, 105 bp, Artificial Sequence
  source: /note=“Description of Artificial Sequence: Synthetic polynucleotide”
acaaaaaagaagtattcatccagtgtgcacgaccctaacggtgaatacatgttcatgagagcagtgaacacagccaaaaaatctagactcacagatgtgacccta

SEQ ID NO: 174 - protein, 35 aa, Artificial Sequence; identical to SEQ ID NO: 20
  source: /note=“Description of Artificial Sequence: Synthetic polypeptide”

SEQ ID NO: 175-288 - skipped (114 entries)

SEQ ID NO: 289 - DNA, 9 bp, Artificial Sequence
  source: /note=“Description of Artificial Sequence: Synthetic oligonucleotide”
atgctcgag

SEQ ID NO: 290 - protein, 3 aa, Artificial Sequence
  source: /note=“Description of Artificial Sequence: Synthetic peptide”
MLE

SEQ ID NO: 291 - DNA, 126 bp, Artificial Sequence
  source: /note=“Description of Artificial Sequence: Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 292 - protein, 42 aa, Artificial Sequence; identical to SEQ ID NO: 16
  source: /note=“Description of Artificial Sequence: Synthetic polypeptide”

SEQ ID NO: 293-500 - skipped (208 entries)

SEQ ID NO: 501 - DNA, 186 bp, Artificial Sequence
  source: /note=“Description of Artificial Sequence: Synthetic polynucleotide”
aaaaaggtggccaagaagccaaccaataaggccccccaccccaagcaggagccccaggagatcaattttcccgacgatcttcctggctccaacactgctgctccagtgcaggagactttacatggatgccagccggtcacccaggaggatggcaaagagagtcgcatctcagtgcaggagagacag

SEQ ID NO: 502 - protein, 62 aa, Artificial Sequence; identical to SEQ ID NO: 12
  source: /note=“Description of Artificial Sequence: Synthetic polypeptide”

SEQ ID NO: 503-664 - skipped (162 entries)

SEQ ID NO: 665 - DNA, 321 bp, Artificial Sequence
  source: /note=“Description of Artificial Sequence: Synthetic polynucleotide”
ggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaa

SEQ ID NO: 666 - protein, 107 aa, Artificial Sequence; identical to SEQ ID NO: 92
  source: /note=“Description of Artificial Sequence: Synthetic polypeptide”

SEQ ID NO: 667 - DNA, 24 bp, Artificial Sequence
  source: /note=“Description of Artificial Sequence: Synthetic oligonucleotide”
agcggaggaggatccggagtggac

SEQ ID NO: 668 - protein, 8 aa, Artificial Sequence
  source: /note=“Description of Artificial Sequence: Synthetic peptide”
SGGGSGVD

SEQ ID NO: 669 - DNA, 858 bp, Homo sapiens
gggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctccgcatctagggcc

SEQ ID NO: 670 - protein, 286 aa, Homo sapiens
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 671 - DNA, 6 bp, Artificial Sequence
  source: /note=“Description of Artificial Sequence: Synthetic oligonucleotide”
ccgcgg

SEQ ID NO: 672 - protein, 2 aa, Artificial Sequence
  source: /note=“Description of Artificial Sequence: Synthetic peptide”
PR

SEQ ID NO: 673 - DNA, 54 bp, Thosea asigna virus
gaaggccgagggagcctgctgacatgtggcgatgtggaggaaaacccaggacca

SEQ ID NO: 674 - protein, 18 aa, Thosea asigna virus
EGRGSLLTCGDVEENPGP

SEQ ID NO: 675 - DNA, 6 bp, Artificial Sequence
  source: /note=“Description of Artificial Sequence: Synthetic oligonucleotide”
ccatgg

SEQ ID NO: 676 - protein, 2 aa, Artificial Sequence
  source: /note=“Description of Artificial Sequence: Synthetic peptide”
PW

SEQ ID NO: 677 - DNA, 63 bp, Artificial Sequence
  source: /note=“Description of Artificial Sequence: Synthetic oligonucleotide”
atggagtttggactttcttggttgtttttggtggcaattctgaagggtgtccagtgtagcagg

SEQ ID NO: 678 - protein, 21 aa, Artificial Sequence
  source: /note=“Description of Artificial Sequence: Synthetic peptide”
MEFGLSWLFLVAILKGVQCSR

SEQ ID NO: 679 - DNA, 321 bp, Artificial Sequence
  source: /note=“Description of Artificial Sequence: Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 680 - protein, 107 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 681 - DNA, 24 bp, Artificial Sequence
  source: /note=“Description of Artificial Sequence: Synthetic oligonucleotide”
ggcggaggaagcggaggtgggggc

SEQ ID NO: 682 - protein, 8 aa, Artificial Sequence
  source: /note=“Description of Artificial Sequence: Synthetic peptide”
GGGSGGGG

SEQ ID NO: 683 - DNA, 360 bp, Artificial Sequence
  source: /note=“Description of Artificial Sequence: Synthetic polynucleotide”
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 684 - protein, 120 aa, Artificial Sequence
  source: /note=“Description of Artificial Sequence: Synthetic polypeptide”
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 685 - DNA, 6 bp, Artificial Sequence
  source: /note=“Description of Artificial Sequence: Synthetic oligonucleotide”
ggatcc

SEQ ID NO: 686 - protein, 2 aa, Artificial Sequence
  source: /note=“Description of Artificial Sequence: Synthetic peptide”
GS

SEQ ID NO: 687 - DNA, 48 bp, Artificial Sequence
  source: /note=“Description of Artificial Sequence: Synthetic oligonucleotide”
gaacttcctactcaggggactttctcaaacgttagcacaaacgtaagt

SEQ ID NO: 688 - protein, 16 aa, Artificial Sequence
  source: /note=“Description of Artificial Sequence: Synthetic peptide”
ELPTQGTFSNVSTNVS

SEQ ID NO: 689 - DNA, 126 bp, Artificial Sequence
  source: /note=“Description of Artificial Sequence: Synthetic polynucleotide”
cccgccccaagaccccccacacctgcgccgaccattgcttctcaacccctgagtttgagacccgaggcctgccggccagctgccggcggggccgtgcatacaagaggactcgatttcgcttgcgac

SEQ ID NO: 690 - protein, 42 aa, Artificial Sequence
  source: /note=“Description of Artificial Sequence: Synthetic polypeptide”
PAPRPPTPAPTIASQPLSLRPEACRPAAGGAVHTRGLDFACD

SEQ ID NO: 691 - DNA, 111 bp, Artificial Sequence
  source: /note=“Description of Artificial Sequence: Synthetic polynucleotide”
atctatatctgggcacctctcgctggcacctgtggagtccttctgctcagcctggttattactctgtactgtaatcaccggaatcgccgccgcgtttgtaagtgtcccagg

SEQ ID NO: 692 - protein, 37 aa, Artificial Sequence
  source: /note=“Description of Artificial Sequence: Synthetic polypeptide”
IYIWAPLAGTCGVLLLSLVITLYCNHRNRRRVCKCPR

SEQ ID NO: 693 - DNA, 6 bp, Artificial Sequence
  source: /note=“Description of Artificial Sequence: Synthetic oligonucleotide”
gtcgac

SEQ ID NO: 694 - protein, 2 aa, Artificial Sequence
  source: /note=“Description of Artificial Sequence: Synthetic peptide”
VD

SEQ ID NO: 695 - DNA, 336 bp, Artificial Sequence
  source: /note=“Description of Artificial Sequence: Synthetic polynucleotide”
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgt

SEQ ID NO: 696 - protein, 112 aa, Artificial Sequence
  source: /note=“Description of Artificial Sequence: Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 697-712 - skipped (16 entries)

SEQ ID NO: 713 - DNA, 321 bp, Artificial Sequence; identical to SEQ ID NO: 679
  source: /note=“Description of Artificial Sequence: Synthetic polynucleotide”

SEQ ID NO: 714 - protein, 107 aa, Artificial Sequence; identical to SEQ ID NO: 680
  source: /note=“Description of Artificial Sequence: Synthetic polypeptide”

SEQ ID NO: 715 - DNA, 24 bp, Artificial Sequence
  source: /note=“Description of Artificial Sequence: Synthetic oligonucleotide”
ggcggaggaagcggaggtgggggc

SEQ ID NO: 716 - protein, 8 aa, Artificial Sequence
  source: /note=“Description of Artificial Sequence: Synthetic peptide”
GGGSGGGG

SEQ ID NO: 717 - DNA, 360 bp, Artificial Sequence; identical to SEQ ID NO: 683
  source: /note=“Description of Artificial Sequence: Synthetic polynucleotide”

SEQ ID NO: 718 - protein, 120 aa, Artificial Sequence; identical to SEQ ID NO: 684
  source: /note=“Description of Artificial Sequence: Synthetic polypeptide”

SEQ ID NO: 719-740 - skipped (22 entries)

SEQ ID NO: 741 - DNA, 18 bp, Artificial Sequence
  source: /note=“Description of Artificial Sequence: Synthetic oligonucleotide”
atgctcgagatgctggag

SEQ ID NO: 742 - protein, 6 aa, Artificial Sequence
  source: /note=“Description of Artificial Sequence: Synthetic peptide”
MLEMLE

SEQ ID NO: 743 - DNA, 321 bp, Artificial Sequence; identical to SEQ ID NO: 665
  source: /note=“Description of Artificial Sequence: Synthetic polynucleotide”

SEQ ID NO: 744 - protein, 107 aa, Artificial Sequence; identical to SEQ ID NO: 92
  source: /note=“Description of Artificial Sequence: Synthetic polypeptide”

SEQ ID NO: 745 - DNA, 24 bp, Artificial Sequence
  source: /note=“Description of Artificial Sequence: Synthetic oligonucleotide”
agcggaggaggatccggagtggac

SEQ ID NO: 746 - protein, 8 aa, Artificial Sequence
  source: /note=“Description of Artificial Sequence: Synthetic peptide”
SGGGSGVD

SEQ ID NO: 747 - DNA, 858 bp, Homo sapiens; identical to SEQ ID NO: 669

SEQ ID NO: 748 - protein, 286 aa, Homo sapiens; identical to SEQ ID NO: 670

SEQ ID NO: 749 - DNA, 6 bp, Artificial Sequence
  source: /note=“Description of Artificial Sequence: Synthetic oligonucleotide”
ccgcgg

SEQ ID NO: 750 - protein, 2 aa, Artificial Sequence
  source: /note=“Description of Artificial Sequence: Synthetic peptide”
PR

SEQ ID NO: 751 - DNA, 54 bp, Thosea asigna virus; identical to SEQ ID NO: 673

SEQ ID NO: 752 - protein, 18 aa, Thosea asigna virus
EGRGSLLTCGDVEENPGP

SEQ ID NO: 753 - DNA, 6 bp, Artificial Sequence
  source: /note=“Description of Artificial Sequence: Synthetic oligonucleotide”
ccatgg

SEQ ID NO: 754 - protein, 2 aa, Artificial Sequence
  source: /note=“Description of Artificial Sequence: Synthetic peptide”
PW

SEQ ID NO: 755 - DNA, 63 bp, Artificial Sequence; identical to SEQ ID NO: 677
  source: /note=“Description of Artificial Sequence: Synthetic oligonucleotide”

SEQ ID NO: 756 - protein, 21 aa, Artificial Sequence
  source: /note=“Description of Artificial Sequence: Synthetic peptide”
MEFGLSWLFLVAILKGVQCSR

SEQ ID NO: 757 - DNA, 321 bp, Artificial Sequence; identical to SEQ ID NO: 679
  source: /note=“Description of Artificial Sequence: Synthetic polynucleotide”

SEQ ID NO: 758 - protein, 107 aa, Artificial Sequence; identical to SEQ ID NO: 680
  source: /note=“Description of Artificial Sequence: Synthetic polypeptide”

SEQ ID NO: 759 - DNA, 24 bp, Artificial Sequence
  source: /note=“Description of Artificial Sequence: Synthetic oligonucleotide”
ggcggaggaagcggaggtgggggc

SEQ ID NO: 760 - protein, 8 aa, Artificial Sequence
  source: /note=“Description of Artificial Sequence: Synthetic peptide”
GGGSGGGG

SEQ ID NO: 761 - DNA, 360 bp, Artificial Sequence; identical to SEQ ID NO: 683
  source: /note=“Description of Artificial Sequence: Synthetic polynucleotide”

SEQ ID NO: 762 - protein, 120 aa, Artificial Sequence; identical to SEQ ID NO: 684
  source: /note=“Description of Artificial Sequence: Synthetic polypeptide”

SEQ ID NO: 763 - DNA, 6 bp, Artificial Sequence
  source: /note=“Description of Artificial Sequence: Synthetic oligonucleotide”
ggatcc

SEQ ID NO: 764 - protein, 2 aa, Artificial Sequence
  source: /note=“Description of Artificial Sequence: Synthetic peptide”
GS

SEQ ID NO: 765 - DNA, 48 bp, Artificial Sequence; identical to SEQ ID NO: 687
  source: /note=“Description of Artificial Sequence: Synthetic oligonucleotide”

SEQ ID NO: 766 - protein, 16 aa, Artificial Sequence
  source: /note=“Description of Artificial Sequence: Synthetic peptide”
ELPTQGTFSNVSTNVS

SEQ ID NO: 767 - DNA, 126 bp, Artificial Sequence; identical to SEQ ID NO: 689
  source: /note=“Description of Artificial Sequence: Synthetic polynucleotide”

SEQ ID NO: 768 - protein, 42 aa, Artificial Sequence; identical to SEQ ID NO: 690
  source: /note=“Description of Artificial Sequence: Synthetic polypeptide”

SEQ ID NO: 769 - DNA, 111 bp, Artificial Sequence; identical to SEQ ID NO: 691
  source: /note=“Description of Artificial Sequence: Synthetic polynucleotide”

SEQ ID NO: 770 - protein, 37 aa, Artificial Sequence; identical to SEQ ID NO: 692
  source: /note=“Description of Artificial Sequence: Synthetic polypeptide”

SEQ ID NO: 771 - DNA, 6 bp, Artificial Sequence
  source: /note=“Description of Artificial Sequence: Synthetic oligonucleotide”
gtcgac

SEQ ID NO: 772 - protein, 2 aa, Artificial Sequence
  source: /note=“Description of Artificial Sequence: Synthetic peptide”
VD

SEQ ID NO: 773 - DNA, 336 bp, Artificial Sequence; identical to SEQ ID NO: 695
  source: /note=“Description of Artificial Sequence: Synthetic polynucleotide”

SEQ ID NO: 774 - protein, 112 aa, Artificial Sequence; identical to SEQ ID NO: 696
  source: /note=“Description of Artificial Sequence: Synthetic polypeptide”

SEQ ID NO: 775 - DNA, 18 bp, Artificial Sequence
  source: /note=“Description of Artificial Sequence: Synthetic oligonucleotide”
atgctcgagatgctggag

SEQ ID NO: 776 - protein, 6 aa, Artificial Sequence
  source: /note=“Description of Artificial Sequence: Synthetic peptide”
MLEMLE

SEQ ID NO: 777 - DNA, 57 bp, Porcine teschovirus-1
gctactaacttcagcctgctgaagcaggctggagacgtggaggagaaccccgggcct

SEQ ID NO: 778 - protein, 19 aa, Porcine teschovirus-1
ATNFSLLKQAGDVEENPGP

SEQ ID NO: 779 - DNA, 42 bp, Artificial Sequence; identical to SEQ ID NO: 3
  source: /note=“Description of Artificial Sequence: Synthetic oligonucleotide”

SEQ ID NO: 780 - protein, 16 aa, Artificial Sequence
  source: /note=“Description of Artificial Sequence: Synthetic peptide”
MGSSKSKPKDPSQRLD

SEQ ID NO: 781 - DNA, 516 bp, Artificial Sequence; identical to SEQ ID NO: 1
  source: /note=“Description of Artificial Sequence: Synthetic polynucleotide”

SEQ ID NO: 782 - protein, 172 aa, Artificial Sequence; identical to SEQ ID NO: 2
  source: /note=“Description of Artificial Sequence: Synthetic polypeptide”

SEQ ID NO: 783 - DNA, 9 bp, Artificial Sequence
  source: /note=“Description of Artificial Sequence: Synthetic oligonucleotide”
gcggccgct

SEQ ID NO: 784 - protein, 3 aa, Artificial Sequence
  source: /note=“Description of Artificial Sequence: Synthetic peptide”
AAA

SEQ ID NO: 785 - DNA, 126 bp, Artificial Sequence; identical to SEQ ID NO: 291
  source: /note=“Description of Artificial Sequence: Synthetic polynucleotide”

SEQ ID NO: 786 - protein, 42 aa, Artificial Sequence; identical to SEQ ID NO: 16
  source: /note=“Description of Artificial Sequence: Synthetic polypeptide”

SEQ ID NO: 787-901 - skipped (115 entries)

SEQ ID NO: 902 - DNA, 267 bp, Artificial Sequence
  source: /note=“Description of Artificial Sequence: Synthetic polynucleotide”
gaaatgtggcatgaagggttggaagaagcttcaaggctgtacttcggagagaggaacgtgaagggcatgtttgaggttcttgaacctctgcacgccatgatggaacggggaccgcagacactgaaagaaacctcttttaatcaggcctacggcagagacctgatggaggcccaagaatggtgtagaaagtatatgaaatccggtaacgtgaaagacctgcttcaggcctgggacctttattaccatgtgttcaggcggatcagtaag

SEQ ID NO: 903 - protein, 89 aa, Artificial Sequence
  source: /note=“Description of Artificial Sequence: Synthetic polypeptide”
EMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 904 - DNA, 267 bp, Artificial Sequence
  source: /note=“Description of Artificial Sequence: Synthetic polynucleotide”
gaaatgtggcatgaagggttggaagaagcttcaaggctgtacttcggagagaggaacgtgaagggcatgtttgaggttcttgaacctctgcacgccatgatggaacggggaccgcagacactgaaagaaacctcttttaatcaggcctacggcagagacctgatggaggcccaagaatggtgtagaaagtatatgaaatccggtaacgtgaaagacctgactcaggcctgggacctttattaccatgtgttcaggcggatcagtaag

SEQ ID NO: 905 - protein, 89 aa, Artificial Sequence
  source: /note=“Description of Artificial Sequence: Synthetic polypeptide”
EMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 906 - DNA, 888 bp, Homo sapiens
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccc

SEQ ID NO: 907 - protein, 296 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 908 - protein, 4 aa, Artificial Sequence
  source: /note=“Description of Artificial Sequence: Synthetic peptide”
YMNM

SEQ ID NO: 909 - protein, 16 aa, Homo sapiens
MGSNKSKPKDASQRRR

SEQ ID NO: 910 - protein, 5 aa, Artificial Sequence
  source: /note=“Description of Artificial Sequence: Synthetic peptide”
  MOD_RES (4)..(4): Any amino acid
MGCXC

SEQ ID NO: 911 - protein, 29 aa, Artificial Sequence
  source: /note=“Description of Artificial Sequence: Synthetic peptide”
KKVAKKPTNKAPHPKQEPQEINFPDDLPG

SEQ ID NO: 912 - protein, 6 aa, Artificial Sequence
  source: /note=“Description of Artificial Sequence: Synthetic peptide”
SGGGSG

SEQ ID NO: 913 - protein, 7 aa, Homo sapiens
TDPTRRF

SEQ ID NO: 914 - protein, 8 aa, Artificial Sequence
  source: /note=“Description of Artificial Sequence: Synthetic peptide”
RAKFKQLL

SEQ ID NO: 915 - protein, 9 aa, Artificial Sequence
  source: /note=“Description of Artificial Sequence: Synthetic peptide”
NLVPMVATV

SEQ ID NO: 916 - protein, 4 aa, Artificial Sequence
  source: /note=“Description of Artificial Sequence: Synthetic peptide”
SGGG

SEQ ID NO: 917 - protein, 5 aa, Homo sapiens
GCFNF

SEQ ID NO: 918 - protein, 5 aa, Homo sapiens
ISAQT

SEQ ID NO: 919 - protein, 4 aa, Homo sapiens
ATPF

SEQ ID NO: 920 - protein, 4 aa, Artificial Sequence
  source: /note=“Description of Artificial Sequence: Synthetic peptide”
AVPI

SEQ ID NO: 921 - protein, 5 aa, Artificial Sequence
  source: /note=“Description of Artificial Sequence: Synthetic peptide”
AAAAA

SEQ ID NO: 922 - protein, 5 aa, Artificial Sequence
  source: /note=“Description of Artificial Sequence: Synthetic peptide”
YCSTL

SEQ ID NO: 923 - protein, 5 aa, Artificial Sequence
  source: /note=“Description of Artificial Sequence: Synthetic peptide”
CIVSM

SEQ ID NO: 924 - protein, 5 aa, Artificial Sequence
  source: /note=“Description of Artificial Sequence: Synthetic peptide”
QPTFT

SEQ ID NO: 925 - protein, 5 aa, Artificial Sequence
  source: /note=“Description of Artificial Sequence: Synthetic peptide”
SGGGS

SEQ ID NO: 926 - DNA, 9 bp, Artificial Sequence
  source: /note=“Description of Artificial Sequence: Synthetic oligonucleotide”
agagcatgc

SEQ ID NO: 927 - DNA, 15 bp, Artificial Sequence
  source: /note=“Description of Artificial Sequence: Synthetic oligonucleotide”
ggatctggccaattg

SEQ ID NO: 928 - DNA, 6 bp, Artificial Sequence
  source: /note=“Description of Artificial Sequence: Synthetic oligonucleotide”
gtcgag

SEQ ID NO: 929 - DNA, 6 bp, Artificial Sequence
  source: /note=“Description of Artificial Sequence: Synthetic oligonucleotide”
ccttgg

SEQ ID NO: 930 - DNA, 63 bp, Artificial Sequence
  source: /note=“Description of Artificial Sequence: Synthetic oligonucleotide”
atggagttcggattgagctggctgttcctggtggcaatactcaagggcgttcaatgttcacgg

SEQ ID NO: 931 - DNA, 318 bp, Artificial Sequence
  source: /note=“Description of Artificial Sequence: Synthetic polynucleotide”
gacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaa

SEQ ID NO: 932 - DNA, 6 bp, Artificial Sequence
  source: /note=“Description of Artificial Sequence: Synthetic oligonucleotide”
caggtg

SEQ ID NO: 933 - DNA, 336 bp, Artificial Sequence
  source: /note=“Description of Artificial Sequence: Synthetic polynucleotide”
gaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttcc

SEQ ID NO: 934 - DNA, 3 bp, Artificial Sequence
  source: /note=“Description of Artificial Sequence: Synthetic oligonucleotide”
tga

SEQ ID NO: 935 - protein, 3 aa, Artificial Sequence
  source: /note=“Description of Artificial Sequence: Synthetic peptide”
RAC

SEQ ID NO: 936 - protein, 5 aa, Artificial Sequence
  source: /note=“Description of Artificial Sequence: Synthetic peptide”
GSGQL

SEQ ID NO: 937 - protein, 2 aa, Artificial Sequence
  source: /note=“Description of Artificial Sequence: Synthetic peptide”
VE

SEQ ID NO: 938 - protein, 106 aa, Artificial Sequence
  source: /note=“Description of Artificial Sequence: Synthetic polypeptide”
DIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIK

SEQ ID NO: 939 - protein, 2 aa, Artificial Sequence
  source: /note=“Description of Artificial Sequence: Synthetic peptide”
QV

SEQ ID NO: 940 - protein, 112 aa, Artificial Sequence
  source: /note=“Description of Artificial Sequence: Synthetic polypeptide”
EVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSS

SEQ ID NO: 941 - DNA, 18 bp, Artificial Sequence
  source: /note=“Description of Artificial Sequence: Synthetic oligonucleotide”
gcggccgctgggcgcgcc

SEQ ID NO: 942 - DNA, 66 bp, Porcine teschovirus-1
ggatctggagcaacgaatttttccctgctgaaacaggcaggggacgtagaggaaaatcctggtcct

SEQ ID NO: 943 - DNA, 6 bp, Artificial Sequence
  source: /note=“Description of Artificial Sequence: Synthetic oligonucleotide”
gcatgc

SEQ ID NO: 944 - DNA, 654 bp, Artificial Sequence
  source: /note=“Description of Artificial Sequence: Synthetic polynucleotide”
atggtcagtgtgatcaagccagaaatgaagatgcggtattatatggatgggtctgtaaacggccacgaatttaccatcgagggcgaggggacgggacgcccctatgagggacaccaggaaatgactctcagggtgacaatggctgagggcggcccaatgccatttgcttttgacctggtttcacacgtgttttgctacggacacagggtgttcactaagtacccagaggagatcccggattattttaaacaggcttttcccgagggcctgagctgggaaagatctctggagtttgaagacgggggtagcgccagcgtctcagcccacatcagtcttaggggaaataccttctatcacaagagcaaattcacaggggtgaattttccagctgatggtccaatcatgcagaaccagagcgtggattgggaacctagcacggagaaaatcacagccagtgatggggtgctcaaaggagatgtgaccatgtatttgaaactggaggggggcggcaatcataagtgtcaaatgaaaactacttataaagctgctaaggaaatcctcgaaatgccgggagatcactacatagggcatcgcctcgtcaggaaaactgaggggaacattacagagcaagtggaggacgccgtggcccatagc

SEQ ID NO: 945 - DNA, 6 bp, Artificial Sequence
  source: /note=“Description of Artificial Sequence: Synthetic oligonucleotide”
ggtacc

SEQ ID NO: 946 - DNA, 924 bp, Artificial Sequence
  source: /note=“Description of Artificial Sequence: Synthetic polynucleotide”
aaggtttatgaccctgaacagcgcaagagaatgataaccggcccacagtggtgggcacgatgtaagcaaatgaatgtactcgacagctttattaactactatgacagcgaaaaacatgctgagaacgccgtgatcttcctgcacggcaacgcaaccagcagttacctgtggagacacgttgtacctcacattgagcctgttgcacgctgcatcatcccagacttgattggcatgggaaaatctggaaagtctgggaatggatcttacagactgctcgaccactataagtatctcactgcttggttcgagctgctgaacctcccaaaaaaaatcatctttgtgggtcacgactggggaagtgccctggcattccactatgcctatgaacatcaggatcgcattaaagccatcgtccatatggaaagtgtcgtggacgtgatcgagagttggatgggatggcctgacatcgaggaggagctggctctcatcaaaagcgaagagggggaaaagatggtgttggagaacaacttctttgtcgagaccctgctgccgtctaagattatgaggaagctggaaccagaggaatttgcagcctatttggagcccttcaaggaaaagggggaggtgagaaggcccaccctctcctggccccgcgagatacccctggtgaagggcgggaagcctgacgtcgtccagatcgtgcggaattacaacgcatatctcagagcctccgacgacctgcccaagctttttattgaaagcgaccctggctttttttcaaatgctattgtcgagggtgccaagaagtttccaaataccgagttcgtgaaggtgaagggactgcatttcttgcaggaggatgcccccgacgagatgggaaaatacatcaagtctttcgtggagcgcgtactgaagaatgagcag

SEQ ID NO: 947 - DNA, 3 bp, Artificial Sequence
  source: /note=“Description of Artificial Sequence: Synthetic oligonucleotide”
taa

SEQ ID NO: 948 - protein, 6 aa, Artificial Sequence
  source: /note=“Description of Artificial Sequence: Synthetic peptide”
AAAGRA

SEQ ID NO: 949 - protein, 22 aa, Porcine teschovirus-1
GSGATNFSLLKQAGDVEENPGP

SEQ ID NO: 950 - protein, 2 aa, Artificial Sequence
  source: /note=“Description of Artificial Sequence: Synthetic peptide”
AC

SEQ ID NO: 951 - protein, 218 aa, Artificial Sequence
  source: /note=“Description of Artificial Sequence: Synthetic polypeptide”
MVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQMKTTYKAAKEILEMPGDHYIGHRLVRKTEGNITEQVEDAVAHS

SEQ ID NO: 952 - protein, 2 aa, Artificial Sequence
  source: /note=“Description of Artificial Sequence: Synthetic peptide”
GT

SEQ ID NO: 953 - protein, 308 aa, Artificial Sequence
  source: /note=“Description of Artificial Sequence: Synthetic polypeptide”
KVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGSALAFHYAYEHQDRIKAIVHMESVVDVIESWMGWPDIEEELALIKSEEGEKMVLENNFFVETLLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 954 - DNA, 66 bp, Artificial Sequence
  source: /note=“Description of Artificial Sequence: Synthetic oligonucleotide”
atggattttcaagtgcagattttcagctttctcttgatcagtgccagcgtgatcatgagcaggggc

SEQ ID NO: 955 - DNA, 327 bp, Artificial Sequence
  source: /note=“Description of Artificial Sequence: Synthetic polynucleotide”
gacatccagatgactcaatcaccctcaagtctgagtgcatcagtgggcgaccgagtaacaattacgtgccgggcctctcaggatgtcaacaccgccgtggcctggtatcaacaaaaacctggaaaggcccctaagctcctcatatatagcgcctcattcctttattctggtgtacctagtcgcttttcagggtcaagatcaggaaccgattttacacttacaatctctagtctgcaaccagaggattttgctacctactattgccagcaacactatacaacgccgcctacattcgggcaggggacgaaggtggagattaaacgcact

SEQ ID NO: 956 - DNA, 45 bp, Artificial Sequence
  source: /note=“Description of Artificial Sequence: Synthetic oligonucleotide”
ggctcaaccagtgggtcaggcaagcctgggtccggcgagggctcc

SEQ ID NO: 957 - DNA, 360 bp, Artificial Sequence
  source: /note=“Description of Artificial Sequence: Synthetic polynucleotide”
gaggtacaactggttgaaagcggaggagggctggttcagccaggcgggtcactccgcctctcctgtgcggcgtccgggttcaacataaaggacacctatatccattgggttcgccaggcccccggaaagggattggagtgggttgctcgaatttatccaactaatgggtacacccgctatgctgattccgtgaagggtcgctttacgattagtgccgacacttcgaaaaacacagcttacttgcaaatgaacagtctccgcgccgaggacaccgcagtatactattgcagtaggtgggggggagacgggttttatgctatggatttctggggacaaggtactctggtaaccgtgagcagt

SEQ ID NO: 958 - protein, 22 aa, Artificial Sequence
  source: /note=“Description of Artificial Sequence: Synthetic peptide”
MDFQVQIFSFLLISASVIMSRG

SEQ ID NO: 959 - protein, 124 aa, Artificial Sequence
  source: /note=“Description of Artificial Sequence: Synthetic polypeptide”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGS

SEQ ID NO: 960 - protein, 15 aa, Artificial Sequence
  source: /note=“Description of Artificial Sequence: Synthetic peptide”
GSTSGSGKPGSGEGS

SEQ ID NO: 961 - protein, 120 aa, Artificial Sequence
  source: /note=“Description of Artificial Sequence: Synthetic polypeptide”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDFWGQGTLVTVSS

SEQ ID NO: 962 - DNA, 123 bp, Artificial Sequence
  source: /note=“Description of Artificial Sequence: Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 963 - protein, 41 aa, Artificial Sequence
  source: /note=“Description of Artificial Sequence: Synthetic polypeptide”
RSKRSRLLHSDYMNMTPRRPGPTRKHYQPYAPPRDFAAYRS

SEQ ID NO: 964 - DNA, 186 bp, Artificial Sequence
  source: /note=“Description of Artificial Sequence: Synthetic polynucleotide”
aaaaaggtggccaagaagccaaccaataaggccccccaccccaagcaggagccccaggagatcaattttcccgacgatcttcctggctccaacactgctgctccagtgcaggagactttacatggatgccaaccggtcacccaggaggatggcaaagagagtcgcatctcagtgcaggagagacag

SEQ ID NO: 965 - DNA, 318 bp, Artificial Sequence
  source: /note=“Description of Artificial Sequence: Synthetic polynucleotide”
ggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctc

SEQ ID NO: 966 - protein, 106 aa, Artificial Sequence
  source: /note=“Description of Artificial Sequence: Synthetic polypeptide”
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKL

SEQ ID NO: 967 - DNA, 57 bp, Porcine teschovirus-1
gctactaacttcagcctgctgaagcaggctggagacgtggaggagaaccctggacct

SEQ ID NO: 968 - DNA, 2253 bp, Artificial Sequence
  source: /note=“Description of Artificial Sequence: Synthetic polynucleotide”
atgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttacataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttgggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgctgagactttgagtgaagtagcctaa

SEQ ID NO: 969 - protein, 750 aa, Artificial Sequence
  source: /note=“Description of Artificial Sequence: Synthetic polypeptide”
MWNLLHETDSAVATARRPRWLCAGALVLAGGFFLLGFLFGWFIKSSNEATNITPKHNMKAFLDELKAENIKKFLHNFTQIPHLAGTEQNFQLAKQIQSQWKEFGLDSVELAHYDVLLSYPNKTHPNYISIINEDGNEIFNTSLFEPPPPGYENVWDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 970 - DNA, 204 bp, Artificial Sequence
  source: /note=“Description of Artificial Sequence: Synthetic polynucleotide”
ttctgggtactggttgtagtcggtggcgtacttgcttgttattctcttcttgttaccgtagccttcattatattctgggtccgatcaaagcgctcaagactcctccattccgattatatgaacatgacacctcgccgacctggtcctacacgcaaacattatcaaccctacgcacccccccgagacttcgctgcttatcgatcc

SEQ ID NO: 971 - protein, 68 aa, Artificial Sequence
  source: /note=“Description of Artificial Sequence: Synthetic polypeptide”
FWVLVVVGGVLACYSLLVTVAFIIFWVRSKRSRLLHSDYMNMTPRRPGPTRKHYQPYAPPRDFAAYRS

SEQ ID NO: 972 - DNA, 135 bp, Artificial Sequence
  source: /note=“Description of Artificial Sequence: Synthetic polynucleotide”
agtgtagttaaaagaggaagaaaaaagttgctgtatatatttaaacaaccatttatgagaccagtgcaaaccacccaagaagaagacggatgttcatgcagattcccagaagaagaagaaggaggatgtgaattg

SEQ ID NO: 973 - protein, 45 aa, Artificial Sequence
  source: /note=“Description of Artificial Sequence: Synthetic polypeptide”
SVVKRGRKKLLYIFKQPFMRPVQTTQEEDGCSCRFPEEEEGGCEL

SEQ ID NO: 974 - DNA, 6 bp, Artificial Sequence
  source: /note=“Description of Artificial Sequence: Synthetic oligonucleotide”
acgcgt

SEQ ID NO: 975 - protein, 2 aa, Artificial Sequence
  source: /note=“Description of Artificial Sequence: Synthetic peptide”
TR

SEQ ID NO: 976 - DNA, 339 bp, Artificial Sequence
  source: /note=“Description of Artificial Sequence: Synthetic polynucleotide”
cgggtcaaattcagccggagtgctgacgccccagcataccaacagggacaaaaccaactctacaacgagctcaacctgggtagacgcgaggagtacgacgttctggataagaggcggggccgggacccagagatggggggcaaacctcagcggcggaagaacccgcaggagggtctttataacgagctccagaaggacaagatggcggaagcctattcagaaattgggatgaaaggcgagagacgcaggggaaaaggtcacgatggtctgtatcaaggactgtcaaccgccaccaaagacacttacgatgcgctccacatgcaggccctccctccccgc

SEQ ID NO: 977 - protein, 113 aa, Artificial Sequence
  source: /note=“Description of Artificial Sequence: Synthetic polypeptide”
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 978 - DNA, 63 bp, Artificial Sequence
  source: /note=“Description of Artificial Sequence: Synthetic oligonucleotide”
atggagtttgggctgtcatggctgttcctcgtggccattctcaaaggggtccagtgttctcgc

SEQ ID NO: 979 - protein, 21 aa, Artificial Sequence
  source: /note=“Description of Artificial Sequence: Synthetic peptide”
MGFGLSWLFLVAILKGVQCSR

SEQ ID NO: 980 - DNA, 45 bp, Artificial Sequence
  source: /note=“Description of Artificial Sequence: Synthetic oligonucleotide”
gggggaggaggttctggaggcggcgggagcggaggaggaggcagc

SEQ ID NO: 981 - protein, 15 aa, Artificial Sequence
  source: /note=“Description of Artificial Sequence: Synthetic peptide”
GGGGSGGGGSGGGGS

SEQ ID NO: 982 - DNA, 714 bp, Artificial Sequence
  source: /note=“Description of Artificial Sequence: Synthetic polynucleotide”
gatccagccgaacccaaatcccccgataaaacacatacttgccccccttgtcccgcaccagaattgcttggcggaccttccgtttttctttttccccccaaacctaaagataccctgatgatttcccgaacccctgaagttacgtgcgtagtcgtagatgtgtctcacgaagatccagaagtaaaatttaactggtacgtagatggagtcgaagttcacaacgcaaagacgaagccccgagaagaacaatataattccacataccgagtagttagcgttctcaccgtactgcatcaggactggcttaacggcaaagaatataaatgtaaggtctcaaacaaagcactcccagcccctatcgaaaagactatctccaaagctaaaggacaaccccgcgaaccccaggtctatacacttcccccctcacgcgatgaactcactaaaaatcaggtttcccttacttgtcttgtcaaaggcttctaccctagcgatatcgcagtcgaatgggaatccaatggccagcccgaaaacaactataaaacaaccccacctgtcctcgattcagatggctcattctttctctattccaaactgactgtagacaaatcccgatggcaacaaggtaacgtgttctcttgctcagtcatgcatgaagcgcttcataaccattacacacaaaaatctctctcactgtctcccggaaagaaggacccc

SEQ ID NO: 983 - protein, 238 aa, Artificial Sequence
  source: /note=“Description of Artificial Sequence: Synthetic polypeptide”
DPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDP

SEQ ID NO: 984 - DNA, 408 bp, Artificial Sequence
  source: /note=“Description of Artificial Sequence: Synthetic polynucleotide”
aaactgtgttacctcctcgatggcatcctctttatttatggcgtgattctgaccgcattgtttctccgagtaaaattctctagatccgcagacgctcccgcatatcagcaaggacaaaatcagctttataacgaacttaacctcggcagacgcgaagaatacgatgtactggacaagagaagaggaagagatcccgaaatgggcggaaaaccccagagaagaaagaatccccaagaaggtctttataacgaactgcagaaagataaaatggccgaagcgtacagtgaaattggtatgaaaggagaaagaagacgcggaaaaggacatgacggactctaccaaggactctcaactgctactaaagatacatacgacgcccttcatatgcaagccctccccccgagataa

SEQ ID NO: 985 - protein, 135 aa, Artificial Sequence
  source: /note=“Description of Artificial Sequence: Synthetic polypeptide”
KLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 986 - DNA, 186 bp, Artificial Sequence
  source: /note=“Description of Artificial Sequence: Synthetic polynucleotide”
gttgccgccatcctgggcctgggcctggtgctggggctgctgggccccctggccatcctgctggccctgtacctgctccgggaccagaggctgccccccgatgcccacaagccccctgggggaggcagtttccggacccccatccaagaggagcaggccgacgcccactccaccctggccaagatc

SEQ ID NO: 987 - protein, 63 aa, Artificial Sequence
  source: /note=“Description of Artificial Sequence: Synthetic polypeptide”
VAAILGLGLVLGLLGPLAILLALYLLRRDQRLPPDAHKPPGGGSFRTPIQEEQADAHSTLAKI

SEQ ID NO: 988 - DNA, 590 bp, Artificial Sequence
  source: /note=“Description of Artificial Sequence: Synthetic polynucleotide”
tgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 989 - DNA, 57 bp, Thosea asigna virus
gccgagggcaggggaagtcttctaacatgcggggacgtggaggaaaatcccgggccc

SEQ ID NO: 990 - protein, 19 aa, Thosea asigna virus
AEGRGSLLTCGDVEENPGP

SEQ ID NO: 991 - DNA, 590 bp, Artificial Sequence
  source: /note=“Description of Artificial Sequence: Synthetic polynucleotide”
tgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 992 - DNA, 696 bp, Artificial Sequence
  source: /note=“Description of Artificial Sequence: Synthetic polynucleotide”
ctcgagtctggcggtggatccggaggcgttcaagtagaaacaatcagcccaggagacggaaggactttccccaaacgaggccaaacatgcgtagttcattatactgggatgctcgaagatggaaaaaaagtagatagtagtagagaccgaaacaaaccatttaaatttatgttgggaaaacaagaagtaataaggggctgggaagaaggtgtagcacaaatgtctgttggccagcgcgcaaaactcacaatttctcctgattatgcttacggagctaccggccaccccggcatcataccccctcatgccacactggtgtttgacgtcgaattgctcaaactggaagtcgagggagtgcaggtggagacgattagtcctggggatgggagaacctttccaaagcgcggtcagacctgtgttgtccactacaccggtatgctggaggacgggaagaaggtggactcttcacgcgatcgcaataagcctttcaagttcatgctcggcaagcaggaggtgatccgggggtgggaggagggcgtggctcagatgtcggtcgggcaacgagcgaagcttaccatctcacccgactacgcgtatggggcaacggggcatccgggaattatccctccccacgctacgctcgtattcgatgtggagctcttgaagcttgagtctggcggtggatccggagtcgac

SEQ ID NO: 993 - protein, 232 aa, Artificial Sequence
  source: /note=“Description of Artificial Sequence: Synthetic polypeptide”
LESGGGSGGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVD

SEQ ID NO: 994 - DNA, 321 bp, Artificial Sequence
  source: /note=“Description of Artificial Sequence: Synthetic polynucleotide”
ggcgttcaagtagaaacaatcagcccaggagacggaaggactttccccaaacgaggccaaacatgcgtagttcattatactgggatgctcgaagatggaaaaaaagtagatagtagtagagaccgaaacaaaccatttaaatttatgttgggaaaacaagaagtaataaggggctgggaagaaggtgtagcacaaatgtctgttggccagcgcgcaaaactcacaatttctcctgattatgcttacggagctaccggccaccccggcatcataccccctcatgccacactggtgtttgacgtcgaattgctcaaactggaa

SEQ ID NO: 995 - DNA, 321 bp, Artificial Sequence
  source: /note=“Description of Artificial Sequence: Synthetic polynucleotide”
ggagtgcaggtggagacgattagtcctggggatgggagaacctttccaaagcgcggtcagacctgtgttgtccactacaccggtatgctggaggacgggaagaaggtggactcttcacgcgatcgcaataagcctttcaagttcatgctcggcaagcaggaggtgatccgggggtgggaggagggcgtggctcagatgtcggtcgggcaacgagcgaagcttaccatctcacccgactacgcgtatggggcaacggggcatccgggaattatccctccccacgctacgctcgtattcgatgtggagctcttgaagcttgag

SEQ ID NO: 996 - DNA, 516 bp, Artificial Sequence
  source: /note=“Description of Artificial Sequence: Synthetic polynucleotide”
atggccgctgggggcccaggcgccggatcagctgctcccgtatcttctacttcttctttgccgctggctgctctgaacatgcgcgtgagaagacgcctctccctgttccttaacgttcgcacacaagtcgctgccgattggaccgcccttgccgaagaaatggactttgaatacctggaaattagacaacttgaaacacaggccgaccccactggcagactcctggacgcatggcagggaagacctggtgcaagcgttggacggctcctggatctcctgacaaaactgggacgcgacgacgtactgcttgaactcggacctagcattgaagaagactgccaaaaatatatcctgaaacaacaacaagaagaagccgaaaaacctctccaagtcgcagcagtggactcatcagtaccccgaacagctgagcttgctgggattactacactcgacgacccactcggacatatgcctgaaagattcgacgctttcatttgctattgcccctctgacata

SEQ ID NO: 997 - DNA, 186 bp, Homo sapiens
aagaaagttgcaaagaaacccacaaataaagccccacaccctaaacaggaaccccaagaaatcaatttcccagatgatctccctggatctaatactgccgccccggtccaagaaaccctgcatggttgccagcctgtcacccaagaggacggaaaagaatcacggattagcgtacaagagagacaa

SEQ ID NO: 998 - DNA, 24 bp, Artificial Sequence
  source: /note=“Description of Artificial Sequence: Synthetic oligonucleotide”
gtcgagtctggcggtggatccgga

SEQ ID NO: 999 - protein, 8 aa, Artificial Sequence
  source: /note=“Description of Artificial Sequence: Synthetic peptide”
VESGGGSG

SEQ ID NO: 1000 - DNA, 24 bp, Artificial Sequence
  source: /note=“Description of Artificial Sequence: Synthetic oligonucleotide”
tctggcggtggatccggagtcgac

SEQ ID NO: 1001 - DNA, 12 bp, Artificial Sequence
  source: /note=“Description of Artificial Sequence: Synthetic oligonucleotide”
cgcgcaaagcgt

SEQ ID NO: 1002 - protein, 4 aa, Artificial Sequence
  source: /note=“Description of Artificial Sequence: Synthetic peptide”
RAKR

SEQ ID NO: 1003 - DNA, 42 bp, Artificial Sequence
  source: /note=“Description of Artificial Sequence: Synthetic oligonucleotide”
ggaaaacctatacctaatccattgctgggcttagactcaaca

SEQ ID NO: 1004 - protein, 14 aa, Artificial Sequence
  source: /note=“Description of Artificial Sequence: Synthetic peptide”
GKPIPNPLLGLDST

SEQ ID NO: 1005 - DNA, 12 bp, Artificial Sequence
  source: /note=“Description of Artificial Sequence: Synthetic oligonucleotide”
ggcagcggaagc

SEQ ID NO: 1006 - protein, 4 aa, Artificial Sequence
  source: /note=“Description of Artificial Sequence: Synthetic peptide”
GSGS

SEQ ID NO: 1007 - DNA, 57 bp, Porcine teschovirus-1
gcaacgaatttttccctgctgaaacaggcaggggacgtagaggaaaatcctggtcct

SEQ ID NO: 1008 - DNA, 1002 bp, Homo sapiens
atgccccctcctagactgctgtttttcctgctctttctcaccccaatggaagttagacctgaggaaccactggtcgttaaagtggaagaaggtgataatgctgtcctccaatgccttaaagggaccagcgacggaccaacgcagcaactgacttggagccgggagtcccctctcaagccgtttctcaagctgtcacttggcctgccaggtcttggtattcacatgcgcccccttgccatttggctcttcatattcaatgtgtctcaacaaatgggtggattctacctttgccagcccggccccccttctgagaaagcttggcagcctggatggaccgtcaatgttgaaggctccggtgagctgtttagatggaatgtgagcgaccttggcggactcggttgcggactgaaaaataggagctctgaaggaccctcttctccctccggtaagttgatgtcacctaagctgtacgtgtgggccaaggaccgccccgaaatctgggagggcgagcctccatgcctgccgcctcgcgattcactgaaccagtctctgtcccaggatctcactatggcgcccggatctactctttggctgtcttgcggcgttcccccagatagcgtgtcaagaggacctctgagctggacccacgtacaccctaagggccctaagagcttgttgagcctggaactgaaggacgacagacccgcacgcgatatgtgggtaatggagaccggccttctgctccctcgcgctaccgcacaggatgcagggaaatactactgtcatagagggaatctgactatgagctttcatctcgaaattacagcacggcccgttctttggcattggctcctccggactggaggctggaaggtgtctgccgtaacactcgcttacttgattttttgcctgtgtagcctggttgggatcctgcatcttcagcgagcccttgtattgcgccgaaaaagaaaacgaatgactgaccctacacgacgattctga

SEQ ID NO: 1009 - protein, 333 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 1010 - DNA, 66 bp, Artificial Sequence
  source: /note=“Description of Artificial Sequence: Synthetic oligonucleotide”
atgctgctgctcgtgacatctctgctgctgtgcgagctgccccaccccgcctttctgctgatcccc

SEQ ID NO: 1011 - DNA, 339 bp, Artificial Sequence
  source: /note=“Description of Artificial Sequence: Synthetic polynucleotide”
gatatcctgctgacccagacccctctgagcctgcctgtgtctctgggcgatcaggccagcatcagctgcagatccagccagagcctggtgcaccggaacggcaacacctacctgcactggtatctgcagaagcccggccagagccccaagctgctgattcacaaggtgtccaaccggttcagcggcgtgcccgacagattttctggcagcggctccggcaccgacttcaccctgaagatcagccgggtggaagccgaggacctgggcgtgtacttctgcagccagtccacccacgtgccccccctgacatttggcgccggaacaaagctggaactgaag

SEQ ID NO: 1012 - DNA, 54 bp, Artificial Sequence
  source: /note=“Description of Artificial Sequence: Synthetic oligonucleotide”
ggcagcacaagcggcagcggcaagcctggatctggcgagggaagcaccaagggc

SEQ ID NO: 1013 - DNA, 339 bp, Artificial Sequence
  source: /note=“Description of Artificial Sequence: Synthetic polynucleotide”
gaagtgaagctgcagcagagcggcccctctctggtggaacctggcgcctctgtgatgatctcctgcaaggccagcggcagctccttcaccggctacaacatgaactgggtgcgccagaacatcggcaagagcctggaatggatcggcgccatcgacccctactacggcggcaccagctacaaccagaagttcaagggcagagccaccctgaccgtggacaagagcagctccaccgcctacatgcacctgaagtccctgaccagcgaggacagcgccgtgtactactgcgtgtccggcatggaatactggggccagggcacaagcgtgaccgtgtcctct

SEQ ID NO: 1014 - protein, 22 aa, Artificial Sequence
  source: /note=“Description of Artificial Sequence: Synthetic peptide”
MLLLVTSLLLCELPHPAFLLIP

SEQ ID NO: 1015 - protein, 113 aa, Artificial Sequence
  source: /note=“Description of Artificial Sequence: Synthetic polypeptide”
DILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 1016 - protein, 18 aa, Artificial Sequence
  source: /note=“Description of Artificial Sequence: Synthetic peptide”
GSTSGSGKPGSGEGSTKG

SEQ ID NO: 1017 - protein, 113 aa, Artificial Sequence
  source: /note=“Description of Artificial Sequence: Synthetic polypeptide”
EVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSS

SEQ ID NO: 1018 - DNA, 348 bp, Artificial Sequence
  source: /note=“Description of Artificial Sequence: Synthetic polynucleotide”
cagatccaactggtgcagtcaggcccggaactgaagaagccaggggagacagtcaaaataagttgtaaagccagcggctacatatttactaattacgggatgaattgggtgaagcaagcgccgggcaaatcctttaaatggatggggtggataaacacatacacaggagagtcaacgtacagcgcggacttcaaaggtcgattcgcgttcagtctcgagaccagcgcgagtacagcttacctccacatcaacgatcttaaaaacgaagacacggcaacctatttttgcgcccggtcaggcggttacgaccctatggactattggggccaagggacctccgttacggta

SEQ ID NO: 1019 - DNA, 51 bp, Artificial Sequence
  source: /note=“Description of Artificial Sequence: Synthetic oligonucleotide”
tcttcaggcggtggcgggagtggtggaggaggctcaggcggcgggggatca

SEQ ID NO: 1020 - DNA, 360 bp, Artificial Sequence
  source: /note=“Description of Artificial Sequence: Synthetic polynucleotide”
gacatcgtactgacccaatctcccgctagccttgcagtatccttgggtcaacgcgctacaataagttgccgggctagtgagtccgtagacaactatggcaacaccttcatgcattggtaccaacaaaaaccaggtcagccacccaaacttctcatttacagagcgtctaatctcgaaagcggcatccctgctcgattctctggaagcggaagtagaaccgactttacactgactataaaccccgtcgaagccgatgatgttgccacttattactgtcaacagagcaatgaggacccaccgacattcggtgctggtaccaagctggagttgaaggagtcaaaatacgggcctccctgtccc

SEQ ID NO: 1021 - protein, 116 aa, Artificial Sequence
  source: /note=“Description of Artificial Sequence: Synthetic polypeptide”
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTV

SEQ ID NO: 1022 - protein, 17 aa, Artificial Sequence
  source: /note=“Description of Artificial Sequence: Synthetic peptide”
SSGGGGSGGGGSGGGGS

SEQ ID NO: 1023 - protein, 120 aa, Artificial Sequence
  source: /note=“Description of Artificial Sequence: Synthetic polypeptide”
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKESKYGPPCP

SEQ ID NO: 1024 - DNA, 339 bp, Artificial Sequence
  source: /note=“Description of Artificial Sequence: Synthetic polynucleotide”
gaaattgtgctgactcagagcccgggtagcctggccgtgtcccccggagagcgagtgaccatgagctgtaaatccagccaatcagtttttttttcatcatctcaaaaaaactatctggcatggtaccaacagatacccgggcagtccccacggctgctgatttactgggcatcaacacgcgagagcggtgtgcccgacagattcaccggaagcgggagcggcacggacttcacacttaccatctcaagcgtacaaccggaggacttggctatctattactgccaccaatatctttcctccagaacattcggacagggaacgaaactggagatcaaaaga

SEQ ID NO: 1025 - DNA, 30 bp, Artificial Sequence
  source: /note=“Description of Artificial Sequence: Synthetic oligonucleotide”
ggcggcgggagtgggggaggaggtcaggtg

SEQ ID NO: 1026 - DNA, 354 bp, Artificial Sequence
  source: /note=“Description of Artificial Sequence: Synthetic polynucleotide”
caggtgcagctgcagcagcctggagccgaggtggtgaagcccggcgcatctgtgaaaatgtcttgcaaggcaagcggatatacatttactagctactacatccattggatcaagcaaacccccggacagggcctcgaatgggtgggagttatttacccggggaacgatgatatctcttataatcagaaattccaagggaaagccaccctgactgcagacaaatcaagtaccacagcctatatgcagctcagctccctgacaagcgaggattccgctgtgtactactgtgccagggaggttagacttcgatattttgatgtttgggggcagggaactaccgtgaccgtgagcagc

SEQ ID NO: 1027 - protein, 113 aa, Artificial Sequence
  source: /note=“Description of Artificial Sequence: Synthetic polypeptide”
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 1028 - protein, 10 aa, Artificial Sequence
  source: /note=“Description of Artificial Sequence: Synthetic peptide”
GGGSGGGGQV

SEQ ID NO: 1029 - protein, 118 aa, Artificial Sequence
  source: /note=“Description of Artificial Sequence: Synthetic polypeptide”
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 1030 - protein, 4 aa, Artificial Sequence
  source: /note=“Description of Artificial Sequence: Synthetic peptide”
IVVY

SEQ ID NO: 1031 - protein, 5 aa, Artificial Sequence
  source: /note=“Description of Artificial Sequence: Synthetic peptide”
PVQET